SEQUENCE LISTING (38 sequences)

SEQ ID NO: 1 - protein, 553 aa, Artificial
  AvrPtoB U-box motif from Pseudomonas syringae
MAGINRAGPSGAYFVGHTDPEPVSGQAHGSGSGASSSNSPQVQPRPSNTPPSNAPAPPPTGRERLSRSTALSRQTREWLEQGMPTAEDASVRRRPQVTADAATPRAEARRTPEATADASAPRRGAVAHANSIVQQLVSEGADISHTRNMLRNAMNGDAVAFSRVEQNIFRQHFPNMPMHGISRDSELAIELRGALRRAVHQQAASAPVRSPTPTPASPAASSSGSSQRSLFGRFARLMAPNQGRSSNTAASQTPVDRSPPRVNQRPIRVDRAAMRNRGNDEADAALRGLVQQGVNLEHLRTALERHVMQRLPIPLDIGSALQNVGINPSIDLGESLVQHPLLNLNVALNRMLGLRPSAERAPRPAVPVAPATASRRPDGTRATRLRVMPEREDYENNVAYGVRLLNLNPGVGVRQAVAAFVTDRAERPAVVANIRAALDPIASQFSQLRTISKADAESEELGFKDAADHHTDDVTHCLFGGELSLSNPDQQVIGLAGNPTDTSQPYSQEGNKDLAFMDMKKLAQFLAGKPEHPMTRETLNAENIAKYAFRIVP

SEQ ID NO: 2 - DNA, 1659 bp, Artificial
  AvrPtoB U-box motif from Pseudomonas syringae
atggcgggtatcaatagagcgggaccatcgggcgcttattttgttggccacacagaccccgagccagtatcggggcaagcacacggatccggcagcggcgccagctcctcgaacagtccgcaggttcagccgcgaccctcgaatactcccccgtcgaacgcgcccgcaccgccgccaaccggacgtgagaggctttcacgatccacggcgctgtcgcgccaaaccagggagtggctggagcagggtatgcctacagcggaggatgccagcgtgcgtcgtaggccacaggtgactgccgatgccgcaacgccgcgtgcagaggcaagacgcacgccggaggcaactgccgatgccagcgcaccgcgtagaggggcggttgcacacgccaacagtatcgttcagcaattggtcagtgagggcgctgatatttcgcatactcgtaacatgctccgcaatgcaatgaatggcgacgcagtcgctttttctcgagtagaacagaacatatttcgccagcatttcccgaacatgcccatgcatggaatcagccgagattcggaactcgctatcgagctccgtggggcgcttcgtcgagcggttcaccaacaggcggcgtcagcgccagtgaggtcgcccacgccaacaccggccagccctgcggcatcatcatcgggcagcagtcagcgttctttatttggacggtttgcccgtttgatggcgccaaaccagggacggtcgtcgaacactgccgcctctcagacgccggtcgacaggagcccgccacgcgtcaaccaaagacccatacgcgtcgacagggctgcgatgcgtaatcgtggcaatgacgaggcggacgccgcgctgcgggggttagtacaacagggggtcaatttagagcacctgcgcacggcccttgaaagacatgtaatgcagcgcctccctatccccctcgatataggcagcgcgttgcagaatgtgggaattaacccaagtatcgacttgggggaaagccttgtgcaacatcccctgctgaatttgaatgtagcgttgaatcgcatgctggggctgcgtcccagcgctgaaagagcgcctcgtccagccgtccccgtggctcccgcgaccgcctccaggcgaccggatggtacgcgtgcaacacgattgcgggtgatgccggagcgggaggattacgaaaataatgtggcttatggagtgcgcttgcttaacctgaacccgggggtgggggtaaggcaggctgttgcggcctttgtaaccgaccgggctgagcggccagcagtggtggctaatatccgggcagccctggaccctatcgcgtcacaattcagtcagctgcgcacaatttcgaaggccgatgctgaatctgaagagctgggttttaaggatgcggcagatcatcacacggatgacgtgacgcactgtctttttggcggagaattgtcgctgagtaatccggatcagcaggtgatcggtttggcgggtaatccgacggacacgtcgcagccttacagccaagagggaaataaggacctggcgttcatggatatgaaaaaacttgcccaattcctcgcaggcaagcctgagcatccgatgaccagagaaacgcttaacgccgaaaatatcgccaagtatgcttttagaatagtcccc

SEQ ID NO: 3 - protein, 587 aa, Artificial
  IpaH0722 novel E3 ligase from Shigella flexneri
MKPAHNPSFFRSFCGLGCISRLSVEEQNITDYHRIWDNWAKEGAATEDRTQAVRLLKICLAFQEPALNLSLLRLRSLPYLPPHIQELNISSNELRSLPELPPSLTVLKASDNRLSRLPALPPHLVALDVSLNRVLTCLPSLPSSLQSLSALLNSLETLPDLPPALQKLSVGNNQLTALPELPCELQELSAFDNRLQELPPLPQNLRLLNVGENQLHRLPELPQRLQSLYIPNNQLNTLPDSIMNLHIYADVNIYNNPLSTRTLQALQRLTSSPDYHGPRIYFSMSDGQQNTLHRPLADAVTAWFPENKQSDVSQIWHAFEHEEHANTFSAFLDRLSDTVSARNTSGFREQVAAWLEKLSASAELRQQSFAVAADATESCEDRVALTWNNLRKTLLVHQASEGLFDNDTGALLSLGREMFRLEILEDIARDKVRTLHFVDEIEVYLAFQTMLAEKLQLSTAVKEMRFYGVSGVTANDLRTAEAMVRSREENEFTDWFSLWGPWHAVLKRTEADRWALAEEQKYEMLENEYPQRVADRLKASGLSGDADAEREAGAQVMRETEQQIYRQLTDEVLALRLPENGSQLHHS

SEQ ID NO: 4 - DNA, 1764 bp, Artificial
  IpaH0722 novel E3 ligase from Shigella flexneri
atgaaacctgcccacaatccttcttttttccgctccttttgtggtttaggatgtatatcccgtttatccgtagaagagcaaaatatcacggattatcaccgcatctgggataactgggccaaggaaggtgctgcaacagaagaccgaacacaggcagttcgattactgaaaatatgtctggcttttcaagagccagccctcaatttaagtttactcagattacgctctctcccatacctgcccccgcacatacaagaacttaacatctctagcaatgagctacgctctctgccagaactccctccgtccttaactgtacttaaagccagcgataacagactgagcaggctcccggctcttccgcctcacctggtcgctcttgatgtttcacttaacagagttttaacatgtttgccttctcttccatcttccttgcagtcactctcagcccttctcaatagcctggagacgctacctgatcttcccccggctctacaaaaactttctgttggcaacaaccagcttactgccttaccagaattaccatgtgaactacaggaactaagtgcttttgataacagattacaagagctaccgccccttcctcaaaatctgaggcttttaaacgttggggaaaaccaactacacagactgcccgaacttccacaacgtctgcaatcactatatatccctaacaatcagctgaacacattgccagacagtatcatgaatctgcacatttatgcagatgttaatatttataacaatccattgtcgactcgcactctgcaagccctgcaaagattaacctcttcgccggactaccacggcccacggatttacttctccatgagtgacggacaacagaatacactccatcgccccctggctgatgccgtgacagcatggttcccggaaaacaaacaatctgatgtatcacagatatggcatgcttttgaacatgaagagcatgccaacaccttttccgcgttccttgaccgcctttccgataccgtctctgcacgcaatacctccggattccgtgaacaggtcgctgcatggctggaaaaactcagtgcctctgcggagcttcgacagcagtctttcgctgttgctgctgatgccactgagagctgtgaggaccgtgtcgcgctcacatggaacaatctccggaaaaccctcctggtccatcaggcatcagaaggccttttcgataatgataccggcgctctgctctccctgggcagggaaatgttccgcctcgaaattctggaggacattgcccgggataaagtcagaactctccattttgtggatgagatagaagtctacctggccttccagaccatgctcgcagagaaacttcagctctctactgccgtgaaggaaatgcgtttctatggcgtgtcgggagtgacagcaaatgacctccgcactgccgaagccatggtcagaagccgtgaagagaatgaatttacggactggttctccctctggggaccatggcatgctgtactgaagcgtacggaagctgaccgctgggcgctggcagaagagcagaaatatgagatgctggagaatgagtaccctcagagggtggctgaccggctgaaagcatcaggtctgagcggtgatgcggatgcggagagggaagccggtgcacaggtgatgcgtgagactgaacagcagatttaccgtcagctgactgacgaggtactggccctgcgattgcctgaaaacggctcacaactgcaccattcataa

SEQ ID NO: 5 - protein, 575 aa, Artificial
  IpaH1.4 novel E3 ligase from Shigella flexneri
MIKSTNIQAIGSGIMHQINNIYSLTPFPLPMELTPSCNEFYLKAWSEWEKNGTPGEQRNIAFNRLKICLQNQEAELNLSELDLKTLPDLPPQITTLEIRKNLLTHLPDLPPMLKVIHAQFNQLESLPALPETLEELNAGDNKIKELPFLPENLTHLRVHNNRLHILPLLPPELKLLVVSGNRLDSIPPFPDKLEGLAMANNFIEQLPELPFSMNRAVLMNNNLTTLPESVLRLAQNAFVNVAGNPLSGHTMRTLQQITTGPDYSGPRIFFSMGNSATISAPEHSLADAVTAWFPENKQSDVSQIWHAFEHEEHANTFSAFLDRLSDTVSARNTSGFREQVAAWLEKLSASAELRQQSFAVAADATESCEDRVALTWNNLRKTLLVHQASEGLFDNDTGALLSLGREMFRLEILEDIARDKVRTLHFVDEIEVYLAFQTMLAEKLQLSTAVKEMRFYGVSGVTANDLRTAEAMVRSREENEFKDWFSLWGPWHAVLKRTEADRWAQAEEQKYEMLENEYSQRVADRLKASGLSGDTDAEREAGAQVMRETEQQIYRQLTDEVLALRLSENGSNHIA

SEQ ID NO: 6 - DNA, 1728 bp, Artificial
  IpaH1.4 novel E3 ligase from Shigella flexneri
atgattaaatcaaccaatatacaggcaatcggttctggtattatgcatcaaataaacaatatatactcgttaactccatttcctttacctatggaactgactccatcttgtaatgaattttatttaaaagcctggagtgaatgggaaaagaacggtaccccaggcgagcaacgcaatatcgccttcaataggctgaaaatatgtttacaaaatcaagaggcagaattaaatttatctgagttagatttaaaaacattaccagatttaccgcctcagataacaacactggaaataagaaaaaacctattaacacatctccctgatttaccaccaatgcttaaggtaatacatgctcaatttaatcaactggaaagcttacctgccttacccgagacgttagaagagcttaatgcgggtgataacaagataaaagaattaccatttcttcctgaaaatctaactcatttacgggttcataataaccgattgcatattctgccactattgccaccggaactaaaattactggtagtttctggaaacagattagacagcattcccccctttccagataagcttgaagggctggctatggctaataattttatagaacaactaccggaattaccttttagtatgaacagggctgtgctaatgaataataatctgacaacacttccggaaagtgtcctgagattagctcagaatgccttcgtaaatgttgcaggtaatccactgtctggccataccatgcgtacactacaacaaataaccaccggaccagattattctggtcctcgaatatttttctctatgggaaattctgccacaatttccgctccagaacactccctggctgatgccgtgacagcatggttcccggaaaacaaacaatctgatgtatcacagatatggcatgcttttgaacatgaagagcacgccaacaccttttccgcgttccttgaccgcctttccgataccgtctctgcacgcaatacctccggattccgtgaacaggtcgctgcatggctggaaaaactcagtgcctctgcggagcttcgacagcagtctttcgctgttgctgctgatgccactgagagctgtgaggaccgtgtcgcgctcacatggaacaatctccggaaaaccctcctggtccatcaggcatcagaaggccttttcgataatgataccggcgctctgctctccctgggcagggaaatgttccgcctcgaaattctggaggacattgcccgggataaagtcagaactctccattttgtggatgagatagaagtctacctggccttccagaccatgctcgcagagaaacttcagctctccactgccgtgaaggaaatgcgtttctatggcgtgtcgggagtgacagcaaatgacctccgcactgccgaagccatggtcagaagccgtgaagagaatgaatttaaggactggttctccctctggggaccatggcatgctgtactgaagcgtacggaagctgaccgctgggcgcaggcagaagagcagaagtatgagatgctggagaatgagtactctcagagggtggctgaccggctgaaagcatcaggtctgagcggtgatacggatgcggagagggaagccggtgcacaggtgatgcgtgagactgaacagcagatttaccgtcagttgactgacgaggtactggccctgcgattgtctgaaaacggctcaaatcatatcgcataa

SEQ ID NO: 7 - protein, 571 aa, Artificial
  IpaH2.5 novel E3 ligase from Shigella flexneri
MLIRILVIMIKSTNIQAIGSGIMHQINNVYSLTPLSLPMELTPSCNEFYLKTWSEWEKNGTPGEQRNIAFNRLKICLQNQEAELNLSELDLKTLPDLPPQITTLEIRKNLLTHLPDLPPMLKVIHAQFNQLESLPALPETLEELNAGDNKIKELPFLPENLTHLRVHNNRLHILPLLPPELKLLVVSGNRLDSIPPFPDKLEGLALANNFIEQLPELPFSMNRAVLMNNNLTTLPESVLRLAQNAFVNVAGNPLSGHTMRTLQQITTGPDYSGPRIFFSMGNSATISAPEHSLADAVTAWFPENKQSDVSQIWHAFEHEEHANTFSAFLDRLSDTVSARNTSGFREQVAAWLEKLSASAELRQQSFAVAADATESCEDRVALTWNNLRKTLLVHQASEGLFDNDTGALLSLGREMFRLEILEDIARDKVRTLHFVDEIEVYLAFQTMLAEKLQLSTAVKEMRFYGVSGVTANDLRTAEAMVRSREENEFTDWFSLWGPWHAVLKRTEADRWAQAEEQKYEMLENEYSQRVADRLKASGLSGDADAEREAGAQVMRETEQQIYRQLTDEVLA

SEQ ID NO: 8 - DNA, 1716 bp, Artificial
  IpaH2.5 novel E3 ligase from Shigella flexneri
atgttgataagaattctagttataatgattaaatcaaccaatatacaggcaatcggttctggcattatgcatcaaataaacaatgtatactcgttaactccattatctttacctatggaactgactccatcttgtaatgaattttatttaaaaacctggagcgaatgggaaaagaacggtaccccaggcgagcaacgcaatatcgccttcaataggctgaaaatatgtttacaaaatcaagaggcagaattaaatttatctgagttagatttaaaaacattaccagatttaccgcctcagataacaacactggaaataagaaaaaacctattaacacatctccctgatttaccaccaatgcttaaggtaatacatgctcaatttaatcaactggaaagcttacctgccttacccgagacgttagaagagcttaatgcgggtgataacaagataaaagaattaccatttcttcctgaaaatctaactcatttacgggttcataataaccgattgcatattctgccactattgccaccggaactaaaattactggtagtttctggaaacagattagacagcattcccccctttccagataagcttgaagggctggctctggctaataattttatagaacaactaccggaattaccttttagtatgaacagggctgtgctaatgaataataatctgacaacacttccggaaagtgtcctgagattagctcagaatgccttcgtaaatgttgcaggtaatccattgtctggccataccatgcgtacactacaacaaataaccaccggaccagattattctggtcctcgaatatttttctctatgggaaattctgccacaatttccgctccagaacactccctggctgatgccgtgacagcatggttcccggaaaacaaacaatctgatgtatcacagatatggcatgcttttgaacatgaagagcatgccaacaccttttccgcgttccttgaccgcctttccgataccgtctctgcacgcaatacctccggattccgtgaacaggtcgctgcatggctggaaaaactcagtgcctctgcggagcttcgacagcagtctttcgctgttgctgctgatgccactgagagctgtgaggaccgtgtcgcgctcacatggaacaatctccggaaaaccctcctggtccatcaggcatcagaaggccttttcgataatgataccggcgctctgctctccctgggcagggaaatgttccgcctcgaaattctggaggatattgcccgggataaagtcagaactctccattttgtggatgagatagaagtctacctggccttccagaccatgctcgcagagaaacttcagctctctactgccgtgaaggaaatgcgtttctatggcgtgtcgggagtgacagcaaatgacctccgcactgccgaagccatggtcagaagccgtgaagagaatgaatttacggactggttctccctctggggaccatggcatgctgtactgaagcgtacggaagctgaccgctgggcgcaggcagaagagcagaagtatgagatgctggagaatgagtactctcagagggtggctgaccggctgaaagcatcaggtctgagcggtgatgcggatgcggagagggaagccggtgcacaggtgatgcgtgagactgaacagcagatttaccgtcagttgactgacgaggtactggcctga

SEQ ID NO: 9 - protein, 574 aa, Artificial
  IpaH4.5 novel E3 ligase from Shigella flexneri
MKPINNHSFFRSLCGLSCISRLSVEEQCTRDYHRIWDDWAREGTTTENRIQAVRLLKICLDTREPVLNLSLLKLRSLPPLPLHIRELNISNNELISLPENSPLLTELHVNGNNLNILPTLPSQLIKLNISFNRNLSCLPSLPPYLQSLSARFNSLETLPELPSTLTILRIEGNRLTVLPELPHRLQELFVSGNRLQELPEFPQSLKYLKVGENQLRRLSRLPQELLALDVSNNLLTSLPENIITLPICTNVNISGNPLSTRVLQSLQRLTSSPDYHGPQIYFSMSDGQQNTLHRPLADAVTAWFPENKQSDVSQIWHAFEHEEHANTFSAFLDRLSDTVSARNTSGFREQVAAWLEKLSASAELRQQSFAVAADATESCEDRVALTWNNLRKTLLVHQASEGLFDNDTGALLSLGREMFRLEILEDIARDKVRTLHFVDEIEVYLAFQTMLAEKLQLSTAVKEMRFYGVSGVTANDLRTAEAMVRSREENEFTDWFSLWGPWHAVLKRTEADRWAQAEEQKYEMLENEYSQRVADRLKASGLSGDADAQREAGAQVMRETEQQIYRQLTDEVLA

SEQ ID NO: 10 - DNA, 1725 bp, Artificial
  IpaH4.5 novel E3 ligase from Shigella flexneri
atgaaaccgatcaacaatcattctttttttcgttccctttgtggcttatcatgtatatctcgtttatcggtagaagaacagtgtaccagagattaccaccgcatctgggatgactgggctagggaaggaacaacaacagaaaatcgcatccaggcggttcgattattgaaaatatgtctggatacccgggagcctgttctcaatttaagcttactgaaactacgttctttaccaccactccctttgcatatacgtgaacttaatatttccaacaatgagttaatctccctacctgaaaattctccgcttttgacagaacttcatgtaaatggtaacaacttgaatatactcccgacacttccatctcaactgattaagcttaatatttcattcaatcgaaatttgtcatgtctgccatcattaccaccatatttacaatcactctcggcacgttttaatagtctggagacgttaccagagcttccatcaacgctaacaatattacgtattgaaggtaatcgccttactgtcttgcctgaattgcctcatagactacaagaactctttgtttccggcaacagactacaggaactaccagaatttcctcagagcttaaaatatttgaaggtaggtgaaaatcaactacgcagattatccagattaccgcaagaactattggctctggatgtttccaataacctactaacttcattacccgaaaatataatcacattgcccatttgtacgaatgttaacatttcagggaatccattgtcgactcgcgttctgcaatccctgcaaagattaacctcttcgccggactaccacggcccgcagatttacttctccatgagtgacggacaacagaatacactccatcgccccctggctgatgccgtgacagcatggttcccggaaaacaaacaatctgatgtatcacagatatggcatgcttttgaacatgaagagcatgccaacaccttttccgcgttccttgaccgcctttccgataccgtctctgcacgcaatacctccggattccgtgaacaggtcgctgcatggctggaaaaactcagtgcctctgcggagcttcgacagcagtctttcgctgttgctgctgatgccactgagagctgtgaggaccgtgtcgcgctcacatggaacaatctccggaaaaccctcctggtccatcaggcatcagaaggccttttcgataatgataccggcgctctgctctccctgggcagggaaatgttccgcctcgaaattctggaggacattgcccgggataaagtcagaactctccattttgtggatgagatagaagtctacctggccttccagaccatgctcgcagagaaacttcagctctccactgccgtgaaggaaatgcgtttctatggcgtgtcgggagtgacagcaaatgacctccgcactgccgaagctatggtcagaagccgtgaagagaatgaatttacggactggttctccctctggggaccatggcatgctgtactgaagcgtacggaagctgaccgctgggcgcaggcagaagagcagaagtatgagatgctggagaatgagtactctcagagggtggctgaccggctgaaagcatcaggtctgagcggtgatgcggatgcgcagagggaagccggtgcacaggtgatgcgtgagactgaacagcagatttaccgtcagctgactgacgaggtactggcctga

SEQ ID NO: 11 - protein, 565 aa, Artificial
  IpaH7.8 novel E3 ligase from Shigella flexneri
MFSVNNTHSSVSCSPSINSNSTSNEHYLRILTEWEKNSSPGEERGIAFNRLSQCFQNQEAVLNLSDLNLTSLPELPKHISALIVENNKLTSLPKLPAFLKELNADNNRLSVIPELPESLTTLSVRSNQLENLPVLPNHLTSLFVENNRLYNLPALPEKLKFLHVYYNRLTTLPDLPDKLEILCAQRNNLVTFPQFSDRNNIRQKEYYFHFNQITTLPESFSQLDSSYRINISGNPLSTRVLQSLQRLTSSPDYHGPQIYFSMSDGQQNTLHRPLADAVTAWFPENKQSDVSQIWHAFEHEEHANTFSAFLDRLSDTVSARNTSGFREQVAAWLEKLSASAELRQQSFAVAADATESCEDRVALTWNNLRKTLLVHQASEGLFDNDTGALLSLGREMFRLEILEDIARDKVRTLHFVDEIEVYLAFQTMLAEKLQLSTAVKEMRFYGVSGVTANDLRTAEAMVRSREENEFTDWFSLWGPWHAVLKRTEADRWAQAEEQKYEMLENEYSQRVADRLKASGLSGDADAEREAGAQVMRETEQQIYRQLTDEVLALRLSENGSRLHHS

SEQ ID NO: 12 - DNA, 1698 bp, Artificial
  IpaH7.8 novel E3 ligase from Shigella flexneri
atgttctctgtaaataatacacactcatcagtttcttgctccccctctattaactcaaactcaaccagtaatgaacattatctgagaatcctgactgaatgggaaaagaactcttctcccggggaagagcgaggcattgcttttaacagactctcccagtgctttcagaatcaagaagcagtattaaatttatcagacctaaatttgacgtctcttcccgaattaccaaagcatatttctgctttgattgtagaaaataataaattaacatcattgccaaagctgcctgcatttcttaaagaacttaatgctgataataacaggctttctgtgataccagaacttcctgagtcattaacaactttaagtgttcgttctaatcaactggaaaaccttcctgttttgccaaaccatttaacatcattatttgttgaaaataacaggctatataacttaccggctcttcccgaaaaattgaaatttttacatgtttattataacaggctgacaacattacccgacttaccggataaactggaaattctctgtgctcagcgcaataatctggttacttttcctcaattttctgatagaaacaatatcagacaaaaggaatattattttcattttaatcagataaccactcttccggagagtttttcacaattagattcaagttacaggattaatatttcagggaatccattgtcgactcgcgttctgcaatccctgcaaagattaacctcttcgccggactaccacggcccacagatttacttctccatgagtgacggacaacagaatacactccatcgccccctggctgatgccgtgacagcatggttcccggaaaacaaacaatctgatgtatcacagatatggcatgcttttgaacatgaagagcatgccaacaccttttccgcgttccttgaccgcctttccgataccgtctctgcacgcaatacctccggattccgtgaacaggtcgctgcatggctggaaaaactcagtgcctctgcggagcttcgacagcagtctttcgctgttgctgctgatgccactgagagctgtgaggaccgtgtcgcgctcacatggaacaatctccggaaaaccctcctggtccatcaggcatcagaaggccttttcgataatgataccggcgctctgctctccctgggcagggaaatgttccgcctcgaaattctggaggacattgcccgggataaagtcagaactctccattttgtggatgagatagaagtctacctggccttccagaccatgctcgcagagaaacttcagctctctactgccgtgaaggaaatgcgtttctatggcgtgtcgggagtgacagcaaatgacctccgcactgccgaagccatggtcagaagccgtgaagagaatgaatttacggactggttctccctctggggaccatggcatgctgtactgaagcgtacggaagctgaccgctgggcgcaggcagaagagcagaagtatgagatgctggagaatgagtactctcagagggtggctgaccggctgaaagcatcaggtctgagcggtgatgcggatgcggagagggaagccggtgcacaggtgatgcgtgagactgaacagcagatttaccgtcagttgactgacgaggtactggccctgcgattgtctgaaaacggctcacgactgcaccattcataa

SEQ ID NO: 13 - protein, 545 aa, Artificial
  IpaH9.8 novel E3 ligase from Shigella flexneri
MLPINNNFSLPQNSFYNTISGTYADYFSAWDKWEKQALPGEERDEAVSRLKECLINNSDELRLDRLNLSSLPDNLPAQITLLNVSYNQLTNLPELPVTLKKLYSASNKLSELPVLPPALESLQVQHNELENLPALPDSLLTMNISYNEIVSLPSLPQALKNLRATRNFLTELPAFSEGNNPVVREYFFDRNQISHIPESILNLRNECSIHISDNPLSSHALQALQRLTSSPDYHGPRIYFSMSDGQQNTLHRPLADAVTAWFPENKQSDVSQIWHAFEHEEHANTFSAFLDRLSDTVSARNTSGFREQVAAWLEKLSASAELRQQSFAVAADATESCEDRVALTWNNLRKTLLVHQASEGLFDNDTGALLSLGREMFRLEILEDIARDKVRTLHFVDEIEVYLAFQTMLAEKLQLSTAVKEMRFYGVSGVTANDLRTAEAMVRSREENEFTDWFSLWGPWHAVLKRTEADRWAQAEEQKYEMLENEYPQRVADRLKASGLSGDADAEREAGAQVMRETEQQIYRQLTDEVLALRLSENGSQLHHS

SEQ ID NO: 14 - DNA, 1638 bp, Artificial
  IpaH9.8 novel E3 ligase from Shigella flexneri
atgttaccgataaataataacttttcattgccccaaaattctttttataacactatttccggtacatatgctgattacttttcagcatgggataaatgggaaaaacaagcgctccccggtgaagagcgtgatgaggctgtctcccgacttaaagaatgtcttatcaataattccgatgaacttcgactggaccgtttaaatctgtcctcgctacctgacaacttaccagctcagataacgctgctcaatgtatcatataatcaattaactaacctacctgaactgcctgttacgctaaaaaaattatattccgccagcaataaattatcagaattgcccgtgctacctcctgcgctggagtcacttcaggtacaacacaatgagctggaaaacctgccagctttacccgattcgttattgactatgaatatcagctataacgaaatagtctccttaccatcgctcccacaggctcttaaaaatctcagagcgacccgtaatttcctcactgagctaccagcattttctgagggaaataatcccgttgtcagagagtatttttttgatagaaatcagataagtcatatcccggaaagcattcttaatctgaggaatgaatgttcaatacatattagtgataacccattatcatcccatgctctgcaagccctgcaaagattaacctcttcgccggactaccacggcccacggatttacttctccatgagtgacggacaacagaatacactccatcgccccctggctgatgccgtgacagcatggttcccggaaaacaaacaatctgatgtatcacagatatggcatgcttttgaacatgaagagcatgccaacaccttttccgcgttccttgaccgcctttccgataccgtctctgcacgcaatacctccggattccgtgaacaggtcgctgcatggctggaaaaactcagtgcctctgcggagcttcgacagcagtctttcgctgttgctgctgatgccactgagagctgtgaggaccgtgtcgcgctcacatggaacaatctccggaaaaccctcctggtccatcaggcatcagaaggccttttcgataatgataccggcgctctgctctccctgggcagggaaatgttccgcctcgaaattctggaggatattgcccgggataaagtcagaactctccattttgtggatgagatagaagtctacctggccttccagaccatgctcgcagagaaacttcagctctccactgccgtgaaggaaatgcgtttctatggcgtgtcgggagtgacagcaaatgacctccgcactgccgaagccatggtcagaagccgtgaagagaatgaatttacggactggttctccctctggggaccatggcatgctgtactgaagcgtacggaagctgaccgctgggcgcaggcagaagagcagaaatatgagatgctggagaatgagtaccctcagagggtggctgaccggctgaaagcatcaggtctgagcggtgatgcggatgcggagagggaagccggtgcacaggtgatgcgtgagactgaacagcagatttaccgtcagctgactgacgaggtactggccctgcgattgtctgaaaacggctcacaactgcaccattcataa

SEQ ID NO: 15 - protein, 172 aa, Artificial
  LegAU13 F-box motif from Legionella pneumophila
MKKNFFSDLPEETIVNTLSFLKANTLARIAQTCQFFNRLANDKHLELHQLRQQHIKRELWGNLMVAARSNNLEEVKKILKKGIDPTQTNSYHLNRTPLLAAIEGKAYQTANYLWRKYTFDPNFKDNYGDSPISLLKKQLANPAFKDKEKKQIRALIRGMQEEKIAQSKCLVC

SEQ ID NO: 16 - DNA, 519 bp, Artificial
  LegAU13 F-box motif from Legionella pneumophila
atgaaaaagaattttttttctgatcttcctgaggaaacaattgtcaatacattgagtttcttaaaagcaaacacactagctcgtatagctcagacatgtcaattttttaatcgcttggctaatgataaacatctggagctgcatcaactaagacaacagcatataaagcgagagctatggggaaatcttatggtggcggcaagaagcaataacctggaagaggtcaaaaagattctaaaaaaaggaatcgatccaacccagaccaatagctaccacttaaatagaacgcctttacttgcagctatcgaaggaaaagcatatcaaactgcaaattacctctggagaaaatacactttcgatcccaattttaaagataactatggtgattcacctatctctcttcttaaaaagcaactggcaaatccagccttcaaggataaggaaaaaaaacaaatacgcgccttaattaggggaatgcaagaagaaaaaatagcacagagcaagtgccttgtttgttaa

SEQ ID NO: 17 - protein, 188 aa, Artificial
  LegU1 F-box motif from Legionella pneumophila
MKAKYDPTKPGLQKLPPEIKVMILEFLDAKSKLALSQTNYGWRDLILDRPEYTKEITNTLFRLDKKRHRQAIAQMMSGRVTASSMAKLFEELLCFSIPSSYVFLIFFASQKSVALIEVLTVILVFAAITSLAHDLVDYFIESDTKAEKQHAHRRAFQFFAQPSQSAAQQNLEEENLSADPKACQCEPL

SEQ ID NO: 18 - DNA, 567 bp, Artificial
  LegU1 F-box motif from Legionella pneumophila
atgaaagcaaaatacgaccccacaaagcctggactccaaaagttacctcctgaaatcaaggtaatgattcttgagtttcttgatgccaaatcaaaactagctctttcacagacaaattatggttggcgtgatttaattctagaccggccagaatataccaaagaaataacgaatacattatttcgtcttgataaaaaacgccatcgtcaagcaatagcacaaatgatgtcaggaagagttacagcaagttctatggctaagctatttgaagaattactatgttttagcataccttcgtcctatgtgtttttaatctttttcgcatcgcaaaaatctgtggcgcttatagaagtcttaaccgtaatccttgtgtttgctgcaataacctctctcgcccatgatctggtggattattttattgaaagtgatacaaaagctgagaaacagcatgcacatcgccgtgcttttcaattctttgcccaacccagtcaaagcgctgcacaacaaaacttggaggaagagaatttaagtgctgatcccaaggcctgccaatgtgagccattgtag

SEQ ID NO: 19 - protein, 240 aa, Artificial
  LubX U-box motif from Legionella pneumophila
MATRNPFDIDHKSKYLREAALEANLSHPETTPTMLTCPIDSGFLKDPVITPEGFVYNKSSILKWLETKKEDPQSRKPLTAKDLQPFPELLIIVNRFVETQTNYEKLKNRLVQNARVAARQKEYTEIPDIFLCPISKTLIKTPVITAQGKVYDQEALSNFLIATGNKDETGKKLSIDDVVVFDELYQQIKVYNFYRKREVQKNQIQPSVSNGFGFFSLNFLTSWLWGTEEKKEKTSSDMTY

SEQ ID NO: 20 - DNA, 723 bp, Artificial
  LubX U-box motif from Legionella pneumophila
atggcgacgcgaaatccttttgatattgatcataaaagtaaatacttaagagaagcagcattagaagccaatttatctcatccagaaacaacaccaacaatgctgacttgccctattgacagcggatttctaaaagatcccgtgatcacacctgaaggttttgtttataataaatcctctattttaaaatggttagaaacgaaaaaagaagacccacaaagccgtaaacccttaacggctaaagatttgcaaccattccccgagttattgattatagtcaatagatttgttgagacacaaacgaactatgaaaaattaaaaaacagattagtgcaaaatgctcgggttgctgcacgccaaaaagaatacactgaaattccggatatatttctttgcccaataagtaaaacgcttatcaaaacacctgtcattactgcccaagggaaagtatatgatcaagaagcattaagtaactttcttatcgcaacgggtaataaagatgaaacaggcaaaaaattatccattgatgatgtagtggtgtttgatgaactctatcaacagataaaagtttataatttttaccgcaaacgcgaagtgcaaaaaaatcaaattcaaccttcagtaagtaatggttttggcttttttagcttgaattttctcacctcatggttatggggaactgaggagaaaaaagaaaagacatcatctgatatgacgtactaa

SEQ ID NO: 21 - protein, 191 aa, Artificial
  NleG2-3 U-box motif from Enterohemorrhagic Escherichia coli (EHEC) O157H7
MPLTSDIRSHSFNLGVEVVRARIVANGRGDITVGGETVSIVYDSTNGRFSSSGGNGGLLSELLLLGFNSGPRALGERMLSMLSDSGEAQSQESIQNKISQCKFSVCPERLQCPLEAIQCPITLEQPEKGIFVKNSDGSDVCTLFDAAAFSRLVGEGLPHPLTREPITASIIVKHEECIYDDTRGNFIIKGN

SEQ ID NO: 22 - DNA, 576 bp, Artificial
  NleG2-3 U-box motif from Enterohemorrhagic Escherichia coli (EHEC) O157H7
atgccattaacctcagatattagatcacattcatttaatcttggggtggaggttgttcgtgcccgaattgtagccaatgggcgcggagatattacagtcggtggtgaaactgtcagtattgtgtatgattctactaatgggcgcttttcatccagtggcggtaatggcggattgctttctgagttattgcttttgggatttaatagtggtcctcgagcccttggtgagagaatgctaagtatgctttcggactcaggtgaagcacaatcgcaagagagtattcagaacaaaatatctcaatgtaagttttctgtttgtccagagagacttcagtgcccgcttgaggctattcagtgtccaattacactggagcagcctgaaaaaggtatttttgtgaagaattcagatggttcagatgtatgtactttatttgatgccgctgcattttctcgtttggttggtgaaggcttaccccacccactgacccgggaaccaataacggcatcaataattgtaaaacatgaagaatgcatttatgacgataccagaggaaacttcattataaagggtaattga

SEQ ID NO: 23 - protein, 213 aa, Artificial
  NleG5-1 U-box motif from Enterohemorrhagic Escherichia coli (EHEC) O157H7
MPVDLTPYILPGVSFLSDIPQETLSEIRNQTIRGEAQIRLGELMVSIRPMQVNGYFMGSLNQDGLSNDNIQIGLQYIEHIERTLNHGSLTSREVTVLREIEMLENMDLLSNYQLEELLDKIEVCAFNVEHAQLQVPESLRTCPVTLCEPEDGVFMRNSMNSNVCMLYDKMALIHLVKTRAAHPLSRESIAVSMIVGRDNCAFDPDRGNFVLKN

SEQ ID NO: 24 - DNA, 642 bp, Artificial
  NleG5-1 U-box motif from Enterohemorrhagic Escherichia coli (EHEC) O157H7
atgcctgtagatttaacgccttatattttacctggggttagttttttgtctgacattcctcaagaaaccttgtctgagatacgtaatcagactattcgtggagaagctcaaataagactgggtgagttgatggtgtcaatacgacctatgcaggtaaatggatattttatgggaagtcttaaccaggatggtttatcgaatgataatatccagattggccttcaatatatagaacatattgaacgtacacttaatcatggtagtttgacaagccgtgaagttacagtactgcgtgaaattgagatgctcgaaaatatggatttgctttctaactaccagttagaggagttgttagataaaattgaagtatgtgcatttaatgtggagcatgcacaattgcaagtgccagagagcttacgaacatgccctgttacattatgtgaaccagaagatggggtatttatgaggaattcaatgaattcaaatgtttgtatgttgtatgataaaatggcattaatacatcttgttaaaacaagggcggctcatcctttgagcagggaatcaatcgcagtttcaatgattgtaggaagagataattgtgcttttgaccctgacagaggtaacttcgttttaaaaaattaa

SEQ ID NO: 25 - protein, 782 aa, Artificial
  NleL HECT motif from Enterohemorrhagic Escherichia coli (EHEC) O157H7
MLPTTNISVNSGVISFESPVDSPSNEDVEVALEKWCAEGEFSENRHEVASKILDVISTNGETLSISEPITTLPDLLPGSLKELVLNGCTELKSINCLPPNLSSLSMVGCSSLEVINCSIPENVINLSLCHCSSLKHIEGSFPEALRNSVYLNGCNSLNESQCQFLAYDVSQGRACLSKAELTADLIWLSANRTGEESAEELNYSGCDLSGLSLVGLNLSSVNFSGAVLDDTDLRMSDLSQAVLENCSFKNSILNECNFCYANLSNCIIRALFENSNFSNSNLKNASFKGSSYIQYPPILNEADLTGAIIIPGMVLSGAILGDVKELFSEKSNTINLGGCYIDLSDIQENILSVLDNYTKSNKSILLTMNTSDDKYNHDKVRAAEELIKKISLDELAAFRPYVKMSLADSFSIHPYLNNANIQQWLEPICDDFFDTIMSWFNNSIMMYMENGSLLQAGMYFERHPGAMVSYNSSFIQIVMNGSRRDGMQERFRELYEVYLKNEKVYPVTQQSDFGLCDGSGKPDWDDDSDLAYNWVLLSSQDDGMAMMCSLSHMVDMLSPNTSTNWMSFFLYKDGEVQNTFGYSLSNLFSESFPIFSIPYHKAFSQNFVSGILDILISDNELKERFIEALNSNKSDYKMIADDQQRKLACVWNPFLDGWELNAQHVDMIMGSHVLKDMPLRKQAEILFCLGGVFCKYSSSDMFGTEYDSPEILRRYANGLIEQAYKTDPQVFGSVYYYNDILDRLQGRNNVFTCTAVLTDMLTEHAKESFPEIFSLYYPVAWR

SEQ ID NO: 26 - DNA, 2349 bp, Artificial
  NleL HECT motif from Enterohemorrhagic Escherichia coli (EHEC) O157H7
atgctgcccactacaaatatctctgtaaattctggagtaatatcttttgaaagtcctgtagattcaccatctaacgaggatgttgaagttgccctcgaaaagtggtgtgctgagggagaatttagcgaaaatcgtcatgaggttgcatcaaaaatacttgatgttataagtactaatggagagactttatcaatcagtgagccaataacaacattaccagacttgcttccaggttctctgaaagaactggttttgaatggatgtacagagcttaaatcaataaactgcttaccccccaacttatcttcattaagtatggttggatgctcatcattagaggttataaattgcagcatacctgaaaatgtcattaatttatctttatgccattgtagttctttgaaacatatagaaggttcctttcctgaggcactcagaaattccgtatatttaaatggctgtaattcattaaatgaatcgcaatgtcaattccttgcatatgatgtcagtcaaggccgtgcctgcctgagcaaagctgagcttactgctgacttaatttggttgtcagctaaccgaacgggtgaagagtctgctgaagaattgaattactctggatgtgacttgtcaggtctaagtcttgtagggctgaatttatcatcagtaaatttttctggagcagtgcttgatgatacagatctcaggatgagtgatttgtctcaggctgtattggaaaactgttcttttaaaaactcgattttgaatgaatgtaatttttgttatgctaatttatctaattgtattattagggctttgtttgaaaactctaatttcagcaattccaatcttaaaaatgcatcatttaaaggatcttcatatatacaatatcctccaattttgaacgaggctgatttaacaggagctattataattcctggaatggttttaagtggtgctatcttaggtgatgtaaaggagctctttagtgaaaaaagtaataccattaatctaggagggtgttacatagatctatctgacatacaggaaaatatattatctgtgttggataactatacaaaatcaaataaatcaattttattgactatgaatacatctgatgataagtataaccatgataaagtaagggccgctgaagaacttatcaaaaaaatatctcttgacgaattagcggcgttccggccctatgttaagatgtctttggctgattcatttagtattcatccttatttgaacaacgcaaatatacagcaatggctcgagcctatatgtgatgacttttttgatactataatgtcttggtttaataattcaataatgatgtatatggagaatggtagtttattgcaggcagggatgtattttgagcgacatccaggtgcgatggtatcttataatagttcctttatacaaattgtaatgaatggttcacggcgtgatggaatgcaggaacgatttagggaactctatgaagtatatttaaaaaatgaaaaagtttatcctgtcacacagcagagtgattttggattgtgcgatggctctgggaagcctgactgggatgatgattccgatttggcttataactgggttttgttatcatcacaggatgatggtatggcaatgatgtgttctttgagtcatatggttgatatgttatctcctaatacatcaactaattggatgtccttttttttatataaggatggagaagttcaaaatacatttgggtattcattgagcaatcttttttctgaatcatttccaattttcagtattccttatcataaagctttttcccagaatttcgtttctggtattctggatatactcatttctgataatgaactcaaagagagatttattgaggcacttaattccaataaatcagattataaaatgattgctgatgatcagcaaaggaaacttgcctgtgtctggaatccctttcttgatggttgggaactgaacgctcagcatgtagatatgattatggggagccatgtattgaaagatatgccactaagaaaacaggctgaaatattattttgtttagggggggttttctgtaaatactcatcgagtgatatgtttggtacagagtatgattctcctgagattctacggagatatgcaaatggattgattgaacaagcttataaaacagatcctcaggtatttggctcagtttattattacaatgatattttagacaggctacaaggaagaaataatgtttttacttgtaccgctgtgctgactgatatgctaacggagcatgcaaaagaatcttttcctgaaatattttcattgtattatcctgttgcgtggcgttga

SEQ ID NO: 27 - protein, 917 aa, Artificial
  SidC unconventional motif from Legionella pneumophila
MVINMVDVIKFKEPERCDYLYVDENNKVHILLPIVGGDEIGLDNTCQTAVELITFFYGSAHSGVTKYSAEHQLSEYKRQLEEDIKAINSQKKISPHAYDDLLKEKIERLQQIEKYIELIQVLKKQYDEQNDIRQLRTGGIPQLPSGVKEIIKSSENAFAVRLSPYDNDKFTRFDDPLFNVKRNISKYDTPSRQAPIPIYEGLGYRLRSTLFPEDKTPTPINKKSLRDKVKSTVLSHYKDEDRIDGEKKDEKLNELITNLQNELVKELVKSDPQYSKLSLSKDPRGKEINYDYLVNSLMLVDNDSEIGDWIDTILDATVDSTVWVAQASSPFYDGAKEISSDRDADKISIRVQYLLAEANIYCKTNKLSDANFGEFFDKEPHATEIAKRVKEGFTQGADIEPIIYDYINSNHAELGLKSPLTGKQQQEITDKFTKHYNTIKESPHFDEFFVADPDKKGNIFSHQGRISCHFLDFFTRQTKGKHPLGDLASHQEALQEGTSNRLHHKNEVVAQGYEKLDQFKKEVVKLLAENKPKELLDYLVATSPTGVPNYSMLSKETQNYIAYNRNWPAIQKELEKATSIPESQKQDLSRLLSRDNLQHDNLSAITWSKYSSKPLLDVELNKIAEGLELTAKIYNEKRGREWWFKGSRNEARKTQCEELQRVSKEINTLLQSESLTKSQVLEKVLNSIETLDKIDRDISAESNWFQSTLQKEVRLFRDQLKDICQLDKYAFKSTKLDEIISLEMEEQFQKIQDPAVQQIVRDLPSHCHNDEAIEFFKTLNPEEAAKVASYLSLEYREINKSTDKKTLLEQDIPRLFKEVNTQLLSKLKEEKAIDEQVHEKLSQLADKIAPEHFTRNNIIKWSTNPEKLEESNLNEPIKSVQSPTTKQTSKQFREAMGEITGRNEPPTDTLYTGIIKK

SEQ ID NO: 28 - DNA, 2754 bp, Artificial
  SidC unconventional motif from Legionella pneumophila
atggtgataaacatggttgacgtaatcaaattcaaagagccggaacgttgtgattatctatatgttgatgaaaacaacaaagttcatatccttttaccgattgtaggaggagatgaaataggcctggataatacctgtcaaacagcagttgagttgatcacatttttctatggtagtgcgcacagtggtgtgactaaatattctgctgaacaccaactcagtgaatacaaaaggcaattggaagaagacatcaaagccatcaatagtcaaaagaaaatttcacctcatgcttatgacgatttattaaaagagaaaatagaacgcttacagcaaattgaaaaatacattgaattaattcaagtactaaaaaaacaatatgatgaacaaaatgatatcaggcaacttcgtactggagggattccgcaattaccctctggggtaaaggaaatcattaaatcctctgaaaatgctttcgctgtgagactttctccatatgacaacgataaattcactcgctttgatgaccctttattcaatgtcaaaagaaacatctcaaaatatgacacgccctcaagacaagctcctattccaatatacgagggattaggttatcgcctgcgttcaacactgttcccggaagataaaacaccaactccaattaataaaaaatcacttagggataaagttaaaagcactgttcttagtcattataaagatgaagatagaattgatggagaaaaaaaagatgaaaaattaaacgaactaattactaatcttcaaaacgaacttgtaaaagagttagtaaaaagtgatcctcaatattcgaaactatctttatctaaagatccaagaggaaaagaaataaattacgattatttagtaaatagtttgatgcttgtagataacgactctgaaattggtgattggattgatactattctcgacgctacagtagattccactgtctgggtagctcaggcatccagccctttctatgatggtgctaaagaaatatcatcagaccgcgatgcggacaagatatccatcagagttcagtacctgttggccgaagccaatatttactgtaaaacaaacaaattatcggatgctaactttggagaatttttcgacaaagagcctcatgctactgaaattgcgaaaagagtaaaggaaggatttacgcaaggtgcagatatagaaccaattatatacgactatattaacagcaaccatgccgagctgggattaaaatctccgttaaccggcaaacaacaacaagaaatcactgataaatttacaaaacattataatacgattaaagaatctccacattttgatgagttttttgtcgctgatccggataaaaaaggcaatatcttttctcatcaaggcagaatcagttgtcattttctggatttctttactcgacaaaccaaaggcaaacatcctcttggtgatcttgcaagtcatcaggaagctctccaggaaggaacctccaatcgcttacatcacaagaatgaggtagtagcccaggggtacgaaaaactggatcaattcaagaaagaggttgtcaaactgctggctgagaataaaccaaaagaattattggattatttggttgctacctcacctacaggtgttccaaattactccatgctttcgaaggaaactcaaaattacattgcttataatcgtaactggccagccattcaaaaagagctggaaaaggctaccagcatcccggagagtcaaaaacaagatctttcaagattgctttctcgtgataatttacaacacgataatctaagcgcaattacctggtcaaaatattcctccaagccattattggatgtggaattaaataaaatcgctgaaggattagaactcactgcaaaaatttacaatgaaaagagaggacgcgaatggtggtttaaaggttcaagaaatgaagctcgtaagacccaatgtgaagaattgcaaagagtatccaaagaaatcaatactcttctgcaaagtgaatctttaacgaaaagccaggtacttgaaaaggttttaaattctatagaaacattagataaaattgacagagacatttctgccgaatccaattggtttcaaagtactctgcaaaaggaagtcaggttatttcgagatcaattgaaagatatttgccaattggacaagtatgcctttaaatcaacaaaacttgatgaaatcatctctctggaaatggaagaacaatttcaaaagatacaagatcctgctgttcaacaaattgtcagggacttgccttctcattgccacaatgatgaagcaattgaattctttaagacattgaaccctgaagaggcagcaaaagtagctagctatttaagcctggaatacagggaaattaataaatcaaccgataagaaaactctcctagaacaagatattcccagactgtttaaagaagtcaatacgcagttactctccaaactcaaagaagaaaaagctattgatgagcaagttcatgaaaaactcagtcaactggctgacaaaattgcccctgagcattttacaagaaataacattataaaatggtctaccaaccctgaaaagcttgaggaatcaaatcttaatgagccaatcaaatcagtccaaagccctactactaaacaaacatcaaaacaattcagggaagcgatgggtgaaatcactggaagaaatgagcctcctacagacactttgtacacgggaattataaagaaatag

SEQ ID NO: 29 - protein, 765 aa, Artificial
  SlrP novel E3 ligase from Enterohemorrhagic Escherichia coli (“EHEC”) O157H7
MFNITNIQSTARHQSISNEASTEVPLKEEIWNKISAFFSSEHQVEAQNCIAYLCHPPETASPEEIKSKFECLRMLAFPAYADNIQYSRGGADQYCILSENSQEILSIVFNTEGYTVEGGGKSVTYTRVTESEQASSASGSKDAVNYELIWSEWVKEAPAKEAANREEPVQRMRDCLKNNKTELRLKILGLTTIPAYIPEQITTLILDNNELKSLPENLQGNIKTLYANSNQLTSIPATLPDTIQEMELSINRITELPERLPSALQSLDLFHNKISCLPENLPEELRYLSVYDNSIRTLPAHLPSEITHLNVQSNSLTALPETLPPGLKTLEAGENALTSLPASLPPELQVLDVSKNQITVLPETLPPTITTLDVSRNALTNLPENLPAALQIMQASRNNLVRLPESLPHFRGEGPQPTRIIVEYNPFSERTIQNMQRLMSSVDYQGPRVLVAMGDFSIVRVTRPLHQAVQGWLTSLEEEDVNQWRAFEAEANAAAFSGFLDYLGDTQNTRHPDFKEQVSAWLMRLAEDSALRETVFIIAMNATISCEDRVTLAYHQMQEATLVHDAERGAFDSHLAELIMAGREIFRLEQIESLAREKVKRLFFIDEVEVFLGFQNQLRESLSLTTMTRDMRFYNVSGITESDLDEAEIRIKMAENRDFHKWFALWGPWHKVLERIAPEEWREMMAKRDECIETDEYQSRVNAELEDLRIADDSDAERTTEVQMDAERAIGIKIMEEINQTLFTEIMENILLKKEVSSLMSAYWR

SEQ ID NO: 30 - DNA, 2298 bp, Artificial
  SlrP novel E3 ligase from Enterohemorrhagic Escherichia coli (“EHEC”) O157H7
atgtttaatattactaatatacaatctacggcaaggcatcaaagtattagcaatgaggcctcaacagaggtgcctttaaaagaagagatatggaataaaataagtgcctttttctcttcagaacatcaggttgaagcacaaaactgcatcgcttatctttgtcatccacctgaaaccgcctcgccagaagagatcaaaagcaagtttgaatgtttaaggatgttagctttcccggcgtatgcggataatattcagtatagtagaggaggggcagaccaatactgtattttgagtgaaaatagtcaggaaattctgtctatagtttttaatacagagggctataccgttgagggagggggaaagtcagtcacctatacccgtgtgacagaaagcgagcaggcgagtagcgcttccggctccaaagatgctgtgaattatgagttaatctggtctgagtgggtaaaagaggcgccagcgaaagaggcagcaaatcgtgaagaacccgtacaacggatgcgtgactgcctgaaaaataataagacggaacttcgtctgaaaatattaggacttaccactatacctgcctatattcctgagcagataactactctgatactcgataacaatgaactgaaaagtttgccggaaaatttacagggaaatataaagaccctgtatgccaacagtaatcagctaaccagtatccctgccacgttaccggataccatacaggaaatggagctgagcattaaccgtattactgaattgccggaacgtttgccttcagcgcttcaatcgctggatcttttccataataaaattagttgcttacctgaaaatctacctgaggaacttcggtacctgagcgtttatgataacagcataaggacactgccagcacatcttccgtcagagattacccatttgaatgtgcagagtaattcgttaaccgctttgcctgaaacattgccgccgggcctgaagactctggaggccggcgaaaatgccttaaccagtctgcccgcatcgttaccaccagaattacaggtcctggatgtaagtaaaaatcagattacggttctgcctgaaacacttcctcccacgataacaacgctggatgtttcccgtaacgcattgactaatctaccggaaaacctcccggcggcattacaaataatgcaggcctctcgcaataacctggtccgtctcccggagtcgttaccccattttcgtggtgaaggacctcaacctacaagaataatcgtagaatataatcctttttcagaacgaacaatacagaatatgcagcggctaatgtcctctgtagattatcagggaccccgggtattggttgccatgggcgacttttcaattgttcgggtaactcgaccactgcatcaagctgtccaggggtggctaaccagtctcgaggaggaagacgtcaaccaatggcgggcgtttgaggcagaggcaaacgcggcggctttcagcggattcctggactatcttggtgatacgcagaatacccgacacccggattttaaggaacaagtctccgcctggctaatgcgcctggctgaagatagcgcactaagagaaaccgtatttattatagcgatgaatgcaacgataagctgtgaagatcgggtcacactggcataccaccaaatgcaggaagcgacgttggttcatgatgctgaaagaggcgcctttgatagccacttagcggaactgattatggcggggcgtgaaatctttcggctggagcaaatagaatcgctcgccagagaaaaggtaaaacggctgttttttattgacgaagtcgaagtatttctggggtttcagaatcagttacgagagtcgctgtcgctgacaacaatgacccgggatatgcgattttataacgtttcgggtatcactgagtctgacctggacgaggcggaaataaggataaaaatggctgaaaatagggattttcacaaatggtttgcgctgtgggggccgtggcataaagtgctggagcgcatagcgccagaagagtggcgtgaaatgatggctaaaagggatgagtgtattgaaacggatgagtatcagagccgggtcaatgctgaactggaagatttaagaatagcagacgactctgacgcagagcgtactactgaggtacagatggatgcagagcgtgctattgggataaaaataatggaagagatcaatcagaccctctttactgagatcatggagaatatattgctgaaaaaagaggtgagctcgctcatgagcgcctactggcgatag

SEQ ID NO: 31 - protein, 782 aa, Artificial
  SopA HECT motif from Salmonella typhimurium
MKISSGAINFSTIPNQVKKLITSIREHTKNGLTSKITSVKNTHTSLNEKFKTGKDSPIEFALPQKIKDFFQPKDKNTLNKTLITVKNIKDTNNAGKKNISAEDVSKMNAAFMRKHIANQTCDYNYRMTGAAPLPGGVSVSANNRPTVSEGRTPPVSPSLSLQATSSPSSPADWAKKLTDAVLRQKAGETLTAADRDFSNADFRNITFSKILPPSFMERDGDIIKGFNFSNSKFTYSDISHLHFDECRFTYSTLSDVVCSNTKFSNSDMNEVFLQYSITTQQQPSFIDTTLKNTLIRHKANLSGVILNEPDNSSPPSVSGGGNFIRLGDIWLQMPLLWTENAVDGFLNHEHNNGKSILMTIDSLPDKYSQEKVQAMEDLVKSLRGGRLTEACIRPVESSLVSVLAHPPYTQSALISEWLGPVQERFFAHQCQTYNDVPLPAPDTYYQQRILPVLLDSFDRNSAAMTTHSGLFNQVILHCMTGVDCTDGTRQKAAALYEQYLAHPAVSPHIHNGLFGNYDGSPDWTTRAADNFLLLSSQDSDTAMMLSTDTLLTMLNPTPDTAWDNFYLLRAGENVSTAQISPVELFRHDFPVFLAAFNQQATQRRFGELIDIILSTEEHGELNQQFLAATNQKHSTVKLIDDASVSRLATIFDPLLPEGKLSPAHYQHILSAYHLTDATPQKQAETLFCLSTAFARYSSSAIFGTEHDSPPALRGYAEALMQKAWELSPAIFPSSEQFTEWSDRFHGLHGAFTCTSVVADSMQRHARKYFPSVLSSILPLAWA

SEQ ID NO: 32 - DNA, 2349 bp, Artificial
  SopA HECT motif from Salmonella typhimurium
atgaagatatcatcaggcgcaattaatttttctactattcctaaccaggttaaaaaattaattacctctattcgtgaacatacgaaaaacgggctcacctcaaaaataaccagtgttaaaaacacgcatacatctttaaatgaaaaatttaaaacaggaaaggactcaccgattgagttcgcgttaccacaaaaaataaaagacttctttcagccgaaagataaaaacaccttaaacaaaacattgattactgttaaaaatattaaagatacaaataatgcaggcaagaaaaatatttcagcagaagatgtctcaaaaatgaatgcagcattcatgcgtaagcatattgcaaatcaaacatgtgattataattacagaatgacaggtgcggccccgctccccggtggagtctctgtatcagccaataacaggcccacggtttctgaaggtagaacaccaccagtatccccctccctctcacttcaggctacctcttccccgtcatcacctgccgactgggctaagaaactcacggatgcagttttacgacagaaagccggagaaacccttacggccgcagatcgcgatttttcaaacgcagatttccgtaatattacattcagcaaaatattgccccccagcttcatggagcgagacggcgatattattaaggggttcaacttttcaaattcaaaatttacttattctgatatatctcatttacattttgacgaatgccgattcacttattcgacactgagtgatgtagtctgcagtaatacgaaatttagtaattcagacatgaatgaagtgtttttacagtattcaattactacacaacaacagccctcgtttattgatacaacattaaaaaatacgcttatacgtcacaaagccaacctctctggcgttattttaaatgaaccggataattcatcacctccgtcagtgtcagggggcggaaattttattcgtctaggtgatatctggctgcaaatgccactcctttggactgagaacgctgtggatggatttttaaatcatgagcacaataatggtaaaagtattctgatgaccattgacagcctgcccgataaatacagtcaggaaaaagtccaggcaatggaagacctggttaagtcattgcggggtggccgcttaacagaggcatgtatccggccagttgaaagttcgctggtaagcgtactggcccaccccccctatacgcaaagtgcgcttatcagcgagtggctcgggcctgttcaggaacgtttttttgcccaccagtgccagacctataatgacgttcccctgccggctcctgacacatattatcagcagcgcatactgcctgtgttgctggattcgtttgacaggaacagcgccgccatgaccactcacagcggactctttaatcaggtgattttacactgtatgacaggcgtggactgcactgatggcacccgccagaaagctgcagcgctttatgaacagtatcttgctcacccggcggtgtctccccacatccataatgggctcttcggcaattatgatggcagcccggactggacaacccgcgctgcagataatttcctgctgctttcctcccaagattcagacacggcgatgatgctctccactgacacgctgttaacaatgctaaaccctactcctgacactgcatgggacaacttttacctgctgcgagccggagagaacgtttccaccgcgcaaatctctccggtagagttattccgtcatgactttccggtgtttctcgccgcatttaatcagcaggccacgcagcgacgctttggggagctgattgatatcatcctcagcactgaagagcacggggagctgaaccagcagtttcttgccgccacgaaccagaaacattccaccgtgaagttgattgatgatgcctcagtgtcgcgtctggccaccatttttgaccccttgcttcctgaaggcaaactcagcccggcacactaccagcacatcctcagtgcttatcacctgacggacgccaccccacagaagcaggcggaaaccctgttctgtctcagtaccgcattcgcacgctattcctccagcgccattttcggcactgagcatgactctccgccggccctgagaggctatgcggaggcgctgatgcagaaagcctgggagctgtctccggcgatattcccatccagcgaacagtttaccgagtggtccgaccgttttcacggcctccatggcgcctttacctgtaccagcgttgtggcggatagtatgcaacgtcatgccagaaaatatttcccgagtgttctgtcatccatcctgccactggcctgggcgtaa

SEQ ID NO: 33 - protein, 700 aa, Artificial
  SspH1 novel E3 ligase from Salmonella typhimurium
MFNIRNTQPSVSMQAIAGAAAPEASPEEIVWEKIQVFFPQENYEEAQQCLAELCHPARGMLPDHISSQFARLKALTFPAWEENIQCNRDGINQFCILDAGSKEILSITLDDAGNYTVNCQGYSEAHDFIMDTEPGEECTEFAEGASGTSLRPATTVSQKAAEYDAVWSKWERDAPAGESPGRAAVVQEMRDCLNNGNPVLNVGASGLTTLPDRLPPHITTLVIPDNNLTSLPELPEGLRELEVSGNLQLTSLPSLPQGLQKLWAYNNWLASLPTLPPGLGDLAVSNNQLTSLPEMPPALRELRVSGNNLTSLPALPSGLQKLWAYNNRLTSLPEMSPGLQELDVSHNQLTRLPQSLTGLSSAARVYLDGNPLSVRTLQALRDIIGHSGIRIHFDMAGPSVPREARALHLAVADWLTSAREGEAAQADRWQAFGLEDNAAAFSLVLDRLRETENFKKDAGFKAQISSWLTQLAEDAALRAKTFAMATEATSTCEDRVTHALHQMNNVQLVHNAEKGEYDNNLQGLVSTGREMFRLATLEQIAREKAGTLALVDDVEVYLAFQNKLKESLELTSVTSEMRFFDVSGVTVSDLQAAELQVKTAENSGFSKWILQWGPLHSVLERKVPERFNALREKQISDYEDTYRKLYDEVLKSSGLVDDTDAERTIGVSAMDSAKKEFLDGLRALVDEVLGSYLTARWRLN

SEQ ID NO: 34 - DNA, 2103 bp, Artificial
  SspH1 novel E3 ligase from Salmonella typhimurium
atgtttaatatccgcaatacacaaccttctgtaagtatgcaggctattgctggtgcagcggcaccagaggcatctccggaagaaattgtatgggaaaaaattcaggtttttttcccgcaggaaaattacgaagaagcgcaacagtgtctcgctgaactttgccatccggcccggggaatgttgcctgatcatatcagcagccagtttgcgcgtttaaaagcgcttaccttccccgcgtgggaggagaatattcagtgtaacagggatggtataaatcagttttgtattctggatgcaggcagcaaggagatattgtcaatcactcttgatgatgccgggaactataccgtgaattgtcaggggtacagtgaagcacatgacttcatcatggacacagaaccgggagaggaatgcacagaattcgcggagggggcatccgggacatccctccgccctgccacaacggtttcacagaaggcagcagagtatgatgctgtctggtcaaaatgggaaagggatgcaccagcaggagagtcacccggccgcgcagcagtggtacaggaaatgcgtgattgcctgaataacggcaatccagtgcttaacgtgggagcgtcaggtcttaccaccttaccagaccgtttaccaccgcatattacaacactggttattcctgataataatctgaccagcctgccggagttgccggaaggactacgggagctggaggtctctggtaacctacaactgaccagcctgccatcgctgccgcagggactacagaagctgtgggcctataataattggctggccagcctgccgacgttgccgccaggactaggggatctggcggtctctaataaccagctgaccagcctgccggagatgccgccagcactacgggagctgagggtctctggtaacaacctgaccagcctgccggcgctgccgtcaggactacagaagctgtgggcctataataatcggctgaccagcctgccggagatgtcgccaggactacaggagctggatgtctctcataaccagctgacccgcctgccgcaaagcctcacgggtctgtcttcagcggcacgcgtatatctggacgggaatccactgtctgtacgcactctgcaggctctgcgggacatcattggccattcaggcatcaggatacacttcgatatggcggggccttccgtcccccgggaagcccgggcactgcacctggcggtcgctgactggctgacgtctgcacgggagggggaagcggcccaggcagacagatggcaggcgttcggactggaagataacgccgccgccttcagcctggtcctggacagactgcgtgagacggaaaacttcaaaaaagacgcgggctttaaggcacagatatcatcctggctgacacaactggctgaagatgctgcgctgagagcaaaaacctttgccatggcaacagaggcaacatcaacctgcgaggaccgggtcacacatgccctgcaccagatgaataacgtacaactggtacataatgcagaaaaaggggaatacgacaacaatctccaggggctggtttccacggggcgtgagatgttccgcctggcaacactggaacagattgcccgggaaaaagccggaacactggctttagtcgatgacgttgaggtctatctggcgttccagaataagctgaaggaatcacttgagctgaccagcgtgacgtcagaaatgcgtttctttgacgtttccggcgtgacggtttcagaccttcaggctgcggagcttcaggtgaaaaccgctgaaaacagcgggttcagtaaatggatactgcagtgggggccgttacacagcgtgctggaacgcaaagtgccggaacgctttaacgcgcttcgtgaaaagcaaatatcggattatgaagacacgtaccggaagctgtatgacgaagtgctgaaatcgtccgggctggtcgacgataccgatgcagaacgtactatcggagtaagtgcgatggatagtgcgaaaaaagaatttctggatggcctgcgcgctcttgtggatgaggtgctgggtagctatctgacagcccggtggcgtcttaactaa

SEQ ID NO: 35 - protein, 788 aa, Artificial
  SspH2 novel E3 ligase from Salmonella typhimurium
MPFHIGSGCLPATISNRRIYRIAWSDTPPEMSSWEKMKEFFCSTHQTEALECIWTICHPPAGTTREDVINRFELLRTLAYAGWEESIHSGQHGENYFCILDEDSQEILSVTLDDAGNYTVNCQGYSETHRLTLDTAQGEEGTGHAEGASGTFRTSFLPATTAPQTPAEYDAVWSAWRRAAPAEESRGRAAVVQKMRACLNNGNAVLNVGESGLTTLPDCLPAHITTLVIPDNNLTSLPALPPELRTLEVSGNQLTSLPVLPPGLLELSIFSNPLTHLPALPSGLCKLWIFGNQLTSLPVLPPGLQELSVSDNQLASLPALPSELCKLWAYNNQLTSLPMLPSGLQELSVSDNQLASLPTLPSELYKLWAYNNRLTSLPALPSGLKELIVSGNRLTSLPVLPSELKELMVSGNRLTSLPMLPSGLLSLSVYRNQLTRLPESLIHLSSETTVNLEGNPLSERTLQALREITSAPGYSGPIIRFDMAGASAPRETRALHLAAADWLVPAREGEPAPADRWHMFGQEDNADAFSLFLDRLSETENFIKDAGFKAQISSWLAQLAEDEALRANTFAMATEATSSCEDRVTFFLHQMKNVQLVHNAEKGQYDNDLAALVATGREMFRLGKLEQIAREKVRTLALVDEIEVWLAYQNKLKKSLGLTSVTSEMRFFDVSGVTVTDLQDAELQVKAAEKSEFREWILQWGPLHRVLERKAPERVNALREKQISDYEETYRMLSDTELRPSGLVGNTDAERTIGARAMESAKKTFLDGLRPLVEEMLGSYLNVQWRRN

SEQ ID NO: 36 - DNA, 2367 bp, Artificial
  SspH2 novel E3 ligase from Salmonella typhimurium
atgccctttcatattggaagcggatgtcttcccgccaccatcagtaatcgccgcatttatcgtattgcctggtctgataccccccctgaaatgagttcctgggaaaaaatgaaggaatttttttgctcaacgcaccagactgaagcgctggagtgcatctggacgatttgtcacccgccggccggaacgacgcgggaggatgtgatcaacagatttgaactgctcaggacgctcgcgtatgccggatgggaggaaagcattcattccggccagcacggggaaaattacttctgtattctggatgaagacagtcaggagatattgtcagtcacccttgatgatgccgggaactataccgtaaattgccaggggtacagtgaaacacatcgcctcaccctggacacagcacagggtgaggagggcacaggacacgcggaaggggcatccgggacattcaggacatccttcctccctgccacaacggctccacagacgccagcagagtatgatgctgtctggtcagcgtggagaagggctgcacccgcagaagagtcacgcggccgtgcagcagtggtacagaaaatgcgtgcctgcctgaataatggcaatgcagtgcttaacgtgggagaatcaggtcttaccaccttgccagactgtttacccgcgcatattaccacactggttattcctgataataatctgaccagcctgccggcgctgccgccagaactgcggacgctggaggtctctggtaaccagctgactagcctgccggtgctgccgccaggactactggaactgtcgatctttagtaacccgctgacccacctgccggcgctgccgtcaggactatgtaagctgtggatctttggtaatcaactgaccagcctgccggtgttgccgccagggctacaggagctgtcggtatctgataaccaactggccagcctgccggcgctgccgtcagaattatgtaagctgtgggcctataataaccagctgaccagcctgccgatgttgccgtcagggctacaggagctgtcggtatctgataaccaactggccagcctgccgacgctgccgtcagaattatataagctgtgggcctataataatcggctgaccagcctgccggcgttgccgtcaggactgaaggagctgattgtatctggtaaccggctgaccagtctgccggtgctgccgtcagaactgaaggagctgatggtatctggtaaccggctgaccagcctgccgatgctgccgtcaggactactgtcgctgtcggtctatcgtaaccagctgacccgcctgccggaaagtctcattcatctgtcttcagagacaaccgtaaatctggaagggaacccactgtctgaacgtactttgcaggcgctgcgggagatcaccagcgcgcctggctattcaggccccataatacgattcgatatggcgggagcctccgccccccgggaaactcgggcactgcacctggcggccgctgactggctggtgcctgcccgggagggggaaccggctcctgcagacagatggcatatgttcggacaggaagataacgccgacgcattcagcctcttcctggacagactgagtgagacggaaaacttcataaaggacgcggggtttaaggcacagatatcgtcctggctggcacaactggctgaagatgaggcgttaagagcaaacacctttgctatggcaacagaggcaacctcaagctgcgaggaccgggtcacattttttttgcaccagatgaagaacgtacagctggtacataatgcagaaaaagggcaatacgataacgatctcgcggcgctggttgccacggggcgtgagatgttccgtctgggaaaactggaacagattgcccgggaaaaggtcagaacgctggctctcgttgatgaaattgaggtctggctggcgtatcagaataagctgaagaaatcactcgggctgaccagcgtgacgtcagaaatgcgtttctttgacgtatccggcgtgacggttacagaccttcaggacgcggagcttcaggtgaaagccgctgaaaaaagcgagttcagggagtggatactgcagtgggggccgttacacagagtgctggagcgcaaagcgccggaacgcgttaacgcgcttcgtgaaaagcaaatatcggattatgaggaaacgtaccggatgctgtctgacacagagctgagaccgtctgggctggtcggtaataccgatgcagagcgcactatcggagcaagagcgatggagagcgcgaaaaagacatttttggatggcctgcgacctcttgtggaggagatgctggggagctatctgaacgttcagtggcgtcgtaactga

SEQ ID NO: 37 - protein, 660 aa, Artificial
  XopL unconventional motif from Xanthomonas campestris
MRRVDQPRPPGTPFGLREQTTSNADAPARTAPPAHPAPERPTGMLGGLTRYVPGDRSGRPPAMPAAAETSRRPTTSARPLPYGGSGSAARMNEAAGHPLRMPQLPQLSDIERARFHSVTTDSQHLRPVRPRMPPPVGASPLRRSTALRPYHDVLSQWQRHYNADRNRWHSAWRQANSNNPQIETRTGRALKATADLLEDATQPGRVALELRSVPLPQFPDQAFRLSHLQHMTIDAAGLMELPDTMQQFAGLETLTLARNPLRALPASIASLNRLRELSIRACPELTELPEPLASTDASGEHQGLVNLQSLRLEWTGIRSLPASIANLQNLKSLKIRNSPLSALGPAIHHLPKLEELDLRGCTALRNYPPIFGGRAPLKRLILKDCSNLLTLPLDIHRLTQLEKLDLRGCVNLSRLPSLIAQLPANCIILVPPHLQAQLDQHRPVARPAEPGRTGPTTPALSPSAAGDRAGPSSSATASELLLTAALERIEDTAQAMLSTVIDEERNPFLEGAPSYLPGKRPTDVTTFGQVPALRDMLAESRDLEFLQRVSDMAGPSPRIEDPSEEGLARHYTNVSNWKAQKSAHLGIVDHLGQFVYHEGSPLDVATLAKAVQMWKTRELIVHAHPQDRARFPELAVHIPEQVSDDSDSEQQTSPEPSGHQ

SEQ ID NO: 38 - DNA, 1983 bp, Artificial
  XopL unconventional motif from Xanthomonas campestris
atgcgacgcgtcgatcaaccacgcccgccgggcacgcctttcggactgcgggagcagactacgtccaatgcggatgcgcccgcgcgcactgccccacccgcacaccccgcgcccgagcgccctaccggcatgctcggcggactgaccagatatgtgcctggcgatcggtccgggcgaccgccagcaatgcctgccgctgccgagacctctcgccggccaaccacctccgcccgcccgcttccctacggcggatccggcagcgccgcgcggatgaacgaggcggctggacatcctttgcggatgccgcaattgccacagctcagcgacatagaacgcgctcgcttccactccgtcaccaccgactcgcaacacttgcggccggtgcgcccccgtatgccaccgcccgtgggcgcttcacccttacggcgctccacagcgctgcgcccgtaccacgacgtgctgtcgcaatggcaacgccactacaacgcagatcgcaatcgctggcacagcgcatggcgccaggccaacagcaacaacccgcagatcgagactcgcacaggccgggcgctgaaggcgacagccgacctgctggaggacgcaacccaaccgggccgggtcgcgctggagctgcgctcagttccgctgccgcaatttcccgaccaggcattccgtctttcgcatctgcagcacatgacgatcgacgcggcagggttgatggagctcccggacaccatgcagcaatttgcgggcctggaaacactcacgctcgcacgcaatccgcttcgcgcgctaccggcatccatcgcaagcctcaaccgattacgcgagctctccatccgcgcctgcccggaattgacggaacttcccgaacccctggcaagcaccgatgcatccggcgagcaccagggcttggtcaacctgcagagcctacggctggaatggaccgggatcagatcgcttccggcgtccatcgccaacctgcaaaatctgaaaagcctgaagatacgcaactcgccgctgtccgcccttggcccggccatccatcacctgccaaagttggaggagcttgatttgcggggctgtaccgcgctgcgcaactatccgccgattttcggcggccgtgcgccactgaagcgactgattctgaaagactgcagcaacctgctcacgctgccactggacattcaccgcctgacgcagctggaaaaactcgatctgcgaggttgcgtcaacctttccagactgccctcgttgatcgcccaattacctgccaattgcatcatcctggtgccgccgcatctccaagcgcagctcgaccagcatcgtccagttgcgcgccccgccgaaccagggcggaccggaccgaccaccccagctctctcgccctctgctgccggcgaccgcgccgggccatcctcttcggcgaccgccagcgaactgcttcttaccgctgcgctcgaacgcatcgaagacaccgcacaggccatgctgagcacggtcatcgatgaagaaagaaatccctttctggaaggtgctccatcctatctcccaggaaaacgccctaccgatgtcaccaccttcggccaagttccggcattgcgggacatgctggcagaaagcagggatcttgagttcctgcaacgggtaagcgacatggcaggcccatcccccagaatcgaagacccgagcgaggaaggcctcgcccgccactacacgaacgtcagcaactggaaggcgcagaagagcgcacacctgggcatcgtcgatcatctcgggcagttcgtttatcacgaaggaagcccgctcgacgtagcgacattggccaaggcagtgcagatgtggaagacccgtgagctgatcgtccacgcacacccgcaagaccgcgcgcgctttcccgagctcgctgtgcacattcccgagcaggtcagcgacgactctgatagcgaacagcagacaagcccggaaccttcaggccatcagtag

(19) United States
(12) Patent Application Publication
(10) Pub. No.: US 2021/0017503 A1
(43) Pub. Date: Jan. 21, 2021

(54) BROAD-SPECTRUM PROTEOME EDITING WITH AN ENGINEERED BACTERIAL UBIQUITIN LIGASE MIMIC

(71) Applicants: Cornell University, Ithaca, NY (US); MASSACHUSETTS INSTITUTE OF TECHNOLOGY, Cambridge, MA (US)

(72) Inventors: Matthew P. DELISA, Ithaca, NY (US); Morgan B. LUDWICKI, Ithaca, NY (US); Paula T. HAMMOND, Newton, MA (US)

(21) Appl. No.: 16/981,626

(22) PCT Filed: Mar. 18, 2019
(86) PCT No.: PCT/US2019/022783

§ 371 Date: Sep. 16, 2020

Related U.S. Application Data
(60) Provisional application No. 62/644,055, filed on Mar. 16, 2018.

(51) Int. Cl. C12N 9/10; C07K 16/28; C12Q 1/48; G01N 33/50

(52) U.S. Cl. CPC C12N 9/104; C07K 16/2863; C12Y 203/02; C07K 2319/95; G01N 33/5005; C07K 2319/01; C12Q 1/48

(57) ABSTRACT

The present application relates to an isolated chimeric molecule comprising a degradation domain comprising an E3 ubiquitin ligase (E3) motif and a targeting domain capable of specifically directing the degradation domain to a substrate, where the targeting domain is heterologous to the degradation domain. A linker couples the degradation domain to the targeting domain. Also disclosed are compositions as well as methods of treating a disease, substrate silencing, screening agents for therapeutic efficacy against a disease, and methods of screening for disease biomarkers.

This application claims the priority benefit of U.S. Provisional Patent Application Ser. No. 62/644,055 filed Mar. 16, 2018, which is hereby incorporated by reference in its entirety.

### FIELD

The present application relates generally to broad-spectrum proteome editing with an engineered bacterial ubiquitin ligase mimic.

### BACKGROUND

Protein function has traditionally been investigated by disrupting the expression of a target gene encoding a protein and analyzing the resulting phenotypic consequences. Such loss-of-function experiments are now routinely performed using gene silencing and genome editing techniques such as antisense oligonucleotides (“ASOs”), RNA interference (“RNAi”), zinc finger nucleases (“ZFNs”), transcription activator-like effector nucleases (“TALENs”), and clustered, regularly interspaced, short palindromic repeat (“CRISPR”)-Cas systems. McManus et al., “Gene Silencing in Mammals by Small Interfering RNAs,” Nat. Rev. Genet. 3(10):737-47 (2002); Deleavey et al., “Designing Chemically Modified Oligonucleotides for Targeted Gene Silencing,” Chemistry & Biology 19(8):937-54 (2012); Boettcher et al., “Choosing the Right Tool for the Job: RNAi, TALEN, or CRISPR,” Mol. Cell 58(4):575-85 (2015); and Gaj et al., “TALEN, and CRISPR/Cas-Based Methods for Genome Engineering,” Trends Biotechnol. 31(7):397-405 (2013). These methods are widely used in basic research and hold promise for treating genetic disorders. Gaj et al., “TALEN, and CRISPR/Cas-Based Methods for Genome Engineering,” Trends Biotechnol. 31(7):397-405 (2013); Cox et al., “Therapeutic Genome Editing: Prospects and Challenges,” Nat. Med. 21(2):121-31 (2015); Soutschek et al., “Therapeutic Silencing of an Endogenous Gene by Systemic Administration of Modified siRNAs,” Nature 432(7014)173-78 (2004); Bumcrot et al., “RNAi Therapeutics: A Potential New Class of Pharmaceutical Drugs,” Nat. Chem. Biol. 2(12):711-19 (2006); and Wang et al., “Non-Viral Delivery of Genome-Editing Nucleases for Gene Therapy,” Gene Ther. 24(3):144-50 (2017). However, a number of challenges remain including: lack of temporal control, unpredictable off-target effects; the inability in the case of genome editing to remove essential genes and the irreversible nature of such knockouts, and the inability in the case of gene silencing to decrease levels of proteins already present within cells, thereby leaving stable, long-lived proteins unaffected.

Proteome editing technology represents an orthogonal approach for studying protein function that operates at the post-translational level and has the potential to dissect complicated protein functions at higher resolution than methods targeting DNA or RNA and with post-translational precision. One of the most notable methods involves “inhibition-by-degradation” whereby the machinery of the cellular ubiquitin-proteasome pathway (“UPP”) is hijacked to specifically degrade proteins of interest. The canonical ubiquitination cascade requires the activities of three enzymes—ubiquitin activating enzyme (E1), ubiquitin-conjugating enzymes (E2), and ubiquitin ligases (E3)—which act sequentially to tag proteins for degradation through the covalent attachment of a poly-ubiquitin chain to lysine residues in an energy-dependent manner.

E3s are the most heterogeneous class of enzymes in the UPP (there are >600 E3s in humans) and can be classified as HECT (homologous to E6AP C-terminus), RING (really interesting new gene), and RBR (RING-between-RING) depending on the presence of characteristic domains and on the mechanism of ubiquitin transfer to the substrate protein. Buetow et al., “Structural Insights into the Catalysis and Regulation of E3 Ubiquitin Ligases,” Nat. Rev. Mol. Cell Biol. 17(10):626-42 (2016). Because they mediate substrate specificity and generally exhibit remarkable plasticity, E3 ubiquitin ligases are the most frequently exploited component in proteome editing strategies described to date. For example, chemical knockdown has been achieved using small molecules called proteolysis targeting chimeras, or PROTACs (Neklesa et al., “Targeted Protein Degradation by PROTACs,” Pharmacol. Ther. 17:4138-144 (2017) and Deshaies, R. J., “Protein Degradation: Prime Time for PROTACs,” Nat. Chem. Biol. 11(9):634-35 (2015)), which are heterobifunctional molecules containing one ligand for an E3 ubiquitin ligase, another ligand for the protein to be degraded, and a linker connecting the two. These molecules bind to both the E3 and the target, promoting the formation of a ternary complex that triggers target polyubiquitination followed by its proteasomal degradation. A growing number of peptide- and small-molecule-based PROTACs have been reported that enable chemical knockout in cells and in mice. Schneekloth et al., “Chemical Genetic Control of Protein Levels: Selective In Vivo Targeted Degradation,” J. Am. Chem. Soc. 126(12):3748-54 (2004); Hines et al., “Posttranslational Protein Knockdown Coupled to Receptor Tyrosine Kinase Activation with PhosphoPROTACs,” Proc. Natl. Acad. Sci. USA 110(22):8942-47(2013); Schneekloth et al., “Targeted Intracellular Protein Degradation Induced by a Small Molecule: En Route to Chemical Proteomics,” Bioorganic Med. Chem. Lett. 18(22):5904-08 (2008); Bondeson et al., “Catalytic In Vivo Protein Knockdown by Small-Molecule PROTACs,” Nat. Chem. Biol. 11(8):611-17 (2015); and Sakamoto et al., “Protacs: Chimeric Molecules that Target Proteins to the Skp1-Cullin-F Box Complex for Ubiquitination and Degradation,” Proc. Natl. Acad. Sci. USA 98(15):8554-59 (2001). An attractive feature of these compounds is their drug-like properties including cell permeability; however, many peptide- and small-molecule-based PROTACs suffer from low potency—often requiring concentrations up to 25 μM to induce sufficient degradation (Buckley et al., “Small-Molecule Control of Intracellular Protein Levels Through Modulation of the Ubiquitin Proteasome System,” Angew Chem. Int. Ed. Engl. 53(9):2312-30 (2014))—and the generation of custom PROTACs is limited by the relative lack of available ligands for both E3 ubiquitin ligases and desired protein targets as well as the technical challenges associated with creating such ligands de novo (Osherovich, L., “Degradation From Within,” Science-Business Exchange 7:10-11 (2014)).

To circumvent these issues, protein-based chimeras have been developed where E3 ubiquitin ligases are genetically fused to a protein that binds the target of interest. Following ectopic expression in cells, the engineered protein chimera recruits the E3 to the target protein, leading to its polyubiquitination and subsequent degradation by the proteasome. In the earliest example, protein knockout was achieved by creating an F-box chimera in which β-TrCP was fused to a peptide derived from the E7 protein encoded by human papillomavirus type 16 that is known to interact with retinoblastoma protein pRB (Zhou et al., “Harnessing the Ubiquitination Machinery to Target the Degradation of Specific Cellular Proteins,” Mol. Cell 6(3):751-56 (2000) and Zhang et al., “Exploring the Functional Complexity of Cellular Proteins by Protein Knockout,” Proc. Natl. Acad. Sci. USA 100(24):14127-32 (2003)). Following ectopic expression, the engineered F-box recruited pRB to the Skp1-Cull-F-box (“SCF”) machinery, a multi-protein E3 complex from the cullin-RING ligase (“CRL”) superfamily, for ubiquitination and destruction. A handful of other studies have similarly leveraged natural protein-protein interactions, whereby fusion of one interacting protein to an E3 yielded a chimera that silenced the corresponding binding partner following expression in cells and mice. Hatakeyama et al., “Targeted Destruction of C-Myc by an Engineered Ubiquitin Ligase Suppresses Cell Transformation and Tumor Formation,” Cancer Res. 65(17):7874-79 (2005); Ma et al., “Targeted Degradation of KRAS by an Engineered Ubiquitin Ligase Suppresses Pancreatic Cancer Cell Growth In Vitro and In Vivo,” Mol. Cancer Ther. 12(3):286-94 (2013); and Kong et al., “Engineering a Single Ubiquitin Ligase for the Selective Degradation of all Activated ErbB Receptor Tyrosine Kinases,” Oncogene 33(8):986-95 (2014).

More recently, it was shown that a universal proteome editing technology could be extended beyond naturally occurring binding pairs. This approach involved fusing an E3 to a synthetic binding protein such as a single-chain antibody fragment (“scFv”), a designed ankyrin repeat protein (“DARPin”), or a fibronectin type III (“FN3”) monobody. Portnoff et al., “Ubiquibodies, Synthetic E3 Ubiquitin Ligases Endowed With Unnatural Substrate Specificity for Targeted Protein Silencing,” J Biol. Chem. 289(11):7844-55 (2014). These bifunctional chimeras, called “ubiquibodies” (“uAbs”), combined the flexible ubiquitin-tagging capacity of the human RING/U-box-type E3 CHIP (carboxyl terminus of Hsc70-interacting protein) with the engineerable affinity and specificity of synthetic binding proteins. The result is a customizable technology for efficiently directing otherwise stable proteins to the UPP for degradation independent of their biological function or interactions. Indeed, one of the greatest advantages of uAbs is their highly modular architecture—simply swapping synthetic binding proteins can generate a new uAb that specifically targets a different substrate protein (Caussinus et al., “Fluorescent Fusion Protein Knockout Mediated by Anti-GFP Nanobody,” Nat. Struct. Mol. Biol. 19(1):117-21 (2011); Fulcher et al., “Targeting Endogenous Proteins For Degradation Through the Affinity-Directed Protein Missile System,” Open Biol. 7(5):170066 (2017); Fulcher et al., “An Affinity-Directed Protein Missile System for Targeted Proteolysis,” Open Biol 6(10):160255 (2016); Shin et al., “Nanobody-Targeted E3-Ubiquitin Ligase Complex Degrades Nuclear Proteins,” Sci. Rep. 5:14269 (2015); and Kanner et al., “Sculpting Ion Channel Functional Expression with Engineered Ubiquitin Ligases,” Elife 6:e29744 (2017)) while swapping E3 domains can alter the kinetics or mechanism of ubiquitin transfer. Moreover, by incorporating synthetic binding proteins that recognize particular protein states (e.g., active vs. inactive conformation, mutant vs. wild-type, post-translationally modified), it becomes possible to deplete certain protein subpopulations while sparing others. Zhang et al., “Exploring the Functional Complexity of Cellular Proteins by Protein Knockout,” Proc. Natl. Acad. Sci. USA 100(24):14127-32 (2003) and Baltz et al., “Design and Functional Characterization of Synthetic E3 Ubiquitin Ligases for Targeted Protein Depletion,” Curr. Prot. Chem. Biol. 10(1):72-90 (2018). At present, however, the development of uAbs has centered around a relatively narrow set of mammalian E3 s, most notably the “stand alone” E3 CHIP or members of the CRL superfamily of multi-protein E3 ligase complexes.

The present application unites expertise in protein-based chimeras whereby novel E3 ubiquitin ligase motifs are genetically fused to a protein that binds the target of interest to address the above challenges and overcome these and other deficiencies in the art.

### SUMMARY

A first aspect of the present application relates to an isolated chimeric molecule. The isolated chimeric molecule comprises a degradation domain comprising an E3 ubiquitin ligase (E3) motif; a targeting domain capable of specifically directing the degradation domain to a substrate, wherein the targeting domain is heterologous to the degradation domain; and a linker coupling the degradation domain to the targeting domain.

A second aspect of the present application relates to a method of forming a ribonucleoprotein. The method includes providing a mRNA encoding the isolated chimeric molecule described herein; providing one or more polyadenosine binding proteins (“PABP”); and assembling a ribonucleoprotein complex from the mRNA and the one or more PABPs.

A third aspect of the present application relates to a composition comprising the chimeric molecule described herein and a pharmaceutically-acceptable carrier.

A fourth aspect of the present application relates to a method of treating a disease. The method includes selecting a subject having a disease and administering the composition described herein to the subject to give the subject an increased expression level of the substrate compared to a subject not afflicted with the disease.

A fifth aspect of the present application relates to a method for substrate silencing. The method includes selecting a substrate to be silenced; providing the chimeric molecule described herein; and contacting the substrate with the chimeric molecule under conditions effective to permit the formation of a substrate-molecule complex, wherein the complex mediates the degradation of the substrate to be silenced.

A sixth aspect of the present application relates to a method of screening agents for therapeutic efficacy against a disease. The method includes providing a biomolecule whose presence mediates a disease state; providing a test agent comprising (i) a degradation domain comprising an E3 ubiquitin ligase (E3) motif, (ii) a targeting domain capable of specifically directing the degradation domain to the biomolecule, wherein the targeting domain is heterologous to the degradation domain, and (iii) a linker coupling the degradation domain to the targeting domain; contacting the biomolecule with the test agent under conditions effective for the test agent to facilitate degradation of the biomolecule; determining the level of the biomolecule as a result of the contacting; and identifying the test agent which, based on the determining, decreases the level of the biomolecule as being a candidate for therapeutic efficacy against the disease.

A seventh aspect of the present application relates to a method of screening for disease biomarkers. The method includes providing a sample of diseased cells expressing one or more ligands; providing a plurality of chimeric molecules comprising (i) a degradation domain comprising an E3 ubiquitin ligase (E3) motif, (ii) a targeting domain capable of specifically directing the degradation domain to the one or more ligands, wherein the targeting domain is heterologous to the degradation domain, and (iii) a linker coupling the degradation domain to the targeting domain; contacting the sample with the plurality of chimeric molecules under conditions effective for the diseased cells to fail to proliferate in the absence of the chimeric molecule; determining which of the chimeric molecules permit the diseased cells to proliferate; and identifying, as biomarkers for the disease, based on the determining the ligands which bind to the chimeric molecules and permit diseased cells to proliferate.

Manipulation of the ubiquitin-proteasome pathway to achieve targeted silencing of cellular proteins has emerged as a reliable and customizable strategy for remodeling the mammalian proteome. One such approach involves engineering bifunctional proteins called ubiquibodies that are comprised of a synthetic binding protein fused to an E3 ubiquitin ligase, thus enabling post-translational ubiquitination and degradation of a target protein independent of its function. Here, a panel of new ubiquibodies was designed based on E3 ubiquitin ligase mimics from bacterial pathogens that are capable of effectively interfacing with the mammalian proteasomal degradation machinery for selective removal of proteins of interest. One of these, the Shigella flexneri effector protein IpaH9.8 fused to a fibronectin type III (FN3) monobody that specifically recognizes green fluorescent protein (GFP), was observed to potently eliminate GFP and its spectral derivatives as well as 15 different FP-tagged mammalian proteins that varied in size (27-179 kDa) and subcellular localization (cytoplasm, nucleus, membrane-associated, and transmembrane). To demonstrate therapeutically-relevant delivery of ubiquibodies, a bioinspired molecular assembly method was leveraged whereby synthetic mRNA encoding the GFP-specific ubiquibody was co-assembled with poly A binding proteins and packaged into nanosized complexes using biocompatible, structurally defined polypolypeptides bearing cationic amine side groups. The resulting nanoplexes delivered ubiquibody mRNA in a manner that caused efficient target depletion in cultured mammalian cells stably expressing GFP as well as in transgenic mice expressing GFP ubiquitously. Overall, the results presented here suggest that IpaH9.8-based ubiquibodies are a highly modular proteome editing technology with the potential for pharmacologically modulating disease-causing proteins.

The present application thus relates to chimeric molecules, compositions, treatments, pharmaceutical compositions, protein silencing techniques, the elucidation of therapeutic agents, and target screening technologies based on a novel class of chimeric molecules. Such chimeras, termed “ubiquibodies” herein, import the ligase function of an E3 ubiquitin enzyme to generate a molecule possessing target specificity. Such engineered chimeras facilitate the redirection and proteolytic degradation of specific substrate targets, which may not otherwise be bound for the proteasome.

In this respect, the targeted elimination of such specific substrates, e.g., intracellular proteins, ascribes a broad range of scientific and clinical indications to the chimeric molecules, compositions, treatments, pharmaceutical compositions, protein silencing techniques, elucidation of therapeutic agents, and screening technologies provided herein. The present application therefore imparts a variety of valuable tools for employing and developing specific prognostic and therapeutic applications based on the proteolytic degradation of aberrantly expressed genes via ubiquitination.

Here, the range of E3s that can be functionally reprogrammed as bifunctional uAb chimeras was sought to be broadened. However, in a notable departure from previous efforts involving mammalian E3s, the focus was instead on a set of effector proteins from microbial pathogens that mimic host E3 ubiquitin ligases and hijack the UPP machinery to dampen the innate immune response during infection. Maculins et al., “Bacteria-Host Relationship: Ubiquitin Ligases as Weapons of Invasion. Cell Res. 26(4):499-510 (2016) and Lin et al., “Exploitation of the Host Cell Ubiquitin Machinery by Microbial Effector Proteins,” J. Cell Sci. 130(12):1985-96 (2017), which are hereby incorporated by reference in their entirety. The intrinsic plasticity of these enzymes led us to hypothesize that bacterial E3s could be manipulated for targeted proteolysis just like their mammalian counterparts. Indeed, robust target silencing was achieved with a uAb comprised of the Shigella flexneri E3 ligase IpaH9.8, which exhibits similarities to eukaryotic HECT-type E3s but is classified as a novel E3 ligase (“NEL”) due to the absence of sequence and structural homology with any eukaryotic E3s. Maculins et al., “Bacteria-Host Relationship: Ubiquitin Ligases as Weapons of Invasion. Cell Res. 26(4):499-510 (2016); Lin et al., “Exploitation of the Host Cell Ubiquitin Machinery by Microbial Effector Proteins,” J. Cell Sci. 130(12):1985-96 (2017); Zhu et al., “Structure of a Shigella Effector Reveals a New Class of Ubiquitin Ligases,” Nat. Struct. Mol. Biol. 15(12):1302-08 (2008); Singer et al., “A Pathogen Type III Effector With a Novel E3 Ubiquitin Ligase Architecture,” PLoS Pathogens 9(1):e1003121 (2013); and Rohde et al., “Type III Secretion Effectors of the IpaH Family are E3 Ubiquitin Ligases,” Cell Host Microbe 1(1):77-83 (2007), which are hereby incorporated by reference in their entirety. When the C-terminal catalytic NEL domain of IpaH9.8 was fused to the GFP-specific FN3 monobody GS2 that specifically recognizes green fluorescent protein (“GFP”), potent degradation of EGFP following both transient and stable expression in cultured mammalian cells was observed. Moreover, the GS2-IpaH9.8 chimera was also able to accelerate the degradation of spectral derivatives of EGFP including Emerald, Venus and Cerulean as well as 15 different FP-tagged mammalian proteins that ranged in size from 27 up to 179 kDa and localized in different subcellular compartments including the cytoplasm, nucleus, and cell membrane. For two of these targets, SHP2 and Ras, efficient silencing was also achieved when IpaH9.8 was fused to SHP2- or Ras-specific FN3 domains, highlighting the ease with which IpaH-based uAbs can be reconfigured.

As was noted previously, a major obstacle for the therapeutic development of uAbs is intracellular delivery. Osherovich, L., “Degradation From Within,” Science-Business Exchange 7:10-11 (2014), which is hereby incorporated by reference in its entirety. Unlike smaller PROTACs, which can be designed for cell-permeability (Buckley et al., “Small-Molecule Control of Intracellular Protein Levels Through Modulation of the Ubiquitin Proteasome System,” Angew Chem. Int. Ed. Engl. 53(9):2312-30 (2014), which is hereby incorporated by reference in its entirety), uAbs are relatively bulky proteins that do not effectively penetrate the cell membrane. To remedy this issue, a bioinspired mRNA delivery strategy was implemented whereby mRNA encoding GS2-IpaH with an additional 3′-terminal polyadenosine (“poly A”) tail was stoichiometrically complexed with poly A binding proteins (“PABPs”), which served to improve mRNA stability and also stimulate mRNA translation in eukaryotic cells (Li et al., “Polyamine-Mediated Stoichiometric Assembly of Ribonucleoproteins for Enhanced mRNA Delivery,” Angew Chem. Int. Ed. Engl. 56(44):13709-12 (2017), which is hereby incorporated by reference in its entirety). The resulting ribonucleoproteins (“RNPs”) were stabilized with cationic polypeptides to protect the mRNA from degradation, enable its uptake by cells, and facilitate its endosomal escape. Importantly, these co-assembled nanoplexes delivered GS2-IpaH9.8 mRNA in a manner that caused efficient GFP silencing after introduction to cultured mammalian cells stably expressing GFP and after administration to transgenic mice expressing GFP ubiquitously. Collectively, the results described herein demonstrate that uAb-mediated proteome editing is an effective strategy for targeted degradation of proteins in cells and mice, thereby setting the stage for uAbs as tools for drug discovery and as therapeutic candidates with potential to pharmacologically hit so-called “undruggable” targets.

### BRIEF DESCRIPTION OF THE DRAWINGS

FIGS. 1A-1C depict the engineering of bacterial E3 ligase IpaH9.8 as a GFP-specific ubiquibody. FIG. 1A shows linear representation of IpaH9.8, IpaH9.8ΔLRR, and GS2-IpaH9.8. Numbers refer to amino acid positions from N terminus (“N”) to C terminus (“C”). The proteins are aligned vertically with the LRR and NEL domains of IpaH9.8. IpaH9.8ΔLRR is a truncated version of IpaH9.8 lacking the LRR domain. FIG. 1B shows flow cytometric analysis of EGFP fluorescence activity in HEK293T cells transfected with plasmid pcDNA3-EGFP alone or co-transfected with pcDNA3-EGFP and a plasmid encoding one of the bacterial E3-based uAbs as indicated. FIG. 1C is the same as in 1C but with mammalian E3-based uAbs as indicated. Data are biological triplicates of the geometric mean fluorescence intensity (“NM”) normalized to MFI measured for HEK283T cells expressing EGFP alone. Error bars represent standard deviation (“SD”) of the mean.

FIGS. 2A-2D illustrate that the catalytic domain of IpaH9.8 is essential for ubiquibody function. FIG. 2A shows representative fluorescence histograms obtained by flow cytometric analysis of EGFP fluorescence activity in HEK293T cells transfected with pcDNA3-EGFP alone or co-transfected with pcDNA3-EGFP and a plasmid encoding one of the following: GS2-IpaH9.8^{C337A}, AS15-IpaH9.8, or GS2-IpaH9.8. FIG. 2B shows flow cytometric quantification of EGFP fluorescence activity for cells described in FIG. 2A where data are biological triplicates of the geometric MFI normalized to WI measured for HEK283T cells expressing EGFP alone. Error bars represent standard deviation (“SD”) of the mean. FIG. 2C depicts a western blot analysis of HEK293T cell lysates transfected as in FIGS. 2A and 2B. Blots were probed with antibodies specific for GFP, 6×-His (that detected tag on each uAb), and GAPDH as indicated. An equivalent amount of total protein was loaded in each lane as confirmed by immunoblotting with anti-GAPDH. Molecular weight (MW) markers are indicated on left. FIG. 2D depicts flow cytometric quantification of EGFP fluorescence activity for HEK293T cells co-transfected with pcDNA3-EGFP and a plasmid encoding GS2 fused to one of the IpaH9.8 homologs as indicated. Data are biological triplicates of the geometric MFI normalized to MFI measured for HEK283T cells expressing EGFP alone. Error bars represent standard deviation (“SD”) of the mean.

FIGS. 3A-3B show that GS2-IpaH9.8 degrades structurally diverse fluorescent protein fusions. FIG. 3A depicts flow cytometric quantification of fluorescence activity in HEK293T cells transfected with a plasmid encoding the indicated FP fusion alone (dark grey) or co-transfected with the FP fusion plasmid and either pcDNA3-GS2-IpaH9.8^{C337A} (white) or pcDNA3-GS2-IpaH9.8 (light grey). Data are biological triplicates of the geometric WI normalized to MFI measured for HEK283T cells expressing the corresponding FP fusion protein alone. Error bars represent standard deviation (“SD”) of the mean. FIG. 3B shows confocal microscopy images corresponding to select FP targets expressed in HEK293T cells transfected/co-transfected as described in FIG. 3A. Hoescht stain (blue) denotes cell nuclei and EGFP signal (green) denotes fluorescent proteins. For the EGFR-mEmerald fusion, immunostaining with an EGFR-specific antibody (red) is also depicted.

FIGS. 4A-4C depict IpaH9.8 ubiquibodies directed against disease-relevant targets. FIG. 4A illustrates flow cytometric quantification of EGFP fluorescence activity in HEK293T cells transfected with pcDNA3-SHP2-EGFP alone or co-transfected with pcDNA3-SHP2-EGFP and a plasmid encoding one of the following: GS2-IpaH9.8, GS2-IpaH9.8^{C337A}, NSa5-IpaH9.8, or NSa5-IpaH9.8^{C337A}. FIG. 4B is the same as in FIG. 4A but with pcDNA3-EGFP-HRas^{G12V} alone or co-transfected with pcDNA3-EGFP-HRas^{G12V} and a plasmid encoding one of the following: GS2-IpaH9.8, GS2-IpaH9.8^{C337A}, RasInII-IpaH9.8, or RasInII-IpaH9.8^{C337A}. Data are biological triplicates of the geometric MFI normalized to MFI measured for HEK283T cells expressing EGFP alone. Error bars represent standard deviation (“SD”) of the mean. FIG. 4C shows flow cytometric quantification of EGFP fluorescence activity in HEK293T cells co-transfected with pcDNA3-RasInII-IpaH9.8 and one of the following: pcDNA3-EGFP-KRas, pcDNA3-EGFP-KRas^{G12C}, pcDNA3-EGFP-KRas^{G12D}, or pcDNA3-EGFP-KRas^{G12V}. MFI ratio was determined by normalizing geometric MFI for cells expressing KRas mutant to geometric MFI for cells expressing wild-type (wt) KRas. Data are the average of biological triplicates and error bars represent standard deviation (SD) of the mean.

FIGS. 5A-5D depict proteome editing in mice via nanoplex delivery of ubiquibody mRNA. FIG. 5A is a schematic of polyamine (TEP (N4))-mediated stoichiometric assembly of mRNA/PABP ribonucleoproteins for enhanced mRNA delivery. Following internalization in cells (grey circle), nanoplex disassembly results in the release of mRNA/PABP that is either degraded or translated to produce uAb proteins. FIG. 5B depicts flow cytometric quantification of EGFP fluorescence activity in HEK293Td2EGFP cells incubated with: mRNA encoding GS2-IpaH9.8, GS2-IpaH9.8^{C337A}, or AS15-IpaH9.8; or with nanoplexes comprised of the same mRNAs formulated with PABP and TEP (N4) polyamine (mRNA:PABP weight ratio=1:5). Measurements were taken at 24, 48, and 72 h post-delivery. Data are expressed as the mean S.D. of biological triplicates. FIG. 5C shows epifluorescence imaging of UBC-GFP mice at 0 h (top) and 24 h (bottom) after ear injection of nanoplexes containing mRNA encoding GS2-IpaH9.8 (solid white circle), GS2-IpaH9.8^{C337A} (dashed white circle, top), or AS15-IpaH9.8 (dashed white circle, bottom). Numbers on the heat bar represent radiant efficiency (p/sec/cm²/sr)/(μW/cm²). FIG. 5D depicts quantification of GFP fluorescence in the ears of Ubi-GFP mice in FIG. 5C. Data are reported as the mean radiant efficiency for each individual ear region (black circle) and the mean radiant efficiency (red bar) of each sample group (n=6 for GS2-IpaH9.8, n=3 for GS2-IpaH9.8^{C337A}, and n=3 for AS15-IpaH9.8). p values were determined by paired sample t-test.

FIGS. 6A-6B depict GFP silencing by uAbs harboring bacterial and mammalian E3 ubiquitin ligase domains. Representative fluorescence histograms obtained by flow cytometric analysis of EGFP fluorescence activity in HEK293T cells transfected with pcDNA3-EGFP alone or co-transfected with pcDNA3-EGFP and a plasmid encoding a uAb comprised of GS2 fused to one of the (as shown in FIG. 6A) bacterial or (as shown in FIG. 6B) mammalian E3 ubiquitin ligases as indicated. Values for geometric mean fluorescence intensity (“MFI”) are shown.

FIGS. 7A-7B show characterization of GS2-IpaH9.8 binding activity and expression. In FIG. 7A, binding activity of GS2-IpaH9.8 is shown compared to GS2 alone, IpaH9.8 lacking the LRR domain (“IpaH9.8 LRR”), or catalytically inactive GS2-IpaH9.8^{C337A} as indicated. Activity was measured by ELISA using GFP as immobilized antigen and 15 mg/mL of each protein applied per well. Detection was performed using anti-FLAG antibody conjugated to horseradish peroxidase (HRP). The quenched plate was read at 450 nm (Abs₄₅₀). Data is the average of three biological replicates and error bars are the standard deviation (“SD”) of the mean. FIG. 7B shows confocal microscopy images corresponding to HEK293T cells transfected with plasmid DNA encoding EGFP or co-transfected with plasmid DNA encoding EGFP and either pcDNA3-GS2-IpaH9.8C337A or pcDNA3-GS2-IpaH9.8 as indicated. Non-transfected HEK293T control cells are also depicted. Hoescht stain (blue) denotes cell nuclei, EGFP signal (green) denotes FP target expression, and -His signal (red) corresponds to immunolabeling of expressed uAb in permeabilized cells.

FIGS. 8A-8B depict uAb-mediated silencing of FP variants and additional FP fusion protein targets. FIG. 8A shows flow cytometric quantification of fluorescence activity in HEK293T cells co-transfected with plasmids encoding the FP variant and either pcDNA3-GS2-IpaH9.8C337A (white) or pcDNA3-GS2-IpaH9.8 (grey) as indicated. mCherry served as negative control. Data are biological triplicates of the geometric MFI normalized to MFI measured for HEK283T cells expressing the corresponding FP alone. Error bars represent standard deviation (SD) of the mean. FIG. 8B shows flow cytometric quantification of fluorescence activity in HEK293T cells transfected with a plasmid encoding the indicated FP fusion alone (dark grey) or co-transfected with the FP fusion plasmid and either pcDNA3-GS2-IpaH9.8C337A (white) or pcDNA3-GS2-IpaH9.8 (light grey). Data are biological triplicates of the geometric MFI normalized to MFI measured for HEK283T cells expressing the corresponding FP alone. Error bars represent standard deviation (SD) of the mean.

FIGS. 9A-9C illustrate modularity of the uAb platform. FIG. 9A shows flow cytometric quantification of EGFP fluorescence activity in HEK293T cells transfected with plasmid DNA encoding EGFP or co-transfected with a plasmid encoding uAb chimeras comprised of IpaH9.8 fused to a different GFP-directed binding protein as indicated. FIG. 9B shows flow cytometric quantification of EGFP fluorescence activity in HEK293T cells that transiently or stably expressed EGFP, ERK2-EGFP, H2B-EGFP, or EGFPHRasG12V as indicated. In all cases, cells were transiently transfected with plasmid DNA encoding either pcDNA3-GS2-IpaH9.8C337A (white) or pcDNA3-GS2-IpaH9.8 (grey). FIG. 9C shows flow cytometric quantification of EGFP fluorescence activity in MCF10a cells stably integrated with DNA encoding only EGFP-HRasG12V, EGFP-HRasG12V and GS2-IpaH9.8, EGFPHRasG12V and GS2-IpaH9.8C337A, or GS2-IpaH9.8 alone. All data are biological triplicates of the geometric MFI normalized to MFI measured for HEK283T cells expressing the EGFP alone. Error bars represent standard deviation (SD) of the mean.

### DETAILED DESCRIPTION

It is to be appreciated that certain aspects, modes, embodiments, variations and features of the present application are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which the present application belongs.

In practicing the subject matter of the present application, many conventional techniques in molecular biology, protein biochemistry, cell biology, immunology, microbiology and recombinant DNA are used. These techniques are well-known and are explained in, e.g., Current Protocols in Molecular Biology, Vols. I-III, Ausubel, Ed. (1997); Sambrook et al., Molecular Cloning: A Laboratory Manual, Second Ed. (Cold Spring Harbor Laboratory Press, Cold Spring Harbor, N.Y. (1989)); DNA Cloning: A Practical Approach, Vols. I and II, Glover, Ed. (1985); Oligonucleotide Synthesis, Gait, Ed. (1984); Nucleic Acid Hybridization, Hames & Higgins, Eds. (1985); Transcription and Translation, Hames & Higgins, Eds. (1984); Animal Cell Culture, Freshney, Ed. (1986); Immobilized Cells and Enzymes (IRL Press, 1986); Perbal, A Practical Guide to Molecular Cloning; the series, Meth. Enzymol., (Academic Press, Inc., 1984); Gene Transfer Vectors for Mammalian Cells, Miller & Calos, Eds. (Cold Spring Harbor Laboratory, New York (1987)); and Meth. Enzymol., Vols. 154 and 155, Wu & Grossman, and Wu, Eds., respectively, all of which are hereby incorporated by reference in their entirety. Methods to detect and measure levels of polypeptide gene expression products, i.e., gene translation level, are well-known in the art and include the use polypeptide detection methods such as antibody detection and quantification techniques. See also, Strachan & Read, Human Molecular Genetics, Second Edition. (John Wiley and Sons, Inc., New York (1999), which is hereby incorporated by reference in its entirety.

A first aspect of the present application relates to an isolated chimeric molecule. The isolated chimeric molecule comprises a degradation domain comprising an E3 ubiquitin ligase (E3) motif; a targeting domain capable of specifically directing the degradation domain to a substrate, wherein the targeting domain is heterologous to the degradation domain; and a linker coupling the degradation domain to the targeting domain.

As used herein, the terms “chimeric molecule”, “chimeric polypeptide”, and “chimeric protein” encompass a molecule having a sequence that includes at least a portion of a full-length sequence of first protein or polypeptide sequence and at least a portion of a full-length sequence of a second protein or polypeptide sequence, where the first and second proteins or polypeptides are different proteins or polypeptides. A chimeric molecule also encompasses proteins or polypeptides that include two or more non-contiguous portions derived from the same protein or polypeptide. A chimeric molecule also encompasses proteins or polypeptides having at least one substitution, wherein the chimeric molecule includes a first protein or polypeptide sequence in which a portion of the first protein or polypeptide sequence has been substituted by a portion of a second protein or polypeptide sequence. As used herein, the term “chimeric molecule” further refers to a molecule possessing a degradation domain and a targeting region, as exemplified herein. The degradation domain and targeting region may be attached in manner known in the art. For example, they may be linked via linker molecule as exemplified herein, fused, covalently attached, non-covalently attached, etc. Moreover, the degradation domain and a targeting region may not be directly attached and/or the attachment may be transient, e.g., if a linker is used, the linker may be cleavable or non-cleavable.

As used herein, the term “ubiquitination” refers to the attachment of the protein ubiquitin to lysine residues of other molecules. Ubiquitination of a molecule, such as a peptide or protein, can act as a signal for its rapid cellular degradation, and for targeting to the proteasome complex.

As used herein, the terms “ubiquibodies” and “chimeric molecules” are used interchangeably and refer to molecules with at least a degradation domain and a target region, linked by a linker region, as exemplified herein.

As used herein the terms “target domain” or “targeting domain” or “targeting moiety” means a polypeptide region bound covalently or non-covalently to a second region within a chimeric molecule, which enhances the concentration of the chimeric molecule or composition in a target sub-cellular location, cell, or tissue relative, as compared to the surrounding locations, cells, and/or tissue.

In accord, the chimeric molecules of the present application possess novel E3 ligase motif (referenced herein, for example, as “E3 ligase (EL)”) ubiquitin regions attached to targeting domains, which are accessible for substrate binding. In some embodiments, the substrate is an intracellular substrate. In order to facilitate versatility, however, the targeting domain is derived from a monobody (for example, fibronectin type III domain (“FN3”)), antibody, polyclonal antibody, monoclonal antibody, recombinant antibody, antibody fragment, Fab′, F(ab′)2, Fv, scFv, tascFvs, bis-scFvs, sdAb, V_{H}, V_{L}, Vₙₐᵣ, scFvD10, scFv13R4, scFvD10, humanized antibody, chimeric antibody, complementary determining region (CDR), IgA antibody, IgD antibody, IgE antibody, IgG antibody, IgM antibody, nanobody, intrabody, unibody, minibody, PROTACs, aptameric domains, a ubiquitin binding domain sequence, an E3 binding domain, a non-antibody protein scaffold, Adnectin, Affibody and their two-helix variants, Anticalin, camelid antibody (for example, V_{H}H), knottin, DARPin, and/or Sso7d.

The skilled artisan will readily appreciate that such targeting domains, in some embodiments, possess cell/tissue specificity in accord with the novel E3 ligase motif regions described herein. In one embodiment, the targeting domain binds to a non-native substrate.

As used herein the term monobody may include any binding portion of an non-immunoglobulin molecule including, for example, FN3 and DARPins, or a polypeptide that contains a binding site, which specifically binds to, or reacts with, a substrate and the like. Monobodies in accordance with the present application include synthetic binding proteins that are constructed using a fibronectin type III domain (“FN3”) as a molecular scaffold. Monobodies are a simple and robust alternative to antibodies for creating target-binding proteins. Monobodies belong to a class of molecules collectively called antibody mimics (or antibody mimetics) and alternative scaffolds that aim to overcome shortcomings of natural antibody molecules. A major advantage of monobodies over conventional antibodies is that monobodies can readily be used as genetically encoded intracellular inhibitors, that is a monobody inhibitor may be expressed in a cell of choice by transfecting the cell with a monobody expression vector. This is attributed to the underlying characteristics of the FN3 scaffold: small (˜90 residues), stable, easy to produce, and its lack of disulfide bonds that makes it possible to produce functional monobodies regardless of the redox potential of the cellular environment, including the reducing environment of the cytoplasm and nucleus. In one embodiment, the targeting domain is a monobody. The monobody may be a fibronectin type III domain (FN3) monobody selected from the group consisting of GS2, Nsa5, and RasInII. The GS2 monobody may, for example, recognize green fluorescent protein (“GFP”). The NSa5 monobody may, for example, be specific for the Src-homology 2 (SH2) domain of SHP2 (Sha et al., “Dissection of the BCR-ABL Signaling Network Using Highly Specific Monobody Inhibitors to the SHP2 SH2 Domains,” Proc. Natl. Acad. Sci. USA 110(37):14924-29 (2013), which is hereby incorporated by reference in its entirety) and RasInII, which is specific for HRas, KRas, and the G12V mutants of each (Cetin et al., “RasIns: Genetically Encoded Intrabodies of Activated Ras Proteins,” J. Mol. Biol. 429(4):562-573 (2017), which is hereby incorporated by reference in its entirety).

A targeting domain that is a monobody may be, for example, a fibronectin type III domain (FN3) monobody. Examples of FN3 monobodies include but are not limited to (with target antigen in parenthesis): GS2 (GFP), Nsa5 (SHP2), RasInI (HRas/KRas), and RasInII (HRas/KRas), 1D10 (CDC34), 1D7 (COPS5), 1C4 (MAP2K5), 2C12 (MAP2K5), 1E2 (SF3A1), 1C2 (USP11), 1A9 (USP11), Ubi4 (ubiquitin), EI1.4.1 (EGFR), EI2.4.6 (EGFR), EI3.4.3 (EGFR), EI4.2.1 (EGFR), EI4.4.2 (EGFR), EI6.2.6 (EGFR), EI6.2.10 (EGFR), E246(EGFR), C743(CEA), IIIa8.2.6 (FcγIIa), IIIa6.2.6 (FcγIIIa), hA2.2.1 (hA33), hA2.2.2 (hA33), hA3.2.1 (hA33), hA3.2.3 (hA33), mA3.2.1 (mA33), mA3.2.2 (mA33), mA3.2.3 (mA33), mA3.2.4 (mA33), mA3.2.5 (mA33), Alb3.2.1 (hAlb), mI2.2.1 (mIgG), HA4 (AblSH2), HA10 (AblSH2), HA16 (AblSH2), HA18 (AblSH2), 159 (vEGFR), MUC16 (MSLN), E2#3 (ERα/EF), E2#4 (ERα/EF), E2#5 (ERα/EF), E2#6 (ERα/EF), E2#7 (ERα/EF), E2#8 (ERα/EF), E2#9 (ERα/EF), E2#10 (ERα/EF), E2#11 (ERα/EF), E2#23 (ERα/EF), E3#2 (ERα/EF), E3#6 (ERα/EF), OHT#31 (ERα/EF), OHT#32 (ERα/EF), OHT#33 (ERα/EF), AB7-A1 (ERα/EF), AB7-B1 (ERα/EF), MBP-74 (MBP), MBP-76 (MBP), MBP-79 (MBP), hSUMO4-33 (hSUMO4), hSUMO-39 (hSUMO4), ySUMO-53 (ySUMO), ySUMO-56 (ySUMO), ySUMO-57 (ySUMO), T14.25 (TNFα), T14.20 (TNFα), FNfn10-3JCL14 (avβ3 integrin), 1C9 (Src SH3), 1F11 (Src SH3), 1F10 (Src SH3), 2G10 (Src SH3), 2B2 (Src SH3), 1E3 (Src SH3), E18 (VEGFR2), E19 (VEGFR2), E26 (VEGFR2), E29 (VEGFR2), FG4.2 (Lysozyme), FG4.1 (Lysozyme), 2L4.1 (Lysozyme), BF4.1 (Lysozyme), BF4.9 (Lysozyme), BF4.4 (Lysozyme), BFs1c4.01 (Lysozyme), BFs1c4.07 (Lysozyme), BFs3_4.02 (Lysozyme), BFs3_4.06 (Lysozyme), BFs3_8.01 (Lysozyme), 10C17C25 (phospho-IκBα), Fn-N22 (SARS N), Fn-N17 (SARS N), FN-N10 (SARS N), gI2.5.3T88I (goat IgG), gI2.5.2 (goat IgG), gI2.5.4 (goat IgG), rI4.5.4 (rabbit IgG), rI4.3.1 (rabbit IgG), rI3.6.6 (rabbit IgG), rI4.3.4 (rabbit IgG), rI3.6.4 (rabbit IgG), and rI4.3.3 (rabbit IgG).

As used herein the term antibody may include an immunoglobulin and any antigen-binding portion of an immunoglobulin, e.g., IgG, IgD, IgA, IgM and IgE, or a polypeptide that contains an antigen binding site, which specifically or “immunospecifically binds” to, or “immunoreacts with”, an immunogen, antigen, substrate, and the like. Antibodies can comprise at least one heavy (H) chain and at least one light (L) chain inter-connected by at least one disulfide bond. The term “V_{H}” refers to a heavy chain variable region of an antibody. The term “V_{L}” refers to a light chain variable region of an antibody. In some embodiments, the term “antibody” specifically covers monoclonal and polyclonal antibodies. A “polyclonal antibody” refers to an antibody which has been derived from the sera of animals immunized with an antigen or antigens. A “monoclonal antibody” refers to an antibody produced by a single clone of hybridoma cells.

Antibody-related molecules, domains, fragments, portions, etc., useful as targeting domains of the present application include, e.g., but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. Examples include: (i) a Fab fragment, a monovalent fragment consisting of the V_{L}, V_{H}, C_{L} and CH₁ domains; (ii) a F(ab′)₂ fragment, a bivalent fragment comprising two Fab fragments linked by a disulfide bridge at the hinge region; (iii) a Fd fragment consisting of the V_{H} and CH₁ domains; (iv) a Fv fragment consisting of the V_{L} and V_{H} domains of a single arm of an antibody, (v) a dAb fragment (Ward et al., “Binding Activities of a Repertoire of Single Immunoglobulin Variable Domains Secreted From Escherichia coli,” Nature 341:544-46 (1989), which is hereby incorporated by reference in its entirety), which consists of a V_{H} domain; and (vi) an isolated complementary determining region (CDR). As such “antibody fragments” can comprise a portion of a full length antibody, generally the antigen binding or variable region thereof. Examples of antibody fragments include Fab, Fab′, F(ab′)₂, and Fv fragments; diabodies; linear antibodies; single-chain antibody molecules; and multispecific antibodies formed from antibody fragments. Single-chain antibody molecules may comprise a polymer with a number of individual molecules, for example, dimer, trimer or other polymers.

The term “monoclonal antibody” as used herein may include an antibody obtained from a population of substantially homogeneous antibodies, i.e., the individual antibodies comprising the population are identical except for possible naturally occurring mutations that may be present in minor amounts. Nevertheless, the monoclonal antibodies to be used in accordance with the present application may be made by the hybridoma method first described by Kohler et al., “Continuous Cultures of Fused Cells Secreting Antibody of Predefined Specificity,” Nature 256:495 (1975), which is hereby incorporated by reference in its entirety, or may be made by recombinant DNA methods. See, e.g., U.S. Pat. No. 4,816,567, which is hereby incorporated by reference in its entirety. The “monoclonal antibodies” may also be isolated from phage antibody libraries using the techniques described in Clackson et al., “Making Antibody Fragments Using Phage Display Libraries,” Nature 352:624-28 (1991) and Marks et al., “By-Passing Immunization. Human Antibodies From V-Gene Libraries Displayed on Phage,” J. Mol. Biol. 222:581-97 (1991), for example, which are hereby incorporated by reference in their entirety.

As used herein, the term “polyclonal antibody” includes, for example, a preparation of antibodies derived from at least two (2) different antibody-producing cell lines. The use of this term includes preparations of at least two (2) antibodies that contain antibodies that specifically bind to different epitopes or regions of an antigen.

As used herein, the terms “single chain antibodies” or “single chain Fv (scFv)” may refer to an antibody fusion molecule of the two domains of the Fv fragment, V_{L} and V_{H}. Although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv). See, e.g., Bird et al., “Single-Chain Antigen-Binding Proteins,” Science 242:423-26 (1988) and Huston et al., “Protein Engineering of Antibody Binding Sites: Recovery of Specific Activity in an Anti-Digoxin Single-Chain Fv Analogue Produced in Escherichia coli,” Proc. Natl. Acad. Sci. USA 85:5879-83 (1988), which are hereby incorporated by reference in their entirety. Such single chain antibodies are included by reference to the term “antibody” fragments, and can be prepared by recombinant techniques or enzymatic or chemical cleavage of intact antibodies.

As used herein, the term “variable” may, for example, refer to the fact that certain segments of the variable domains differ extensively in sequence among antibodies. The V domain mediates antigen binding and defines specificity of a particular antibody for its particular antigen. However, the variability is not evenly distributed across the amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies. See Kabat et al., Sequences of Proteins of Immunological Interest, 5th Ed. Public Health Service, National Institutes of Health, Bethesda, Md. (1991), which is hereby incorporated by reference in its entirety. The constant domains are not involved directly in binding an antibody to an antigen, but exhibit various effector functions, such as participation of the antibody in antibody dependent cellular cytotoxicity (“ADCC”).

The targeting domains of the present application can be, for example, monospecific, bispecific, trispecific or of greater multispecificity. Multispecific targeting domains can be specific for different epitopes of a substrate or can be specific for both a substrate polypeptide of the present application as well as for heterologous compositions, such as a heterologous polypeptide or solid support material. See, e.g., WO 93/17715; WO 92/08802; WO 91/00360; WO 92/05793; Tutt et al., “Trispecific F(ab′)3 Derivatives That Use Cooperative Signaling Via the TCR/CD3 Complex and CD2 to Activate and Redirect Resting Cytotoxic T Cells,” J. Immunol. 147:60-69 (1991); U.S. Pat. Nos. 5,573,920, 4,474,893, 5,601,819, 4,714,681, 4,925,648; 6,106,835; Kostelny et al., “Formation of a Bispecific Antibody by the Use of Leucine Zippers,” J. Immunol. 148:1547-53 (1992), which are hereby incorporated by reference in their entirety. The targeting domains of the application can be from any animal origin, including birds and mammals. For example, the targeting domains may be from human, marine, rabbit, goat, guinea pig, camel, horse, or chicken.

Techniques for generating targeting domains directed to target substrates are well known to those skilled in the art. Examples of such techniques include, but are not limited to, e.g., those involving display libraries, xeno or humab mice, hybridomas, and the like. Target polypeptides—from which a targeting domain is derived—within the scope of the present application include any polypeptide or polypeptide derivative which is capable of exhibiting antigenicity. Examples include, but are not limited to, substrate and fragments thereof. In some embodiments, the targeting domain is a single-chain antibody.

Single chain antibodies (“scFv”) are genetically engineered antibodies that consist of the variable domain of a heavy chain at the amino terminus joined to the variable domain of a light chain by a flexible region. In some embodiments, scFv are generated by PCR from hybridoma cell lines that express monoclonal antibodies (mAbs) with known target specificity, or they are selected by phage display from libraries isolated from spleen cells or lymphocytes, and preserve the affinity of the parent antibody. Employing a protocol to identify intracellular substrates, the yeast two-hybrid technology serves to identify candidate scFv—protein interactions. Such a system is useful to predict whether or not a scFv will be able to recognize its target substrate in vivo. See Portner-Taliana et al., “Identification of Protein Single chain Antibody Interactions In Vivo Using Two-hybrid Protocols,” Protein—Protein Interactions: A Molecular Cloning Manual, Cold Spring Harbor Laboratory Press, Chapter 24 (©2002), which is hereby incorporated by reference in its entirety.

Typically, scFv, hybrid antibodies or hybrid antibody fragments that are cloned into a display vector can be selected against the appropriate antigen in order to identify variants that maintained good binding activity, because the antibody or antibody fragment will be present on the surface of the phage or phagemid particle. See e.g., Barbas III et al., Phage Display, A Laboratory Manual (Cold Spring Harbor Laboratory Press, Cold Spring Harbor, N.Y., 2001), which is hereby incorporated by reference in its entirety. However, other vector formats could be used for this process, such as cloning the antibody fragment library into a lytic phage vector (modified T7 or Lambda Zap systems) for selection and/or screening.

In general, expression vectors useful in recombinant DNA techniques are often in the form of plasmids. However, the present application is intended to include such other forms of expression vectors that are not technically plasmids, such as viral vectors, e.g., replication defective retroviruses, adenoviruses and adeno-associated viruses, which serve equivalent functions. Such viral vectors permit infection of a subject and expression in that subject of a compound. The expression control sequences are typically eukaryotic promoter systems in vectors capable of transforming or transfecting eukaryotic host cells. Once the vector has been incorporated into the appropriate host, the host is maintained under conditions suitable for high level expression of the nucleotide sequences encoding the target domain, and the collection and purification of the substrate binding agent, e.g., cross-reacting anti-substrate antibodies. See, generally, U.S. Patent Publication No. 2002/0199213, which is hereby incorporated by reference in its entirety. Vectors can also encode signal peptide, e.g., pectate lyase, useful to direct the secretion of extracellular antibody fragments. See U.S. Pat. No. 5,576,195, which is hereby incorporated by reference in its entirety.

As used herein, the terms “degradation domain” or “degradation region” includes a portion of a chimeric molecule that is capable of facilitating the ubiquitination of a substrate. The degradation domain may have a second “binding” region for interaction with a native binding protein. The binding region can be modified as to possess one or more mutations, substitutions, deletions, or may be deleted entirely.

The degradation domain may contain E3 mimics with folds similar to eukaryotic E3s such as HECT-type, RING or U-box (RING/U-box)-type, and F-box domains, as well as unconventional E3s with folds unlike any other eukaryotic E3s such as NEL, XL-box-containing, and SidC. The degradation domain relates to polypeptides or polypeptide regions capable of modifying substrates by attaching one or more ubiquitin molecules and/or ubiquitin-like molecules to the substrates. In this regard, such a region comports with the well-known ubiquitination cascade—involving the coordinated action of the E1, E2, and E3 enzymes—which functions to activate and concomitantly conjugate ubiquitin to a substrate. In some embodiments, the motif is a ubiquitin region composed of a novel E3 ligase, or fragment thereof, which catalyzes the transfer of ubiquitin in a substrate-specific manner. See Qian et al., “Engineering a Ubiquitin Ligase Reveals Conformational Flexibility Required for Ubiquitin Transfer,” J. Biol. Chem. 284(39):26797-802 (2009), which is hereby incorporated by reference in its entirety.

As used herein, the terms “modification(s)” or “amino acid modification” of a polypeptide, protein, region, domain, or the like, refers to a change in the native sequence such as a deletion, addition or substation of a desired residue. Such modified polypeptides are prepared by introducing appropriate nucleotide changes into the antibody nucleic acid, or by peptide synthesis. Any combination of deletion, insertion, and substitution is made to obtain the antibody of interest, as long as the obtained antibody possesses the desired properties. The modification also includes the change of the pattern of glycosylation of the protein. A useful method for identification of preferred locations for mutagenesis is called “alanine scanning mutagenesis” as described by Cunningham and Wells, “High-Resolution Epitope Mapping of hGH-Receptor Interactions by Alanine-Scanning Mutagenesis,” Science 244:1081-85 (1989), which is hereby incorporated by reference in its entirety. The mutated antibody is then screened for the desired activity.

The terms “polypeptide,” “protein,” and “peptide” are used herein interchangeably herein to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, ubiquitinated forms, etc. Modifications also include intra-molecular crosslinking and covalent attachment to various moieties such as lipids, flavin, biotin, polyethylene glycol or derivatives thereof, etc. In addition, modifications may also include cyclization, branching and cross-linking. Further, amino acids other than the conventional twenty amino acids encoded by genes may also be included in a polypeptide. In one embodiment, the E3 ubiquitin ligase motif (E3), also referred to herein as EL or NEL, comprises a modified binding region which inhibits or decreases binding to said substrate compared to said E3 motif without the modified binding region. In another embodiment, the modification is a mutation or deletion in the binding region.

As used herein, the terms “variant” or “mutant” are used to refer to a protein or peptide which differs from a naturally occurring protein or peptide, i.e., the “prototype” or “wild-type” protein, by modifications to the naturally occurring protein or peptide, but which maintains the basic protein and side chain structure of the naturally occurring form. Such changes include, but are not limited to: changes in one, few, or even several amino acid side chains; changes in one, few or several amino acids, including deletions, e.g., a truncated version of the protein or peptide, insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A “variant” or “mutant” can have enhanced, decreased, changed, or substantially similar properties as compared to the naturally occurring protein or peptide.

In some embodiments, the degradation domain of the chimeric molecule lacks an endogenous substrate recognition region, i.e., a portion of the polypeptide that interacts with a natural or native binding partner. The E3 motif of the degradation domain may possess a modified binding domain which inhibits or decreases binding to a substrate compared to the E3 motif without the modified binding region. Nevertheless, the E3 motif permits proteolysis of a substrate in some embodiments. In some embodiments, the modification is a mutation, substitution, or deletion of the binding region. The substitution can be an amino acid substitution such as a conservative or a non-conservative amino acid substitution.

Non-conservative amino acid substitutions of the E3 motif, are substitutions in which an alkyl amino acid is substituted for an amino acid other than an alkyl amino acid in the sequence, an aromatic amino acid is substituted for an amino acid other than an aromatic amino acid in the E3 motif, a sulfur-containing amino acid is substituted for an amino acid other than a sulfur-containing amino acid in the E3 motif, a hydroxy-containing amino acid is substituted for an amino acid other than a hydroxy-containing amino acid in the E3 motif, an acidic amino acid is substituted for an amino acid other than an acidic amino acid in the E3 motif, a basic amino acid is substituted for an amino acid other than a basic amino acid in the E3 motif, or a dibasic monocarboxylic amino acid is substituted for an amino acid other than a dibasic monocarboxylic amino acid in the E3 motif.

Among the common amino acids, for example, “non-conservative amino acid substitutions” are illustrated by a substitution of an amino acids from one of the following groups with an amino acid that is not from the same group, as follows: (1) glycine, alanine, (2) valine, leucine, and isoleucine, (3) phenylalanine, tyrosine, and tryptophan, (4) cysteine and methionine, (5) serine and threonine, (6) aspartate and glutamate, (7) glutamine and asparagine, and (8) lysine, arginine and histidine.

Conservative or non-conservative amino acid changes in, e.g., the E3 motif, can be introduced by substituting appropriate nucleotides for the nucleotides encoding such a region. These modifications can be obtained, for example, by oligonucleotide-directed mutagenesis, linker-scanning mutagenesis, mutagenesis using the polymerase chain reaction, and the like. Ausubel et al. (eds.), Short Protocols in Molecular Biology, 5th Edition, John Wiley & Sons, Inc. (2002); see generally, McPherson (ed.), Directed Mutagenesis: A Practical Approach, IRL Press (1991), which are hereby incorporated by reference in their entirety. A useful method for identification of locations for sequence variation is called “alanine scanning mutagenesis” a described by Cunningham and Wells “Protein Engineering of Antibody Binding Sites: Recovery of Specific Activity in an Anti-Digoxin Single-Chain Fv Analogue Produced in Escherichia coli,” Science 244:1081-85 (1989), which is hereby incorporated by reference in its entirety.

Ubiquitin ligase families include, but are not limited to, the homologous to E6-associated protein C-terminus (“HECT”) domain ligases, which concerns the transfer of ubiquitin from the E2 conjugase to the substrate, the Really Interesting New Gene (“RING”) domain ligases, which bind E2, may mediate enzymatic activity in the E2-E3 complex, and the U-box ubiquitin family of ligases (“UULs”), which constitute a family of modified RING motif ligases without the full complement of Zn²⁺-binding ligands. See Colas et al., “Targeted Modification and Transportation of Cellular Proteins.” Proc. Natl. Acad. Sci. USA 97(25):13720-25 (2005), which is hereby incorporated by reference in its entirety.

U-box ubiquitin ligases (“ULLs”) are characterized as having a protein domain, the U-box, which is structurally related to the RING finger, typical of many other ubiquitin ligases. In humans, the UUL-encoding genes include, but are not limited to, UBE4A and UBE4B genes (also respectively termed UFD2b and UFD2a), CHIP (also termed STUB1), UIPS (also termed UBOXS), PRP19 (also termed PRPF19 or SNEV), CYC4 (also termed PPIL2 or Cyp-60), WDSUB1, and ACT1 (also termed TRAF3IP2). See Marin, I., “Ancient Origin of Animal U-box Ubiquitin Ligases.” BMC Evolutionary Biology 10:331, pp. 1-15 (2010), which is hereby incorporated by reference in its entirety.

While HECT E3 ligases have a direct role in catalysis during ubiquitination, RING and U-box E3 proteins facilitate protein ubiquitination by acting as adaptor molecules that recruit E2 and substrate molecules to promote substrate ubiquitination. Although many RING-type E3 ligases, such as MDM2 (murine double minute clone 2 oncoprotein) and c-Cbl, may act alone, others are found as components of much larger multi-protein complexes, such as the anaphase-promoting complex (“APC”). Taken together, these multifaceted properties and interactions enable E3 enzymes to provide a powerful, and specific, mechanism for protein clearance within all cells of eukaryotic organisms. Ardley et al., “E3 Ubiquitin Ligases.” Essays Biochem. 41:15-30 (2005), which is hereby incorporated by reference in its entirety.

Functional information concerning the E3 gene products is variable, nonetheless. The U-box protein CHIP acts both as a co-chaperone, together with chaperones such as, e.g., Hsc70, Hsp70 and Hsp90, and as a ubiquitin ligase, alone or as part of complexes that may include other E3 proteins. See id. The selectivity of the ubiquitin proteasome system for a particular substrate nevertheless relies on the interaction between a ubiquitin-conjugating enzyme, e.g., E2, and a ubiquitin-protein ligase. Post-translational modifications of the protein substrate, such as, e.g., phosphorylation or hydroxylation, are often required prior to ubiquitination. In this way, the precise spatio-temporal targeting and degradation for a particular substrate can be achieved.

The E3 motif of the degradation domain disclosed herein possesses a functional E3 ligase that is capable of ubiquitinating a substrate without steric disruption from native binding partners. In addition to the E3 motif, in some embodiments, the degradation domain possesses a ligase that is an E3 mimic with folds similar to eukaryotic E3s such as HECT-type, RING or U-box (RING/U-box)-type, and F-box domains, as well as unconventional E3s with folds unlike any other eukaryotic E3s such as NEL, XL-box-containing, and SidC. Such domains may possess cell or tissue specificity.

The E3 motif of the chimeric molecule may, in one embodiment, possess cell-type specific or tissue specific ligase function for, but not limited to, skin cells, muscle cells, epithelial cells, endothelial cells, stem cells, umbilical vessel cells, corneal cells, cardiomyocytes, aortic cells, corneal epithelial cells, somatic cells, fibroblasts, keratinocytes, melanocytes, adipose cells, bone cells, osteoblasts, airway cells, microvascular cells, mammary cells, vascular cells, chondrocytes, placental cells, hepatocytes, glial cells, epidermal cells, limbal stem cells, periodontal stem cells, bone marrow stromal cells, hybridoma cells, kidney cells, pancreatic islets, articular chondrocytes, neuroblasts, lymphocytes, and erythrocytes, and/or any combination thereof.

In one embodiment, the degradation domain is from a bacterial pathogen, the pathogen being optionally selected from Shigella, Salmonella, Bacillus, Bartonella, Bordetella, Borrelia, Brucella, Campylobacter, Chlamydia and Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Staphylococcus, Streptococcus, Treponema, Ureaplasma, Vibrio, and Yersinia. More particularly, in another embodiment, the bacterial pathogen is Shigella flexneri. The degradation domain, which may be derived from any bacteria may be from, for example, Shigella flexneri E3 ligase, SspH1, SspH2, SlrP, AvrPtoB, LubX, NLeG5-1, NleG5-1, NleG2-3, LegU1, LegAU13, NIeL, SopA, SidC, XopL, GobX, VirF, GALA, AnkB, and/or SidE.

In one embodiment, the degradation domain is a member of the Shigella IpaH protein family and may be IpaH9.8, IpaH1.4, IpaH2.5, IpaH4.5, IpaH7.8, IpaH0887, IpaH1389, IpaH2022, IpaH2202, IpaH2610, and/or IpaH0722. Shigella species are highly adapted human pathogens that cause bacillary dysentery (shigellosis). Via the type III secretion system (T3 SS), Shigella deliver a subset of virulence proteins (effectors) that are responsible for pathogenesis, with functions including pyroptosis, invasion of the epithelial cells, intracellular survival, and evasion of host immune responses.

Shigella possesses 12 ipaH genes, which reside on both the large plasmid and the chromosome. See, e.g., Ashida & Sasakawa, “Shigella IpaH Family Effectors as a Versatile Model for Studying Pathogenic Bacteria,” Front. Cell. Infect. Microbiol. 5:100 (2016), which is hereby incorporated by reference in its entirety. IpaH family proteins contain N-terminal leucine-rich repeats (LRRs) and have E3 ubiquitin ligase activity in their conserved C-terminal regions (Rohde et al., “Type III Secretion Effectors of the IpaH Family are E3 Ubiquitin Ligase,” Cell Host Microbe. 1:77-83 (2007) and Ashida et al., “Exploitation of the Host Ubiquitin System by Human Bacterial Pathogens,” Nat. Rev. Microbiol. 12:399-413 (2014), both of which are hereby incorporated by reference in their entirety). Ubiquitination is accomplished via a series of reactions catalyzed by a multienzymatic cascade: E1 (ubiquitin-activating enzyme), E2 (ubiquitin-conjugating enzyme), and E3 (ubiquitin ligase). Ashida & Sasakawa, “Shigella IpaH Family Effectors as a Versatile Model for Studying Pathogenic Bacteria,” Front. Cell. Infect. Microbiol. 5:100 (2016), which is hereby incorporated by reference in its entirety. The ubiquitination cascade starts with ATP-dependent activation of ubiquitin via formation of a thioester linkage between the carboxyl-terminal Gly of ubiquitin and a Cys of E1. Id. Activated ubiquitin is transferred to the active-site Cys of E2, and finally E3 ligase mediates the transfer of ubiquitin from the E2 to specific substrate proteins (mainly via substrate Lys residues). Id. E3 ligases can be categorized into two groups based on their structures and functions: HECT (Homologous to the E6-AP Carboxyl Terminus)-type and RING (Really Interesting New Gene)/U-box-type. Id. HECT-type E3 ligases catalyze ubiquitin transfer by accepting ubiquitin from E2 via formation of a thioester bond with their catalytic cysteine residue, and then transfer ubiquitin to their target substrates. Id. On the other hand, RING/U-box-type E3 ligases catalyze direct ubiquitin transfer by acting as scaffold molecules to bind and recruit the E2-ubiquitin complex, and then directly transfer ubiquitin from E2 to E3-bound substrates. Id.

IpaH family proteins are widely conserved among animal and plant pathogens, including Shigella (IpaH), Salmonella (SspH1, SspH2, and SlrP), Edwardsiella, Bradyrhizobium, Rhizobium, and some Pseudomonas species, illustrating the importance of these effectors in bacterial infection. Id. Although IpaH family proteins have E3 ubiquitin ligase activity and their C-terminal domains contain a single conserved Cys that form a Cys-ubiquitin intermediate similar to that of HECT-type ligases, the catalytic domains of IpaH family members differ at the sequence and structural levels from eukaryotic E3 ubiquitin ligases. Id. Consequently, IpaH family proteins are now considered to constitute a new class of E3 ubiquitin ligases, NEL (Novel E3 ligase), distinct from typical RING-, and HECT-types of E3 ubiquitin ligases (Singer et al., “Structure of the Shigella T3 SS effector IpaH Defines a New Class of E3 Ubiquitin Ligases,” Nat. Struct. Mol. Biol. 15:1293-1301 (2008); Zhu et al., “Structure of a Shigella Effector Reveals a New Class of Ubiquitin Ligases,” Nat. Struct. Mol. Biol. 15:1302-08 (2008); Quezada et al., “A Family of Salmonella Virulence Factors Functions as a Distinct Class of Autoregulated E3 Ubiquitin Ligases,” Proc. Natl. Acad. Sci. USA 106:4864-69 (2009), all of which are hereby incorporated by reference in their entirety). Although IpaH family proteins are highly similar to one another, the sequences of their LRR regions, regarded as substrate recognition sites, and subcellular localizations (e.g., nucleus, cytoplasm, or plasma membrane) are different. Ashida & Sasakawa, “Shigella IpaH Family Effectors as a Versatile Model for Studying Pathogenic Bacteria,” Front. Cell. Infect. Microbiol. 5:100 (2016), which is hereby incorporated by reference in its entirety.

Ubiquitin ligase families also include the “F-box” ligases-as in the Skp1-Cullin1-F-box (“SCF”) protein complex—which binds to a ubiquitinated substrate, such as, e.g., Cdc 4, which subsequently interacts with a target protein, such as, Sic1 or Grr1, which then binds Cln. See Bai et al., “SKP1 Connects Cell Cycle Regulators to the Ubiquitin Proteolysis Machinery through a Novel Motif, the F-Box,” Cell 86 (2):263-74 (1997), which is hereby incorporated by reference in its entirety.

The F-box is a protein motif of approximately 50 amino acids that functions as a site of protein-protein interaction. See, e.g., Kipreos et al., “The F-box Protein family.” Genome Biol. 1(5) (2000), which is hereby incorporated by reference in its entirety. F-box proteins were first characterized as components of SCF ubiquitin-ligase complexes, in which they bind substrates for ubiquitin-mediated proteolysis. The F-box motif links the F-box protein to other components of the SCF complex by binding the core SCF component Skp I. F-box proteins have more recently been discovered to function through non-SCF protein complexes in a variety of cellular functions. See id. F-box proteins often include additional carboxy-terminal motifs capable of protein-protein interaction; the most common secondary motifs in yeast and human F-box proteins are WD repeats and leucine-rich repeats, both of which have been found to bind phosphorylated substrates to the SCF complex. See id. The majority of F-box proteins have other associated motifs, and the functions of most of these proteins have not yet been defined. See id.

The least variant positions within the F-box motif include positions 8 (92% of the 234 F-box proteins used for the consensus have leucine or methionine), 9 (92% proline), 16 (86% isoleucine or valine), 20 (81% leucine or methionine), and 32 (92% serine or cysteine). Id. This lack of a strict consensus guides the skilled artisan to employ multiple search algorithms for detecting F-box sequences. Two algorithms, for example, can be found in the Prosite and Pfam databases. Occasionally, one database will give a significant score to an F-box in a given protein when the other does not detect it, so both databases should be searched. Id.

Expression of the chimeric molecules of the present application in prokaryotes is most often carried out in E. coli with vectors containing constitutive or inducible promoters directing the expression of either fusion or non-fusion polypeptides. Fusion vectors add a number of amino acids to a polypeptide encoded therein, usually to the amino terminus of the recombinant polypeptide. Such fusion vectors typically serve three purposes: (i) to increase expression; (ii) to increase the solubility; and (iii) to aid in purification by acting as a ligand in affinity purification. Often, in fusion expression vectors, a proteolytic cleavage site is introduced at the junction of the fusion moiety and the recombinant polypeptide to enable separation of the recombinant polypeptide from the fusion moiety subsequent to purification of the fusion polypeptide. Such enzymes, and their endogenous recognition sequences, include Factor Xa, thrombin and enterokinase. Typical fusion expression vectors include pGEX (Smith and Johnson, “Single-Step Purification of Polypeptides Expressed in Escherichia coli as Fusions With Glutathione S-Transferase,” Gene 67:31-40 (1988), which is hereby incorporated by reference in its entirety), pMAL (New England Biolabs, Beverly, Mass.) and pRIT5 (Pharmacia, Piscataway, N.J.) that fuse glutathione S-transferase (“GST”), maltose E binding polypeptide, or polypeptide A, respectively, to the target recombinant polypeptide.

Examples of suitable inducible non-fusion E. coli expression vectors include pTrc (Amrann et al., “Tightly Regulated tac Promoter Vectors Useful for the Expression of Unfused and Fused Proteins in Escherichia coli,” Gene 69:301-15 (1988) and pET lid (Studier et al., Gene Expression Technology: Methods In Enzymology 185, Academic Press, San Diego, Calif. 60-89 (1990)), which are hereby incorporated by reference in their entirety. Methods for targeted assembly of distinct active peptide or protein domains to yield multifunctional polypeptides via polypeptide fusion has been described by Pack et al., U.S. Pat. Nos. 6,294,353; 6,692,935, which are hereby incorporated by reference in their entirety. One strategy to maximize recombinant polypeptide expression, e.g., a chimeric molecule of the present application, in E. coli is to express the polypeptide in host bacteria with an impaired capacity to proteolytically cleave the recombinant chimera. See, e.g., Gottesman, Gene Expression Technology: Methods In Enzymology 185, Academic Press, San Diego, Calif. 119-128 (1990), which is hereby incorporated by reference in its entirety.

In some embodiments, a nucleic acid encoding a chimeric molecule of the present application—including a degradation domain and a targeting region—is expressed in mammalian cells using a mammalian expression vector. Examples of mammalian expression vectors include, e.g., but are not limited to, pcDNA3, pCDM8 (Seed, “An LFA-3 cDNA Encodes a Phospholipid-Linked Membrane Protein Homologous to its Receptor CD2,” Nature 329:840 (1987), which is hereby incorporated by reference in its entirety), and pMT2PC. When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells useful for expression of the targeting domains, degradation domains of the chimeric molecule. See, e.g., Chapters 16 and 17 of Sambrook et al., Molecular Cloning: A Laboratory Manual. 2nd ed., Cold Spring Harbor Laboratory, Cold Spring Harbor Laboratory Press, Cold Spring Harbor, N.Y., (1989), which are hereby incorporated by reference in their entirety.

Notwithstanding chimeric molecule E3 degradation domains and targeting domain expression, the function of such domains/regions imparts the specificity of the present application. A known or unknown substrate is bound by the targeting domain for subsequent ubiquitination via the degradation domain. In some embodiments, the substrates include, but are not limited to, intracellular substrates, extracellular substrates, modified substrates, glycosylated substrates, farnesylated substrates, post translationally modified substrates, phosphorylated substrates, and other modifications known in the art.

In some embodiments, the substrates include, but are not limited to, fluorescent protein, histone protein, nuclear localization signal (NLS), H-Ras protein, Src-homology 2 domain-containing phosphatase 2 (SHP2), β-galactosidase, gpD, Hsp70, MBP, CDC34, COPS5, MAP2K5, SF3A1, USP11, ubiquitin, EGFR, CEA, FcγIIa, FcγIIIa, hA33, mA33, hAlb, mIgG, AblSH2, vEGFR, MSLN, ERα/EF, hSUMO4, ySUMO, TNFα, avβ3 integrin, Src SH3, Lysozyme, phospho-IκBα, SARS N, goat IgG, rabbit IgG, post-translationally modified proteins, fibrillin, huntingtin, tumorigenic proteins, p53, Rb, adhesion proteins, receptors, cell-cycle proteins, checkpoint proteins, HFE, ATP7B, prion proteins, viral proteins, bacterial proteins, parasitic proteins, fungal proteins, DNA binding proteins, metabolic proteins, regulatory proteins, structural proteins, enzymes, immunogenic proteins, autoimmunogenic proteins, immunogens, antigens, pathogenic proteins, and the like. In one embodiment, the substrate is a fluorescent protein, for example, green fluorescent protein, emerald fluorescent protein, venus fluorescent protein, cerulean fluorescent protein, and enhanced cyan fluorescent protein.

As used herein, the term “amino acid” includes naturally-occurring amino acids, L-amino acids, D-amino acids, and synthetic amino acids, as well as amino acid analogs and amino acid mimetics that function in a manner similar to the naturally-occurring amino acids. Naturally-occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally-occurring amino acid, e.g., an a-carbon that is bound to a hydrogen, a carboxyl group, an amino group, and an R group, e.g., homoserine, norleucine, methionine sulfoxide, methionine methyl sulfonium. Such analogs have modified R-groups, e.g., norleucine, or modified peptide backbones, but retain the same basic chemical structure as a naturally-occurring amino acid. Amino acid mimetics refers to chemical compounds that have a structure that is different from the general chemical structure of an amino acid, but that functions in a manner similar to a naturally-occurring amino acid. Amino acids can be referred to herein by either their commonly known three letter symbols or by the one-letter symbols recommended by the IUPAC-IUB Biochemical Nomenclature Commission.

An exemplary E3 ligase that is useful as a degradation domain in accordance with the present application includes the E3 ubiquitin ligase AvrPtoB, which is a U-box motif, from Pseudomonas syringae which has the amino acid sequence of SEQ ID NO: 1:

MAGINRAGPSGAYFVGHTDPEPVSGQAHGSGSGASSSNSPQVQPRPSNTPP
SNAPAPPPTGRERLSRSTALSRQTREWLEQGMPTAEDASVRRRPQVTADAA
TPRAEARRTPEATADASAPRRGAVAHANSIVQQLVSEGADISHTRNMLRNA
MNGDAVAFSRVEQNIFRQHFPNMPMHGISRDSELAIELRGALRRAVHQQAA
SAPVRSPTPTPASPAASSSGSSQRSLFGRFARLMAPNQGRSSNTAASQTPV
DRSPPRVNQRPIRVDRAAMRNRGNDEADAALRGLVQQGVNLEHLRTALERH
VMQRLPIPLDIGSALQNVGINPSIDLGESLVQHPLLNLNVALNRMLGLRPS
AERAPRPAVPVAPATASRRPDGTRATRLRVMPEREDYENNVAYGVRLLNLN
PGVGVRQAVAAFVTDRAERPAVVANIRAALDPIASQFSQLRTISKADAESE
ELGFKDAADHHTDDVTHCLFGGELSLSNPDQQVIGLAGNPTDTSQPYSQEG
NKDLAFMDMKKLAQFLAGKPEHPMTRETLNAENIAKYAFRIVP

The E3 ubiquitin ligase AvrPtoB from Pseudomonas syringae has the nucleotide sequence of SEQ ID NO: 2 as follows:

ATGGCGGGTATCAATAGAGCGGGACCATCGGGCGCTTATTTTGTTGGCCAC
ACAGACCCCGAGCCAGTATCGGGGCAAGCACACGGATCCGGCAGCGGCGCC
AGCTCCTCGAACAGTCCGCAGGTTCAGCCGCGACCCTCGAATACTCCCCCG
TCGAACGCGCCCGCACCGCCGCCAACCGGACGTGAGAGGCTTTCACGATCC
ACGGCGCTGTCGCGCCAAACCAGGGAGTGGCTGGAGCAGGGTATGCCTACA
GCGGAGGATGCCAGCGTGCGTCGTAGGCCACAGGTGACTGCCGATGCCGCA
ACGCCGCGTGCAGAGGCAAGACGCACGCCGGAGGCAACTGCCGATGCCAGC
GCACCGCGTAGAGGGGCGGTTGCACACGCCAACAGTATCGTTCAGCAATTG
GTCAGTGAGGGCGCTGATATTTCGCATACTCGTAACATGCTCCGCAATGCA
ATGAATGGCGACGCAGTCGCTTTTTCTCGAGTAGAACAGAACATATTTCGC
CAGCATTTCCCGAACATGCCCATGCATGGAATCAGCCGAGATTCGGAACTC
GCTATCGAGCTCCGTGGGGCGCTTCGTCGAGCGGTTCACCAACAGGCGGCG
TCAGCGCCAGTGAGGTCGCCCACGCCAACACCGGCCAGCCCTGCGGCATCA
TCATCGGGCAGCAGTCAGCGTTCTTTATTTGGACGGTTTGCCCGTTTGATG
GCGCCAAACCAGGGACGGTCGTCGAACACTGCCGCCTCTCAGACGCCGGTC
GACAGGAGCCCGCCACGCGTCAACCAAAGACCCATACGCGTCGACAGGGCT
GCGATGCGTAATCGTGGCAATGACGAGGCGGACGCCGCGCTGCGGGGGTTA
GTACAACAGGGGGTCAATTTAGAGCACCTGCGCACGGCCCTTGAAAGACAT
GTAATGCAGCGCCTCCCTATCCCCCTCGATATAGGCAGCGCGTTGCAGAAT
GTGGGAATTAACCCAAGTATCGACTTGGGGGAAAGCCTTGTGCAACATCCC
CTGCTGAATTTGAATGTAGCGTTGAATCGCATGCTGGGGCTGCGTCCCAGC
GCTGAAAGAGCGCCTCGTCCAGCCGTCCCCGTGGCTCCCGCGACCGCCTCC
AGGCGACCGGATGGTACGCGTGCAACACGATTGCGGGTGATGCCGGAGCGG
GAGGATTACGAAAATAATGTGGCTTATGGAGTGCGCTTGCTTAACCTGAAC
CCGGGGGTGGGGGTAAGGCAGGCTGTTGCGGCCTTTGTAACCGACCGGGCT
GAGCGGCCAGCAGTGGTGGCTAATATCCGGGCAGCCCTGGACCCTATCGCG
TCACAATTCAGTCAGCTGCGCACAATTTCGAAGGCCGATGCTGAATCTGAA
GAGCTGGGTTTTAAGGATGCGGCAGATCATCACACGGATGACGTGACGCAC
TGTCTTTTTGGCGGAGAATTGTCGCTGAGTAATCCGGATCAGCAGGTGATC
GGTTTGGCGGGTAATCCGACGGACACGTCGCAGCCTTACAGCCAAGAGGGA
AATAAGGACCTGGCGTTCATGGATATGAAAAAACTTGCCCAATTCCTCGCA
GGCAAGCCTGAGCATCCGATGACCAGAGAAACGCTTAACGCCGAAAATATC
GCCAAGTATGCTTTTAGAATAGTCCCC

A further exemplary E3 ubiquitin ligase that is useful as a degradation domain in accordance with the present application includes the E3 ubiquitin ligase IpaH0722, which is a novel E3 ligase (also referred to herein as NEL or EL), from Shigella flexneri which has the amino acid sequence of SEQ ID NO: 3:

MKPAHNPSFFRSFCGLGCISRLSVEEQNITDYHRIWDNWAKEGAATEDRTQ
AVRLLKICLAFQEPALNLSLLRLRSLPYLPPHIQELNISSNELRSLPELPP
SLTVLKASDNRLSRLPALPPHLVALDVSLNRVLTCLPSLPSSLQSLSALLN
SLETLPDLPPALQKLSVGNNQLTALPELPCELQELSAFDNRLQELPPLPQN
LRLLNVGENQLHRLPELPQRLQSLYIPNNQLNTLPDSIMNLHIYADVNIYN
NPLSTRTLQALQRLTSSPDYHGPRIYFSMSDGQQNTLHRPLADAVTAWFPE
NKQSDVSQIWHAFEHEEHANTFSAFLDRLSDTVSARNTSGFREQVAAWLEK
LSASAELRQQSFAVAADATESCEDRVALTWNNLRKTLLVHQASEGLFDNDT
GALLSLGREMFRLEILEDIARDKVRTLHFVDEIEVYLAFQTMLAEKLQLST
AVKEMRFYGVSGVTANDLRTAEAMVRSREENEFTDWFSLWGPWHAVLKRTE
ADRWALAEEQKYEMLENEYPQRVADRLKASGLSGDADAEREAGAQVMRETE
QQIYRQLTDEVLALRLPENGSQUIHS

The E3 ubiquitin ligase IpaH0722, which is a novel E3 ligase, from Shigella flexneri has the nucleotide sequence of SEQ ID NO: 4 as follows:

ATGAAACCTGCCCACAATCCTTCTTTTTTCCGCTCCTTTTGTGGTTTAGGA
TGTATATCCCGTTTATCCGTAGAAGAGCAAAATATCACGGATTATCACCGC
ATCTGGGATAACTGGGCCAAGGAAGGTGCTGCAACAGAAGACCGAACACAG
GCAGTTCGATTACTGAAAATATGTCTGGCTTTTCAAGAGCCAGCCCTCAAT
TTAAGTTTACTCAGATTACGCTCTCTCCCATACCTGCCCCCGCACATACAA
GAACTTAACATCTCTAGCAATGAGCTACGCTCTCTGCCAGAACTCCCTCCG
TCCTTAACTGTACTTAAAGCCAGCGATAACAGACTGAGCAGGCTCCCGGCT
CTTCCGCCTCACCTGGTCGCTCTTGATGTTTCACTTAACAGAGTTTTAACA
TGTTTGCCTTCTCTTCCATCTTCCTTGCAGTCACTCTCAGCCCTTCTCAAT
AGCCTGGAGACGCTACCTGATCTTCCCCCGGCTCTACAAAAACTTTCTGTT
GGCAACAACCAGCTTACTGCCTTACCAGAATTACCATGTGAACTACAGGAA
CTAAGTGCTTTTGATAACAGATTACAAGAGCTACCGCCCCTTCCTCAAAAT
CTGAGGCTTTTAAACGTTGGGGAAAACCAACTACACAGACTGCCCGAACTT
CCACAACGTCTGCAATCACTATATATCCCTAACAATCAGCTGAACACATTG
CCAGACAGTATCATGAATCTGCACATTTATGCAGATGTTAATATTTATAAC
AATCCATTGTCGACTCGCACTCTGCAAGCCCTGCAAAGATTAACCTCTTCG
CCGGACTACCACGGCCCACGGATTTACTTCTCCATGAGTGACGGACAACAG
AATACACTCCATCGCCCCCTGGCTGATGCCGTGACAGCATGGTTCCCGGAA
AACAAACAATCTGATGTATCACAGATATGGCATGCTTTTGAACATGAAGAG
CATGCCAACACCTTTTCCGCGTTCCTTGACCGCCTTTCCGATACCGTCTCT
GCACGCAATACCTCCGGATTCCGTGAACAGGTCGCTGCATGGCTGGAAAAA
CTCAGTGCCTCTGCGGAGCTTCGACAGCAGTCTTTCGCTGTTGCTGCTGAT
GCCACTGAGAGCTGTGAGGACCGTGTCGCGCTCACATGGAACAATCTCCGG
AAAACCCTCCTGGTCCATCAGGCATCAGAAGGCCTTTTCGATAATGATACC
GGCGCTCTGCTCTCCCTGGGCAGGGAAATGTTCCGCCTCGAAATTCTGGAG
GACATTGCCCGGGATAAAGTCAGAACTCTCCATTTTGTGGATGAGATAGAA
GTCTACCTGGCCTTCCAGACCATGCTCGCAGAGAAACTTCAGCTCTCTACT
GCCGTGAAGGAAATGCGTTTCTATGGCGTGTCGGGAGTGACAGCAAATGAC
CTCCGCACTGCCGAAGCCATGGTCAGAAGCCGTGAAGAGAATGAATTTACG
GACTGGTTCTCCCTCTGGGGACCATGGCATGCTGTACTGAAGCGTACGGAA
GCTGACCGCTGGGCGCTGGCAGAAGAGCAGAAATATGAGATGCTGGAGAAT
GAGTACCCTCAGAGGGTGGCTGACCGGCTGAAAGCATCAGGTCTGAGCGGT
GATGCGGATGCGGAGAGGGAAGCCGGTGCACAGGTGATGCGTGAGACTGAA
CAGCAGATTTACCGTCAGCTGACTGACGAGGTACTGGCCCTGCGATTGCCT
GAAAACGGCTCACAACTGCACCATTCATAA

A further exemplary E3 ligase that is useful as a degradation domain in accordance with the present application includes the E3 ubiquitin ligase IpaH1.4, which is a novel E3 ligase, from Shigella flexneri has the amino acid sequence of SEQ ID NO: 5 as follows:

MIKSTNIQAIGSGIMHQINNIYSLTPFPLPMELTPSCNEFYLKAWSEWEKN
GTPGEQRNIAFNRLKICLQNQEAELNLSELDLKTLPDLPPQITTLEIRKNL
LTHLPDLPPMLKVIHAQFNQLESLPALPETLEELNAGDNKIKELPFLPENL
THLRVHNNRLHILPLLPPELKLLVVSGNRLDSIPPFPDKLEGLAMANNFIE
QLPELPFSMNRAVLMNNNLTTLPESVLRLAQNAFVNVAGNPLSGHTMRTLQ
QITTGPDYSGPRIFFSMGNSATISAPEHSLADAVTAWFPENKQSDVSQIWH
AFEHEEHANTFAFLDRLSDTVSARNTSGFREQVAAWLEKLSASAELRQQSF
AVAADATESCEDRVALTWNNLRKTLLVHQASEGLFDNDTGALLSLGREMFR
LEILEDIARDKVRTLHFVDEIEVYLAFQTMLAEKLQLSTAVKEMRFYGVSG
VTANDLRTAEAMVRSREENEFKDWFSLWGPWHAVLKRTEADRWAQAEEQKY
EMLENEYSQRVADRLKASGLSGDTDAEREAGAQVMRETEQQIYRQLTDEVL
ALRLSENGSNHIA

The E3 ubiquitin ligase IpaH1.4, which is a novel E3 ligase, from Shigella flexneri has the nucleotide sequence of SEQ ID NO: 6 as follows:

ATGATTAAATCAACCAATATACAGGCAATCGGTTCTGGTATTATGCATCAA
ATAAACAATATATACTCGTTAACTCCATTTCCTTTACCTATGGAACTGACT
CCATCTTGTAATGAATTTTATTTAAAAGCCTGGAGTGAATGGGAAAAGAAC
GGTACCCCAGGCGAGCAACGCAATATCGCCTTCAATAGGCTGAAAATATGT
TTACAAAATCAAGAGGCAGAATTAAATTTATCTGAGTTAGATTTAAAAACA
TTACCAGATTTACCGCCTCAGATAACAACACTGGAAATAAGAAAAAACCTA
TTAACACATCTCCCTGATTTACCACCAATGCTTAAGGTAATACATGCTCAA
TTTAATCAACTGGAAAGCTTACCTGCCTTACCCGAGACGTTAGAAGAGCTT
AATGCGGGTGATAACAAGATAAAAGAATTACCATTTCTTCCTGAAAATCTA
ACTCATTTACGGGTTCATAATAACCGATTGCATATTCTGCCACTATTGCCA
CCGGAACTAAAATTACTGGTAGTTTCTGGAAACAGATTAGACAGCATTCCC
CCCTTTCCAGATAAGCTTGAAGGGCTGGCTATGGCTAATAATTTTATAGAA
CAACTACCGGAATTACCTTTTAGTATGAACAGGGCTGTGCTAATGAATAAT
AATCTGACAACACTTCCGGAAAGTGTCCTGAGATTAGCTCAGAATGCCTTC
GTAAATGTTGCAGGTAATCCACTGTCTGGCCATACCATGCGTACACTACAA
CAAATAACCACCGGACCAGATTATTCTGGTCCTCGAATATTTTTCTCTATG
GGAAATTCTGCCACAATTTCCGCTCCAGAACACTCCCTGGCTGATGCCGTG
ACAGCATGGTTCCCGGAAAACAAACAATCTGATGTATCACAGATATGGCAT
GCTTTTGAACATGAAGAGCACGCCAACACCTTTTCCGCGTTCCTTGACCGC
CTTTCCGATACCGTCTCTGCACGCAATACCTCCGGATTCCGTGAACAGGTC
GCTGCATGGCTGGAAAAACTCAGTGCCTCTGCGGAGCTTCGACAGCAGTCT
TTCGCTGTTGCTGCTGATGCCACTGAGAGCTGTGAGGACCGTGTCGCGCTC
ACATGGAACAATCTCCGGAAAACCCTCCTGGTCCATCAGGCATCAGAAGGC
CTTTTCGATAATGATACCGGCGCTCTGCTCTCCCTGGGCAGGGAAATGTTC
CGCCTCGAAATTCTGGAGGACATTGCCCGGGATAAAGTCAGAACTCTCCAT
TTTGTGGATGAGATAGAAGTCTACCTGGCCTTCCAGACCATGCTCGCAGAG
AAACTTCAGCTCTCCACTGCCGTGAAGGAAATGCGTTTCTATGGCGTGTCG
GGAGTGACAGCAAATGACCTCCGCACTGCCGAAGCCATGGTCAGAAGCCGT
GAAGAGAATGAATTTAAGGACTGGTTCTCCCTCTGGGGACCATGGCATGCT
GTACTGAAGCGTACGGAAGCTGACCGCTGGGCGCAGGCAGAAGAGCAGAAG
TATGAGATGCTGGAGAATGAGTACTCTCAGAGGGTGGCTGACCGGCTGAAA
GCATCAGGTCTGAGCGGTGATACGGATGCGGAGAGGGAAGCCGGTGCACAG
GTGATGCGTGAGACTGAACAGCAGATTTACCGTCAGTTGACTGACGAGGTA
CTGGCCCTGCGATTGTCTGAAAACGGCTCAAATCATATCGCATAA

A further exemplary E3 ubiquitin ligase that is useful as a degradation domain in accordance with the present application includes the E3 ubiquitin ligase IpaH2.5, which is a novel E3 ligase, from Shigella flexneri which has the amino acid sequence of SEQ ID NO: 7:

MLIRILVIMIKSTNIQAIGSGIMHQINNVYSLTPLSLPMELTPSCNEFYLK
TWSEWEKNGTPGEQRNIAFNRLKICLQNQEAELNLSELDLKTLPDLPPQIT
TLEIRKNLLTHLPDLPPMLKVIHAQFNQLESLPALPETLEELNAGDNKIKE
LPFLPENLTHLRVHNNRLHILPLLPPELKLLVVSGNRLDSIPPFPDKLEGL
ALANNFIEQLPELPFSMNRAVLMNNNLTTLPESVLRLAQNAFVNVAGNPLS
GHTMRTLQQITTGPDYSGPRIFFSMGNSATISAPEHSLADAVTAWFPENKQ
SDVSQIWHAFEHEEHANTFSAFLDRLSDTVSARNTSGFREQVAAWLEKLSA
SAELRQQSFAVAADATESCEDRVALTWNNLRKTLLVHQASEGLFDNDTGAL
LSLGREMFRLEILEDIARDKVRTLHFVDEIEVYLAFQTMLAEKLQLSTAVK
EMRFYGVSGVTANDLRTAEAMVRSREENEFTDWFSLWGPWHAVLKRTEADR
WAQAEEQKYEMLENEYSQRVADRLKASGLSGDADAEREAGAQVMRETEQQI
YRQLTDEVLA

The E3 ubiquitin ligase IpaH2.5, which is a novel E3 ligase, from Shigella flexneri has the nucleotide sequence of SEQ ID NO: 8 as follows:

atgTTGATAAGAATTCTAGTTATAATGATTAAATCAACCAATATACAGGCA
ATCGGTTCTGGCATTATGCATCAAATAAACAATGTATACTCGTTAACTCCA
TTATCTTTACCTATGGAACTGACTCCATCTTGTAATGAATTTTATTTAAAA
ACCTGGAGCGAATGGGAAAAGAACGGTACCCCAGGCGAGCAACGCAATATC
GCCTTCAATAGGCTGAAAATATGTTTACAAAATCAAGAGGCAGAATTAAAT
TTATCTGAGTTAGATTTAAAAACATTACCAGATTTACCGCCTCAGATAACA
ACACTGGAAATAAGAAAAAACCTATTAACACATCTCCCTGATTTACCACCA
ATGCTTAAGGTAATACATGCTCAATTTAATCAACTGGAAAGCTTACCTGCC
TTACCCGAGACGTTAGAAGAGCTTAATGCGGGTGATAACAAGATAAAAGAA
TTACCATTTCTTCCTGAAAATCTAACTCATTTACGGGTTCATAATAACCGA
TTGCATATTCTGCCACTATTGCCACCGGAACTAAAATTACTGGTAGTTTCT
GGAAACAGATTAGACAGCATTCCCCCCTTTCCAGATAAGCTTGAAGGGCTG
GCTCTGGCTAATAATTTTATAGAACAACTACCGGAATTACCTTTTAGTATG
AACAGGGCTGTGCTAATGAATAATAATCTGACAACACTTCCGGAAAGTGTC
CTGAGATTAGCTCAGAATGCCTTCGTAAATGTTGCAGGTAATCCATTGTCT
GGCCATACCATGCGTACACTACAACAAATAACCACCGGACCAGATTATTCT
GGTCCTCGAATATTTTTCTCTATGGGAAATTCTGCCACAATTTCCGCTCCA
GAACACTCCCTGGCTGATGCCGTGACAGCATGGTTCCCGGAAAACAAACAA
TCTGATGTATCACAGATATGGCATGCTTTTGAACATGAAGAGCATGCCAAC
ACCTTTTCCGCGTTCCTTGACCGCCTTTCCGATACCGTCTCTGCACGCAAT
ACCTCCGGATTCCGTGAACAGGTCGCTGCATGGCTGGAAAAACTCAGTGCC
TCTGCGGAGCTTCGACAGCAGTCTTTCGCTGTTGCTGCTGATGCCACTGAG
AGCTGTGAGGACCGTGTCGCGCTCACATGGAACAATCTCCGGAAAACCCTC
CTGGTCCATCAGGCATCAGAAGGCCTTTTCGATAATGATACCGGCGCTCTG
CTCTCCCTGGGCAGGGAAATGTTCCGCCTCGAAATTCTGGAGGATATTGCC
CGGGATAAAGTCAGAACTCTCCATTTTGTGGATGAGATAGAAGTCTACCTG
GCCTTCCAGACCATGCTCGCAGAGAAACTTCAGCTCTCTACTGCCGTGAAG
GAAATGCGTTTCTATGGCGTGTCGGGAGTGACAGCAAATGACCTCCGCACT
GCCGAAGCCATGGTCAGAAGCCGTGAAGAGAATGAATTTACGGACTGGTTC
TCCCTCTGGGGACCATGGCATGCTGTACTGAAGCGTACGGAAGCTGACCGC
TGGGCGCAGGCAGAAGAGCAGAAGTATGAGATGCTGGAGAATGAGTACTCT
CAGAGGGTGGCTGACCGGCTGAAAGCATCAGGTCTGAGCGGTGATGCGGAT
GCGGAGAGGGAAGCCGGTGCACAGGTGATGCGTGAGACTGAACAGCAGATT
TACCGTCAGTTGACTGACGAGGTACTGGCCTGA

A further exemplary E3 ubiquitin ligase that is useful as a degradation domain in accordance with the present application includes the E3 ubiquitin ligase IpaH4.5, which is a novel E3 ligase, from Shigella flexneri and has the amino acid sequence of SEQ ID NO: 9:

MKPINNHSFFRSLCGLSCISRLSVEEQCTRDYHRIWDDWARE
GTTTENRIQAVRLLKICLDTREPVLNLSLLKLRSLPPLPLHI
RELNISNNELISLPENSPLLTELHVNGNNLNILPTLPSQLIK
LNISFNRNLSCLPSLPPYLQSLSARFNSLETLPELPSTLTIL
RIEGNRLTVLPELPHRLQELFVSGNRLQELPEFPQSLKYLKV
GENQLRRLSRLPQELLALDVSNNLLTSLPENIITLPICTNVN
ISGNPLSTRVLQSLQRLTSSPDYHGPQIYFSMSDGQQNTLII
RPLADAVTAWFPENKQSDVSQIWHAFEHEEHANTFSAFLDRL
SDTVSARNTSGFREQVAAWLEKLSASAELRQQSFAVAADATE
SCEDRVALTWNNLRKTLLVHQASEGLFDNDTGALLSLGREMF
RLEILEDIARDKVRTLHFVDEIEVYLAFQTMLAEKLQLSTAV
KEMRFYGVSGVTANDLRTAEAMVRSREENEFTDWFSLWGPWH
AVLKRTEADRWAQAEEQKYEMLENEYSQRVADRLKASGLSGD
ADAQREAGAQVMRETEQQIYRQLTDEVLA

The E3 ubiquitin ligase IpaH4.5, which is a novel E3 ligase, from Shigella flexneri has the nucleotide sequence of SEQ ID NO: 10 as follows:

ATGAAACCGATCAACAATCATTCTTTTTTTCGTTCCCTTTGT
GGCTTATCATGTATATCTCGTTTATCGGTAGAAGAACAGTGT
ACCAGAGATTACCACCGCATCTGGGATGACTGGGCTAGGGAA
GGAACAACAACAGAAAATCGCATCCAGGCGGTTCGATTATTG
AAAATATGTCTGGATACCCGGGAGCCTGTTCTCAATTTAAGC
TTACTGAAACTACGTTCTTTACCACCACTCCCTTTGCATATA
CGTGAACTTAATATTTCCAACAATGAGTTAATCTCCCTACCT
GAAAATTCTCCGCTTTTGACAGAACTTCATGTAAATGGTAAC
AACTTGAATATACTCCCGACACTTCCATCTCAACTGATTAAG
CTTAATATTTCATTCAATCGAAATTTGTCATGTCTGCCATCA
TTACCACCATATTTACAATCACTCTCGGCACGTTTTAATAGT
CTGGAGACGTTACCAGAGCTTCCATCAACGCTAACAATATTA
CGTATTGAAGGTAATCGCCTTACTGTCTTGCCTGAATTGCCT
CATAGACTACAAGAACTCTTTGTTTCCGGCAACAGACTACAG
GAACTACCAGAATTTCCTCAGAGCTTAAAATATTTGAAGGTA
GGTGAAAATCAACTACGCAGATTATCCAGATTACCGCAAGAA
CTATTGGCTCTGGATGTTTCCAATAACCTACTAACTTCATTA
CCCGAAAATATAATCACATTGCCCATTTGTACGAATGTTAAC
ATTTCAGGGAATCCATTGTCGACTCGCGTTCTGCAATCCCTG
CAAAGATTAACCTCTTCGCCGGACTACCACGGCCCGCAGATT
TACTTCTCCATGAGTGACGGACAACAGAATACACTCCATCGC
CCCCTGGCTGATGCCGTGACAGCATGGTTCCCGGAAAACAAA
CAATCTGATGTATCACAGATATGGCATGCTTTTGAACATGAA
GAGCATGCCAACACCTTTTCCGCGTTCCTTGACCGCCTTTCC
GATACCGTCTCTGCACGCAATACCTCCGGATTCCGTGAACAG
GTCGCTGCATGGCTGGAAAAACTCAGTGCCTCTGCGGAGCTT
CGACAGCAGTCTTTCGCTGTTGCTGCTGATGCCACTGAGAGC
TGTGAGGACCGTGTCGCGCTCACATGGAACAATCTCCGGAAA
ACCCTCCTGGTCCATCAGGCATCAGAAGGCCTTTTCGATAAT
GATACCGGCGCTCTGCTCTCCCTGGGCAGGGAAATGTTCCGC
CTCGAAATTCTGGAGGACATTGCCCGGGATAAAGTCAGAACT
CTCCATTTTGTGGATGAGATAGAAGTCTACCTGGCCTTCCAG
ACCATGCTCGCAGAGAAACTTCAGCTCTCCACTGCCGTGAAG
GAAATGCGTTTCTATGGCGTGTCGGGAGTGACAGCAAATGAC
CTCCGCACTGCCGAAGCTATGGTCAGAAGCCGTGAAGAGAAT
GAATTTACGGACTGGTTCTCCCTCTGGGGACCATGGCATGCT
GTACTGAAGCGTACGGAAGCTGACCGCTGGGCGCAGGCAGAA
GAGCAGAAGTATGAGATGCTGGAGAATGAGTACTCTCAGAGG
GTGGCTGACCGGCTGAAAGCATCAGGTCTGAGCGGTGATGCG
GATGCGCAGAGGGAAGCCGGTGCACAGGTGATGCGTGAGACT
GAACAGCAGATTTACCGTCAGCTGACTGACGAGGTACTGGCC
TGA

A further exemplary E3 ubiquitin ligase that is useful as a degradation domain in accordance with the present application includes the E3 ubiquitin ligase IpaH7.8, which is a novel E3 ligase, from Shigella flexneri and has the amino acid sequence of SEQ ID NO: 11:

MFSVNNTHSSVSCSPSINSNSTSNEHYLRILTEWEKNSSPGE
ERGIAFNRLSQCFQNQEAVLNLSDLNLTSLPELPKHISALIV
ENNKLTSLPKLPAFLKELNADNNRLSVIPELPESLTTLSVRS
NQLENLPVLPNHLTSLFVENNRLYNLPALPEKLKFLHVYYNR
LTTLPDLPDKLEILCAQRNNLVTFPQFSDRNNIRQKEYYFHF
NQITTLPESFSQLDSSYRINISGNPLSTRVLQSLQRLTSSPD
YHGPQIYFSMSDGQQNTLHRPLADAVTAWFPENKQSDVSQIW
HAFEHEEHANTFSAFLDRLSDTVSARNTSGFREQVAAWLEKL
SASAELRQQSFAVAADATESCEDRVALTWNNLRKTLLVHQAS
EGLFDNDTGALLSLGREMFRLEILEDIARDKVRTLHFVDEIE
VYLAFQTMLAEKLQLSTAVKEMRFYGVSGVTANDLRTAEAMV
RSREENEFTDWFSLWGPWHAVLKRTEADRWAQAEEQKYEMLE
NEYSQRVADRLKASGLSGDADAEREAGAQVMRETEQQIYRQL
TDEVLALRLSENGSRLHHS

The E3 ubiquitin ligase IpaH7.8, which is a novel E3 ubiquitin ligase, from Shigella flexneri has the nucleotide sequence of SEQ ID NO: 12 as follows:

ATGTTCTCTGTAAATAATACACACTCATCAGTTTCTTGCTCC
CCCTCTATTAACTCAAACTCAACCAGTAATGAACATTATCTG
AGAATCCTGACTGAATGGGAAAAGAACTCTTCTCCCGGGGAA
GAGCGAGGCATTGCTTTTAACAGACTCTCCCAGTGCTTTCAG
AATCAAGAAGCAGTATTAAATTTATCAGACCTAAATTTGACG
TCTCTTCCCGAATTACCAAAGCATATTTCTGCTTTGATTGTA
GAAAATAATAAATTAACATCATTGCCAAAGCTGCCTGCATTT
CTTAAAGAACTTAATGCTGATAATAACAGGCTTTCTGTGATA
CCAGAACTTCCTGAGTCATTAACAACTTTAAGTGTTCGTTCT
AATCAACTGGAAAACCTTCCTGTTTTGCCAAACCATTTAACA
TCATTATTTGTTGAAAATAACAGGCTATATAACTTACCGGCT
CTTCCCGAAAAATTGAAATTTTTACATGTTTATTATAACAGG
CTGACAACATTACCCGACTTACCGGATAAACTGGAAATTCTC
TGTGCTCAGCGCAATAATCTGGTTACTTTTCCTCAATTTTCT
GATAGAAACAATATCAGACAAAAGGAATATTATTTTCATTTT
AATCAGATAACCACTCTTCCGGAGAGTTTTTCACAATTAGAT
TCAAGTTACAGGATTAATATTTCAGGGAATCCATTGTCGACT
CGCGTTCTGCAATCCCTGCAAAGATTAACCTCTTCGCCGGAC
TACCACGGCCCACAGATTTACTTCTCCATGAGTGACGGACAA
CAGAATACACTCCATCGCCCCCTGGCTGATGCCGTGACAGCA
TGGTTCCCGGAAAACAAACAATCTGATGTATCACAGATATGG
CATGCTTTTGAACATGAAGAGCATGCCAACACCTTTTCCGCG
TTCCTTGACCGCCTTTCCGATACCGTCTCTGCACGCAATACC
TCCGGATTCCGTGAACAGGTCGCTGCATGGCTGGAAAAACTC
AGTGCCTCTGCGGAGCTTCGACAGCAGTCTTTCGCTGTTGCT
GCTGATGCCACTGAGAGCTGTGAGGACCGTGTCGCGCTCACA
TGGAACAATCTCCGGAAAACCCTCCTGGTCCATCAGGCATCA
GAAGGCCTTTTCGATAATGATACCGGCGCTCTGCTCTCCCTG
GGCAGGGAAATGTTCCGCCTCGAAATTCTGGAGGACATTGCC
CGGGATAAAGTCAGAACTCTCCATTTTGTGGATGAGATAGAA
GTCTACCTGGCCTTCCAGACCATGCTCGCAGAGAAACTTCAG
CTCTCTACTGCCGTGAAGGAAATGCGTTTCTATGGCGTGTCG
GGAGTGACAGCAAATGACCTCCGCACTGCCGAAGCCATGGTC
AGAAGCCGTGAAGAGAATGAATTTACGGACTGGTTCTCCCTC
TGGGGACCATGGCATGCTGTACTGAAGCGTACGGAAGCTGAC
CGCTGGGCGCAGGCAGAAGAGCAGAAGTATGAGATGCTGGAG
AATGAGTACTCTCAGAGGGTGGCTGACCGGCTGAAAGCATCA
GGTCTGAGCGGTGATGCGGATGCGGAGAGGGAAGCCGGTGCA
CAGGTGATGCGTGAGACTGAACAGCAGATTTACCGTCAGTTG
ACTGACGAGGTACTGGCCCTGCGATTGTCTGAAAACGGCTCA
CGACTGCACCATTCATAA

A further exemplary E3 ubiquitin ligase that is useful as a degradation domain in accordance with the present application includes the E3 ubiquitin ligase IpaH9.8, which is a novel E3 ligase, from Shigella flexneri and has the amino acid sequence of SEQ ID NO: 13:

MLPINNNFSLPQNSFYNTISGTYADYFSAWDKWEKQALPGEE
RDEAVSRLKECLINNSDELRLDRLNLSSLPDNLPAQITLLNV
SYNQLTNLPELPVTLKKLYSASNKLSELPVLPPALESLQVQH
NELENLPALPDSLLTMNISYNEIVSLPSLPQALKNLRATRNF
LTELPAFSEGNNPVVREYFFDRNQISHIPESILNLRNECSIH
ISDNPLSSHALQALQRLTSSPDYHGPRIYFSMSDGQQNTLHR
PLADAVTAWFPENKQSDVSQIWHAFEHEEHANTFSAFLDRLS
DTVSARNTSGFREQVAAWLEKLSASAELRQQSFAVAADATES
CEDRVALTWNNLRKTLLVHQASEGLFDNDTGALLSLGREMTR
LEILEDIARDKVRTLHIFVDEIEVYLAFQTMLAEKLQLSTAV
KEMRFYGVSGVTANDLRTAEAMVRSREENEFTDWFSLWGPWH
AVLKRTEADRWAQAEEQKYEMLENEYPQRVADRLKASGLSGD
ADAEREAGAQVMRETEQQIYRQLTDEVLALRLSENGSQLHHS

The E3 ubiquitin ligase IpaH9.8, which is a novel E3 ligase, from Shigella flexneri has the nucleotide sequence of SEQ ID NO: 14 as follows:

ATGTTACCGATAAATAATAACTTTTCATTGCCCCAAAATTCT
TTTTATAACACTATTTCCGGTACATATGCTGATTACTTTTCA
GCATGGGATAAATGGGAAAAACAAGCGCTCCCCGGTGAAGAG
CGTGATGAGGCTGTCTCCCGACTTAAAGAATGTCTTATCAAT
AATTCCGATGAACTTCGACTGGACCGTTTAAATCTGTCCTCG
CTACCTGACAACTTACCAGCTCAGATAACGCTGCTCAATGTA
TCATATAATCAATTAACTAACCTACCTGAACTGCCTGTTACG
CTAAAAAAATTATATTCCGCCAGCAATAAATTATCAGAATTG
CCCGTGCTACCTCCTGCGCTGGAGTCACTTCAGGTACAACAC
AATGAGCTGGAAAACCTGCCAGCTTTACCCGATTCGTTATTG
ACTATGAATATCAGCTATAACGAAATAGTCTCCTTACCATCG
CTCCCACAGGCTCTTAAAAATCTCAGAGCGACCCGTAATTTC
CTCACTGAGCTACCAGCATTTTCTGAGGGAAATAATCCCGTT
GTCAGAGAGTATTTTTTTGATAGAAATCAGATAAGTCATATC
CCGGAAAGCATTCTTAATCTGAGGAATGAATGTTCAATACAT
ATTAGTGATAACCCATTATCATCCCATGCTCTGCAAGCCCTG
CAAAGATTAACCTCTTCGCCGGACTACCACGGCCCACGGATT
TACTTCTCCATGAGTGACGGACAACAGAATACACTCCATCGC
CCCCTGGCTGATGCCGTGACAGCATGGTTCCCGGAAAACAAA
CAATCTGATGTATCACAGATATGGCATGCTTTTGAACATGAA
GAGCATGCCAACACCTTTTCCGCGTTCCTTGACCGCCTTTCC
GATACCGTCTCTGCACGCAATACCTCCGGATTCCGTGAACAG
GTCGCTGCATGGCTGGAAAAACTCAGTGCCTCTGCGGAGCTT
CGACAGCAGTCTTTCGCTGTTGCTGCTGATGCCACTGAGAGC
TGTGAGGACCGTGTCGCGCTCACATGGAACAATCTCCGGAAA
ACCCTCCTGGTCCATCAGGCATCAGAAGGCCTTTTCGATAAT
GATACCGGCGCTCTGCTCTCCCTGGGCAGGGAAATGTTCCGC
CTCGAAATTCTGGAGGATATTGCCCGGGATAAAGTCAGAACT
CTCCATTTTGTGGATGAGATAGAAGTCTACCTGGCCTTCCAG
ACCATGCTCGCAGAGAAACTTCAGCTCTCCACTGCCGTGAAG
GAAATGCGTTTCTATGGCGTGTCGGGAGTGACAGCAAATGAC
CTCCGCACTGCCGAAGCCATGGTCAGAAGCCGTGAAGAGAAT
GAATTTACGGACTGGTTCTCCCTCTGGGGACCATGGCATGCT
GTACTGAAGCGTACGGAAGCTGACCGCTGGGCGCAGGCAGAA
GAGCAGAAATATGAGATGCTGGAGAATGAGTACCCTCAGAGG
GTGGCTGACCGGCTGAAAGCATCAGGTCTGAGCGGTGATGCG
GATGCGGAGAGGGAAGCCGGTGCACAGGTGATGCGTGAGACT
GAACAGCAGATTTACCGTCAGCTGACTGACGAGGTACTGGCC
CTGCGATTGTCTGAAAACGGCTCACAACTGCACCATTCATAA

A further exemplary E3 ubiquitin ligase that is useful as a degradation domain in accordance with the present application includes the E3 ubiquitin ligase LegAU13, which is a F-box motif, from Legionella pneumophila and has the amino acid sequence of SEQ ID NO: 15:

MKKNFFSDLPEETIVNTLSFLKANTLARIAQTCQFFNRLAND
KHLELHQLRQQHIKRELWGNLMVAARSNNLEEVKKILKKGID
PTQTNSYHLNRTPLLAAIEGKAYQTANYLWRKYTFDPNFKDN
YGDSPISLLKKQLANPAFKDKEKKQIRALIRGMQEEKIAQSK
CLVC

The E3 ubiquitin ligase LegAU13, which is a F-box motif, from Legionella pneumophila has the nucleotide sequence of SEQ ID NO: 16 as follows:

ATGAAAAAGAATTTTTTTTCTGATCTTCCTGAGGAAACAATT
GTCAATACATTGAGTTTCTTAAAAGCAAACACACTAGCTCGT
ATAGCTCAGACATGTCAATTTTTTAATCGCTTGGCTAATGAT
AAACATCTGGAGCTGCATCAACTAAGACAACAGCATATAAAG
CGAGAGCTATGGGGAAATCTTATGGTGGCGGCAAGAAGCAAT
AACCTGGAAGAGGTCAAAAAGATTCTAAAAAAAGGAATCGAT
CCAACCCAGACCAATAGCTACCACTTAAATAGAACGCCTTTA
CTTGCAGCTATCGAAGGAAAAGCATATCAAACTGCAAATTAC
CTCTGGAGAAAATACACTTTCGATCCCAATTTTAAAGATAAC
TATGGTGATTCACCTATCTCTCTTCTTAAAAAGCAACTGGCA
AATCCAGCCTTCAAGGATAAGGAAAAAAAACAAATACGCGCC
TTAATTAGGGGAATGCAAGAAGAAAAAATAGCACAGAGCAAG
TGCCTTGTTTGTTAA

A further exemplary E3 ubiquitin ligase that is useful as a degradation domain in accordance with the present application includes the E3 ubiquitin ligase LegU1, which is a F-box motif, from Legionella pneumophila and has the amino acid sequence of SEQ ID NO: 17:

MKAKYDPTKPGLQKLPPEIKVMILEFLDAKSKLALSQTNYGW
RDLILDRPEYTKEITNTLFRLDKKRHRQAIAQMMSGRVTASS
MAKLFEELLCFSIPSSYVFLIFFASQKSVALIEVLTVILVFA
AITSLAHDLVDYFIESDTKAEKQHAHRRAFQFFAQPSQSAAQ
QNLEEENLSADPKACQCEPL

The E3 ubiquitin ligase LegU1, which is a F-box motif, from Legionella pneumophila has the nucleotide sequence of SEQ ID NO: 18 as follows:

ATGAAAGCAAAATACGACCCCACAAAGCCTGGACTCCAAAAG
TTACCTCCTGAAATCAAGGTAATGATTCTTGAGTTTCTTGAT
GCCAAATCAAAACTAGCTCTTTCACAGACAAATTATGGTTGG
CGTGATTTAATTCTAGACCGGCCAGAATATACCAAAGAAATA
ACGAATACATTATTTCGTCTTGATAAAAAACGCCATCGTCAA
GCAATAGCACAAATGATGTCAGGAAGAGTTACAGCAAGTTCT
ATGGCTAAGCTATTTGAAGAATTACTATGTTTTAGCATACCT
TCGTCCTATGTGTTTTTAATCTTTTTCGCATCGCAAAAATCT
GTGGCGCTTATAGAAGTCTTAACCGTAATCCTTGTGTTTGCT
GCAATAACCTCTCTCGCCCATGATCTGGTGGATTATTTTATT
GAAAGTGATACAAAAGCTGAGAAACAGCATGCACATCGCCGT
GCTTTTCAATTCTTTGCCCAACCCAGTCAAAGCGCTGCACAA
CAAAACTTGGAGGAAGAGAATTTAAGTGCTGATCCCAAGGCC
TGCCAATGTGAGCCATTGTAG

A further exemplary E3 ubiquitin ligase that is useful as a degradation domain in accordance with the present application includes the E3 ubiquitin ligase LubX, which is a U-box motif, from Legionella pneumophila and has the amino acid sequence of SEQ ID NO: 19:

MATRNPFDIDEIKSKYLREAALEANLSHIPETTPTMLTCPID
SGFLKDPVITPEGFVYNKSSILKWLETKKEDPQSRKPLTAKD
LQPFPELLIIVNRFVETQTNYEKLKNRLVQNARVAARQKEYT
EIPDIFLCPISKTLIKTPVITAQGKVYDQEALSNFLIATGNK
DETGKKLSIDDVVVFDELYQQIKVYNFYRKREVQKNQIQPSV
SNGFGFFSLNFLTSWLWGTEEKKEKTSSDMTY

The E3 ubiquitin ligase LubX, which is a U-box motif, from Legionella pneumophila has the nucleotide sequence of SEQ ID NO: 20 as follows:

ATGGCGACGCGAAATCCTTTTGATATTGATCATAAAAGTAAA
TACTTAAGAGAAGCAGCATTAGAAGCCAATTTATCTCATCCA
GAAACAACACCAACAATGCTGACTTGCCCTATTGACAGCGGA
TTTCTAAAAGATCCCGTGATCACACCTGAAGGTTTTGTTTAT
AATAAATCCTCTATTTTAAAATGGTTAGAAACGAAAAAAGAA
GACCCACAAAGCCGTAAACCCTTAACGGCTAAAGATTTGCAA
CCATTCCCCGAGTTATTGATTATAGTCAATAGATTTGTTGAG
ACACAAACGAACTATGAAAAATTAAAAAACAGATTAGTGCAA
AATGCTCGGGTTGCTGCACGCCAAAAAGAATACACTGAAATT
CCGGATATATTTCTTTGCCCAATAAGTAAAACGCTTATCAAA
ACACCTGTCATTACTGCCCAAGGGAAAGTATATGATCAAGAA
GCATTAAGTAACTTTCTTATCGCAACGGGTAATAAAGATGAA
ACAGGCAAAAAATTATCCATTGATGATGTAGTGGTGTTTGAT
GAACTCTATCAACAGATAAAAGTTTATAATTTTTACCGCAAA
CGCGAAGTGCAAAAAAATCAAATTCAACCTTCAGTAAGTAAT
GGTTTTGGCTTTTTTAGCTTGAATTTTCTCACCTCATGGTTA
TGGGGAACTGAGGAGAAAAAAGAAAAGACATCATCTGATATG
ACGTACTAA

A further exemplary E3 ubiquitin ligase that is useful as a degradation domain in accordance with the present application includes the E3 ubiquitin ligase NleG2-3, which is a U-box motif, Enterohemorrhagic Escherichia coli (EHEC) O157:H7 and has the amino acid sequence of SEQ ID NO: 21:

MPLTSDIRSHSFNLGVEVVRARIVANGRGDITVGGETVSIVY
DSTNGRFSSSGGNGGLLSELLLLGFNSGPRALGERMLSMLSD
SGEAQSQESIQNKISQCKFSVCPERLQCPLEAIQCPITLEQP
EKGIFVKNSDGSDVCTLFDAAAFSRLVGEGLPHPLTREPITA
SIIVKHEECIYDDTRGNFIIKGN

The E3 ubiquitin ligase NleG2-3, which is a U-box motif, from Enterohemorrhagic Escherichia coli (“EHEC”) O157:H7, has the nucleotide sequence of SEQ ED NO: 22 as follows:

ATGCCATTAACCTCAGATATTAGATCACATTCATTTAATCTT
GGGGTGGAGGTTGTTCGTGCCCGAATTGTAGCCAATGGGCGC
GGAGATATTACAGTCGGTGGTGAAACTGTCAGTATTGTGTAT
GATTCTACTAATGGGCGCTTTTCATCCAGTGGCGGTAATGGC
GGATTGCTTTCTGAGTTATTGCTTTTGGGATTTAATAGTGGT
CCTCGAGCCCTTGGTGAGAGAATGCTAAGTATGCTTTCGGAC
TCAGGTGAAGCACAATCGCAAGAGAGTATTCAGAACAAAATA
TCTCAATGTAAGTTTTCTGTTTGTCCAGAGAGACTTCAGTGC
CCGCTTGAGGCTATTCAGTGTCCAATTACACTGGAGCAGCCT
GAAAAAGGTATTTTTGTGAAGAATTCAGATGGTTCAGATGTA
TGTACTTTATTTGATGCCGCTGCATTTTCTCGTTTGGTTGGT
GAAGGCTTACCCCACCCACTGACCCGGGAACCAATAACGGCA
TCAATAATTGTAAAACATGAAGAATGCATTTATGACGATACC
AGAGGAAACTTCATTATAAAGGGTAATTGA

A further exemplary E3 ubiquitin ligase that is useful as a degradation domain in accordance with the present application includes the E3 Ubiquitin Ligase NleG5-1, which is a U-box motif, from Enterohemorrhagic Escherichia coli (“EHEC”) O157:H7, and has the amino acid sequence of SEQ ID NO: 23:

MPVDLTPYILPGVSFLSDIPQETLSEIRNQTIRGEAQIRLGE
LMVSIRPMQVNGYFMGSLNQDGLSNDNIQIGLQYIEHIERTL
NHGSLTSREVTVLREIEMLENMDLLSNYQLEELLDKIEVCAF
NVEHAQLQVPESLRTCPVTLCEPEDGVFMRNSMNSNVCMLYD
KMALIHLVKTRAAHPLSRESIAVSMIVGRDNCAFDPDRGNFV
LKN

The E3 ubiquitin ligase NleG5-1, which is a U-box motif, from Enterohemorrhagic Escherichia coli (“EHEC”) O157:H7, has the nucleotide sequence of SEQ ID NO: 24 as follows:

ATGCCTGTAGATTTAACGCCTTATATTTTACCTGGGGTTAGTTTTTTGTC
TGACATTCCTCAAGAAACCTTGTCTGAGATACGTAATCAGACTATTCGTG
GAGAAGCTCAAATAAGACTGGGTGAGTTGATGGTGTCAATACGACCTATG
CAGGTAAATGGATATTTTATGGGAAGTCTTAACCAGGATGGTTTATCGAA
TGATAATATCCAGATTGGCCTTCAATATATAGAACATATTGAACGTACAC
TTAATCATGGTAGTTTGACAAGCCGTGAAGTTACAGTACTGCGTGAAATT
GAGATGCTCGAAAATATGGATTTGCTTTCTAACTACCAGTTAGAGGAGTT
GTTAGATAAAATTGAAGTATGTGCATTTAATGTGGAGCATGCACAATTGC
AAGTGCCAGAGAGCTTACGAACATGCCCTGTTACATTATGTGAACCAGAA
GATGGGGTATTTATGAGGAATTCAATGAATTCAAATGTTTGTATGTTGTA
TGATAAAATGGCATTAATACATCTTGTTAAAACAAGGGCGGCTCATCCTT
TGAGCAGGGAATCAATCGCAGTTTCAATGATTGTAGGAAGAGATAATTGT
GCTTTTGACCCTGACAGAGGTAACTTCGTTTTAAAAAATTAA

A further exemplary E3 ubiquitin ligase that is useful as a degradation domain in accordance with the present application includes the E3 ubiquitin ligase NleL, which is a HECT motif, from Enterohemorrhagic Escherichia coli (“EHEC”) O157:H7, and has the amino acid sequence of SEQ ID NO: 25:

MLPTTNISVNSGVISFESPVDSPSNEDVEVALEKWCAEGEFSENRHEVAS
KILDVISTNGETLSISEPITTLPDLLPGSLKELVLNGCTELKSINCLPPN
LSSLSMVGCSSLEVINCSIPENVINLSLCHCSSLKHIEGSFPEALRNSVY
LNGCNSLNESQCQFLAYDVSQGRACLSKAELTADLIWLSANRTGEESAEE
LNYSGCDLSGLSLVGLNLSSVNFSGAVLDDTDLRMSDLSQAVLENCSFKN
SILNECNFCYANLSNCIIRALFENSNFSNSNLKNASFKGSSYIQYPPILN
EADLTGAIIIPGMVLSGAILGDVKELFSEKSNTINLGGCYIDLSDIQENI
LSVLDNYTKSNKSILLTMNTSDDKYNHDKVRAAEELIKKISLDELAAFRP
YVKMSLADSFSIHPYLNNANIQQWLEPICDDFFDTIIVISWFNNSIMMYM
ENGSLLQAGMYFERHPGAMVSYNSSFIQIVMNGSRRDGMQERFRELYEVY
LKNEKVYPVTQQSDFGLCDGSGKPDWDDDSDLAYNWVLLSSQDDGMAMMC
SLSHMVDMLSPNTSTNWMSFFLYKDGEVQNTFGYSLSNLFSESFPIFSIP
YHKAFSQNFVSGILDILISDNELKERFIEALNSNKSDYKMIADDQQRKLA
CVWNPFLDGWELNAQHVDMIMGSHVLKDMPLRKQAEILFCLGGVFCKYSS
SDMFGTEYDSPEILRRYANGLIEQAYKTDPQVFGSVYYYNDILDRLQGRN
NVFTCTAVLTDMLTEHAKESFPEIFSLYYPVAWR

A further exemplary E3 ubiquitin ligase that is useful as a degradation domain in accordance with the present application includes the E3 ubiquitin ligase NleL, which is a HECT motif, from Enterohemorrhagic Escherichia coli (“EHEC”) O157:H7, and has the nucleotide sequence of SEQ ID NO: 26:

ATGCTGCCCACTACAAATATCTCTGTAAATTCTGGAGTAATATCTTTTGAAAGTCCT
GTAGATTCACCATCTAACGAGGATGTTGAAGTTGCCCTCGAAAAGTGGTGTGCTGAG
GGAGAATTTAGCGAAAATCGTCATGAGGTTGCATCAAAAATACTTGATGTTATAAGT
ACTAATGGAGAGACTTTATCAATCAGTGAGCCAATAACAACATTACCAGACTTGCTT
CCAGGTTCTCTGAAAGAACTGGTTTTGAATGGATGTACAGAGCTTAAATCAATAAAC
TGCTTACCCCCCAACTTATCTTCATTAAGTATGGTTGGATGCTCATCATTAGAGGTTA
TAAATTGCAGCATACCTGAAAATGTCATTAATTTATCTTTATGCCATTGTAGTTCTTT
GAAACATATAGAAGGTTCCTTTCCTGAGGCACTCAGAAATTCCGTATATTTAAATGG
CTGTAATTCATTAAATGAATCGCAATGTCAATTCCTTGCATATGATGTCAGTCAAGG
CCGTGCCTGCCTGAGCAAAGCTGAGCTTACTGCTGACTTAATTTGGTTGTCAGCTAA
CCGAACGGGTGAAGAGTCTGCTGAAGAATTGAATTACTCTGGATGTGACTTGTCAG
GTCTAAGTCTTGTAGGGCTGAATTTATCATCAGTAAATTTTTCTGGAGCAGTGCTTG
ATGATACAGATCTCAGGATGAGTGATTTGTCTCAGGCTGTATTGGAAAACTGTTCTT
TTAAAAACTCGATTTTGAATGAATGTAATTTTTGTTATGCTAATTTATCTAATTGTAT
TATTAGGGCTTTGTTTGAAAACTCTAATTTCAGCAATTCCAATCTTAAAAATGCATC
ATTTAAAGGATCTTCATATATACAATATCCTCCAATTTTGAACGAGGCTGATTTAAC
AGGAGCTATTATAATTCCTGGAATGGTTTTAAGTGGTGCTATCTTAGGTGATGTAAA
GGAGCTCTTTAGTGAAAAAAGTAATACCATTAATCTAGGAGGGTGTTACATAGATCT
ATCTGACATACAGGAAAATATATTATCTGTGTTGGATAACTATACAAAATCAAATAA
ATCAATTTTATTGACTATGAATACATCTGATGATAAGTATAACCATGATAAAGTAAG
GGCCGCTGAAGAACTTATCAAAAAAATATCTCTTGACGAATTAGCGGCGTTCCGGCC
CTATGTTAAGATGTCTTTGGCTGATTCATTTAGTATTCATCCTTATTTGAACAACGCA
AATATACAGCAATGGCTCGAGCCTATATGTGATGACTTTTTTGATACTATAATGTCTT
GGTTTAATAATTCAATAATGATGTATATGGAGAATGGTAGTTTATTGCAGGCAGGGA
TGTATTTTGAGCGACATCCAGGTGCGATGGTATCTTATAATAGTTCCTTTATACAAAT
TGTAATGAATGGTTCACGGCGTGATGGAATGCAGGAACGATTTAGGGAACTCTATG
AAGTATATTTAAAAAATGAAAAAGTTTATCCTGTCACACAGCAGAGTGATTTTGGAT
TGTGCGATGGCTCTGGGAAGCCTGACTGGGATGATGATTCCGATTTGGCTTATAACT
GGGTTTTGTTATCATCACAGGATGATGGTATGGCAATGATGTGTTCTTTGAGTCATA
TGGTTGATATGTTATCTCCTAATACATCAACTAATTGGATGTCCTTTTTTTTATATAA
GGATGGAGAAGTTCAAAATACATTTGGGTATTCATTGAGCAATCTTTTTTCTGAATC
ATTTCCAATTTTCAGTATTCCTTATCATAAAGCTTTTTCCCAGAATTTCGTTTCTGGTA
TTCTGGATATACTCATTTCTGATAATGAACTCAAAGAGAGATTTATTGAGGCACTTA
ATTCCAATAAATCAGATTATAAAATGATTGCTGATGATCAGCAAAGGAAACTTGCCT
GTGTCTGGAATCCCTTTCTTGATGGTTGGGAACTGAACGCTCAGCATGTAGATATGA
TTATGGGGAGCCATGTATTGAAAGATATGCCACTAAGAAAACAGGCTGAAATATTA
TTTTGTTTAGGGGGGGTTTTCTGTAAATACTCATCGAGTGATATGTTTGGTACAGAGT
ATGATTCTCCTGAGATTCTACGGAGATATGCAAATGGATTGATTGAACAAGCTTATA
AAACAGATCCTCAGGTATTTGGCTCAGTTTATTATTACAATGATATTTTAGACAGGC
TACAAGGAAGAAATAATGTTTTTACTTGTACCGCTGTGCTGACTGATATGCTAACGG
AGCATGCAAAAGAATCTTTTCCTGAAATATTTTCATTGTATTATCCTGTTGCGTGGCG
TTGA

A further exemplary E3 ubiquitin ligase that is useful as a degradation domain in accordance with the present application includes the E3 ubiquitin ligase SidC, which is an unconventional motif, from L. pneumophila, and has the amino acid sequence of SEQ ID NO: 27:

MVINMVDVIKFKEPERCDYLYVDENNKVHILLPIVGGDEIGLDNTCQTAV
ELITFFYGSAHSGVTKYSAEHQLSEYKRQLEEDIKAINSQKKISPHAYDD
LLKEKIERLQQIEKYIELIQVLKKQYDEQNDIRQLRTGGIPQLPSGVKEI
IKSSENAFAVRLSPYDNDKFTRFDDPLFNVKRNISKYDTPSRQAPIPIYE
GLGYRLRSTLFPEDKTPTPINKKSLRDKVKSTVLSHYKDEDRIDGEKKDE
KLNELITNLQNELVKELVKSDPQYSKLSLSKDPRGKEINYDYLVNSLMLV
DNDSEIGDWIDTILDATVDSTVWVAQASSPFYDGAKEISSDRDADKISIR
VQYLLAEANIYCKTNKLSDANFGEFFDKEPHATEIAKRVKEGFTQGADIE
PIIYDYINSNHAELGLKSPLTGKQQQEITDKFTKHYNTIKESPHFDEFFV
ADPDKKGNIFSHQGRISCHFLDFFTRQTKGKHPLGDLASHQEALQEGTSN
RLHHKNEVVAQGYEKLDQFKKEVVKLLAENKPKELLDYLVATSPTGVPNY
SMLSKETQNYIAYNRNWPAIQKELEKATSIPESQKQDLSRLLSRDNLQHD
NLSAITWSKYSSKPLLDVELNKIAEGLELTAKIYNEKRGREWWFKGSRNE
ARKTQCEELQRVSKEINTLLQSESLTKSQVLEKVLNSIETLDKIDRDISA
ESNWFQSTLQKEVRLFRDQLKDICQLDKYAFKSTKLDEIISLEMEEQFQK
IQDPAVQQIVRDLPSHCHNDEAIEFFKTLNPEEAAKVASYLSLEYREINK
STDKKTLLEQDIPRLFKEVNTQLLSKLKEEKAIDEQVHEKLSQLADKIAP
EHFTRNNIIKWSTNPEKLEESNLNEPIKSVQSPTTKQTSKQFREAMGEIT
GRNEPPTDTLYTGIIKK

The E3 ubiquitin ligase SidC, which is an unconventional motif, from L. pneumophila, has the nucleotide sequence of SEQ ID NO: 28 as follows:

ATGGTGATAAACATGGTTGACGTAATCAAATTCAAAGAGCCGGAACGTTGTGATTA
TCTATATGTTGATGAAAACAACAAAGTTCATATCCTTTTACCGATTGTAGGAGGAGA
TGAAATAGGCCTGGATAATACCTGTCAAACAGCAGTTGAGTTGATCACATTTTTCTA
TGGTAGTGCGCACAGTGGTGTGACTAAATATTCTGCTGAACACCAACTCAGTGAATA
CAAAAGGCAATTGGAAGAAGACATCAAAGCCATCAATAGTCAAAAGAAAATTTCAC
CTCATGCTTATGACGATTTATTAAAAGAGAAAATAGAACGCTTACAGCAAATTGAA
AAATACATTGAATTAATTCAAGTACTAAAAAAACAATATGATGAACAAAATGATAT
CAGGCAACTTCGTACTGGAGGGATTCCGCAATTACCCTCTGGGGTAAAGGAAATCA
TTAAATCCTCTGAAAATGCTTTCGCTGTGAGACTTTCTCCATATGACAACGATAAAT
TCACTCGCTTTGATGACCCTTTATTCAATGTCAAAAGAAACATCTCAAAATATGACA
CGCCCTCAAGACAAGCTCCTATTCCAATATACGAGGGATTAGGTTATCGCCTGCGTT
CAACACTGTTCCCGGAAGATAAAACACCAACTCCAATTAATAAAAAATCACTTAGG
GATAAAGTTAAAAGCACTGTTCTTAGTCATTATAAAGATGAAGATAGAATTGATGG
AGAAAAAAAAGATGAAAAATTAAACGAACTAATTACTAATCTTCAAAACGAACTTG
TAAAAGAGTTAGTAAAAAGTGATCCTCAATATTCGAAACTATCTTTATCTAAAGATC
CAAGAGGAAAAGAAATAAATTACGATTATTTAGTAAATAGTTTGATGCTTGTAGAT
AACGACTCTGAAATTGGTGATTGGATTGATACTATTCTCGACGCTACAGTAGATTCC
ACTGTCTGGGTAGCTCAGGCATCCAGCCCTTTCTATGATGGTGCTAAAGAAATATCA
TCAGACCGCGATGCGGACAAGATATCCATCAGAGTTCAGTACCTGTTGGCCGAAGC
CAATATTTACTGTAAAACAAACAAATTATCGGATGCTAACTTTGGAGAATTTTTCGA
CAAAGAGCCTCATGCTACTGAAATTGCGAAAAGAGTAAAGGAAGGATTTACGCAAG
GTGCAGATATAGAACCAATTATATACGACTATATTAACAGCAACCATGCCGAGCTG
GGATTAAAATCTCCGTTAACCGGCAAACAACAACAAGAAATCACTGATAAATTTAC
AAAACATTATAATACGATTAAAGAATCTCCACATTTTGATGAGTTTTTTGTCGCTGA
TCCGGATAAAAAAGGCAATATCTTTTCTCATCAAGGCAGAATCAGTTGTCATTTTCT
GGATTTCTTTACTCGACAAACCAAAGGCAAACATCCTCTTGGTGATCTTGCAAGTCA
TCAGGAAGCTCTCCAGGAAGGAACCTCCAATCGCTTACATCACAAGAATGAGGTAG
TAGCCCAGGGGTACGAAAAACTGGATCAATTCAAGAAAGAGGTTGTCAAACTGCTG
GCTGAGAATAAACCAAAAGAATTATTGGATTATTTGGTTGCTACCTCACCTACAGGT
GTTCCAAATTACTCCATGCTTTCGAAGGAAACTCAAAATTACATTGCTTATAATCGT
AACTGGCCAGCCATTCAAAAAGAGCTGGAAAAGGCTACCAGCATCCCGGAGAGTCA
AAAACAAGATCTTTCAAGATTGCTTTCTCGTGATAATTTACAACACGATAATCTAAG
CGCAATTACCTGGTCAAAATATTCCTCCAAGCCATTATTGGATGTGGAATTAAATAA
AATCGCTGAAGGATTAGAACTCACTGCAAAAATTTACAATGAAAAGAGAGGACGCG
AATGGTGGTTTAAAGGTTCAAGAAATGAAGCTCGTAAGACCCAATGTGAAGAATTG
CAAAGAGTATCCAAAGAAATCAATACTCTTCTGCAAAGTGAATCTTTAACGAAAAG
CCAGGTACTTGAAAAGGTTTTAAATTCTATAGAAACATTAGATAAAATTGACAGAG
ACATTTCTGCCGAATCCAATTGGTTTCAAAGTACTCTGCAAAAGGAAGTCAGGTTAT
TTCGAGATCAATTGAAAGATATTTGCCAATTGGACAAGTATGCCTTTAAATCAACAA
AACTTGATGAAATCATCTCTCTGGAAATGGAAGAACAATTTCAAAAGATACAAGAT
CCTGCTGTTCAACAAATTGTCAGGGACTTGCCTTCTCATTGCCACAATGATGAAGCA
ATTGAATTCTTTAAGACATTGAACCCTGAAGAGGCAGCAAAAGTAGCTAGCTATTTA
AGCCTGGAATACAGGGAAATTAATAAATCAACCGATAAGAAAACTCTCCTAGAACA
AGATATTCCCAGACTGTTTAAAGAAGTCAATACGCAGTTACTCTCCAAACTCAAAGA
AGAAAAAGCTATTGATGAGCAAGTTCATGAAAAACTCAGTCAACTGGCTGACAAAA
TTGCCCCTGAGCATTTTACAAGAAATAACATTATAAAATGGTCTACCAACCCTGAAA
AGCTTGAGGAATCAAATCTTAATGAGCCAATCAAATCAGTCCAAAGCCCTACTACTA
AACAAACATCAAAACAATTCAGGGAAGCGATGGGTGAAATCACTGGAAGAAATGA
GCCTCCTACAGACACTTTGTACACGGGAATTATAAAGAAATAG

A further exemplary E3 ubiquitin ligase that is useful as a degradation domain in accordance with the present application includes the E3 ubiquitin ligase SlrP, which is a NEL motif, from EHEC O157:H7, and has the amino acid sequence of SEQ ID NO: 29:

-
MFNITNIQSTARHQSISNEASTEVPLKEEIWNKISAFFSSEHQVEAQNCI
AYLCEIPPETASPEEIKSKFECLRMLAFPAYADNIQYSRGGADQYCILSE
NSQEILSIVFNTEGYTVEGGGKSVTYTRVTESEQASSASGSKDAVNYELI
WSEWVKEAPAKEAANREEPVQRMRDCLKNNKTELRLKILGLTTIPAYIPE
QITTLILDNNELKSLPENLQGNIKTLYANSNQLTSIPATLPDTIQEMELS
INRITELPERLPSALQSLDLFHNKISCLPENLPEELRYLSVYDNSIRTLP
AHLPSEITHLNVQSNSLTALPETLPPGLKTLEAGENALTSLPASLPPELQ
VLDVSKNQITVLPETLPPTITTLDVSRNALTNLPENLPAALQIMQASRNN
LVRLPESLPHFRGEGPQPTRIIVEYNPFSERTIQNMQRLMSSVDYQGPRV
LVAMGDFSIVRVTRPLHQAVQGWLTSLEEEDVNQWRAFEAEANAAAFSGF
LDYLGDTQNTRHPDFKEQVSAWLMRLAEDSALRETVFIIAMNATISCEDR
VTLAYHQMQEATLVHDAERGAFDSHLAELIMAGREIFRLEQIESLAREKV
KRLFFIDEVEVFLGFQNQLRESLSLTTMTRDMRFYNVSGITESDLDEAEI
RIKMAENRDFHKWFALWGPWHKVLERIAPEEWREMMAKRDECIETDEYQS
RVNAELEDLRIADDSDAERTTEVQMDAERAIGIKIMEEINQTLFTEIMEN
ILLKKEVSSLMSAYWR

The E3 ubiquitin ligase SlrP, which is a NEL motif, from EHEC O157:H7, has the nucleotide sequence of SEQ ID NO: 30 as follows:

ATGTTTAATATTACTAATATACAATCTACGGCAAGGCATCAAAGTATTAGCAATGAG
GCCTCAACAGAGGTGCCTTTAAAAGAAGAGATATGGAATAAAATAAGTGCCTTTTT
CTCTTCAGAACATCAGGTTGAAGCACAAAACTGCATCGCTTATCTTTGTCATCCACC
TGAAACCGCCTCGCCAGAAGAGATCAAAAGCAAGTTTGAATGTTTAAGGATGTTAG
CTTTCCCGGCGTATGCGGATAATATTCAGTATAGTAGAGGAGGGGCAGACCAATAC
TGTATTTTGAGTGAAAATAGTCAGGAAATTCTGTCTATAGTTTTTAATACAGAGGGC
TATACCGTTGAGGGAGGGGGAAAGTCAGTCACCTATACCCGTGTGACAGAAAGCGA
GCAGGCGAGTAGCGCTTCCGGCTCCAAAGATGCTGTGAATTATGAGTTAATCTGGTC
TGAGTGGGTAAAAGAGGCGCCAGCGAAAGAGGCAGCAAATCGTGAAGAACCCGTA
CAACGGATGCGTGACTGCCTGAAAAATAATAAGACGGAACTTCGTCTGAAAATATT
AGGACTTACCACTATACCTGCCTATATTCCTGAGCAGATAACTACTCTGATACTCGA
TAACAATGAACTGAAAAGTTTGCCGGAAAATTTACAGGGAAATATAAAGACCCTGT
ATGCCAACAGTAATCAGCTAACCAGTATCCCTGCCACGTTACCGGATACCATACAGG
AAATGGAGCTGAGCATTAACCGTATTACTGAATTGCCGGAACGTTTGCCTTCAGCGC
TTCAATCGCTGGATCTTTTCCATAATAAAATTAGTTGCTTACCTGAAAATCTACCTGA
GGAACTTCGGTACCTGAGCGTTTATGATAACAGCATAAGGACACTGCCAGCACATCT
TCCGTCAGAGATTACCCATTTGAATGTGCAGAGTAATTCGTTAACCGCTTTGCCTGA
AACATTGCCGCCGGGCCTGAAGACTCTGGAGGCCGGCGAAAATGCCTTAACCAGTC
TGCCCGCATCGTTACCACCAGAATTACAGGTCCTGGATGTAAGTAAAAATCAGATTA
CGGTTCTGCCTGAAACACTTCCTCCCACGATAACAACGCTGGATGTTTCCCGTAACG
CATTGACTAATCTACCGGAAAACCTCCCGGCGGCATTACAAATAATGCAGGCCTCTC
GCAATAACCTGGTCCGTCTCCCGGAGTCGTTACCCCATTTTCGTGGTGAAGGACCTC
AACCTACAAGAATAATCGTAGAATATAATCCTTTTTCAGAACGAACAATACAGAAT
ATGCAGCGGCTAATGTCCTCTGTAGATTATCAGGGACCCCGGGTATTGGTTGCCATG
GGCGACTTTTCAATTGTTCGGGTAACTCGACCACTGCATCAAGCTGTCCAGGGGTGG
CTAACCAGTCTCGAGGAGGAAGACGTCAACCAATGGCGGGCGTTTGAGGCAGAGGC
AAACGCGGCGGCTTTCAGCGGATTCCTGGACTATCTTGGTGATACGCAGAATACCCG
ACACCCGGATTTTAAGGAACAAGTCTCCGCCTGGCTAATGCGCCTGGCTGAAGATA
GCGCACTAAGAGAAACCGTATTTATTATAGCGATGAATGCAACGATAAGCTGTGAA
GATCGGGTCACACTGGCATACCACCAAATGCAGGAAGCGACGTTGGTTCATGATGC
TGAAAGAGGCGCCTTTGATAGCCACTTAGCGGAACTGATTATGGCGGGGCGTGAAA
TCTTTCGGCTGGAGCAAATAGAATCGCTCGCCAGAGAAAAGGTAAAACGGCTGTTT
TTTATTGACGAAGTCGAAGTATTTCTGGGGTTTCAGAATCAGTTACGAGAGTCGCTG
TCGCTGACAACAATGACCCGGGATATGCGATTTTATAACGTTTCGGGTATCACTGAG
TCTGACCTGGACGAGGCGGAAATAAGGATAAAAATGGCTGAAAATAGGGATTTTCA
CAAATGGTTTGCGCTGTGGGGGCCGTGGCATAAAGTGCTGGAGCGCATAGCGCCAG
AAGAGTGGCGTGAAATGATGGCTAAAAGGGATGAGTGTATTGAAACGGATGAGTAT
CAGAGCCGGGTCAATGCTGAACTGGAAGATTTAAGAATAGCAGACGACTCTGACGC
AGAGCGTACTACTGAGGTACAGATGGATGCAGAGCGTGCTATTGGGATAAAAATAA
TGGAAGAGATCAATCAGACCCTCTTTACTGAGATCATGGAGAATATATTGCTGAAA
AAAGAGGTGAGCTCGCTCATGAGCGCCTACTGGCGATAG

A further exemplary E3 ubiquitin ligase that is useful as a degradation domain in accordance with the present application includes the E3 ubiquitin ligase SopA, which is a HECT motif, from Salmonella typhimurium, and has the amino acid sequence of SEQ ID NO: 31:

MKISSGAINFSTIPNQVKKLITSIREHTKNGLTSKITSVKNTHTSLNEKF
KTGKDSPIEFALPQKIKDFFQPKDKNTLNKTLITVKNIKDTNNAGKKNIS
AEDVSKMNAAFMRKHIANQTCDYNYRMTGAAPLPGGVSVSANNRPTVSEG
RTPPVSPSLSLQATSSPSSPADWAKKLTDAVLRQKAGETLTAADRDFSNA
DFRNITFSK1LPPSFMERDGDIIKGFNFSNSKFTYSDISHILHFDECRFT
YSTLSDVVCSNTKFSNSDMNEVFLQYSITTQQQPSFIDTTLKNTLIRIIK
ANLSGVILNEPDNSSPPSVSGGGNFIRLGDIWLQMPLLWTENAVDGFLNH
EHNNGKSILMTIDSLPDKYSQEKVQAMEDLVKSLRGGRLTEACIRPVESS
LVSVLAHPPYTQSALISEWLGPVQERFFAHQCQTYNDVPLPAPDTYYQQR
ILPVLLDSFDRNSAAMTTHSGLFNQVILHCMTGVDCTDGTRQKAAALYEQ
YLAHPAVSPHIHNGLFGNYDGSPDWTTRAADNFLLLSSQDSDTAMMLSTD
TLLTMLNPTPDTAWDNFYLLRAGENVSTAQISPVELFRHDFPVFLAAFNQ
QATQRRFGELIDIILSTEEHGELNQQFLAATNQKHSTVKLIDDASVSRLA
TIFDPLLPEGKLSPAHYQHILSAYHLTDATPQKQAETLFCLSTAFARYSS
SAIFGTEHDSPPALRGYAEALMQKAWELSPAIFPSSEQFTEWSDRFHGLH
GAFTCTSVVADSMQRHARKYFPSVLSSILPLAWA

The E3 ubiquitin ligase SopA, which is a HECT motif, from Salmonella typhimurium, has the nucleotide sequence of SEQ ID NO: 32 as follows:

ATGAAGATATCATCAGGCGCAATTAATTTTTCTACTATTCCTAACCAGGTTAAAAAA
TTAATTACCTCTATTCGTGAACATACGAAAAACGGGCTCACCTCAAAAATAACCAGT
GTTAAAAACACGCATACATCTTTAAATGAAAAATTTAAAACAGGAAAGGACTCACC
GATTGAGTTCGCGTTACCACAAAAAATAAAAGACTTCTTTCAGCCGAAAGATAAAA
ACACCTTAAACAAAACATTGATTACTGTTAAAAATATTAAAGATACAAATAATGCA
GGCAAGAAAAATATTTCAGCAGAAGATGTCTCAAAAATGAATGCAGCATTCATGCG
TAAGCATATTGCAAATCAAACATGTGATTATAATTACAGAATGACAGGTGCGGCCC
CGCTCCCCGGTGGAGTCTCTGTATCAGCCAATAACAGGCCCACGGTTTCTGAAGGTA
GAACACCACCAGTATCCCCCTCCCTCTCACTTCAGGCTACCTCTTCCCCGTCATCACC
TGCCGACTGGGCTAAGAAACTCACGGATGCAGTTTTACGACAGAAAGCCGGAGAAA
CCCTTACGGCCGCAGATCGCGATTTTTCAAACGCAGATTTCCGTAATATTACATTCA
GCAAAATATTGCCCCCCAGCTTCATGGAGCGAGACGGCGATATTATTAAGGGGTTC
AACTTTTCAAATTCAAAATTTACTTATTCTGATATATCTCATTTACATTTTGACGAAT
GCCGATTCACTTATTCGACACTGAGTGATGTAGTCTGCAGTAATACGAAATTTAGTA
ATTCAGACATGAATGAAGTGTTTTTACAGTATTCAATTACTACACAACAACAGCCCT
CGTTTATTGATACAACATTAAAAAATACGCTTATACGTCACAAAGCCAACCTCTCTG
GCGTTATTTTAAATGAACCGGATAATTCATCACCTCCGTCAGTGTCAGGGGGCGGAA
ATTTTATTCGTCTAGGTGATATCTGGCTGCAAATGCCACTCCTTTGGACTGAGAACG
CTGTGGATGGATTTTTAAATCATGAGCACAATAATGGTAAAAGTATTCTGATGACCA
TTGACAGCCTGCCCGATAAATACAGTCAGGAAAAAGTCCAGGCAATGGAAGACCTG
GTTAAGTCATTGCGGGGTGGCCGCTTAACAGAGGCATGTATCCGGCCAGTTGAAAG
TTCGCTGGTAAGCGTACTGGCCCACCCCCCCTATACGCAAAGTGCGCTTATCAGCGA
GTGGCTCGGGCCTGTTCAGGAACGTTTTTTTGCCCACCAGTGCCAGACCTATAATGA
CGTTCCCCTGCCGGCTCCTGACACATATTATCAGCAGCGCATACTGCCTGTGTTGCT
GGATTCGTTTGACAGGAACAGCGCCGCCATGACCACTCACAGCGGACTCTTTAATCA
GGTGATTTTACACTGTATGACAGGCGTGGACTGCACTGATGGCACCCGCCAGAAAG
CTGCAGCGCTTTATGAACAGTATCTTGCTCACCCGGCGGTGTCTCCCCACATCCATA
ATGGGCTCTTCGGCAATTATGATGGCAGCCCGGACTGGACAACCCGCGCTGCAGAT
AATTTCCTGCTGCTTTCCTCCCAAGATTCAGACACGGCGATGATGCTCTCCACTGAC
ACGCTGTTAACAATGCTAAACCCTACTCCTGACACTGCATGGGACAACTTTTACCTG
CTGCGAGCCGGAGAGAACGTTTCCACCGCGCAAATCTCTCCGGTAGAGTTATTCCGT
CATGACTTTCCGGTGTTTCTCGCCGCATTTAATCAGCAGGCCACGCAGCGACGCTTT
GGGGAGCTGATTGATATCATCCTCAGCACTGAAGAGCACGGGGAGCTGAACCAGCA
GTTTCTTGCCGCCACGAACCAGAAACATTCCACCGTGAAGTTGATTGATGATGCCTC
AGTGTCGCGTCTGGCCACCATTTTTGACCCCTTGCTTCCTGAAGGCAAACTCAGCCC
GGCACACTACCAGCACATCCTCAGTGCTTATCACCTGACGGACGCCACCCCACAGA
AGCAGGCGGAAACCCTGTTCTGTCTCAGTACCGCATTCGCACGCTATTCCTCCAGCG
CCATTTTCGGCACTGAGCATGACTCTCCGCCGGCCCTGAGAGGCTATGCGGAGGCGC
TGATGCAGAAAGCCTGGGAGCTGTCTCCGGCGATATTCCCATCCAGCGAACAGTTTA
CCGAGTGGTCCGACCGTTTTCACGGCCTCCATGGCGCCTTTACCTGTACCAGCGTTG
TGGCGGATAGTATGCAACGTCATGCCAGAAAATATTTCCCGAGTGTTCTGTCATCCA
TCCTGCCACTGGCCTGGGCGTAA

A further exemplary E3 ubiquitin ligase that is useful as a degradation domain in accordance with the present application includes the E3 ubiquitin ligase SspH1, which is a novel E3 ligase motif, from Salmonella typhimurium, and has the amino acid sequence of SEQ ID NO: 33:

MFNIRNTQPSVSMQAIAGAAAPEASPEEIVWEKIQVFFPQENYEEAQQCL
AELCHPARGMLPDHISSQFARLKALTFPAWEENIQCNRDGINQFCILDAG
SKEILSITLDDAGNYTVNCQGYSEAHDFIMDTEPGEECTEFAEGASGTSL
RPATTVSQKAAEYDAVWSKWERDAPAGESPGRAAVVQEMRDCLNNGNPVL
NVGASGLTTLPDRLPPHITTLVIPDNNLTSLPELPEGLRELEVSGNLQLT
SLPSLPQGLQKLWAYNNWLASLPTLPPGLGDLAVSNNQLTSLPEMPPALR
ELRVSGNNLTSLPALPSGLQKLWAYNNRLTSLPEMSPGLQELDVSHNQLT
RLPQSLTGLSSAARVYLDGNPLSVRTLQALRDIIGHSGIRIHFDMAGPSV
PREARALEILAVADWLTSAREGEAAQADRWQAFGLEDNAAAFSLVLDRLR
ETENFKKDAGFKAQISSWLTQLAEDAALRAKTFAMATEATSTCEDRVTHA
LHQMNNVQLVHNAEKGEYDNNLQGLVSTGREMFRLATLEQIAREKAGTLA
LVDDVEVYLAFQNKLKESLELTSVTSEMRFFDVSGVTVSDLQAAELQVKT
AENSGFSKWILQWGPLHSVLERKVPERFNALREKQISDYEDTYRKLYDEV
LKSSGLVDDTDAERTIGVSAMDSAKKEFLDGLRALVDEVLGSYLTARWRL
N

The E3 ubiquitin ligase SspH1, which is a novel E3 ligase motif, from Salmonella typhimurium, has the nucleotide sequence of SEQ ID NO: 34 as follows:

ATGTTTAATATCCGCAATACACAACCTTCTGTAAGTATGCAGGCTATTGCTGGTGCA
GCGGCACCAGAGGCATCTCCGGAAGAAATTGTATGGGAAAAAATTCAGGTTTTTTTC
CCGCAGGAAAATTACGAAGAAGCGCAACAGTGTCTCGCTGAACTTTGCCATCCGGC
CCGGGGAATGTTGCCTGATCATATCAGCAGCCAGTTTGCGCGTTTAAAAGCGCTTAC
CTTCCCCGCGTGGGAGGAGAATATTCAGTGTAACAGGGATGGTATAAATCAGTTTTG
TATTCTGGATGCAGGCAGCAAGGAGATATTGTCAATCACTCTTGATGATGCCGGGAA
CTATACCGTGAATTGTCAGGGGTACAGTGAAGCACATGACTTCATCATGGACACAG
AACCGGGAGAGGAATGCACAGAATTCGCGGAGGGGGCATCCGGGACATCCCTCCGC
CCTGCCACAACGGTTTCACAGAAGGCAGCAGAGTATGATGCTGTCTGGTCAAAATG
GGAAAGGGATGCACCAGCAGGAGAGTCACCCGGCCGCGCAGCAGTGGTACAGGAA
ATGCGTGATTGCCTGAATAACGGCAATCCAGTGCTTAACGTGGGAGCGTCAGGTCTT
ACCACCTTACCAGACCGTTTACCACCGCATATTACAACACTGGTTATTCCTGATAAT
AATCTGACCAGCCTGCCGGAGTTGCCGGAAGGACTACGGGAGCTGGAGGTCTCTGG
TAACCTACAACTGACCAGCCTGCCATCGCTGCCGCAGGGACTACAGAAGCTGTGGG
CCTATAATAATTGGCTGGCCAGCCTGCCGACGTTGCCGCCAGGACTAGGGGATCTGG
CGGTCTCTAATAACCAGCTGACCAGCCTGCCGGAGATGCCGCCAGCACTACGGGAG
CTGAGGGTCTCTGGTAACAACCTGACCAGCCTGCCGGCGCTGCCGTCAGGACTACA
GAAGCTGTGGGCCTATAATAATCGGCTGACCAGCCTGCCGGAGATGTCGCCAGGAC
TACAGGAGCTGGATGTCTCTCATAACCAGCTGACCCGCCTGCCGCAAAGCCTCACGG
GTCTGTCTTCAGCGGCACGCGTATATCTGGACGGGAATCCACTGTCTGTACGCACTC
TGCAGGCTCTGCGGGACATCATTGGCCATTCAGGCATCAGGATACACTTCGATATGG
CGGGGCCTTCCGTCCCCCGGGAAGCCCGGGCACTGCACCTGGCGGTCGCTGACTGG
CTGACGTCTGCACGGGAGGGGGAAGCGGCCCAGGCAGACAGATGGCAGGCGTTCG
GACTGGAAGATAACGCCGCCGCCTTCAGCCTGGTCCTGGACAGACTGCGTGAGACG
GAAAACTTCAAAAAAGACGCGGGCTTTAAGGCACAGATATCATCCTGGCTGACACA
ACTGGCTGAAGATGCTGCGCTGAGAGCAAAAACCTTTGCCATGGCAACAGAGGCAA
CATCAACCTGCGAGGACCGGGTCACACATGCCCTGCACCAGATGAATAACGTACAA
CTGGTACATAATGCAGAAAAAGGGGAATACGACAACAATCTCCAGGGGCTGGTTTC
CACGGGGCGTGAGATGTTCCGCCTGGCAACACTGGAACAGATTGCCCGGGAAAAAG
CCGGAACACTGGCTTTAGTCGATGACGTTGAGGTCTATCTGGCGTTCCAGAATAAGC
TGAAGGAATCACTTGAGCTGACCAGCGTGACGTCAGAAATGCGTTTCTTTGACGTTT
CCGGCGTGACGGTTTCAGACCTTCAGGCTGCGGAGCTTCAGGTGAAAACCGCTGAA
AACAGCGGGTTCAGTAAATGGATACTGCAGTGGGGGCCGTTACACAGCGTGCTGGA
ACGCAAAGTGCCGGAACGCTTTAACGCGCTTCGTGAAAAGCAAATATCGGATTATG
AAGACACGTACCGGAAGCTGTATGACGAAGTGCTGAAATCGTCCGGGCTGGTCGAC
GATACCGATGCAGAACGTACTATCGGAGTAAGTGCGATGGATAGTGCGAAAAAAGA
ATTTCTGGATGGCCTGCGCGCTCTTGTGGATGAGGTGCTGGGTAGCTATCTGACAGC
CCGGTGGCGTCTTAACTAA

A further exemplary E3 ubiquitin ligase that is useful as a degradation domain in accordance with the present application includes the E3 ubiquitin ligase SspH2, which is a novel E3 ligase motif, from Salmonella typhimurium, and has the amino acid sequence of SEQ ID NO: 35:

MPFHIGSGCLPATISNRRIYRIAWSDTPPEMSSWEKMKEFFCSTHQTEAL
ECIWTICIIPPAGTTREDVINRFELLRTLAYAGWEESIHSGQHGENYFCI
LDEDSQEILSVTLDDAGNYTVNCQGYSETHRLTLDTAQGEEGTGHAEGAS
GTFRTSFLPATTAPQTPAEYDAVWSAWRRAAPAEESRGRAAVVQKMRACL
NNGNAVLNVGESGLTTLPDCLPAHITTLVIPDNNLTSLPALPPELRTLEV
SGNQLTSLPVLPPGLLELSIFSNPLTHLPALPSGLCKLWIFGNQLTSLPV
LPPGLQELSVSDNQLASLPALPSELCKLWAYNNQLTSLPMLPSGLQELSV
SDNQLASLPTLPSELYKLWAYNNRLTSLPALPSGLKELIVSGNRLTSLPV
LPSELKELMVSGNRLTSLPMLPSGLLSLSVYRNQLTRLPESLIHLSSETT
VNLEGNPLSERTLQALREITSAPGYSGPIIRFDMAGASAPRETRALHLAA
ADWLVPAREGEPAPADRWHMIFGQEDNADAFSLFLDRLSETENFIKDAGF
KAQISSWLAQLAEDEALRANTFAMATEATSSCEDRVTFFLHQMKNVQLVH
NAEKGQYDNDLAALVATGREMFRLGKLEQIAREKVRTLALVDEIEVWLAY
QNKLKKSLGLTSVTSEMRFFDVSGVTVTDLQDAELQVKAAEKSEFREWIL
QWGPLEIRVLERKAPERVNALREKQISDYEETYRMLSDTELRPSGLVGNT
DAERTIGARAMESAKKTFLDGLRPLVEEMLGSYLNVQWRRN

The E3 ubiquitin ligase SspH2, which is a novel E3 ligase motif, from Salmonella typhimurium, has the nucleotide sequence of SEQ ID NO: 36 as follows:

ATGCCCTTTCATATTGGAAGCGGATGTCTTCCCGCCACCATCAGTAATCGCCGCATT
TATCGTATTGCCTGGTCTGATACCCCCCCTGAAATGAGTTCCTGGGAAAAAATGAAG
GAATTTTTTTGCTCAACGCACCAGACTGAAGCGCTGGAGTGCATCTGGACGATTTGT
CACCCGCCGGCCGGAACGACGCGGGAGGATGTGATCAACAGATTTGAACTGCTCAG
GACGCTCGCGTATGCCGGATGGGAGGAAAGCATTCATTCCGGCCAGCACGGGGAAA
ATTACTTCTGTATTCTGGATGAAGACAGTCAGGAGATATTGTCAGTCACCCTTGATG
ATGCCGGGAACTATACCGTAAATTGCCAGGGGTACAGTGAAACACATCGCCTCACC
CTGGACACAGCACAGGGTGAGGAGGGCACAGGACACGCGGAAGGGGCATCCGGGA
CATTCAGGACATCCTTCCTCCCTGCCACAACGGCTCCACAGACGCCAGCAGAGTATG
ATGCTGTCTGGTCAGCGTGGAGAAGGGCTGCACCCGCAGAAGAGTCACGCGGCCGT
GCAGCAGTGGTACAGAAAATGCGTGCCTGCCTGAATAATGGCAATGCAGTGCTTAA
CGTGGGAGAATCAGGTCTTACCACCTTGCCAGACTGTTTACCCGCGCATATTACCAC
ACTGGTTATTCCTGATAATAATCTGACCAGCCTGCCGGCGCTGCCGCCAGAACTGCG
GACGCTGGAGGTCTCTGGTAACCAGCTGACTAGCCTGCCGGTGCTGCCGCCAGGACT
ACTGGAACTGTCGATCTTTAGTAACCCGCTGACCCACCTGCCGGCGCTGCCGTCAGG
ACTATGTAAGCTGTGGATCTTTGGTAATCAACTGACCAGCCTGCCGGTGTTGCCGCC
AGGGCTACAGGAGCTGTCGGTATCTGATAACCAACTGGCCAGCCTGCCGGCGCTGC
CGTCAGAATTATGTAAGCTGTGGGCCTATAATAACCAGCTGACCAGCCTGCCGATGT
TGCCGTCAGGGCTACAGGAGCTGTCGGTATCTGATAACCAACTGGCCAGCCTGCCG
ACGCTGCCGTCAGAATTATATAAGCTGTGGGCCTATAATAATCGGCTGACCAGCCTG
CCGGCGTTGCCGTCAGGACTGAAGGAGCTGATTGTATCTGGTAACCGGCTGACCAGT
CTGCCGGTGCTGCCGTCAGAACTGAAGGAGCTGATGGTATCTGGTAACCGGCTGAC
CAGCCTGCCGATGCTGCCGTCAGGACTACTGTCGCTGTCGGTCTATCGTAACCAGCT
GACCCGCCTGCCGGAAAGTCTCATTCATCTGTCTTCAGAGACAACCGTAAATCTGGA
AGGGAACCCACTGTCTGAACGTACTTTGCAGGCGCTGCGGGAGATCACCAGCGCGC
CTGGCTATTCAGGCCCCATAATACGATTCGATATGGCGGGAGCCTCCGCCCCCCGGG
AAACTCGGGCACTGCACCTGGCGGCCGCTGACTGGCTGGTGCCTGCCCGGGAGGGG
GAACCGGCTCCTGCAGACAGATGGCATATGTTCGGACAGGAAGATAACGCCGACGC
ATTCAGCCTCTTCCTGGACAGACTGAGTGAGACGGAAAACTTCATAAAGGACGCGG
GGTTTAAGGCACAGATATCGTCCTGGCTGGCACAACTGGCTGAAGATGAGGCGTTA
AGAGCAAACACCTTTGCTATGGCAACAGAGGCAACCTCAAGCTGCGAGGACCGGGT
CACATTTTTTTTGCACCAGATGAAGAACGTACAGCTGGTACATAATGCAGAAAAAG
GGCAATACGATAACGATCTCGCGGCGCTGGTTGCCACGGGGCGTGAGATGTTCCGT
CTGGGAAAACTGGAACAGATTGCCCGGGAAAAGGTCAGAACGCTGGCTCTCGTTGA
TGAAATTGAGGTCTGGCTGGCGTATCAGAATAAGCTGAAGAAATCACTCGGGCTGA
CCAGCGTGACGTCAGAAATGCGTTTCTTTGACGTATCCGGCGTGACGGTTACAGACC
TTCAGGACGCGGAGCTTCAGGTGAAAGCCGCTGAAAAAAGCGAGTTCAGGGAGTGG
ATACTGCAGTGGGGGCCGTTACACAGAGTGCTGGAGCGCAAAGCGCCGGAACGCGT
TAACGCGCTTCGTGAAAAGCAAATATCGGATTATGAGGAAACGTACCGGATGCTGT
CTGACACAGAGCTGAGACCGTCTGGGCTGGTCGGTAATACCGATGCAGAGCGCACT
ATCGGAGCAAGAGCGATGGAGAGCGCGAAAAAGACATTTTTGGATGGCCTGCGACC
TCTTGTGGAGGAGATGCTGGGGAGCTATCTGAACGTTCAGTGGCGTCGTAACTGA

A further exemplary E3 ubiquitin ligase that is useful as a degradation domain in accordance with the present application includes the E3 ubiquitin ligase XopL, which is an unconventional motif, from Xanthomonas campestris, and has the amino acid sequence of SEQ ID NO: 37:

MRRVDQPRPPGTPFGLREQTTSNADAPARTAPPAHPAPERPTGMLGGLTR
YVPGDRSGRPPAMPAAAETSRRPTTSARPLPYGGSGSAARMNEAAGHPLR
MPQLPQLSDIERARFHSVTTDSQHLRPVRPRMPPPVGASPLRRSTALRPY
HDVLSQWQRHYNADRNRWHSAWRQANSNNPQIETRTGRALKATADLLEDA
TQPGRVALELRSVPLPQFPDQAFRLSHLQHMTIDAAGLMELPDTMQQFAG
LETLTLARNPLRALPASIASLNRLRELSIRACPELTELPEPLASTDASGE
HQGLVNLQSLRLEWTGIRSLPASIANLQNLKSLKIRNSPLSALGPAIHHL
PKLEELDLRGCTALRNYPPIFGGRAPLKRLILKDCSNLLTLPLDIHRLTQ
LEKLDLRGCVNLSRLPSLIAQLPANCIILVPPHLQAQLDQHRPVARPAEP
GRTGPTTPALSPSAAGDRAGPSSSATASELLLTAALERIEDTAQAMLSTV
IDEERNPFLEGAPSYLPGKRPTDVTTFGQVPALRDMLAESRDLEFLQRVS
DMAGPSPRIEDPSEEGLARHYTNVSNWKAQKSAHLGIVDHLGQFVYHEGS
PLDVATLAKAVQMWKTRELIVHAHPQDRARFPELAVHIPEQVSDDSDSEQ
QTSPEPSGHQ

The E3 ubiquitin ligase XopL, which is an unconventional motif, from Xanthomonas campestris, has the nucleotide sequence of SEQ ID NO: 38 as follows:

ATGCGACGCGTCGATCAACCACGCCCGCCGGGCACGCCTTTCGGACTGCGGGAGCA
GACTACGTCCAATGCGGATGCGCCCGCGCGCACTGCCCCACCCGCACACCCCGCGC
CCGAGCGCCCTACCGGCATGCTCGGCGGACTGACCAGATATGTGCCTGGCGATCGG
TCCGGGCGACCGCCAGCAATGCCTGCCGCTGCCGAGACCTCTCGCCGGCCAACCAC
CTCCGCCCGCCCGCTTCCCTACGGCGGATCCGGCAGCGCCGCGCGGATGAACGAGG
CGGCTGGACATCCTTTGCGGATGCCGCAATTGCCACAGCTCAGCGACATAGAACGC
GCTCGCTTCCACTCCGTCACCACCGACTCGCAACACTTGCGGCCGGTGCGCCCCCGT
ATGCCACCGCCCGTGGGCGCTTCACCCTTACGGCGCTCCACAGCGCTGCGCCCGTAC
CACGACGTGCTGTCGCAATGGCAACGCCACTACAACGCAGATCGCAATCGCTGGCA
CAGCGCATGGCGCCAGGCCAACAGCAACAACCCGCAGATCGAGACTCGCACAGGCC
GGGCGCTGAAGGCGACAGCCGACCTGCTGGAGGACGCAACCCAACCGGGCCGGGTC
GCGCTGGAGCTGCGCTCAGTTCCGCTGCCGCAATTTCCCGACCAGGCATTCCGTCTT
TCGCATCTGCAGCACATGACGATCGACGCGGCAGGGTTGATGGAGCTCCCGGACAC
CATGCAGCAATTTGCGGGCCTGGAAACACTCACGCTCGCACGCAATCCGCTTCGCGC
GCTACCGGCATCCATCGCAAGCCTCAACCGATTACGCGAGCTCTCCATCCGCGCCTG
CCCGGAATTGACGGAACTTCCCGAACCCCTGGCAAGCACCGATGCATCCGGCGAGC
ACCAGGGCTTGGTCAACCTGCAGAGCCTACGGCTGGAATGGACCGGGATCAGATCG
CTTCCGGCGTCCATCGCCAACCTGCAAAATCTGAAAAGCCTGAAGATACGCAACTC
GCCGCTGTCCGCCCTTGGCCCGGCCATCCATCACCTGCCAAAGTTGGAGGAGCTTGA
TTTGCGGGGCTGTACCGCGCTGCGCAACTATCCGCCGATTTTCGGCGGCCGTGCGCC
ACTGAAGCGACTGATTCTGAAAGACTGCAGCAACCTGCTCACGCTGCCACTGGACA
TTCACCGCCTGACGCAGCTGGAAAAACTCGATCTGCGAGGTTGCGTCAACCTTTCCA
GACTGCCCTCGTTGATCGCCCAATTACCTGCCAATTGCATCATCCTGGTGCCGCCGC
ATCTCCAAGCGCAGCTCGACCAGCATCGTCCAGTTGCGCGCCCCGCCGAACCAGGG
CGGACCGGACCGACCACCCCAGCTCTCTCGCCCTCTGCTGCCGGCGACCGCGCCGGG
CCATCCTCTTCGGCGACCGCCAGCGAACTGCTTCTTACCGCTGCGCTCGAACGCATC
GAAGACACCGCACAGGCCATGCTGAGCACGGTCATCGATGAAGAAAGAAATCCCTT
TCTGGAAGGTGCTCCATCCTATCTCCCAGGAAAACGCCCTACCGATGTCACCACCTT
CGGCCAAGTTCCGGCATTGCGGGACATGCTGGCAGAAAGCAGGGATCTTGAGTTCC
TGCAACGGGTAAGCGACATGGCAGGCCCATCCCCCAGAATCGAAGACCCGAGCGAG
GAAGGCCTCGCCCGCCACTACACGAACGTCAGCAACTGGAAGGCGCAGAAGAGCGC
ACACCTGGGCATCGTCGATCATCTCGGGCAGTTCGTTTATCACGAAGGAAGCCCGCT
CGACGTAGCGACATTGGCCAAGGCAGTGCAGATGTGGAAGACCCGTGAGCTGATCG
TCCACGCACACCCGCAAGACCGCGCGCGCTTTCCCGAGCTCGCTGTGCACATTCCCG
AGCAGGTCAGCGACGACTCTGATAGCGAACAGCAGACAAGCCCGGAACCTTCAGGC
CATCAGTAG

Although targeting domains possess intrinsic binding interactions, e.g., secondary, tertiary or quaternary flexibility, there must still be flexibility with respect to the association with the E3 motif ubiquitin region. In this regard, absence adequate spacing, it is possible for the E3 motif to sterically hinder the substrate-target domain interaction. As such, the present application employs polypeptide linkers of sufficient length to prevent the steric disruption of binding between the targeting domain and the substrate, in some embodiments.

In some embodiments, the targeting domain is covalently attached to the ubiquitin region via a linker that may be cleavable or non-cleavable under physiological conditions. The linker can entail an organic moiety comprising a nucleophilic or electrophilic reacting group which allows covalent attachment to the targeting domain to the ubiquitin region agent. In some embodiments, the linker is an enol ether, ketal, imine, oxime, hydrazone, semicarbazone, acylimide, or methylene radical. The linker may be an acid-cleavable linker, a hydrolytically cleavable linker, or enzymatically-cleavable linker, in some embodiments.

Peptide-based linking groups are cleaved by enzymes such as peptidases and proteases in cells. Peptide-based cleavable linking groups are peptide bonds formed between amino acids to yield oligopeptides, e.g., dipeptides, tripeptides, and poly-peptides. Peptide-based cleavable groups do not include the amide group (—C(O)NH—). The amide group can be formed between any alkylene, alkenylene or alkynelene. A peptide bond is a special type of amide bond formed between amino acids to yield peptides and proteins. The peptide based cleavage group is generally limited to the peptide bond, i.e., the amide bond, formed between amino acids yielding peptides and proteins and does not include the entire amide functional group. Peptide cleavable linking groups have the general formula —NHCHR1C(O)NHCHR2C(O)—, where R1 and R2 are the R groups of the two adjacent amino acids. These candidates can be evaluated using methods analogous to those described above.

For in vitro applications, appropriate linkers, which can be cross-linking agents for use for conjugating a polypeptide to a solid support, include a variety of agents that can react with a functional group present on a surface of the support, or with the polypeptide, or both. Reagents useful as cross-linking agents include homo-bi-functional and, in particular, hetero-bi-functional reagents. Useful bi-functional cross-linking agents include, but are not limited to, N-SIAB, dimaleimide, DTNB, N-SATA, N-SPDP, SMCC and 6-HYNIC. A cross-linking agent can be selected to provide a selectively cleavable bond between a polypeptide and the solid support. For example, a photolabile cross-linker, such as 3-amino-(2-nitrophenyl)propionic acid can be employed as a means for cleaving a polypeptide from a solid support. See Brown et al., “A Single-Bead Decode Strategy Using Electrospray Ionization Mass Spectrometry and a New Photolabile Linker: 3-amino-3-(2-nitrophenyl)propionic Acid,” Mol. Divers 4-12 (1995) and U.S. Pat. No. 5,643,722, which are hereby incorporated by reference in their entirety.

An antibody, polypeptide, or fragment thereof, such as a targeting domain, can be immobilized on a solid support, such as a bead, through a covalent amide bond formed between a carboxyl group functionalized bead and the amino terminus of the polypeptide or, conversely, through a covalent amide bond formed between an amino group functionalized bead and the carboxyl terminus of the polypeptide. In addition, a bi-functional trityl linker can be attached to the support, e.g, to the 4-nitrophenyl active ester on a resin, such as a Wang resin, through an amino group or a carboxyl group on the resin via an amino resin. Using a bi-functional trityl approach, the solid support can require treatment with a volatile acid, such as formic acid or trifluoracetic acid to ensure that the polypeptide is cleaved and can be removed. In such a case, the polypeptide can be deposited as a beadless patch at the bottom of a well of a solid support or on the flat surface of a solid support. After addition of a matrix solution, the polypeptide can be desorbed into a MS.

It will be readily apparent to the skilled artisan that the methods and techniques described above can be employed for the chimeric molecule of the present application, including its constituent parts, e.g., degradation domains, ubiquitin regions, target regions, and modifications thereof, as well as the linker molecules, as described above.

A second aspect of the present application relates to a method of forming a ribonucleoprotein. The method includes providing a mRNA encoding the isolated chimeric molecule described herein; providing one or more polyadenosine binding proteins (“PABP”); and assembling a ribonucleoprotein complex from the mRNA and the one or more PABPs. In one embodiment, the mRNA comprises a 3′-terminal polyadenosine (poly A) tail.

The chimeric molecule described in this aspect is carried out in accordance with the previously described aspect of the application.

The polyadenosine binding proteins (“PABP”) (also referred to as poly(A)-binding proteins) as described herein refer to a RNA-binding protein which binds to the poly(A) tail of mRNA. The poly(A) tail is located on the 3′ end of mRNA and is 200-250 nucleotides long. The binding protein is also involved in mRNA precursors by helping polyadenylate polymerase add the poly(A) nucleotide tail to the pre-mRNA before translation. The nuclear isoform selectively binds to around 50 nucleotides and stimulates the activity of polyadenylate polymerase by increasing its affinity towards RNA. Poly(A)-binding protein is also present during stages of mRNA metabolism including nonsense-mediated decay and nucleocytoplasmic trafficking. The poly(A)-binding protein may also protect the tail from degradation and regulate mRNA production.

The ribonucleoprotein, which is also referred to herein as a nanoplex, may, in one embodiment, be a nanoparticle. In a preferred embodiment, the nanoplex includes a nanoparticle. The ribonucleoprotein or nanoplex is a complex formed by a drug nanoparticle with an oppositely charged polyelectrolyte. Both cationic and anionic drugs form complexes with oppositely charged polyelectrolytes. Compared with other nanostructures, the yield of Nanoplex is generally greater and the complexation efficiency is generally better. Nanoplexes are also easier to prepare as compared to other nanostructures. Ribonucleoprotein or nanoplex formulation according to the present application is characterized through the production yield, complexation efficiency, drug loading, particle size and zeta potential using scanning electron microscopy, differential scanning calorimetry, X-ray diffraction and dialysis studies. Nanoplexes have wide-ranging applications in different fields such as cancer therapy, gene drug delivery, drug delivery to the brain and protein and peptide drug delivery.

The ribonucleoprotein or nanoplex can have any suitable size. In at least one embodiment, ribonucleoprotein or nanoplex is less than about 200 nm in diameter, less than about 100 nm in diameter, less than about 95 nm, less than about 90 nm, less than about 85 nm, less than about 80 nm, less than about 75 nm, less than about 70 nm, less than about 65 nm, less than about 60 nm, less than about 55 nm, less than about 50 nm, less than about 45 nm, less than about 40 nm, less than about 35 nm, less than about 30 nm, less than about 25 nm, less than about 20 nm, less than about 15 nm, less than about 10 nm, less than about 9 nm, less than about 8 nm, less than about 7 nm, less than about 6 nm, less than about 5 nm, less than about 4 nm, less than about 3 nm, less than about 2 nm, or less than about 1 nm. Nanoparticles having a diameter in a range having an upper limit of about 100 nm, about 95 nm, about 90 nm, about 85 nm, about 80 nm, about 75 nm, about 70 nm, about 65 nm, about 60 nm, about 55 nm, about 50 nm, about 45 nm, about 40 nm, about 35 nm, about 30 nm, about 25 nm, about 20 nm, about 15 nm, about 10 nm, about 9 nm, about 8 nm, about 7 nm, about 6 nm, about 5 nm, about 4 nm, about 3 nm, or about 2 nm and a lower limit of about 95 nm, about 90 nm, about 85 nm, about 80 nm, about 75 nm, about 70 nm, about 65 nm, about 60 nm, about 55 nm, about 50 nm, about 45 nm, about 40 nm, about 35 nm, about 30 nm, about 25 nm, about 20 nm, about 15 nm, about 10 nm, about 9 nm, about 8 nm, about 7 nm, about 6 nm, about 5 nm, about 4 nm, about 3 nm, about 2 nm, or about 1 nm, and any combination thereof, are also contemplated. The present application further provides that in certain embodiments the nanoplex ranges in size from about 50 nm to about 200 nm or from about 10 nm to about 100 nm. In certain embodiments, the size of the nanoplex is about 50 nm to about 150 nm. In other embodiments, the size of the nanoplex can be about 50 nm, about 75 nm, or about 100 nm.

The size of the ribonucleoprotein and/or nanoplex may be optimized to localize at a particular location within a subject. For example, the ribonucleoprotein and/or nanoplex may be optimized so that it can move into various organs of a subject.

In one embodiment, the ribonucleoprotein and/or nanoplex contains an organic gel.

The ribonucleoprotein and/or nanoplex of the present application can have any suitable shape. For example, the present ribonucleoprotein and/or nanoplex can have a shape of a mesh, sphere, square, rectangle, triangle, circular disc, cube-like shape, cube, rectangular parallelepiped (cuboid), cone, cylinder, prism, polyhedral, pyramid, right-angled circular cylinder, rod, branched cylindrical, and other regular or irregular shape. The polymer matrix of the ribonucleoprotein and/or nanoplex may, in one embodiment, contain entangled and covalently bound polymers. In one embodiment, the matrix is a hydrogel.

In one embodiment, the number of polymeric units in the ribonucleoprotein and/or nanoplex matrix ranges from 10 to 5000, for instance from 20 to 400, for each particle formed from the polymeric units. In another embodiment, the number of polymeric units ranges from 10,000 to 200,000, for instance from 15,000 to 200,000 polymeric units.

The ribonucleoprotein and/or nanoplex according to the present application may include a functionalized surface. In one embodiment, the ribonucleoprotein and/or nanoplex is negatively functionalized. Alternatively, the ribonucleoprotein and/or nanoplex may be positively functionalized. In other embodiments, the ribonucleoprotein and/or nanoplex has a no charge or is neutral.

In one embodiment, the ribonucleoprotein and/or nanoplex is biodegradable. In another embodiment, the ribonucleoprotein and/or nanoplex is non-toxic.

In one embodiment, the ribonucleoprotein and/or nanoplex may further include at least one stabilizer. The stabilizer may be adsorbed on the surfaces of the ribonucleoprotein and/or nanoplex. The ribonucleoprotein and/or nanoplex may be dispersed into a liquid medium, and the stabilizer may be employed as an adjuvant to aid in the separation of the individual ribonucleoprotein and/or nanoplex during a dispersion process. The ability of a stabilizer to aid in the separation of the individual ribonucleoprotein and/or nanoplex may be determined by comparing the dispersion processes for a composition containing the stabilizer and a control composition without the stabilizer. The ability of a stabilizer to aid in the separation of individual nanoparticles may be indicated by shorter dispersion times. Alternatively, the stabilizer may be employed to promote stability of the dispersed nanoplex in the liquid medium, preferably an aqueous medium.

A third aspect of the present application relates to a composition comprising the chimeric molecule described herein and a pharmaceutically-acceptable carrier.

According to the methods of the present application, the chimeric molecule can be incorporated into pharmaceutical compositions suitable for administration. As used herein, the term “pharmaceutically-acceptable carrier” is intended to include any and all solvents, dispersion media, coatings, antibacterial and antifungal compounds, isotonic and absorption delaying compounds, and the like, compatible with pharmaceutical administration.

The chimeric molecule described in this aspect is carried out in accordance with the previously described aspects of the present application.

The pharmaceutical compositions generally entail recombinant or substantially purified chimeric molecules and a pharmaceutically-acceptable carrier in a form suitable for administration to a subject. Pharmaceutically-acceptable carriers are determined in part by the particular composition being administered, as well as by the particular method used to administer the composition. Accordingly, there is a wide variety of suitable formulations of pharmaceutical compositions for administering the protein compositions. See, e.g., Remington's Pharmaceutical Sciences, Mack Publishing Co., Easton, Pa. 18^{th} ed. (1990), which is hereby incorporated by reference in its entirety. The pharmaceutical compositions are generally formulated as sterile, substantially isotonic and in full compliance with all Good Manufacturing Practice (GMP) regulations of the U.S. Food and Drug Administration.

As used herein, the term pharmaceutically-acceptable carrier includes, for example, a non-toxic, inert solid, semi-solid or liquid filler, diluent, encapsulating material or formulation auxiliary of any type. Remington's Pharmaceutical Sciences Ed. by Gennaro, Mack Publishing, Easton, Pa., 1995, which is hereby incorporated by reference in its entirety, discloses various carriers used in formulating pharmaceutical compositions and known techniques for the preparation thereof. Some examples of materials which can serve as pharmaceutically acceptable carriers include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as TWEEN™ 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate. Coloring agents, releasing agents, coating agents, sweetening, flavoring and perfuming agents, preservatives and/or antioxidants can also be present in the composition, according to the judgment of the formulator.

The compounds of the present application can be administered orally, parenterally, for example, subcutaneously, intravenously, intramuscularly, intraperitoneally, by intranasal instillation, or by application to mucous membranes, such as, that of the nose, throat, and bronchial tubes. They may be administered alone or with suitable pharmaceutical carriers, and can be in solid or liquid form such as, tablets, capsules, powders, solutions, suspensions, or emulsions.

The compositions of the present application may be orally administered, for example, with an inert diluent, or with an assimilable edible carrier, or they may be enclosed in hard or soft shell capsules, or they may be compressed into tablets, or they may be incorporated directly with the food of the diet. For oral therapeutic administration, these compositions may be incorporated with excipients and used in the form of tablets, capsules, elixirs, suspensions, syrups, and the like. Such compositions and preparations should contain at least 0.1% of active compound. The percentage of the composition in these compositions may, of course, be varied and may conveniently be between about 2% to about 60% of the weight of the unit. Preferred compositions according to the present application are prepared so that an oral dosage unit contains between about 1 and 250 mg of active compound.

The tablets, capsules, and the like may also contain a binder such as gum tragacanth, acacia, corn starch, or gelatin; excipients such as dicalcium phosphate; a disintegrating agent such as corn starch, potato starch, alginic acid; a lubricant such as magnesium stearate; and a sweetening agent such as sucrose, lactose, or saccharin. When the dosage unit form is a capsule, it may contain, in addition to materials of the above type, a liquid carrier, such as a fatty oil.

These compositions may also be administered parenterally. Solutions or suspensions of the present compositions can be prepared in water suitably mixed with a surfactant, such as hydroxypropylcellulose. Dispersions can also be prepared in glycerol, liquid polyethylene glycols, and mixtures thereof in oils. Illustrative oils are those of petroleum, animal, vegetable, or synthetic origin, for example, peanut oil, soybean oil, or mineral oil. In general, water, saline, aqueous dextrose and related sugar solution, and glycols such as, propylene glycol or polyethylene glycol, are preferred liquid carriers, particularly for injectable solutions. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol), suitable mixtures thereof, and vegetable oils.

The composition of the present application may also be administered directly to the airways in the form of an aerosol. For use as aerosols, the compositions of the present application in solution or suspension may be packaged in a pressurized aerosol container together with suitable propellants, for example, hydrocarbon propellants like propane, butane, or isobutane with conventional adjuvants. The materials of the present application also may be administered in a non-pressurized form such as in a nebulizer or atomizer.

The compositions of the present application further contain, in some embodiments, a second agent or pharmaceutical composition selected from the non-limiting group of anti-inflammatory agents, antidiabetic agents, hpyolipidemic agents, chemotherapeutic agents, antiviral agents, antibiotics, metabolic agents, small molecule inhibitors, protein kinase inhibitors, adjuvants, apoptotic agents, proliferative agents, organotropic targeting agents, immunological agents, antigens from pathogens, such as viruses, bacteria, fungi and parasites, optionally in the form of whole inactivated organisms, peptides, proteins, glycoproteins, carbohydrates, or combinations thereof, any examples of pharmacological or immunological agents that fall within the above-mentioned categories and that have been approved for human use that may be found in the published literature, any other bioactive component, or any combination of any of these.

In some embodiments, a second agent or pharmaceutical composition selected from the non-limiting group of anti-inflammatory agents, antidiabetic agents, hpyolipidemic agents, chemotherapeutic agents, antiviral agents, antibiotics, metabolic agents, small molecule inhibitors, protein kinase inhibitors, adjuvants, apoptotic agents, proliferative agents, organotropic targeting agents.

The importance of E3 ubiquitin ligases, and functional domains thereof, is highlighted by the number of normal cellular processes they regulate, and underlies the attendant diseases associated with loss of function or inappropriate targeting. See Ardley et al., “E3 Ubiquitin Ligases. Essays Biochem.” 41:15-30 (2005), which is hereby incorporated by reference in its entirety.

A fourth aspect of the present application relates to a method of treating a disease. The method includes selecting a subject having a disease and administering the composition described herein to the subject to give the subject an increased expression level of the substrate compared to a subject not afflicted with the disease.

The chimeric molecule described in this aspect is carried out in accordance with the previously described aspects of the present application.

As used herein, the term “subject” refers to a mammal, such as a human, but can also be another animal such as a domestic animal (e.g., a dog, cat, or the like), a farm animal (e.g., a cow, a sheep, a pig, a horse, or the like) or a laboratory animal (e.g., a monkey, a rat, a mouse, a rabbit, a guinea pig, or the like). The term “patient” refers to a “subject” who is, or is suspected to be, afflicted with a disease or condition.

The methods may involve administering compositions to a subject, where the disease possesses a measurable phenotype. The phenotype of the disease involves an increased expression level of a substrate compared to the phenotype from a subject not afflicted with the disease, in some embodiments. In this respect, chimeric molecules contained in the pharmaceutical compositions of the present application are efficacious against treating or alleviating the symptoms from a disease characterized by a phenotypic increase in the expression level of one or more substrates compared to the phenotype from a subject not afflicted with the disease.

Non-limiting examples of diseases that can be treated or prevented in the context of the present application, include, cancer, metastatic cancer, solid cancers, invasive cancers, disseminated cancers, breast cancer, lung cancer, NSCLC cancer, liver cancer, prostate cancer, brain cancer, pancreatic cancer, lymphatic cancer, ovarian cancer, endometrial cancer, cervical cancer, and other solid cancers known in the art, blood cell malignancies, lymphomas, leukemias, myelomas, stroke, ischemia, myocardial infarction, congestive heart failure, stroke, ischemia, peripheral vascular disease, alcoholic liver disease, cirrhosis, Parkinson's disease, Alzheimer's disease, diabetes, cancer, arthritis, ALS, pathogenic diseases, idiopathic diseases, viral diseases, bacterial, diseases, prionic diseases, fungal diseases, parasitic diseases, arthritis, wound healing, immunodeficiency, inflammatory disease, aplastic anemia, anemia, genetic disorders, congenital disorders, type 1 diabetes, type 2 diabetes, gestational diabetes, high blood glucose, metabolic syndrome, lipodystrophy syndrome, dyslipidemia, insulin resistance, leptin resistance, atherosclerosis, vascular disease, hypercholesterolemia, hypertriglyceridemia, non-alcoholic fatty liver disease, septic shock, multiple organ dysfunction syndrome, rheumatoid arthritis, trauma, stroke, heart infarction, systemic autoimmune disease, chronic hepatitis, overweight, and/or obesity, or any combination thereof.

In some embodiments, the disease is cancer, metastatic cancer, stroke, ischemia, peripheral vascular disease, alcoholic liver disease, hepatitis, cirrhosis, Parkinson's disease, Alzheimer's disease, cystic fibrosis diabetes, ALS, pathogenic diseases, idiopathic diseases, viral diseases, bacterial, diseases, prionic diseases, fungal diseases, parasitic diseases, arthritis, wound healing, immunodeficiency, inflammatory disease, aplastic anemia, anemia, genetic disorders, congenital disorders, type 1 diabetes, type 2 diabetes, gestational diabetes, high blood glucose, metabolic syndrome, lipodystrophy syndrome, dyslipidemia, insulin resistance, leptin resistance, atherosclerosis, vascular disease, hypercholesterolemia, hypertriglyceridemia, non-alcoholic fatty liver disease, overweight, or obesity, and any combination thereof.

When used in vivo for therapy, the compositions are administered to the subject in effective amounts, i.e., amounts that have desired therapeutic effect. The dose and dosage regimen will depend upon the degree of the disease in the subject, the characteristics of the particular peptide used, e.g., its therapeutic index, the subject, and the subject's history. The effective amount may be determined during pre-clinical trials and clinical trials by methods familiar to physicians and clinicians. An effective amount of a peptide useful in the methods may be administered to a mammal in need thereof by any of a number of well-known methods for administering pharmaceutical compounds.

Dosage, toxicity and therapeutic efficacy of the compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices may be desirable. While compositions that exhibit toxic side effects may be used, care should be taken to design a delivery system that targets such compositions to the site of affected tissue in order to minimize potential damage to uninfected cells and, thereby, reduce side effects.

In some embodiments, the compositions of the present application are administered orally, parenterally, subcutaneously, intravenously, intramuscularly, intraperitoneally, by intranasal instillation, by implantation, by intracavitary or intravesical instillation, intraocularly, intraarterially, intralesionally, transdermally, or by application to mucous membranes.

A fifth aspect of the present application relates to a method for substrate silencing. The method includes selecting a substrate to be silenced; providing the chimeric molecule described herein; and contacting the substrate with the chimeric molecule under conditions effective to permit the formation of a substrate-molecule complex, wherein the complex mediates the degradation of the substrate to be silenced.

The chimeric molecule described in this aspect is carried out in accordance with the previously described aspects of the present application.

A sixth aspect of the present application relates to a method of screening agents for therapeutic efficacy against a disease. The method includes providing a biomolecule whose presence mediates a disease state; providing a test agent comprising (i) a degradation domain comprising an E3 ubiquitin ligase (E3) motif, (ii) a targeting domain capable of specifically directing the degradation domain to the biomolecule, wherein the targeting domain is heterologous to the degradation domain, and (iii) a linker coupling the degradation domain to the targeting domain; contacting the biomolecule with the test agent under conditions effective for the test agent to facilitate degradation of the biomolecule; determining the level of the biomolecule as a result of the contacting; and identifying the test agent which, based on the determining, decreases the level of the biomolecule as being a candidate for therapeutic efficacy against the disease.

The chimeric molecule described in this aspect is carried out in accordance with the previously described aspects of the present application.

As used herein, the terms “reference level” or “control level” refer to an amount or concentration of biomarker (or biomolecule, ligand, substrate and the like) which may be of interest for comparative purposes. In some embodiments, a reference level may be the level of at least one biomarker expressed as an average of the level of at least one biomarker taken from a control population of healthy subjects or from a diseased population possessing aberrant expression of a protein or substrate. In another embodiment, the reference level may be the level of at least one biomarker in the same subject at an earlier time, i.e., before the present assay. In even another embodiment, the reference level may be the level of at least one biomarker in the subject prior to receiving a treatment regime.

As used herein, the term “sample” may include, but is not limited to, bodily tissue or a bodily fluid such as blood (or a fraction of blood such as plasma or serum), lymph, mucus, tears, saliva, sputum, urine, semen, stool, CSF, ascities fluid, or whole blood, and including biopsy samples of body tissue. A sample may also include an in vitro culture of microorganisms grown from a sample from a subject. A sample may be obtained from any subject, e.g., a subject/patient having or suspected to have a disease or condition characterized by a disease.

As used herein, the term “screening” means determining whether a chimeric molecule or composition has capabilities or characteristics of preventing or slowing down (lessening) the targeted pathologic condition stated herein, namely a disease or condition characterized by defects in specified disease.

As used herein, the terms “effective amount” or “therapeutically effective amount” of a chimeric molecule or composition is a quantity sufficient to achieve a desired therapeutic and/or prophylactic effect, for example, an amount which results in the prevention of or a decrease in the symptoms associated with a disease that is being treated. The amount of compound administered to the subject will depend on the type and severity of the disease and on the characteristics of the individual, such as general health, age, sex, body weight and tolerance to drugs. It will also depend on the degree, severity or stage of disease. The skilled artisan will be able to determine appropriate dosages depending on these and other factors.

The term “enzyme linked immunosorbent assay” (ELISA) as used herein includes an antibody-based assay in which detection of the antigen of interest is accomplished via an enzymatic reaction producing a detectable signal. An ELISA can be run as a competitive or non-competitive format. ELISA also includes a 2-site or “sandwich” assay in which two antibodies to the antigen are used, one antibody to capture the antigen and one labeled with an enzyme or other detectable label to detect captured antibody-antigen complex. In a typical 2-site ELISA, the antigen has at least one epitope to which unlabeled antibody and an enzyme-linked antibody can bind with high affinity. An antigen can thus be affinity captured and detected using an enzyme-linked antibody. Typical enzymes of choice include alkaline phosphatase or horseradish peroxidase, both of which generate a detectable product when contacted by appropriate substrates.

As used herein, the term “epitope” includes a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents. Typically, an epitope will be a determinant region form a substrate, which can be recognized by one or more target domains.

To screen for targeting domains or substrates which possess an epitope, a routine cross-blocking assay such as that described in Antibodies, A Laboratory Manual, Cold Spring Harbor Laboratory, Ed Harlow and David Lane (1988), which is hereby incorporated by reference in its entirety, can be performed. This assay can be used to determine if a target domain binds the same site or epitope of a substrate as a different targeting domain, antibody, antibody fragment and the like. Alternatively, or additionally, epitope mapping can be performed by methods known in the art. For example, the antibody sequence can be mutagenized such as by alanine scanning, to identify contact residues. In a different method, peptides corresponding to different regions of substrate can be used in competition assays with a test target domain or with a test antibody and a target domain or an antibody with a characterized epitope.

As used herein, the term “hypervariable region” refers to the amino acid residues of an antibody which are responsible for antigen-binding. The hypervariable region generally comprises amino acid residues from a “complementarity determining region” or “CDR”, e.g., around about residues 24-34 (L1), 50-56 (L2) and 89-97 (L3) in the V_{L}, and around about 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, Md. (1991), which is hereby incorporated by reference in its entirety), and/or those residues from a “hypervariable loop” (e.g., residues 26-32 (L1), 50-52 (L2) and 91-96 (L3) in the V_{L}, and 26-32 (H1), 52A-55 (H2) and 96-101 (H3) in the V_{H} (Chothia and Lesk, “Canonical Structures for the Hypervariable Regions of Immunoglobulins,” J. Mol. Biol. 196:901-17 (1987)), which is hereby incorporated by reference in its entirety).

As used herein, the terms “isolated” or “purified” polypeptide, peptide, molecule, or chimeric molecule, is substantially free of cellular material or other contaminating polypeptides from the cell or tissue source from which the agent is derived, or substantially free from chemical precursors or other chemicals when chemically synthesized. For example, a chimeric molecule would be free of materials that would interfere with such a molecules intended function, diagnostic or therapeutic uses. Such interfering materials may include proteins or fragments other than the materials encompassed by the chimeric molecule, enzymes, hormones and other proteinaceous and nonproteinaceous solutes.

In one embodiment, the identifying is carried out with respect to a standard biomolecule level in a subject not afflicted with the disease. The identifying may also be carried out with respect to the biomolecule level absent the contacting, in some embodiments. A control level, in the regard, can be employed to compare to the level of the biomolecule in a sample. In some embodiments, the control level is the level of the biomolecule from a subject not afflicted with the disease. An overabundance of the biomolecule in the sample obtained from the subject suspected of having the disease or condition affecting substrate levels compared with the sample obtained from the healthy subject is indicative of the biomolecule-associated disease or condition in the subject being tested.

There are a myriad of diseases in which the degree of overabundance of certain substrate biomolecules are known to be indicative of whether a subject is afflicted with a disease or is likely to develop a disease. See, e.g., Anderson et al., “Discovering Robust Protein Biomarkers for Disease from Relative Expression Reversals in 2-D DIGE Data,” Proteomics 7:1197-1207 (2007), which is hereby incorporated by reference in its entirety. Examples of conditions in which biomolecules are increased compared to control subjects include the diseases described above.

Accordingly, the chimeric molecules and compositions of the present application are administered to a subject in need of treatment. E3 ubiquitin ligases, such as the E3 gene products encoding a E3 motif, are described above, and can be used in the present screening methods for determining the efficacy of the chimeric molecules disclosed herein. In some embodiments, suitable in vitro or in vivo assays are performed to determine the effect of the chimeric molecules and compositions of the present application and whether administration is indicated for treatment. Compositions for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

Any method known to those in the art for contacting a cell, organ or tissue with a composition may be employed. In vivo methods typically include the administration of a chimeric molecule or composition, such as those described above, to a mammal, suitably a human. When used in vivo for therapy, the chimeric molecules or compositions are administered to the subject in effective amounts, as described herein. Results can be ascertained as per the empirical variables set forth at the outset of the methods described herein.

In vitro methods typically include the assaying the effect of chimeric molecule or composition, such as those described above, on a sample or extract. In some embodiments, chimeric molecule efficacy can be determined by assessing the affect on substrate degradation, i.e., the ability of the chimeric molecules and compositions to exert a phenotypic change in a sample. Such methods include, but are not limited to, immunohistochemistry, immunofluorescence, ELISPOT, ELISA, or RIA. The steps of various useful immunodetection methods have been described in the scientific literature, such as, e.g., Nakamura et al., Enzyme Immunoassays: Heterogeneous and Homogeneous Systems, Handbook of Experimental Immunology, Vol. 1: Immunochemistry 27.1-27.20 (1986), each of which is incorporated herein by reference in its entirety and specifically for its teaching regarding immunodetection methods.

Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), enzyme linked immunospot assay (ELISPOT), radioimmunoassays (RIA), radioimmune precipitation assays (RIPA), immunobead capture assays, Western blotting, dot blotting, gel-shift assays, Flow cytometry, immunohistochemistry, fluorescence microscopy, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomolecule) with an antibody to the molecule of interest or contacting an antibody to a molecule of interest (such as antibodies to the disclosed biomolecule) with a molecule that can be bound by the antibody, as the case may be, under conditions effective to allow the formation of immunocomplexes. In this regard, the skilled artisan will be able to assess the presence and or level of specific biomolecules in a given sample. Subsequently, the chimeric molecule compositions of the present application are added to the assay. Thereafter, the level of biomolecule can be assessed, i.e., the presence or level thereof, using the immunoassays described herein to determine the post-treatment phenotypic effect.

Immunoassays can include methods for detecting or quantifying the amount of a biomolecule of interest in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label. See, for example, U.S. Pat. Nos. 3,817,837; 3,850,752; 3,939,350; 3,996,345; 4,277,437; 4,275,149 and 4,366,241, each of which is incorporated herein by reference in its entirety and specifically for teachings regarding immunodetection methods and labels.

The treatment methods of the present application possess ubiquitin regions attached to targeting domains, as described above. In some embodiments, the targeting domain binds an intracellular biomolecule, as described above. Likewise, the treatment methods of the present application employ polypeptide linkers of sufficient length to prevent the steric disruption of binding between the targeting domain and the substrate. In some embodiments, the biomolecule is associated with a disease as described above.

In one embodiment, the method is carried out with a plurality of test agents.

A seventh aspect of the present application relates to a method of screening for disease biomarkers. The method includes providing a sample of diseased cells expressing one or more ligands; providing a plurality of chimeric molecules comprising (i) a degradation domain comprising an E3 ubiquitin ligase (E3) motif, (ii) a targeting domain capable of specifically directing the degradation domain to the one or more ligands, wherein the targeting domain is heterologous to the degradation domain, and (iii) a linker coupling the degradation domain to the targeting domain; contacting the sample with the plurality of chimeric molecules under conditions effective for the diseased cells to fail to proliferate in the absence of the chimeric molecule; determining which of the chimeric molecules permit the diseased cells to proliferate; and identifying, as biomarkers for the disease, based on the determining the ligands which bind to the chimeric molecules and permit diseased cells to proliferate.

The chimeric molecule described in this aspect is carried out in accordance with the previously described aspects of the present application.

As used herein, the terms “biomarker” or “biomolecule” or “molecule” refer to a polypeptide (of a particular expression level) which is differentially present in a sample taken from patients having a disease as compared to a comparable sample taken from a control subject or a population of control subjects.

As used herein, the terms “ligand” or “substrate” refer to substance that are able to bind to and form transient or stable complexes with a protein, molecule, chimeric molecule, ligand (dimer), substrate (dimer), a second substrate, a second ligand, target domain, regions, potions, and fragments thereof, ubiquitin or E3 motif regions, domains, or portions thereof, biomolecules, biomarkers, and the like, to serve a biological purpose, for example a substrate which interacts with an enzyme in the process of an enzymatic reaction. Ligands also include signal triggering molecules which bind to sites on a target protein, by intermolecular forces such as ionic bonds, hydrogen bonds and Van der Waals forces. In some embodiments, substrates bind ligands and/or ligands bind substrates.

Many, if not all diseases, are complex and multifactorial. When considering neurodegeneration, for example, substantial neuronal cell loss occurs before pathologic presentation. Screening for and developing such drugs—to treat neurodegenerative diseases—is further stymied by ancillary therapies which ameliorate the symptoms. Thus, target detection is obfuscated by prior therapeutic administration, which, may in turn, slow disease progression and further confound treatment regimes. In this way, the present application provides new, inventive, screening methods for elucidation of disease biomarkers by employing phenotypic screening analyses. See, e.g., Pruss, R. M., “Phenotypic Screening Strategies for Neurodegenerative Diseases: A Pathway to Discover Novel Drug Candidates and Potential Disease Targets or Mechanisms.” CNS & Neurological Disorders—Drug Targets, 9, 693-700 (2010), which is hereby incorporated by reference in its entirety.

Phenotypic screening involves using an appropriate sample, e.g., class of cells, cell extract, neurons, tissue, and the like, from a patient afflicted with a disease and subjecting the sample to one or more chimeric molecules as described herein. Subsequently, the sample is screened for viability, proliferation, cell processes and/or phenotypic characteristic of the diseased cell, e.g., shrinking, loss of membrane potential, morphological changes, and the like. Image analysis software allows for cell bodies or other objects to empirically assess the results. Hits coming from the screen may maintain cell survival by stimulating survival pathways, mimicking trophic factors, or inhibiting death signaling. Higher content screening and profiling in target-directed secondary assays can then be used to identify targets and mechanisms of action of promising hits.

Examples of diseases conditions from which a biomarker screening analysis can be performed include the diseases described above. In some embodiments, the method of screening for disease biomarkers includes a plurality of molecules, where the molecules possess a E3 motif as described above. In some embodiments, the biomarker screening methods include molecules possessing a targeting domain as described above. The screening methods of the present application employ polypeptide linkers of sufficient length to prevent the steric disruption of binding between the targeting domain and the biomolecule and/or ligand.

Once a chimeric molecule is determined to provide a therapeutic indication, the biomarker is isolated using the targeting domain region (or the entire chimeric molecule) to immunoprecipitate the biomarker, from a sample, which is subsequently identified using methods well known in the art. Biomarker isolation and purification methods include, but are not limited to, for example, HPLC or FPLC chromatography using size-exclusion or affinity-based column resins. See, e.g., Sambrook et al. 1989, Cold Spring Harbor Laboratory Press, which is hereby incorporated by reference in its entirety.

Active fragments, derivatives, or variants of the polypeptides of the present application may be recognized by, for example, the deletion or addition of amino acids that have minimal influence on the properties, secondary structure, and biological activity of the polypeptide. For example, a polypeptide may be joined to a signal (or leader) sequence at the N-terminal end of the protein which co-translationally or post-translationally directs sub-cellular or extracellular localization of the protein.

The biomarker can then be elucidated using techniques known in the art. In some embodiments, determining the identity of the biomarker is performed using MALDI-TOF, mass spectrometry, mass spectroscopy, protein sequencing, antibody interactions, western blot, immunoassay, ELISA, chromatographic techniques, reverse proteomics, immunoprecipitations, radioimmunoassay, and immunofluorescence, or any combinations thereof.

Suitable mass spectrometric techniques for the study and identification of proteins include, laser desorption ionization mass spectrometry and electrospray ionization mass spectrometry. Within the category of laser desorption ionization (LDI) mass spectrometry (MS), both matrix assisted LDI (MALDI) and surface assisted LDI (SELDI) time-of-flight (TOF) MS may be employed. SELDI TOF-MS is particularly well-suited for use in the present methods because it provides attomole sensitivity for analysis, quantification of low abundant proteins (pg-ng/ml) and highly reproducible results.

The methods described herein can be performed, e.g., by utilizing pre-packaged kits comprising at least one reagent, e.g., a chimeric molecule or composition described herein, which can be conveniently used, e.g., in clinical settings to treat subjects exhibiting symptoms of a disease or illness involving an overexpressed substrate, biomolecule, or biomarker. Furthermore, any cell type or tissue in which the chimeric molecule of the present application can be expressed is suitable for use in the kits described herein.

In another aspect of the present application, a kit or reagent system for using the chimeric molecules and compositions of the present application. Such kits will contain a reagent combination including the particular elements required to conduct an assay according to the methods disclosed herein. The reagent system is presented in a commercially packaged form, as a composition or admixture where the compatibility of the reagents will allow, in a test device configuration, or more typically as a test kit, i.e., a packaged combination of one or more containers, devices, or the like holding the necessary reagents, and preferably including written instructions for the performance of assays. The kit may be adapted for any configuration of an assay and may include compositions for performing any of the various assay formats described herein.

Reagents useful for the disclosed methods can be stored in solution or can be lyophilized. When lyophilized, some or all of the reagents can be readily stored in microtiter plate wells for easy use after reconstitution. It is contemplated that any method for lyophilizing reagents known in the art would be suitable for preparing dried down reagents useful for the disclosed methods.

Also within the scope of the present application are kits comprising the chimeric molecules/compositions and second agents of the application and instructions for use. The kits are useful for detecting the presence of a substrate in a biological sample e.g., any body fluid including, but not limited to, e.g., serum, plasma, lymph, cystic fluid, urine, stool, cerebrospinal fluid, acitic fluid or blood and including biopsy samples of body tissue. For example, the kit can comprise one or more chimeric molecules composed of a E3 motif ubiquitin region linked to a targeting domain capable of binding a substrate in a biological sample.

### EXAMPLES

The following examples are provided to illustrate embodiments of the present application but they are by no means intended to limit its scope.

The following examples are included to demonstrate illustrative embodiments of the present application. It should be appreciated by those of skill in the art that the techniques disclosed in the examples which follow represent techniques discovered by the inventors to function well in the practice of the present application, and thus can be considered to constitute preferred modes for its practice. However, those of skill in the art should, in light of the present application, appreciate that many changes can be made in the specific embodiments which are disclosed and still obtain a like or similar result without departing from the spirit and scope of the present application.

### Experimental Methods

Plasmids.

All plasmids used in this study are provided in Table 1.

TABLE 1
Bacterial strains, cell lines and plasmids used in this study.

| Bacterial strain | | |
|---|---|---|
| DH5α | F- (Φ80Δ/acZΔM15,) Δ(lac/ZYA-argF)U169 | Laboratory stock |
| | recA1 endA1 hsdR17(rₖ_{^{−}}, mₖ+) phoA | |
| | supE44 λ- thi-1 gyrA96 relA1 | |
| BL21 (DE3) | F- ompT gal dcm lon hsdS_{B}(r_{B}_{^{−}} m_{B}_{^{−}}) λ(DE3) | Laboratory stock |
| Rosetta(DE3) | F- ompT gal dcm lon hsdS_{B}(r_{B}_{^{−}} m_{B}_{^{−}}) λ(DE3) | Laboratory stock |
| | pRARE (Cm^{R}) | |

Cell line

| | | |
|---|---|---|
| HEK293T | | Laboratory stock |
| HEK293T-EGFP | HEK293T cells stably expressing EGFP | This study |
| HEK293T-ERK2-EGFP | HEK293T cells stably expressing ERK2-EGFP | This study |
| HEK293T-EGFP-HRas^{G12V} | HEK293T cells stably expressing EGFP-HRas^{G12V} | This study |
| HEK293T-d2EGFP | HEK293T cells stably expressing d2EGFP | This study |
| HeLa H2B-EGFP | HeLa cells stably expressing H2B-EGFP fusion | [1] |
| MCF-10A | | Laboratory stock |
| MCF-10A rtTA | | Laboratory stock |
| MCF-10A rtTA EGFP- | MCF-10A cells stably expressing EGFP- | This study |
| HRAS^{G12V} | HRAS^{G12V} fusion and rtTA | |
| MCF-10A rtTA GS2-IpaH9.8 | MCF-10A cells stably expressing GS2- | This study |
| | IpaH9.8 fusion and rtTA | |
| MCF-10A rtTA EGFP- | MCF-10A cells stably expressing EGFP- | This study |
| HRAS^{G12V} + GS2-IpaH9.8 | HRAS^{G12V} fusion, GS2-IpaH9.8, and rtTA | |
| MCF-10A rtTA EGFP | MCF-10A cells stably expressing EGFP- | This study |
| HRAS^{G12V} + GS2-IpaH9.8^{C337A} | HRAS^{G12V} fusion, GS2-IpaH9.8^{C337A}, and | |
| | rtTA | |

| Plasmid | Relevant features | Source |
|---|---|---|
| pcDNA3 | CMV promoter, Amp^{R} | Laboratory stock |
| pCDH1-CMV-MCS-EF1α- | CMV promoter; Pur^{R}, Amp^{R} | Laboratory stock |
| Puro | | |
| pET28a(+) | T7lac promoter; Kan^{R} | Novagen |
| pET24d(+) | T7lac promoter; Kan^{R} | Novagen |
| pTriEx-3 | CMV promoter; T7 promoter; Amp^{R} | Novagen |
| psPAX2 | Lentiviral packaging vector; CMV promoter; Amp^{R} | Laboratory stock |
| pMD2.G | Lentiviral packaging vector; CMV promoter; Amp^{R} | Laboratory stock |
| pPB TetOn Hygro | Transposon vector; SV40 promoter; Amp^{R} | Laboratory stock |
| pLV rtTA-NeoR | Tetracycline inducible reverse transcriptional | Laboratory stock |
| | transactivator; CMV promoter; EF1α promoter; | |
| | Neo^{R}; Amp^{R} | |
| pCMV-hyPBase | PiggyBac transposase; CMV promoter; Amp^{R} | [2] |
| pHIV-d2EGFP | Lentiviral vector expressing d2EGFP EF-1^{α} | [3] |
| | promoter, IRES; Amp^{R} | |
| pGEM4Z/GFP/A64 | In vitro transcription of GFP with 3′ 64 residue | [4] |
| | poly A tail; T7 promoter; Amp^{R} | |
| pHFT2-GS2 | GS2 monobody in pHFT2 plasmid; T7 promoter, | [5] |
| | Kan^{R} | |
| pHFT2-GS5 | GS5 monobody in pHFT2 plasmid; T7 promoter, | [5] |
| | Kan^{R} | |
| pHFT2-GL4 | GL4 monobody in pHFT2 plasmid; T7 promoter, | [5] |
| | Kan^{R} | |
| PHFT2-GL6 | GL6 monobody in pHFT2 plasmid; T7 promoter, | [5] |
| | Kan^{R} | |
| pGalAga-GL8 | GL8 monobody in pGalAgaCamR; Cam^{R} | [5] |
| pHFT2-AblSH2MB#AS15 | AS15 monobody in pHFT2 plasmid; T7 | [4] |
| pHFT2-SHP2NSa5 | NSa5 monobody in pHFT2 plasmid; T7 | [6] |
| pET24a(+)-RasInll | RasInll with C-terminal GS linker, 6xHis, Avi, | [7] |
| | and Flag tags; T7lac promoter; Kan^{R} | |
| pcDNA3-R4-CHIPΔTPR | scFv13-R4 fused to CHIP lacking TPR | [8] |
| | domain with C- terminal Flag and 6x-His | |
| | tags cloned in pcDNA3; CMV promoter; | |
| pcDNA3_NSlmb-vhhGFP4 | vhhGFP4 fused to F-box domain from | [1]; Addgene |
| | Drosophila melanogaster Slmb cloned in | #35579 |
| pETHis6MEK1 R4F + ERK2 | pET-based expression of two proteins: | [9] |
| | constitutively active MEK1 and wild-type, | |
| pHFT2-SHP2 | Full-length SHP2 in pHFT2 plasmid; T7 | [6] |
| pcDNA3-EGFP | EGFP cloned in pcDNA3; CMV promoter, Amp^{R}; | This study |
| pcDNA3-HF-GS2 | GS2 with N-terminal Kozak, Flag and 6xHis | This study |
| | tags,and BamHI and EcoRI sites; CMV | |
| pcDNA3-GS2-FH | GS2 with C-terminal Nhel and Sbfl sites, Flag | This study |
| | tag, and 6xHis tag; CMV promoter; Amp^{R}. | |
| pcDNA3-GS2-AvrPtoB | GS2 fused to Pseudomonas syringae AvrPtoB | This study |
| | lacking N- terminal domain with C-terminal | |
| | Flag and 6x-His tags cloned in pcDNA3; CMV | |
| pcDNA3-GS2-IpaH9.8 | GS2 fused to Shigella flexneri IpaH9.8 lacking | This study |
| | LRR domain (IpaH9.8ΔLRR) with C-terminal | |
| | Flag and 6x-His tags cloned in pcDNA3; CMV | |
| pcDNA3-GS2-IpaH9.8^{C337A} | pcDNA3-GS2-IpaH9.8 containing Cys337Ala | This study |
| pcDNA3-AS15-IpaH9.8 | AS15 fused to IpaH9.8 lacking LRR domain | This study |
| | with C-terminal Flag and 6x-His tags cloned | |
| | in pcDNA3; CMV promoter; Kozak; Amp^{R} | |
| pcDNA3-GS2-IpaH1.4 | GS2 fused to S. flexneri IpaH1.4 lacking LRR | This study |
| | domain with C- terminal Flag and 6x-His tags | |
| | cloned in pcDNA3; CMV promoter; Kozak; | |
| pcDNA3-GS2-IpaH2.5 | GS2 fused to S. flexneri IpaH2.5 lacking LRR | This study |
| | domain with C- terminal Flag and 6x-His tags | |
| | cloned in pcDNA3; CMV promoter; Kozak; | |
| pcDNA3-GS2-IpaH4.5 | GS2 fused to S. flexneri IpaH4.5 lacking LRR | This study |
| | domain with C- terminal Flag and 6x-His tags | |
| | cloned in pcDNA3; CMV promoter; Kozak; | |
| pcDNA3-GS2-IpaH7.8 | GS2 fused to S. flexneri IpaH7.8 lacking LRR | This study |
| | domain with C- terminal Flag and 6x-His tags | |
| | cloned in pcDNA3; CMV promoter; Kozak; | |
| pcDNA3-GS2-IpaH0722 | GS2 fused to S. flexneri IpaH0722 lacking | This study |
| | LRR domain with C-terminal Flag and 6x-His | |
| | tags cloned in pcDNA3; CMV promoter; | |
| pcDNA3-LegAU13-GS2 | GS2 fused to L. pneumophila LegAU13 F-box | This study |
| | with N-terminal Flag and 6x-His tags cloned in | |
| | pcDNA3; CMV promoter; Kozak; Amp^{R} | |
| pcDNA3-LegU1-GS2 | GS2 fused to L. pneumophila LegU1 F-box | This study |
| | with N-terminal Flag and 6x-His tags cloned | |
| | in pcDNA3; CMV promoter; Kozak; Amp^{R} | |
| pcDNA3-LubX-GS2 | GS2 fused to Legionella pneumophila | This study |
| | LubX lacking CTD domain with N-terminal | |
| | Flag and 6x-His tags cloned in pcDNA3; | |
| pcDNA3-GS2-NleG2-3 | GS2 fused to EHEC O157:H7 NleG2-3 | This study |
| | lacking N-terminal domain with C-terminal | |
| | Flag and 6x-His tags cloned in pcDNA3; | |
| pcDNA3-GS2-NleG5-1 | GS2 fused to EHEC O157:H7 NleG5-1 | This study |
| | lacking N-terminal domain with C-terminal | |
| | Flag and 6x-His tags cloned in pcDNA3; | |
| pcDNA3-GS2-NleL | GS2 fused to EHEC O157:H7 NleL lacking β- | This study |
| | helix domain with C-terminal Flag and 6x-His | |
| | tags cloned in pcDNA3; CMV promoter; Kozak; | |
| pcDNA3-SidC-GS2 | GS2 fused to L. pneumophila SidC lacking N- | This study |
| | terminal domain with N-terminal Flag and 6x- | |
| | His tags cloned in pcDNA3; CMV promoter; | |
| pcDNA3-GS2-SlrP | GS2 fused to Enterohemorrhagic Escherichia | This study |
| | coli (EHEC) O157:H7 SlrP lacking LRR | |
| | domain with C-terminal Flag and 6x-His tags | |
| pcDNA3-GS2-SopA | GS2 fused to S. typhimurium SopA lacking β- | This study |
| | helix domain with C-terminal Flag and 6x-His | |
| | tags cloned in pcDNA3; CMV promoter; Kozak; | |
| pcDNA3-GS2-SspH1 | GS2 fused to Salmonella typhimurium | This study |
| | SspH1 lacking LRR domain with C-terminal | |
| | Flag and 6x-His tags cloned in pcDNA3; | |
| pcDNA3-GS2-SspH2 | GS2 fused to S. typhimurium SspH2 lacking | This study |
| | LRR domain with C-terminal Flag and 6x-His | |
| | tags cloned in pcDNA3; CMV promoter; Kozak; | |
| pcDNA3-GS2-XopL | GS2 fused to Xanthomonas campestris | This study |
| | XopL lacking LRR domain with C-terminal | |
| | Flag and 6x-His tags cloned in pcDNA3; | |
| pcDNA-βTrCP-GS2 | GS2 fused to full length H. sapiens βTrCP | This study |
| | with C-terminal Flag and 6x-His tags cloned | |
| | in pcDNA3; CMV promoter; Kozak; Amp^{R} | |
| pcDNA3-GS2-CHIPΔTPR | GS2 cloned in place of scFvR4 in pcDNA3- | This study |
| | R4-CHIPΔTPR; CMV promoter; Kozak; | |
| pcDNA3-Slmb-GS2 | GS2 cloned in place of vhhGFP4 in | This study |
| | pcDNA3-NSlmb- vhhGFP4; CMV | |
| pcDNA3-GS2-SPOP | GS2 fused to Homo sapiens SPOP F-box | This study |
| | with C-terminal Flag and 6x-His tags | |
| | cloned in pcDNA3; CMV promoter; Kozak; | |
| pcDNA3-VHL-GS2 | GS2 fused to H. sapiens VHL F-box with N- | This study |
| | terminal Flag and 6x-His tags cloned in | |
| pcDNA3-GS5-IpaH9.8 | GS5 fused to IpaH9.8 lacking LRR domain | This study |
| | with C-terminal Flag and 6x-His tags cloned | |
| | in pcDNA3; CMV promoter; Kozak; Amp^{R} | |
| pcDNA3-GL4-IpaH9.8 | GL4 fused to IpaH9.8 lacking LRR domain | This study |
| | with C-terminal Flag and 6x-His tags cloned | |
| | in pcDNA3; CMV promoter; Kozak; Amp^{R} | |
| pcDNA3-GL6TpaH9.8 | GL6 fused to IpaH9.8 lacking LRR domain | This study |
| | with C-terminal Flag and 6x-His tags cloned | |
| | in pcDNA3; CMV promoter; Kozak; Amp^{R} | |
| pcDNA3-GL8-IpaH9.8 | GL8 fused to IpaH9.8 lacking LRR domain | This study |
| | with C-terminal Flag and 6x-His tags cloned | |
| | in pcDNA3; CMV promoter; Kozak; Amp^{R} | |
| pcDNA3-vhhGFP4-IpaH9.8 | vhhGFP4 fused to IpaH9.8 lacking LRR | This study |
| | domain with C- terminal Flag and 6x-His | |
| | tags cloned in pcDNA3; CMV promoter; | |
| pcDNA3-NSa5-IpaH9.8 | NSa5 fused to IpaH9.8 lacking LRR domain | This study |
| | with C-terminal Flag and 6x-His tags cloned | |
| | in pcDNA3; CMV promoter; Kozak; Amp^{R} | |
| pcDNA3-NSa5- | NSa5 fused to IpaH9.8^{C337A} lacking LRR | This study |
| IpaH9.8^{C337A} | domain with C- terminal Flag and 6x-His | |
| | tags cloned in pcDNA3; CMV promoter; | |
| pcDNA3-RasInll-IpaH9.8 | RasInll fused to IpaH9.8 lacking LRR domain | This study |
| | with C-terminal Flag and 6x-His tags cloned in | |
| | pcDNA3; CMV promoter; Kozak; Amp^{R} | |
| pcDNA3-RasInll- | RasInll fused to IpaH9.8^{C337A} lacking LRR | This study |
| IpaH9.8^{C337A} | domain with C- terminal Flag and 6x-His | |
| | tags cloned in pcDNA3; CMV promoter; | |
| mEmerald-C1 | CMV promoter; Kan^{R} | Laboratory stock |
| mVenus-N1 | CMV promoter; Kan^{R} | Laboratory stock |
| pcDNA3-mCerulean | CMV promoter; Kozak; Amp^{R} | This study |
| pcDNA3-CFP | CMV promoter; Kozak; Amp^{R} | This study |
| pcDNA3-sfGFP | CMV promoter; Kozak; Amp^{R} | This study |
| pcDH--mCherry | CMV promoter; EF1α promoter; Puro^{R}; Amp^{R} | Laboratory stock |
| pPB-GS2-IpaH9.8 | GS2-IpaH9.8 with C-terminal Flag and 6x-His | This study |
| | tags cloned in pPB TetOn Hygro; CMV | |
| pPB-GS2-IpaH9.8^{C337A} | GS2-IpaH9.8^{C337A} with C-terminal Flag and 6x- | This study |
| | His tags cloned in pPB TetOn Hygro; CMV | |
| | promoter; SV40 promoter; Amp^{R} | |
| pCDH1-ERK2-EGFP | ERK2 fused to N-terminus of EGFP cloned in | This study |
| | pCDH1-CMV- MCS-EF1α-Puro; CMV | |
| pCDHI-EGFP | EGFP cloned in pCDH1-CMV-MCS- | This study |
| | EF1α-Puro; CMV promoter; Pur^{R}, | |
| pCDH1-EGFP-HRas^{G12V} | HRas^{G12V} fused to C-terminus of EGFP | This study |
| | cloned in pCDH1- CMV-MCS-EF1α-Puro; | |
| mEmerald-α-actinin-19 | α-actinin fused to N-terminus of mEmerald; | Laboratory stock |
| | CMV promoter; Kan^{R} | |
| pcDNA3.1(−)-α-synuclein- | α-synuclein fused to the N-terminus of | [10] |
| EGFP | EGFP cloned in pcDNA3.1; CMV | |
| mEmerald-FAK-5 | FAK fused to C-terminus of mEmerald; CMV | Laboratory stock |
| pEGFP-C1 F-tractin-EGFP | F-tractin fused to N-terminus of EGFP; CMV | Addgene #58473 |
| EGFR-mEmerald | EGFR fused to N-terminus of mEmerald; | Laboratory stock |
| | CMV promoter; Amp^{R} | |
| pErbB2-EGFP | ErbB2 fused to N-terminus of EGFP; CMV | Addgene #39321 |
| pcDNA3-ERK2-EGFP | ERK2 fused to EGFP in pcDNA3; CMV | This study |
| | promoter; Kozak; Amp^{R} | |
| pLV pGK-H2B-EGFP | H2B fused to C-terminus of EGFP; pGK | Laboratory stock |
| mEGFP-HRas^{G12V} | HRas^{G12V} (constitutively active) fused to | [11]; Addgene |
| | mEGFP cloned in pCI; CMV promoter; | #18666 |
| mEmerald-Muc1-FL | Muc1 fused to N-terminus of mEmerald; CMV | Laboratory stock |
| pcDNA3-EGFP-NLS | NLS sequence derived from C-terminus of | This study |
| | SV40 fused to C- terminus of EGFP and | |
| | cloned in pcDNA3; CMV promoter; Amp^{R} | |
| mEmerald-Paxillin-22 | Paxillin fused to N-terminus of mEmerald; | Laboratory stock |
| | CMV promoter; Kan^{R} | |
| pcDNA3-SHP2-EGFP | SHP2 fused to C-terminus of EGFP; CMV | This study |
| mEmerald-Vinculin-23 | Vinculin fused to C-terminus of mEmerald; | Laboratory stock |
| | CMV promoter; Kan^{R} | |
| pET28-EGFP | EGFP with C-terminal 6x-His tag in | This study |
| | pET28a(+); T7lac promoter; Kan^{R} | |
| pET28(+)-GS2 | GS2 with C-terminal Flag and 6x-His tags | This study |
| | in pET28a(+); T7lac promoter; Kan^{R} | |
| pET24d(+)-GS2-IpaH9.8 | GS2 fused to IpaH9.8 lacking LRR domain | This study |
| | with C-terminal Flag and 6x-His tags in | |
| pET24d(+)-IpaH9.8ALRR | IpaH9.8 lacking LRR domain with C-terminal | This study |
| | Flag and 6x-His tags in pET28a(+); T7lac | |
| pTriEX-3-GS2-IpaH9.8^{C337A} | GS2 fused to IpaH9.8 lacking LRR domain | This study |
| | and containing Cys337Ala mutation in | |
| | pTriEx-3; CMV promoter; T7 promoter; | |
| pGEM4Z/GS2-IpaH9.8/A64 | In vitro transcription of GS2-IpaH9.8 with 3′ | This study |
| | human globin UTR and 64 residue poly A | |
| pGEM4Z/GS2- | In vitro transcription of GS2-IpaH9.8^{C337A} | This study |
| IpaH9.8^{C337A}/A64 | with 3′ human globin UTR and 64 residue | |
| pGEM4Z/AS15- | In vitro transcription of AS15-IpaH9.8 with 3′ | This study |
| IpaH9.8/A64 | human globin UTR and 64 residue poly A | |

[1] Caussinus et al., “Fluorescent Fusion Protein Knockout Mediated by Anti-GFP Nanobody,” Nat. Struct. Mol. Biol. 19: 117-21 (2011), which is hereby incorporated by reference in its entirety.
[2] Yusa et al., “A Hyperactive PiggyBac Transposase for Mammalian Applications,” Proc. Natl. Acad. Sei. USA 108: 1531-6 (2011), which is hereby incorporated by reference in its entirety.
[3] Li et al., “Structurally Modulated Codelivery of siRNA and Argonautc 2 for Enhanced RNA interference,” Proc. Natl. Acad. Sci. USA 115: E2696-e2705 (2018), which is hereby incorporated by reference in its entirety.
[4] Boczkowski et al., “Induction of Tumor Immunity and Cytotoxic T Lymphocyte Responses Using Dendritic Cells Transfected With Messenger RNA Amplified From Tumor Cells.” Cancer Res. 60: 1028-34 (2000), which is hereby incorporated by reference in its entirety.
[5] Koide et al., “Teaching an Old Scaffold New Tricks: Monobodies Constructed Using Alternative Surfaces of the FN3 Scaffold,” J. Mol. Biol. 415: 393-405 (2012), which is hereby incorporated by reference in its entirety.
[6] Sha et al., “Dissection of the BCR-ABL Signaling Network Using Highly Specific Monobody Inhibitors to the SHP2 SH2 Domains,” Proc. Natl. Acad. Sci. USA 110: 14924-29 (2013). which is hereby incorporated by reference in its entirety.
[7] Cetin et al., “Raslns: Genetically Encoded Intrabodies of Activated Ras Proteins,” J. Mol. Biol. 429: 562-73 (2017), which is hereby incorporated by reference in its entirety.
[8] Portnoff et al., “Ubiquibodies, Synthetic E3 Ubiquitin Ligases Endowed With Unnatural Substrate Specificity for Targeted Protein Silencing,” J. Biol. Chem. 289: 7844-55 (2014). which is hereby incorporated by reference in its entirety.
[9] Khokhlatchev et al., “Reconstitution of Mitogenactivated Protein Kinase Phosphorylation Cascades in Bacteria. Efficient Synthesis of Active Protein Kinases,” J. Biol. Chem. 272: 11057-62 (1997). which is hereby incorporated by reference in its entirety.
[10] Butler et al., “Bifunctional Anti-Nonamyloid Component α-synuclein Nanobodies are Protective In Situ” PLoS ONE 11: e0165964 (2016), which is hereby incorporated by reference in its entirety.
[11] Yasuda et al., “Supersensitive Ras Activation in Dendrites and Spines Revealed by Two-Photon Fluorescence Lifetime Imaging,” Nat. Neurosci. 9: 283-91 (2006). which is hereby incorporated by reference in its entirety.

E. coli strain DH5α was used for the construction and propagation of all plasmids. To construct pcDNA3-EGFP, EGFP was PCR amplified using primers that introduced a 5′ Kozak sequence and the resulting PCR product was ligated into pcDNA3. Plasmid pCDH1-ERK2-EGFP was created by gene assembly of ERK2 and EGFP using overlap extension PCR with primers that introduced a 5′ Kozak sequence followed by ligation into pCDH1. Plasmid pcDNA3-EGFP-NLS was created by PCR amplification of EGFP with primers that added a 5′ Kozak sequence and 3′ SV40 NLS sequence and then ligation of the PCR product into pcDNA3. Plasmid pcDNA3-SHP2-EGFP was created by PCR amplification of SHP2 with a 5′ Kozak sequence followed by ligation into pcDNA3-EGFP. Plasmid pcDNA3-EGFP-HRas^{G12V} was generated by PCR amplification of EGFP-HRas^{G12V} from plasmid mEGFP-HRas G12V and the PCR product was subsequently ligated into pCDH1.

For creation of GFP-directed uAbs, plasmid pcDNA3-HF-GS2 was created by PCR amplification of GS2 from pHFT2-GS2 (Koide et al., “Teaching an Old Scaffold New Tricks: Monobodies Constructed Using Alternative Surfaces of the FN3 Scaffold,” J. Mol. Biol. 415(2):393-405 (2012), which is hereby incorporated by reference in its entirety) using primers that introduced upstream Kozak, 6×-His, and FLAG sequences followed by ligation into pcDNA3 such that BamHI and EcoRI restriction sites were available upstream of GS2 for generating N-terminal fusions. For C-terminal fusions, plasmid pcDNA3-GS2-FH was created by PCR amplifying GS2 with primers that introduced an upstream Kozak sequence and downstream NheI and SbfI restriction sites followed by ligation into pcDNA3. The genes encoding AvrPtoB, IpaH9.8, NleG2-3, NleG5-1, NleL, SlrP, SopA, SPOP, SspH1, SspH2, and XopL were PCR amplified with primers introducing NheI and SbfI sites, after which the resulting PCR products were ligated in pcDNA3-GS2-FH. The genes encoding LegAU13, LegU1, and SidC were PCR amplified with primers that introduced BamHI and EcoRI sites, after which the resulting PCR products were ligated in pcDNA3-HF-GS2. Plasmid pcDNA3-GS2-CHIP was created by PCR amplification of GS2 from pHFT2-GS2 using primers that introduced an upstream HindIII and Kozak sequence and downstream NheI site, followed by ligation into pcDNA3-R4-CHIPATPR in place of scFvR4. Plasmids pcDNA3-VHL-GS2 and pcDNA3-βTrCP-GS2 were created by PCR amplification of genes encoding VHL and βTrCP with primers that introduced HindII and XhoI (VHL) or BamHI and XhoI (βTrCP) sites after which the resulting PCR products were ligated in place of NSlmb in pcDNA3-NSlmb-GS2. Plasmids pcDNA3-GS2-IpaH9.8^{C337A}, pcDNA3-GS2-IpaH0722, pcDNA3-GS2-IpaH1.4, pcDNA3-GS2-IpaH2.5, pcDNA3-GS2-IpaH4.5, and pcDNA3-GS2-IpaH7.8 were created by site-directed mutagenesis of pcDNA3-GS2-IpaH9.8. The following genes were purchased: SspH1 (Twist Biosciences), IpaH9.8 (Twist Biosciences), VHL (GenScript, Ohu23297D), LubX (Twist Biosciences), LegU1(DT), and LegAU13 (IDT). All others were amplified from existing plasmids in laboratory stocks or from genomic DNA.

Plasmid pET24d-GS2-IpaH9.8 and pET24d-IpaH9.8ΔLRR were created by PCR amplification of full-length GS2-IpaH9.8 and truncated IpaH9.8ΔLRR, respectively, with primers that introduced NcoI and NotI (GS2-IpaH9.8) or NheI and NotI (IpaH9.8 ΔLRR) sites, after which the resulting PCR products were ligated into pET24d(+). Plasmid pET28a-GS2 was created by PCR amplification of GS2 from pHFT2-GS2 using primers that introduced an upstream NcoI site and downstream FLAG, 6×-His, and HindIII sequences, after which the resulting PCR product was ligated into pET28a(+). Plasmid pTriEx-3-GS2-IpaH9.8^{C337A} was created by PCR amplification of GS2-IpaH9.8^{C337A} from pcDNA3-GS2-IpaH9.8^{C337A} with primers that introduced EcoRV and HindIII sites, after which the resulting PCR product was ligated into pTriEx-3. Plasmid pET28a-EGFP was created by PCR amplification of GFP with primers adding C-terminal 6×-His tag, after which the resulting PCR product was ligated in pET28a(+). All plasmids were verified by DNA sequencing at the Cornell Biotechnology Resource Center (BRC).

Cell Lines, Culture and Transfection.

All cell lines used in this study are provided in Table 1. Briefly, HEK293T and HeLa cells were obtained from ATCC, HeLa H2B-EGFP cells were kindly provided by Elena Nigg, and MCF10A rtTA cells were kindly provided by Matthew Paszek, HEK293T, HeLa, and HeLa H2B-EGFP cells were cultured in DMEM with 4.5 g/L glucose and L-glutamine (VWR) supplemented with 10% FetalCloneI (VWR) and 1% penicillin-streptomycin-amphotericin B (ThermoFisher) MCF-10a cells were grown in DMEM/F12 media (ThermoFisher) supplemented with 5% horse serum (ThermoFisher), 20 ng/mL EGF (Peprotech), 0.5 mg/mL hydrocortisone (Sigma), 100 ng/mL cholera toxin (Sigma), 10 μg/mL insulin (Sigma), and 1% penicillin-streptomycin-amphotericin B (ThermoFisher). All cells were maintained at 37° C., 5% CO₂ and and 90% relative humidity (RH).

Stable MCF10A rtTA cell lines were generated using Nucleofection Kit V (Lonza) and HyPBase, an expression plasmid for the hyperactive version of the PiggyBac transposase. Transposition of MCF10A rtTA cells was performed to generate the following stable lines: MCF10A EGFP-HRas^{G12V}; MCF10A EGFP-HRas^{G12V}:GS2-IpaH9.8; MCF10A EGFP-HRas^{G12V}:GS2-IpaH9.8^{C337A}; and MCF10A GS2-IpaH9.8. Stable cell lines were selected using 200 μg/mL hygromycin B (ThermoFisher).

Stable HEK293T cell lines expressing EGFP, EGFP-HRas^{G12V}, ERK2-EGFP, d2EGFP were generated by lentiviral transformation. Specifically, pLV IRES eGFP, pcDH1 eGFP-HRas^{G12V}, pcDH1 ERK2-EGFP, or pHIV-d2EGFP were transfected into HEK293T cells along with psPAX2 and pMD2.G by calcium phosphate transfection. Media was replaced after ˜16 h, followed by a 48-h incubation to allow virus production. Viral supernatant was removed and Polybrene (Sigma-Aldrich) added to a final concentration of 8 μg/mL, followed by clearance of cell debris by centrifugation at 2,000 rpm for 5 min. Resultant supernatant was diluted 1:6 with cell media and added to previously plated HEK293T cells for stable integration. HEK293T EGFP and HEK293T ERK2-EGFP cell lines were selected by fluorescence activated cell sorting (BD FACSAria). The HEK293T EGFP-HRas^{G12V} cell line was selected using 1 μg/mL puromycin (Sigma-Aldrich).

Western Blot Analysis.

HEK293T cells were plated at 10,000 cells/cm2 and transfected as described above before lysis with RIPA lysis buffer (Thermo Fisher). MCF10A cells were plated at 20,000 cells/cm2 and induced with 0.2 μg/mL doxycycline for 24 h before lysis with cell lysis buffer. Lysates were separated on Any kD polyacrylamide gels (Bio-Rad) and transferred to PVDF membranes. α-HIS-HRP (Abcam), α-GFP (Krackeler) and α-GAPDH (Millipore) antibodies were diluted 1:5,000 and in TBST+1% milk and incubated for 1 h at room temperature. Secondary antibody goat anti-mouse IgG with HRP conjugation (Promega) was diluted at 1:2,500 and used as needed.

Flow Cytometric Analysis.

Cells were passed into 12-well plates at 10,000 cells/cm². 16-24 h after seeding, cells were transiently transfected with 1 μg total DNA at a 1:2 ratio of DNA:jetPrime (Polyplus Transfection). Cells were transfected with 0.05 μg of target, 0.25 μg of ubiquibody or control, and balanced with empty pcDNA3 vector. Culture media was replaced 4-6 h post-transfection. Then, 24 h post-transfection, cells were harvested and resuspended in phosphate buffered saline (PBS) for analysis using a FACSCalibur or FACSAria Fusion (BD Biosciences). FlowJo Version 10 was used to analyze samples by geometric mean fluorescence determined from 10,000 events.

Microscopy.

Cells were plated at 10,000 cells/cm² on a glass bottom 12-well plate pre-treated with poly-L-lysine (Sigma-Aldrich). After seeding for 16-24 h, cells were transfected with 1 μg total DNA at a 1:2 ratio of DNA:jetPrime (Polyplus Transfection). Cells were transfected with 0.05 μg of target, 0.25 μg of ubiquibody or control, and balanced with empty pcDNA3 vector. Culture media was replaced 4-6 h post-transfection. Then, 24 h post-transfection, cells were fixed with 4% paraformaldehyde. For EGFR-EGFP samples, cells were blocked with 5% normal goat serum in PBS for 2 h at room temperature. The anti-EGFR antibody (Cell Signalling #4267) was diluted 1:200 in 5% normal goat serum in PBS and incubated overnight at 4° C. Cells were washed three times with PBS, then incubated for 1 h at room temperature with anti-rabbit-AF647 diluted 1:200 in 5% normal goat serum in PBS. Cells were washed three times with PBS. Cell nuclei were stained with Hoescht diluted 1:10,000 in PBS for 10 min, then washed three times in PBS. Samples were imaged on an inverted Zeiss LSM88-confocal/multiphoton microscope (i880) using a 40× water immersion objective. Images were analyzed with FIJI.

Protein Expression and Purification.

Purified proteins were obtained by growing E. coli BL21(DE3) cells containing a pET28a-based plasmid encoding the desired protein or Rosetta(DE3) cells containing a pTriEx-3-based plasmid in 200 mL of Luria-Bertani (LB) medium at 37° C. Expression was induced with 0.1 mM IPTG when the culture density (Abs₆₀₀) reached 0.6-0.8 and growth continued for 6 h at 30° C. Cultures were harvested by centrifugation at 4,000×g for 30 min at 4° C. Cell pellets were stored at −20° C. overnight. Thawed pellets were resuspended in 10 mL equilibration buffer (25 mM Tris-HCl, pH 7.4, 500 mM NaCl and 20 mM imidazole) and lysed with a high-pressure homogenizer (Avestin Emulsi-Flex C5). The insoluble fraction was cleared by centrifugation at 12,000×g for 30 min at 4° C. His-tagged protein was purified by gravity flow using 500 μL HisPur Ni-NTA resin (ThermoFisher). The soluble fraction was passed through the resin, after which the resin was washed with 3 mL of wash buffer (25 mM Tris-HCl, pH 7.4, 500 mM NaCl and 50 mM imidazole). Protein was eluted with 1.5 mL elution buffer (25 mM Tris-HCl, pH 7.4, 500 mM NaCl and 250 mM imidazole). Purified fractions were desalted and concentrated (Pierce PES Protein Concentrators).

ELISA.

A 96-well enzyme immunoassay plate was coated with 100 μL of EGFP at 10 μg/mL in 0.05 M NaCO₃ buffer, pH 9.6 at 4° C. overnight. The plate was then washed three times 200 μL PBST (1×PBS+0.1% Tween 20) per well and blocked with 250 μL PBS with 3% milk per well at room temperature for 3 h, slowly mixing. The plate was washed three more times, followed by the addition of serial dilutions of purified proteins in blocking buffer at 60 μL per well. Plate was incubated at room temperature, slowly mixing for 1 h. The plate was washed three times to remove un-bound protein and then incubated with 50 μL/well of anti-FLAG (DDDYK) antibody conjugated to horseradish peroxidase (HRP) diluted 1:10,000 in PBST+1% milk for 1 h with slow mixing. The plate was washed three times before the addition of 50 μL/well 1-Step Ultra TMB (3,3′,5,5′-tetramethylbenzidine) (ThermoFisher). The reaction was allowed to incubate with slow mixing and then quenched with 50 μL/well of 3N H₂SO₄. The quenched plate was then read at 450 nm.

Synthesis and Characterization of Cationic Polypeptides.

N4 (TEP) polyamines were synthesized as the researchers of the present group described recently (Li et al., “Polyamine-Mediated Stoichiometric Assembly of Ribonucleoproteins for Enhanced mRNA Delivery,” Angew Chem. Int. Ed. Engl. 56(44):13709-12 (2017), which is hereby incorporated by reference in its entirety) according to a modified procedure of Uchida and coworkers. Uchida et al., “Modulated Protonation of Side Chain Aminoethylene Repeats in N-Substituted Polyaspartamides Promotes mRNA Transfection,” J. Amer. Chem. Soc. 136(35):12396-405 (2014), which is hereby incorporated by reference in its entirety. Briefly, to a chilled solution of poly(β-benzyl-L-aspartate) in N-methyl-2-pyrrolidone (NMP) (Sigma) (2 mL) was added dropwise with stirring 50 equivalents of tetraethylenepentamine (Sigma) diluted two-fold with NMP. After stirring for 2 h at 0° C., the pH was adjusted to 1 with dropwise addition while stirring of cold 6 N HCl. The resulting solution was dialyzed from a regenerated cellulose membrane bag (Spectrum Laboratories, 1 kDa MWCO) against 0.01 N HCl followed by distilled water, frozen, and lyophilized to give a white powder. Polyamines used in this study were characterized by ¹H NMR spectra in deuterium oxide (Cambridge Isotope Laboratories) using a Broker Avance 400 MHz NMR spectrometer at 25° C.: ¹H NMR (400 MHz, D2O) δ 4.72 (s, 1H), 3.64-3.39 (m, 9H), 3.37-3.05 (m, 5H), 3.00-2.62 (m, 4H).

Preparation of mRNA by In Vitro Transcription.

cDNA encoding uAbs was cloned into pGEM4Z/GFP/A64 by replacing the GFP fragment with XbaI and NotI sites. Additionally, the human α-globin 3′ UTR sequence was placed between the cDNA and the poly A tail using NotI and EcoRI to improve mRNA translation. Linearization with SpeI, followed by in vitro transcription (IVT) with HiScribe™ T7 High Yield RNA Synthesis Kit (NEB), yielded a transcript containing 64 nucleotides of vector-derived sequence, the coding sequence, α-globin 3′ UTR, and 64 A residues. In a typical 20 μl reaction, the following nucleotides were prepared: ATP (10 mM), pseudo-UTP (10 mM), methyl-CTP (10 mM), GTP (2 mM), anti-reverse Cap analog (8 mM, NEB). RNA was purified by RNeasy purification kit (Qiagen, Hilden, Germany). RNA quality was confirmed by running a 1% agarose gel. Concentration was determined by Abs₂₆₀.

Nanoplex Transfection.

Polyamines were dissolved in 10 mM HEPES buffer (pH 7.4). For each well of a 96-well plate, 200 ng mRNA diluted in 5 μl OptiMEM (Thermo Fisher) was mixed with 5 μl OptiMEM containing PABP (mRNA:PABP weight ratio=1:5) at room temperature for 10 min. Afterwards, 5 μl OptiMEM containing polyamines was added and incubated at room temperature for 15 min prior to transfection into HEK293T stably expressing d2EGFP. Polyamines were adjusted to achieve 50 to 1 (N/P) ratio for transfection. EGFP expression was measured by BD FACSCelesta (Becton Dickinson) at different time points after transfection.

Animal Experiments.

Mouse care and experimental procedures were performed under pathogen-free conditions in accordance with established institutional guidelines and approved protocols from the MIT Division of Comparative Medicine. C57BL/6-Tg(UBC-GFP)30Scha/J mice were purchased from Jackson Laboratory. 8-10 week-old mice were injected subcutaneously in ears with 5 μg mRNA and 25 μg PABP packaged with N4 (TEP) polyamines in a volume of 25 μl OptiMEM under anesthesia. Fluorescent imaging was performed with a CCD camera mounted in a light-tight specimen box (Xenogen). The exposure time was 1 s. Imaging and quantification of signals were controlled by Living Image acquisition and analysis software (Xenogen).

### Example 1—Engineered IpaH9.8 Potently Silences GFP in Mammalian Cells

To determine whether E3 ubiquitin ligase mimics from pathogenic bacteria could be redesigned for silencing of non-native targets, the focus was on a panel of 14 candidate enzymes representing the major classes of E3s found in bacteria to date (Table 2, infra). Maculins et al., “Bacteria-Host Relationship: Ubiquitin Ligases as Weapons of Invasion. Cell Res. 26(4):499-510 (2016) and Lin et al., “Exploitation of the Host Cell Ubiquitin Machinery by Microbial Effector Proteins,” J. Cell Sci. 130(12):1985-96 (2017), which are hereby incorporated by reference in their entirety.

TABLE 2
Bacterial E3 ubiquitin ligases evaluated in this study.

| E3 ubiquitin | | | | |
|---|---|---|---|---|
| ligase | Classification | Organism | Construction | Ref.¹ |
| AvrPtoB | U-box | Pseudomonas | AvrPtoB₁₋₄₃₆ fused to N-terminus of GS2 | [12] |
| | | syringae | | |
| βTrCP | F-box | Homo sapiens | βTrCP₁₋₅₆₈ fused to N-terminus of GS2 | [13] |
| | | | with (GlySer)₁₀ linker | |
| CHIP | U-box | H. sapiens | CHIP₁₂₈₋₃₀₃ fused to C-terminus of GS2 | [14] |
| IpaH0722 | NEL | Shigella flexneri | IpaH0722₂₉₅₋₅₈₇ fused to C-terminus of GS2 | [15] |
| IpaH1.4 | NEL | S. flexneri | IpaH1.4₂₈₅₋₅₇₅ fused to C-terminus of GS2 | [15] |
| IpaH2.5 | NEL | S. flexneri | IpaH2.5₂₉₂₋₅₇₀ fused to C-terminus of GS2 | [15] |
| IpaH4.5 | NEL | S. flexneri | IpaH4.5₂₉₆₋₅₇₄ fused to C-terminus of GS2 | [15] |
| IpaH7.8 | NEL | S. flexneri | IpaH7.8₂₇₄₋₅₆₅ fused to C-terminus of GS2 | [15] |
| IpaH9.8 | NEL | S. flexneri | IpaH9.8₂₅₄₋₅₄₅ fused to C-terminus of GS2 | [16] |
| LegAU13 | F-box | Legionella | LegAU13₁₋₅₀ fused to N-terminus of GS2 | [17] |
| | | nneumonhila | | |
| LegU1 | F-box | L. pneumophila | LegU1₁₋₅₆ fused to N-terminus of GS2 | [17] |
| LubX | U-box | L. pneumophila | LubX₁₋₂₁₅ fused to N-terminus of GS2 | [18] |
| NleG2-3 | U-box | Enterohemorrhagic | NleG2-3₉₀₋₁₉₁ fused to C-terminus of GS2 | [19] |
| | | Escherichia coli | | |
| | | (EHEC) O157:H7 | | |
| NleG5-1 | U-box | EHEC O157:H7 | NleG5-1₁₁₃₋₂₁₃ fused to C-terminus of GS2 | [19] |
| NleL | HECT | EHEC O157:H7 | NleL₃₇₁₋₇₈₂ fused to C-terminus of GS2 | [20] |
| SidC | Unconventional | L. pneumophila | SidC₁₋₅₄₂ fused to N-terminus of GS2 | [21] |
| SlrP | NEL | EHEC O157:H7 | SlrP₄₆₅₋₇₆₅ fused to N-terminus of GS2 | [22] |
| SopA | HECT | Salmonella | SopA₃₇₀₋₇₈₂ fused to C-terminus of GS2 | [23] |
| | | typhimurium | | |
| SPOP | F-box | H. sapiens | SPOP₁₆₇₋₃₇₄ fused to C-terminus of GS2 | [24] |
| SspH1 | NEL | S. typhimurium | SspH1₄₀₄₋₇₀₁ fused to N-terminus of GS2 | [25] |
| SspH2 | NEL | S. typhimurium | SspH2₄₉₂₋₇₈₈ fused to C-terminus of GS2 | [26] |
| VHL | SCF-like ECV | H. sapiens | VHL₁₋₂₁₃ fused to N-terminus of GS2 | [27] |
| XopL | Unconventional | Xanthomonas | XopL₄₇₄₋₆₆₀ fused to C-terminus of GS2 | [28] |
| | | campestris | | |

¹References listed in Table 2 provide clear proof or annotation of the catalytic domains of each E3 ubiquitin ligase.

Abbreviations in Table 2: NEL, novel E3 ligase; HECT, homologous to E6AP C terminus; SCF, Skp1/Cdc53 or Cullen-1/F-box protein; SPOP, speckle-type POZ protein; VHL, von Hippel-Lindau; ECV, Elongin B/C, Cullen-2, VHL
[12] Janjusevic et al., “A Bacterial Inhibitor of Host Programmed Cell Death Defenses is an E3 ubiquitin ligase,” Science 311:222-26 (2006), which is hereby incorporated by reference in its entirety
[13] Zhou et al., “Harnessing the Ubiquitination Machinery to Target the Degradation of Specific Cellular Proteins,” Mol. Cell 6:751-56 (2000), which is hereby incorporated by reference in its entirety
[14] Portnoff et al., “Ubiquibodies, Synthetic E3 Ubiquitin Ligases Endowed With Unnatural Substrate Specificity for Targeted Protein Silencing,” J. Biol. Chem. 289:7844-55 (2014), which is hereby incorporated by reference in its entirety
[15] Ashida et al, “Shigella Chromosomal IpaH Proteins are Secreted Via the Type III Secretion System and act as effectors,” Mol. Microbiol. 63:680-93 (2007), which is hereby incorporated by reference in its entirety
[16] Okuda et al. Shigella Effector IpaH9.8 Binds to a Splicing Factor U2AF(35) to Modulate Host Immune Responses,” Biochem. Biophys. Res. Commun. 333:531-39 (2005), which is hereby incorporated by reference in its entirety
[17] Ensminger, A. W., “RR E3 Ubiquitin Ligase Activity and Targeting of BAT3 by Multiple Legionella pneumophila Translocated Substrates,” Infect. Immun. 78:3905-19 (2010), which is hereby incorporated by reference in its entirety
[18] Quaile et al., “Molecular Characterization of LubX: Functional Divergence of the U-Box Fold by Legionella pneumophila,” Structure 23:1459-69 (2015), which is hereby incorporated by reference in its entirety
[19] Wu et al., “NleG Type 3 Effectors From Enterohaemorrhagic Escherichia coli are U-Box E3 Ubiquitin Ligases,” PLoS Pathog. 6:e1000960 (2010), which is hereby incorporated by reference in its entirety
[20] Lin et al., “Biochemical and Structural Studies of a HECT-like Ubiquitin Ligase From Escherichia coli O157:H7,” J. Biol. Chem. 286:441-49 (2011), which is hereby incorporated by reference in its entirety
[21] Hsu et al., “The Legionella Effector SidC Defines a Unique Family of Ubiquitin Ligases Important for Bacterial Phagosomal Remodeling,” Proc. Natl. Acad. Sci. USA 111:10538-43 (2014), which is hereby incorporated by reference in its entirety
[22] Zouhir et al., “The Structure of the Slrp-Trx1 Complex Sheds Light on the Autoinhibition Mechanism of the Type III Secretion System Effectors of the NEL family,” Biochem. J. 464:135-44 (2014), which is hereby incorporated by reference in its entirety
[23] Diao et al., “Crystal Structure of SopA, a Salmonella Effector Protein Mimicking a Eukaryotic Ubiquitin Ligase,” Nat. Struct. Mol. Biol. 15:65-70 (2008), which is hereby incorporated by reference in its entirety
[24] Shin et al., “Nanobody-Targeted E3-Ubiquitin Ligase Complex Degrades Nuclear Proteins,” Sci. Rep. 5:14269 (2015), which is hereby incorporated by reference in its entirety
[25] Keszei et al., “Structure of an SspH1-PKN1 Complex Reveals the Basis for Host Substrate Recognition and Mechanism of Activation for a Bacterial E3 Ubiquitin Ligase,” Mol. Cell Biol. 34:362-73 (2014), which is hereby incorporated by reference in its entirety
[26] Bhaysar et al., “The Salmonella Type III Effector SspH2 Specifically Exploits the NLR Co-Chaperone Activity of SGT1 to Subvert Immunity,” PLoS Pathog. 9:e1003518 (2013), which is hereby incorporated by reference in its entirety
[27] Maniaci et al., “Homo-PROTACs: Bivalent Small-Molecule Dimerizers of the VHL E3 Ubiquitin Ligase to Induce Self-Degradation,” Nat. Commun. 8:830 (2017), which is hereby incorporated by reference in its entirety
[28] Singer et al., “A Pathogen Type III Effector With a Novel E3 Ubiquitin Ligase Architecture,” PLoS Pathog. 9:e1003121 (2013), which is hereby incorporated by reference in its entirety

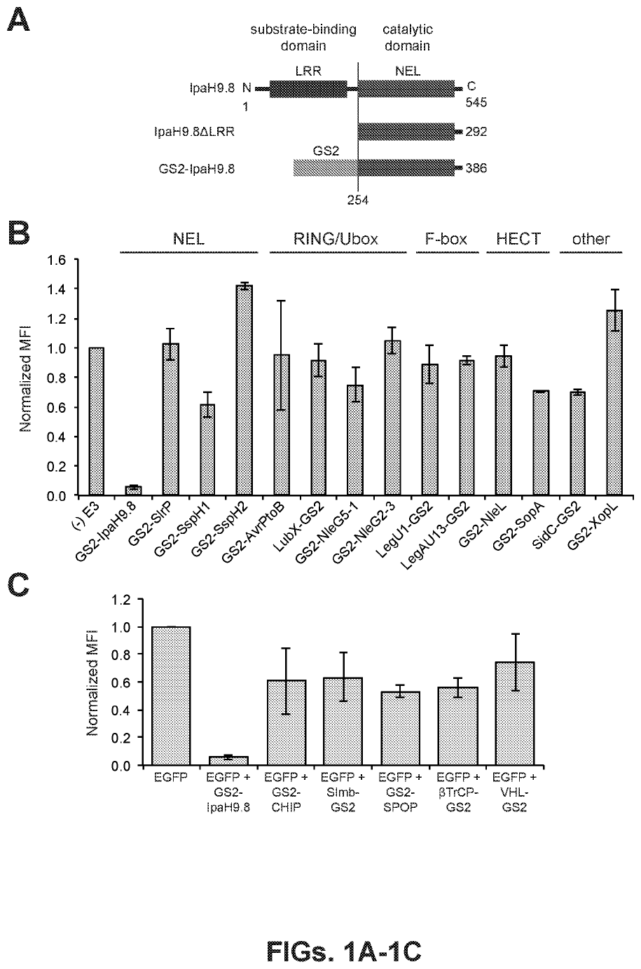

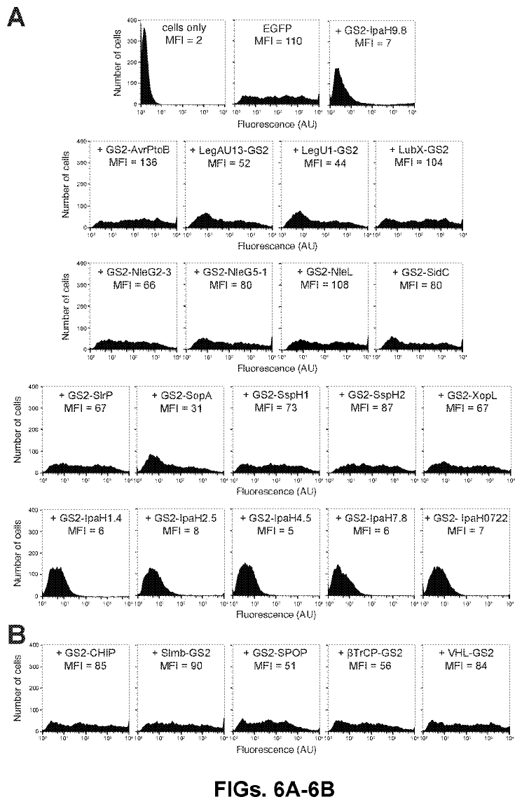

This panel included E3 mimics with folds similar to eukaryotic E3s such as HECT-type, RING or U-box (RING/U-box)-type, and F-box domains, as well as unconventional E3s with folds unlike any other eukaryotic E3s such as novel E3 ligase (NEL), XL-box-containing, and SidC. In general, uAbs were engineered by removing the native substrate-binding domain from each E3 mimic and replacing it with a synthetic binding protein (FIG. 1A), akin to the previously designed uAbs based on human CHIP. Portnoff et al., “Ubiquibodies, Synthetic E3 Ubiquitin Ligases Endowed With Unnatural Substrate Specificity for Targeted Protein Silencing,” J. Biol. Chem. 289(11):7844-55 (2014), which is hereby incorporated by reference in its entirety. For example, S. flexneri IpaH9.8 consists of an N-terminal domain with eight 20-residue leucine-rich repeats (LRRs) that mediate binding and specificity to native substrate proteins such as NF-κB essential modulator (NEMO) (Ashida et al., “A Bacterial E3 Ubiquitin Ligase IpaH9.8 Targets NEMO/IKKgamma to Dampen the Host NF-KapPab-Mediated Inflammatory Response,” Nat Cell Biol. 12(1):66-73, sup. pp. 1-9 (2010), which is hereby incorporated by reference in its entirety) and guanylate-binding proteins (GBPs) (Li et al., “Ubiquitination and Degradation of GBPs by a Shigella Effector to Suppress Host Defence,” Nature 551(7680):378-83 (2017), which is hereby incorporated by reference in its entirety), while the C-terminal domain adopts a novel E3 ubiquitin ligase architecture. Zhu et al., “Structure of a Shigella Effector Reveals a New Class of Ubiquitin Ligases,” Nat. Struct. Mol. Biol. 15(12):1302-08 (2008) and Singer et al., “A Pathogen Type III Effector With a Novel E3 Ubiquitin Ligase Architecture,” PLoS Pathogens 9(1):e1003121 (2013), which are hereby incorporated by reference in their entirety. Hence, the N-terminal LRR domain of IpaH9.8 was replaced with GS2, an FN3 monobody that binds GFP with nanomolar affinity (K_{d}=31 nM). Koide et al., “Teaching an Old Scaffold New Tricks: Monobodies Constructed Using Alternative Surfaces of the FN3 Scaffold,” J. Mol. Biol. 415(2):393-405 (2012), which is hereby incorporated by reference in its entirety. By swapping the natural substrate recognition function of these enzymes with the GS2 monobody, synthetic E3 ligases were created that was hypothesized to target GFP and promote its proteasomal degradation. To test this hypothesis, the different GS2-E3 chimeras were transiently co-expressed along with enhanced GFP (EGFP) in mammalian cells and fluorescence activity was monitored by flow cytometric analysis. By far, the most striking depletion of EGFP was achieved with GS2-IpaH9.8, which reduced EGFP fluorescence to near background levels (FIG. 1B and FIGS. 6A-6B). All of the other uAbs showed relatively weak silencing activity under the conditions tested here. GS2-NleG5-1, GS2-SspH1, SidC-GS2, and GS2-SopA were the most active among these, reducing EGFP fluorescence by ˜20-40% (FIG. 1B and FIGS. 6A-6B).

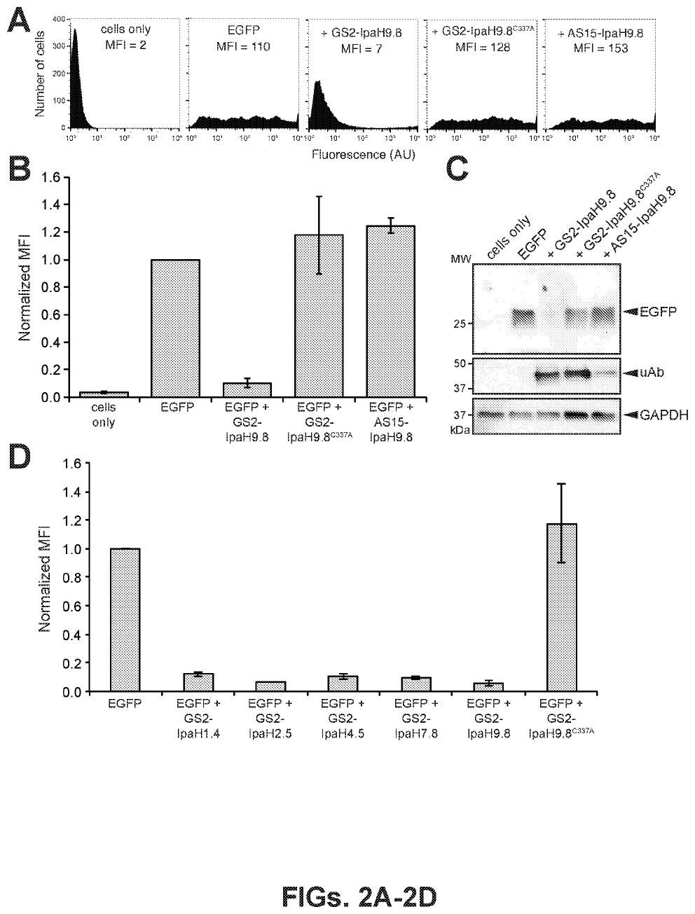

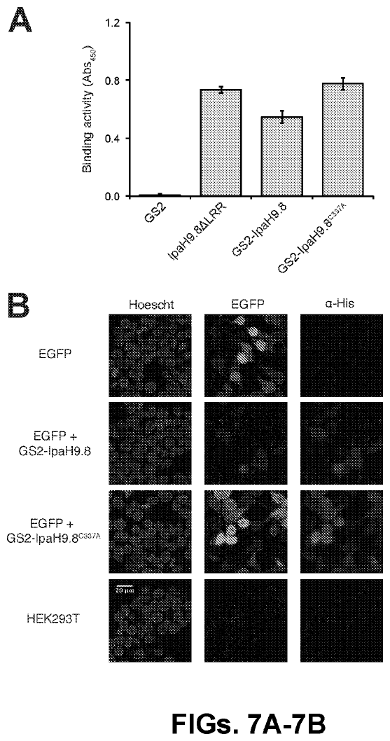

In light of this robust silencing activity, the attention was focused on the GS2-IpaH9.8. In cells expressing this chimera, the elimination of EGFP was efficient, with removal of up to 90% of the fluorescence activity (FIGS. 2A and 2B) and no detectable EGFP protein in cell lysates (FIG. 2C). Importantly, silencing activity was completely abrogated when the catalytic cysteine of IpaH9.8 (Rohde et al., “Type III Secretion Effectors of the IpaH Family are E3 Ubiquitin Ligases,” Cell Host Microbe 1(1):77-83 (2007), which is hereby incorporated by reference in its entirety) was mutated to alanine (GS2-IpaH9.8^{C337A}) and when the non-cognate FN3 monobody AS15, which is specific for the Abl SH2 domain (Koide et al., “High-Affinity Single-Domain Binding Proteins with a Binary-Code Interface,” Proc. Natl. Acad. Sci. USA 104(16):6632-37 (2007), which is hereby incorporated by reference in its entirety), was substituted for GS2 (FIGS. 2A-2C), indicating that target degradation was dependent on cooperation of both uAb domains. In the case of GS2-IpaH9.8^{C337A}, expression in mammalian cells and EGFP binding activity in vitro were unaffected by the alanine substitution (FIGS. 7A-7B), confirming that loss of silencing activity was due to catalytic inactivation. It should also be noted that removal of the LRR domain was essential for knockdown activity, as direct fusion of GS2 to full-length IpaH9.8 that had not been truncated resulted in no measurable silencing activity. Interestingly, the genome sequences of S. flexneri strains indicate that several IpaH family members, namely IpaH1.4, IpaH2.5, IpaH4.5, IpaH7.8 and IpaH9.8, are encoded on the 220-kb virulence plasmid pWR100 while seven additional ipaH cognate genes are present on the chromosome. Maculins et al., “Bacteria-Host Relationship: Ubiquitin Ligases as Weapons of Invasion. Cell Res. 26(4):499-510 (2016), which is hereby incorporated by reference in its entirety. To determine whether these family members were as proficient as IpaH9.8 at degrading EGFP in the uAb context, chimeras were generated between GS2 and the catalytic domains derived from each of the pWR100-encoded IpaH family members as well as one chromosomally encoded member, IpaH0722. When expressed ectopically in cultured cells, all of the IpaH-based uAbs were capable of efficient (˜90% or greater) EGFP knockdown in mammalian cells (FIG. 2D). This result was not entirely surprising in light of the high homology shared by the different catalytic domains. Indeed, whereas the different IpaH family members were only ˜70% similar to IpaH9.8 overall, the catalytic domains were much more similar (>99%) with just 1-3 amino acid substitutions and, in the case of IpaH1.4 and IpaH4.5, minor C-terminal truncations (Table 2).

To benchmark the potency of the present engineered bacterial ligase, the GFP silencing activity catalyzed by GS2-IpaH9.8 was compared with that of other synthetic ligases based on eukaryotic E3 machinery that have previously been reconfigured for targeted proteolysis. Zhou et al., “Harnessing the Ubiquitination Machinery to Target the Degradation of Specific Cellular Proteins,” Mol. Cell 6(3):751-56 (2000); Zhang et al., “Exploring the Functional Complexity of Cellular Proteins by Protein Knockout,” Proc. Natl. Acad. Sci. USA 100(24):14127-32 (2003); Hatakeyama et al., “Targeted Destruction of C-Myc by an Engineered Ubiquitin Ligase Suppresses Cell Transformation and Tumor Formation,” Cancer Res. 65(17):7874-79 (2005); Ma et al., “Targeted Degradation of KRAS by an Engineered Ubiquitin Ligase Suppresses Pancreatic Cancer Cell Growth In Vitro and In Vivo,” Mol. Cancer Ther. 12(3):286-94 (2013); Kong et al., “Engineering a Single Ubiquitin Ligase for the Selective Degradation of all Activated ErbB Receptor Tyrosine Kinases,” Oncogene 33(8):986-95 (2014); Caussinus et al., “Fluorescent Fusion Protein Knockout Mediated by Anti-GFP Nanobody,” Nat. Struct. Mol. Biol. 19(1):117-21 (2011); Fulcher et al., “Targeting Endogenous Proteins For Degradation Through the Affinity-Directed Protein Missile System,” Open Biol. 7(5). 170066 (2017); Fulcher et al., “An Affinity-Directed Protein Missile System for Targeted Proteolysis,” Open Biol 6(10):160255 (2016); Shin et al., “Nanobody-Targeted E3-Ubiquitin Ligase Complex Degrades Nuclear Proteins,” Sci. Rep. 5:14269 (2015); and Kanner et al., “Sculpting Ion Channel Functional Expression with Engineered Ubiquitin Ligases,” Elife 6:e29744 (2017), which are hereby incorporated by reference in their entirety. Specifically, the natural substrate-binding domains for several eukaryotic E3 ubiquitin ligases from humans including carboxyl terminus of Hsc70-interacting protein (CHIP), speckle-type POZ protein (SPOP), β-transducing repeat-containing protein (βTrCP), and von Hippel-Lindau protein (VHL), as well as the Drosophila melanogaster supernumerary limbs (Slmb) protein were replaced with the GS2 monobody, resulting in a panel of synthetic ligases analogous to GS2-IpaH9.8. When the resulting panel of GFP-specific uAbs was transiently co-expressed with EGFP in mammalian cells, all were capable of measurably reducing EGFP levels, but silencing activity for each was relatively inefficient (˜25-45%) under the conditions tested here (FIG. 1C and FIGS. 6A-6B), reminiscent of previous results with a Slmb-nanobody chimera that was similarly ineffective at reducing unfused GFP levels. Caussinus et al., “Fluorescent Fusion Protein Knockout Mediated by Anti-GFP Nanobody,” Nat. Struct. Mol. Biol. 19(1):117-21 (2011), which is hereby incorporated by reference in its entirety. The weak EGFP knockdown observed here for Slmb-GS2 was actually an improvement over previous results obtained with a chimera between Slmb and a GFP-specific VHH nanobody, cAbGFP4, that was incapable of promoting degradation of unfused GFP. Caussinus et al., “Fluorescent Fusion Protein Knockout Mediated by Anti-GFP Nanobody,” Nat. Struct. Mol. Biol. 19(1):117-21 (2011), which is hereby incorporated by reference in its entirety. It should be noted, however, that the Slmb-cAbGFP4 fusion eliminated the fluorescence associated with larger GFP fusion proteins, suggesting that the data reported here are not necessarily indicative of uAb dysfunction but instead may reflect differences in substrate preference/compatibility or extent of ubiquitin decoration. Regardless, none of the engineered chimeras involving eukaryotic E3s displayed the potency and robustness of GS2-IpaH9.8, which reproducibly degraded 90-95% of cellular fluorescence.

### Example 2—a Broad Range of Substrate Proteins is Degraded by GS2-IpaH9.8

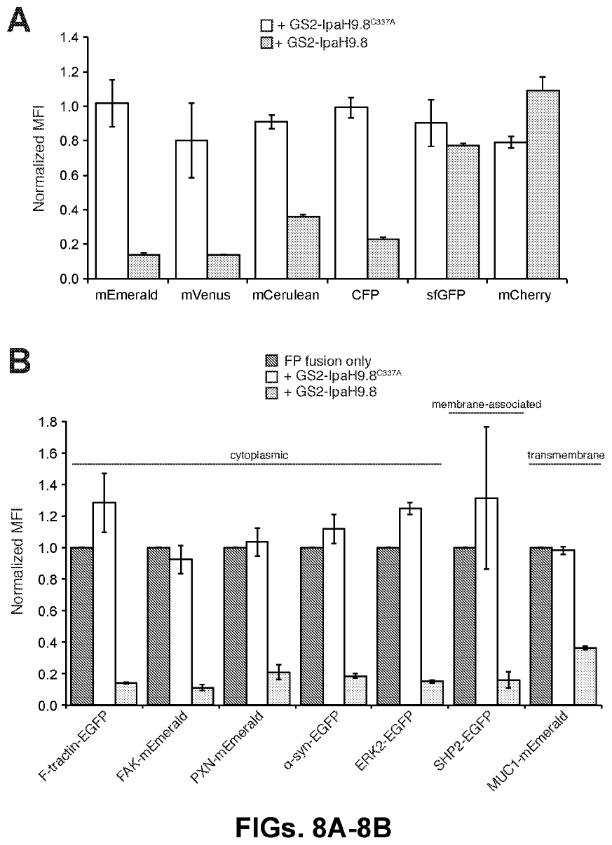

To more deeply explore the substrate compatibility issue, the ability of GS2-IpaH9.8 to degrade a range of different substrates was tested. A growing number of GFP-derived fluorescent proteins (FPs) have been developed and optimized over the years, providing a diverse collection of new tools for biological imaging. Tsien, R. Y., “The Green Fluorescent Protein,” Ann. Rev. Biochem. 67:509-44 (1998) and Shaner et al., “A Guide to Choosing Fluorescent Proteins,” Nat. Methods 2(12):905-09 (2005), which are hereby incorporated by reference in their entirety. To determine the extent to which different FP targets could be degraded, GS2-IpaH9.8 was transiently co-expressed in mammalian cells with monomeric versions of Emerald, Venus and Cerulean, as well as enhanced cyan fluorescent protein (ECFP). Approximately 65-85% of the cellular fluorescence activity associated with each of the FPs was ablated by GS2-IpaH9.8, whereas the structurally unrelated mCherry protein was not targeted by GS2-IpaH9.8, which was expected given the specificity of GS2 for the GFP fold (FIG. 8A) Interestingly, the fluorescence activity of superfolder GFP (sfGFP), a rapidly folding and robustly stable mutant of EGFP, was unaffected by GS2-IpaH9.8, consistent with recent findings that sfGFP is resistant to proteasomal degradation. Khmelinskii et al., “Incomplete Proteasomal Degradation of Green Fluorescent Proteins in the Context of Tandem Fluorescent Protein Timers,” Mol. Biol. Cell 27(2):360-70 (2016), which is hereby incorporated by reference in its entirety.

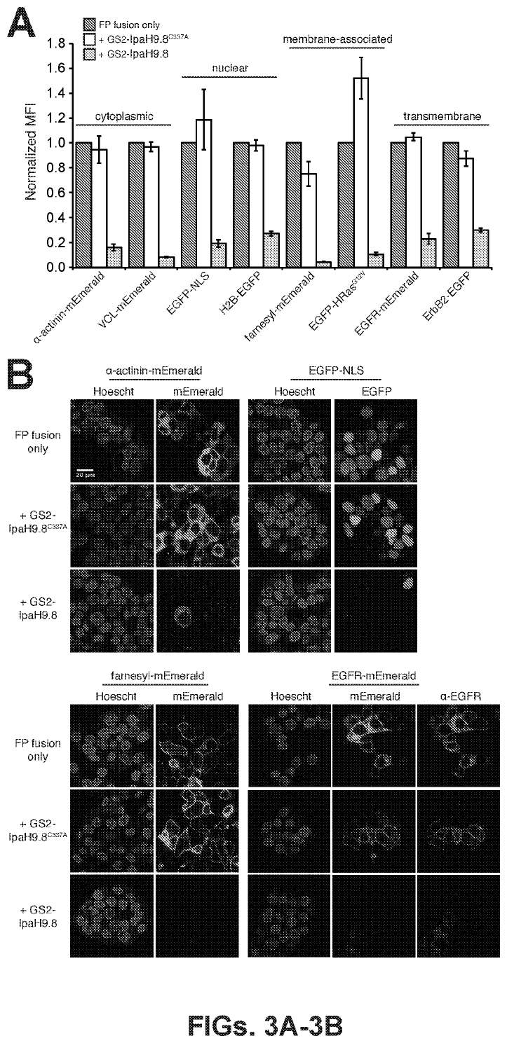

Encouraged by the ability of GS2-IpaH9.8 to degrade different FPs, the ability of GS2-IpaH9.8 to degrade structurally diverse, FP-tagged substrate proteins was next evaluated. GS2-IpaH9.8 proficiently degraded 15 unique target proteins that varied in terms of their molecular weight (27-179 kDa) and subcellular localization (i.e., cytoplasm, nucleus, membrane-associated, and transmembrane) (FIG. 3A and FIG. 8B). For example, GS2-IpaH9.8 triggered degradation of 80-92% of the fluorescence activity associated with FP fusions involving the cytoplasmic proteins α-actinin, α-synuclein (α-syn), extracellular signal-regulated kinase 2 (ERK2), focal adhesion kinase (FAK), F-tractin, paxillin (PXN), and vinculin (VCL) as determined by flow cytometric analysis (FIG. 3A and FIG. 8B). Similarly robust silencing was observed for: nuclear-targeted FP fusions involving histone H2B and the nuclear localization signal (NLS) derived from SV40 Large T-antigen; membrane-associated FP fusions involving Harvey rat sarcoma virus oncogene homolog carrying the oncogenic G12V mutation (HRas^{G12V}), Src-homology 2 domain-containing phosphatase 2 (SHP2), and the farnesyl sequence derived from HRas; and transmembrane FP fusions involving epidermal growth factor receptor (EGFR), avian erythroblastic leukemia viral oncogene homolog 2 (ErbB2), and mucin 1 (MUC1) (FIG. 3A and FIG. 8B). Microscopy analysis of representative substrate proteins α-actinin-mEmerald, EGFP-NLS, farnesyl-mEmerald, and EGFR-mEmerald confirmed the expected subcellular localization of each fusion and corroborated the efficient degradation activity measured by flow cytometric analysis (FIG. 3B). The transmembrane protein EGFR-mEmerald was examined by immunolabeling with an antibody specific to the extracellular domain of EGFR. Importantly, the α-EGFR signal decreased concomitantly with GFP disappearance (FIG. 3B), indicating that degradation of the entire transmembrane protein was achieved. Taken together, these results establish GS2-IpaH9.8 as a robust proteome editing tool that is capable of silencing a broad spectrum of substrates that span several distinct subcellular locations.

### Example 3—GS2-IpaH9.8-Mediated Proteome Editing is Flexible and Modular

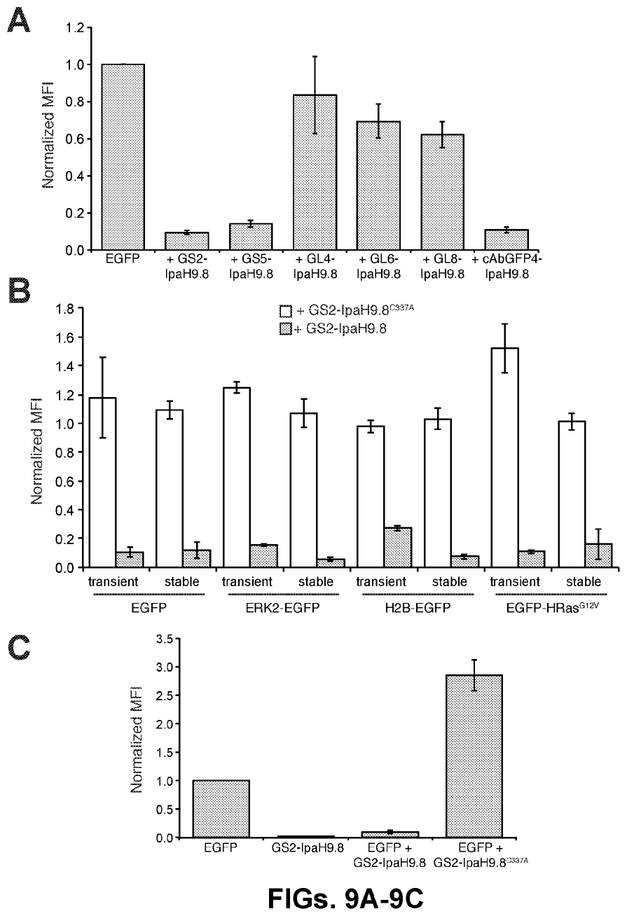

An attractive feature of uAbs is their highly modular architecture—the E3 catalytic domain and synthetic binding protein domain can be interchanged to reprogram the activity and specificity. Indeed, the results above revealed the ease with which different bacterial and eukaryotic E3 domains can be chimerized to form functional uAbs. To investigate the interchangeability of the synthetic binding protein domain in IpaH9.8-based uAbs, GS2 was first replaced with other high-affinity GFP-binding proteins such as the FN3 monobody GS5 (K_{d}=62 nM) (Koide et al., “Teaching an Old Scaffold New Tricks: Monobodies Constructed Using Alternative Surfaces of the FN3 Scaffold,” J. Mol. Biol. 415(2):393-405 (2012), which is hereby incorporated by reference in its entirety) or cAbGFP4 (K_{d}=0.32 nM) (Saerens et al., “Identification of a Universal VHH Framework to Graft Non-Canonical Antigen-Binding Loops of Camel Single-Domain Antibodies,” J. Mol. Biol. 352(3):597-607 (2005), which is hereby incorporated by reference in its entirety). For these constructs, efficient EGFP silencing activity was observed that rivaled that seen with the GS2 monobody (FIG. 9A). Interestingly, introduction of lower affinity (˜200-500 nM) FN3 monobodies (Koide et al., “Teaching an Old Scaffold New Tricks: Monobodies Constructed Using Alternative Surfaces of the FN3 Scaffold,” J. Mol. Biol. 415(2):393-405 (2012), which is hereby incorporated by reference in its entirety) resulted in less efficient EGFP elimination (FIG. 9A), suggesting that silencing activity may be a function of the affinity for the target protein. Although, because spatial arrangements and surface complementarity prioritize lysine sites for ubiquitination (Buetow et al., “Structural Insights into the Catalysis and Regulation of E3 Ubiquitin Ligases,” Nat. Rev. Mot Cell Biol. 17(10):626-42 (2016), which is hereby incorporated by reference in its entirety), an equally plausible explanation for these findings is that the various FN3 domains may differentially orient the uAb with respect to GFP in a manner that affects how the substrate is ubiquitinated.

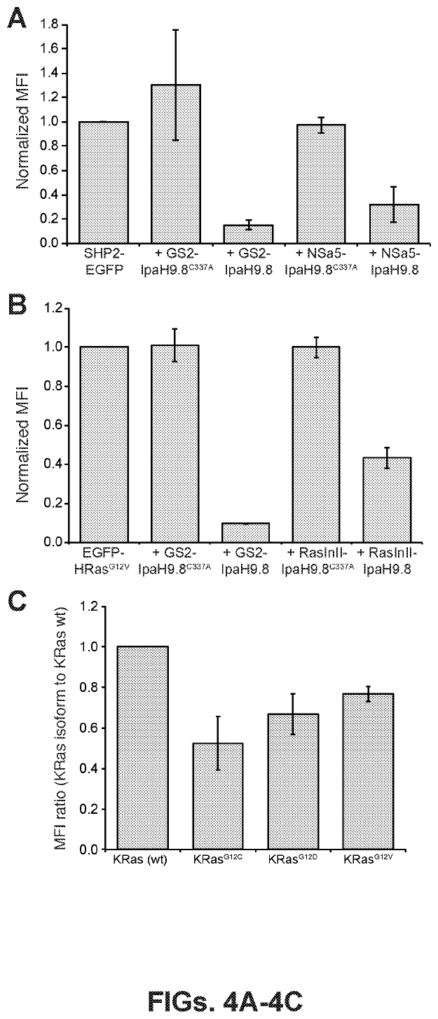

Next, the compatibility of the IpaH9.8 catalytic domain with two different FN3 monobodies was investigated: NSa5 that is specific for the Src-homology 2 (SH2) domain of SHP2 (Sha et al., “Dissection of the BCR-ABL Signaling Network Using Highly Specific Monobody Inhibitors to the SHP2 SH2 Domains,” Proc. Natl. Acad. Sci. USA 110(37):14924-29 (2013), which is hereby incorporated by reference in its entirety) and RasInII that is specific for HRas, KRas, and the G12V mutants of each (Cetin et al., “RasIns: Genetically Encoded Intrabodies of Activated Ras Proteins,” J. Mol. Biol. 429(4):562-573 (2017), which is hereby incorporated by reference in its entirety). The resulting NSa5-IpaH9.8 and RasInII-IpaH9.8 chimeras were tested for their ability to silence SHP2-EGFP and EGFP-HRas^{G12V}, respectively, by flow cytometric analysis. Both exhibited strong silencing activity, degrading their EGFP-tagged targets almost as efficiently as the GFP-directed GS2-IpaH9.8 (FIGS. 4A and 4B). Interestingly, RasInII-IpaH9.8 degraded EGFP-KRas^{G12C} and other KRas mutants (e.g., G12C, G12D) more efficiently than EGFP-KRas (FIG. 4C), in line with its selectivity for the G12V mutant over wild-type Ras isoforms (Cetin et al., “RasIns: Genetically Encoded Intrabodies of Activated Ras Proteins,” J. Mol. Biol. 429(4):562-573 (2017), which is hereby incorporated by reference in its entirety) and thus providing a potential route for mutant selective silencing of Ras. Collectively, these results reveal a remarkable plasticity for IpaH9.8, enabling its use as a “one-size fits all” degrader of diverse target proteins in transiently and stably transfected cell lines.

In all the experiments described above, efficient knockdown was achieved when GS2-IpaH9.8 and its corresponding target were transiently expressed. However, transient expression is not always an option, due to the experimental timescale, necessity for a precise expression profile, or the use of a recalcitrant mammalian cell line. Thus, to demonstrate the flexibility of GS2-IpaH9.8-mediated silencing, degradation activity was evaluated against target proteins that were expressed as stably integrated transgenes. Specifically, when GS2-IpaH9.8 was transiently expressed in cells that stably co-expressed EGFP, reduction of fluorescence activity was virtually identical to that observed for transiently expressed EGFP (FIG. 9B). Robust degradation was also observed for ERK2-EGFP, H2B-EGFP, and EGFP-HRas^{G12V}, regardless of their mode of expression (FIG. 9B). When the uAb and the target were both expressed as stable transgenes, thereby eliminating the need for transfection entirely, strong silencing activity was again observed for GS2-IpaH9.8 but not its inactive GS2-IpaH9.8^{C337A} counterpart (FIG. 9C).

### Example 4—Delivery of mRNA Encoding GS2-IpaH9.8 Enables Proteome Editing in Mice

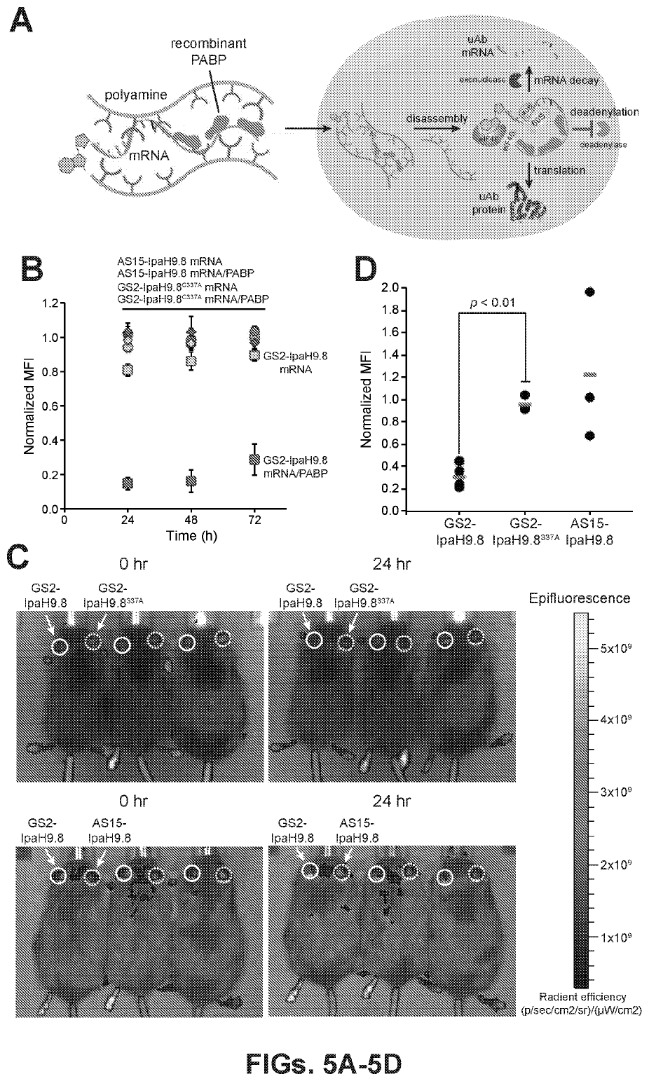

From a therapeutic standpoint, one of the biggest challenges facing protein-based technologies such as uAbs is intracellular delivery. Osherovich, L., “Degradation From Within,” Science-Business Exchange 7:10-11 (2014), which is hereby incorporated by reference in its entirety. The researchers of the present group previously showed that co-assembled nanoplexes comprised of synthetic mRNA containing a poly A tail, PABPs, and biocompatible cationic polypeptides (FIG. 5A) resulted in greatly enhanced mRNA expression in vitro and in mice. Li et al., “Polyamine-Mediated Stoichiometric Assembly of Ribonucleoproteins for Enhanced mRNA Delivery,” Angew Chem. Int. Ed. Engl. 56(44):13709-12 (2017), which is hereby incorporated by reference in its entirety. Here, it was hypothesized that delivery of GS2-IpaH9.8 mRNA/PABP nanoplexes to mammalian cells would result in significantly greater uAb expression relative to mRNA transfection alone by the same polyamine in HEK293T cells, thereby leading to potent protein degradation. To test this hypothesis, GS2-IpaH9.8 mRNA/PABP nanoplex delivery was first evaluated in vitro by quantifying the degradation of d2EGFP, a destabilized GFP variant that was expressed as a stable transgene in HEK293T cells. As expected, only when the cationic nanoplexes contained the active, target-specific GS2-IpaH9.8 mRNA and PABP was robust d2EGFP degradation achieved (FIG. 5B). All other controls including catalytically inactive GS2-IpaH9.8^{C337A} mRNA/PABP nanoplexes, non-specific AS15-IpaH9.8 nanoplexes, and naked GS2-IpaH9.8 mRNA that was delivered without PABPs showed little to no silencing activity (FIG. 5B). At 24 hours post-treatment, HEK293Td2EGFP cells receiving GS2-IpaH9.8 mRNA/PABP nanoplexes exhibited an 85% decrease in fluorescence activity, which was directly comparable to the knockdown activity achieved following DNA transfection seen above.

Encouraged by these results, uAb nanoplex-mediated delivery and silencing activity in vivo was next evaluated. Transgenic UBI-GFP/BL6 mice, which constitutively express EGFP in all tissues (Schaefer et al., “Observation of Antigen-Dependent CD8+ T-Cell/Dendritic Cell Interactions In Vivo,” Cell Immunol. 214(2):110-22 (2001), which is hereby incorporated by reference in its entirety), were given subcutaneous injections of GS2-IpaH9.8 mRNA/PABP nanoplexes in ears. Note that although this mouse strain ubiquitously expresses EGFP, fluorescence is absorbed and undetectable in areas that are covered by hairs. Fluorescent imaging at 24 hours post-injection revealed that EGFP fluorescence in the left ears, which received GS2-IpaH9.8 mRNA/PABP nanoplex injections, was robustly ablated with a 70% decrease in ear fluorescence (FIGS. 5C and 5D). In stark contrast, fluorescence in the right ears, which received either catalytically inactive GS2-IpaH9.8^{C337A} or non-specific AS15-IpaH9.8 nanoplex injections, was unaffected (FIGS. 5C and 5D). Importantly, these results set the stage for therapeutic delivery of uAbs as a viable strategy to post-translationally silence aberrantly expressed proteins in cancer and other human diseases.

### Example 5—Discussion of Examples 1-4

Ubiquibodies are a relatively new proteome editing modality that enable selective removal of otherwise stable proteins in somatic cells (Portnoff et al., “Ubiquibodies, Synthetic E3 Ubiquitin Ligases Endowed With Unnatural Substrate Specificity for Targeted Protein Silencing,” J. Biol. Chem. 289(11):7844-55 (2014), which is hereby incorporated by reference in its entirety), with potential applications in basic research, drug discovery, and therapy. In this study, a new class of uAbs that feature bacterial E3 ubiquitin ligases was created, thereby opening the door to a previously untapped source of ubiquitination activity for uAb development. Specifically, 14 bacterial E3 ligases belonging to a growing class of effector proteins that mimic host cell E3 ligases to exploit the ubiquitination pathway was evaluated. Maculins et al., “Bacteria-Host Relationship: Ubiquitin Ligases as Weapons of Invasion. Cell Res. 26(4):499-510 (2016) and Lin et al., “Exploitation of the Host Cell Ubiquitin Machinery by Microbial Effector Proteins,” J. Cell Sci. 130(12):1985-96 (2017), which are hereby incorporated by reference in their entirety. Most notable among these was IpaH9.8 from S. flexneri, which proved to be a remarkable catalyst of protein turnover when directed to target substrates via a genetically fused synthetic binding domain. This silencing activity was found to be independent of the substrate's subcellular localization (i.e., cytoplasm, nucleus, plasma membrane) or expression modality (i.e., transient versus stable). The only other E3 ligases that functioned comparably were homologs of IpaH9.8 found in S. flexneri, either on the pWR100 virulence plasmid or the chromosome. Maculins et al., “Bacteria-Host Relationship: Ubiquitin Ligases as Weapons of Invasion. Cell Res. 26(4):499-510 (2016), which is hereby incorporated by reference in its entirety. The N-terminal catalytic NEL domains of these enzymes share striking homology (99-100%), which explains their similar performance in the uAb context. Accordingly, the next best functioning bacterial E3 ubiquitin ligase was S. typhimurium SspH1, which is also a NEL type enzyme with 38% identity to IpaH9.8 overall and 42% identity within the NEL domain. Norkowski et al., The Species-Spanning Family of LPX-Motif Harbouring Effector Proteins,” Cell Microbiol. 20(11):e12945 (2018), which is hereby incorporated by reference in its entirety. It should also be pointed out that none of the mammalian E3 ubiquitin ligases were able to reduce EGFP levels below 60% under the conditions tested here. While the reasons for this are not entirely clear, given the successful knockdown results reported previously for these different E3 ligases in the uAb format (Portnoff et al., “Ubiquibodies, Synthetic E3 Ubiquitin Ligases Endowed With Unnatural Substrate Specificity for Targeted Protein Silencing,” J. Biol. Chem. 289(11):7844-55 (2014); Caussinus et al., “Fluorescent Fusion Protein Knockout Mediated by Anti-GFP Nanobody,” Nat. Struct. Mol. Biol. 19(1):117-21 (2011); Fulcher et al., “Targeting Endogenous Proteins For Degradation Through the Affinity-Directed Protein Missile System,” Open Biol. 7(5):170066 (2017); Fulcher et al., “An Affinity-Directed Protein Missile System for Targeted Proteolysis,” Open Biol 6(10):160255 (2016); Shin et al., “Nanobody-Targeted E3-Ubiquitin Ligase Complex Degrades Nuclear Proteins,” Sci. Rep. 5:14269 (2015); and Kanner et al., “Sculpting Ion Channel Functional Expression with Engineered Ubiquitin Ligases,” Elife 6:e29744 (2017), all of which are hereby incorporated by reference in their entirety), it is suspected that EGFP may represent a poor substrate for these engineered chimeras.

While the work here was predominantly focused on silencing FPs and FP-tagged substrates, IpaH9.8-based uAbs that potently degraded disease-related targets including HRas was designed, which together with KRas and NRas comprise the most commonly mutated oncoproteins in cancer, and SHP2, a regulator of the Ras/MAPK signaling pathway. Importantly, the ability to deplete these clinically important targets along with all of the other FP fusions serves to highlight the extraordinary modularity of the uAb technology. Simply swapping the native substrate-binding domain of the E3 ubiquitin ligase can generate a made-to-order uAb with specificity for a different substrate protein. Interestingly, Shigella have evolved a similar strategy for subverting host defenses during infection whereby plasmid and chromosomally-encoded IpaH proteins play a key role in dampening the host inflammatory response by mediating proteasomal degradation of NF-κB-related proteins. Ashida et al., “A Bacterial E3 Ubiquitin Ligase IpaH9.8 Targets NEMO/IKKgamma to Dampen the Host NF-KapPab-Mediated Inflammatory Response,” Nat Cell Biol. 12(1):66-73, sup. pp. 1-9 (2010) and Ashida et al., “Shigella IpaH Family Effectors as a Versatile Model for Studying Pathogenic Bacteria,” Front Cell Infect. Microbiol. 5:100 (2015), which are hereby incorporated by reference in their entirety. Specifically, by employing different LRR domains, which only share ˜50% similarity (Norkowski et al., “The Species-Spanning Family of LPX-Motif Harboring Effector Proteins,” Cell Microbial. e12945 (2018), which is hereby incorporated by reference in its entirety), Shigella are able to redirect virtually identical catalytic NEL domains to an array of host proteins (e.g., NEMO, U2AF53 for IpaH9.8; Glomulin for IpaH7.8; p65 for IpaH4.5; HOIP for IpaH2.5 and IpaH1.4; TRAF2 for IpaH0722). Maculins et al., “Bacteria-Host Relationship: Ubiquitin Ligases as Weapons of Invasion. Cell Res. 26(4):499-510 (2016); Lin et al., “Exploitation of the Host Cell Ubiquitin Machinery by Microbial Effector Proteins,” J. Cell Sci. 130(12):1985-96 (2017); and Ashida et al., “Shigella IpaH Family Effectors as a Versatile Model for Studying Pathogenic Bacteria,” Front Cell Infect. Microbiol. 5:100 (2015), all of which are hereby incorporated by reference in their entirety. It is believed that the inherent conformational flexibility required to ubiquitinate these structurally diverse substrates helps to explain the NEL motif's remarkable ability for customizable target degradation. It should also be pointed out that while the work here leveraged previously confirmed E3 ubiquitin ligases, an analogous swapping strategy could be used to create GS2-based uAbs for identifying novel E3 ligases. Such an approach could enable systematic identification of E3 ligases, which is an important objective given that the human genome encodes over 600 putative E3 ligases (Metzger et al., “HECT and RING Finger Families of E3 Ubiquitin Ligases at a Glance,” J. Cell Sci. 125(Pt 3):531-37 (2012), which is hereby incorporated by reference in its entirety) and bacterial genomes likely encode hundreds of others, many of which remain to be validated as catalysts of ubiquitin transfer.

From a drug development standpoint, pharmacological control of gene products has traditionally been achieved using small molecule inhibitors that target enzymes and receptors having well-defined hydrophobic pockets where the small molecules are tightly bound. Unfortunately, a majority (˜80-85%) of the human proteome is comprised of intractable targets, such as transcription factors, scaffold proteins, and non-enzymatic proteins, that cannot be inhibited pharmacologically and thus have been deemed ‘undruggable’. Crews, C. M., “Targeting the Undruggable Proteome: The Small Molecules of My Dreams,” Chem. Biol. 17(6):551-55 (2010) and Arkin et al., “Small-Molecule Inhibitors of Protein-Protein Interactions: Progressing Towards The Dream,” Nat. Rev. Drug Discov. 3(4):301-17 (2004), which are hereby incorporated by reference in its entirety. As an alternative, a number of techniques for silencing proteins at the DNA or RNA level are now available such as CRISPR, RNAi, TALENs, and ZFNs, with the first RNAi therapy, patisiran, gaining approval in 2018 for hereditary transthyretin amyloidosis. Adams et al., “Patisiran, An RNAi Therapeutic, For Hereditary Transthyretin Amyloidosis,” N. Engl. J. Med. 379(1):11-21 (2018), which is hereby incorporated by reference in its entirety. Nonetheless, new adaptable technologies, such as uAbs and the related PROTACs technology, that offer temporal and post-translational control over protein silencing are desirable especially because of their potential to overcome some of the limitations associated with nucleic acid targeting-based approaches such as irreversibility, lack of temporal control, and off-target effects. Deleavey et al., “Designing Chemically Modified Oligonucleotides for Targeted Gene Silencing,” Chemistry & Biology 19(8):937-54 (2012); Gaj et al., “TALEN, and CRISPR/Cas-Based Methods for Genome Engineering,” Trends Biotechnol. 31(7):397-405 (2013); Fu et al., “High-Frequency Off-Target Mutagenesis Induced by CRISPR-Cas Nucleases in Human Cells,” Nat. Biotechnol. 31(9):822-26 (2013); and Fedorov et al., “Off-Target Effects by siRNA Can Induce Toxic Phenotype,” RNA 12(7):1188-96 (2006), which are hereby incorporated by reference in its entirety. In principle, both uAbs and PROTACs can degrade proteins regardless of their function, including the currently undruggable proteome. Moreover, unlike conventional ‘occupancy-based’ therapeutics, uAbs and PROTACs act catalytically, making them substantially more potent than the target-binding antibody mimetics and small molecule inhibitors, respectively, from which they are built.

A major advantage of uAbs is the ease with which they can be rapidly adapted to hit a variety of intracellular targets due to their recombinant, modular design, which capitalizes on a large, preexisting repertoire of synthetic binding proteins as well as systematic, genome-wide efforts to generate and validate protein binders de novo against the human proteome. Colwill et at., “A Roadmap to Generate Renewable Protein Binders to the Human Proteome,” Nat. Methods 8(7):551-58 (2011), which is hereby incorporated by reference in its entirety. Because obtaining antibody mimetics that bind with high specificity and affinity to a target should be easier than obtaining small molecules with the same properties, making custom-designed PROTACs is likely to be a much more challenging task. Osherovich, L., “Degradation From Within,” Science-Business Exchange 7:10-11 (2014), which is hereby incorporated by reference in its entirety. Nonetheless, PROTACs holds great promise as a therapeutic approach because it is based on small molecules that have strong odds of getting into cells. Indeed, impressive preclinical in vitro and in vivo data are propelling the development of clinically viable PROTACs as evidenced by the founding of Arvinas in 2013 and C4 Therapeutics in 2016. It should be pointed out, however, that traditional medicinal chemistry approaches will be needed to improve the oral bioavailability, pharmacokinetics, and absorption, distribution, metabolism, excretion and toxicity (ADMET) properties of PROTACs. Neklesa et al., “Targeted Protein Degradation by PROTACs,” Pharmacol. Ther. 17:4138-144 (2017) and Deshaies, R. J., “Protein Degradation: Prime Time for PROTACs,” Nat. Chem. Biol. 11(9):634-35 (2015), which are hereby incorporated by reference in their entirety. Compared to PROTACs, intracellular delivery of uAb-based therapeutics is a much bigger hurdle as most globular protein drugs do not spontaneously cross plasma membranes due to their relatively large size and biochemical properties. Osherovich, L., “Degradation From Within,” Science-Business Exchange 7:10-11 (2014), which is hereby incorporated by reference in its entirety. One possible solution that is investigated here is the use of mRNA as a source of therapeutic gene product in vivo. In recent years, impediments to the use of mRNA, including its instability and immunogenicity, have been largely overcome through structural modifications, while issues related to delivery and protein expression profiles have been addressed through advances in nanotechnology and material science. Guan et al., “Nanotechnologies in Delivery of mRNA Therapeutics Using Nonviral Vector-Based Delivery Systems,” Gene Ther. 24(3):133-43 (2017), which is hereby incorporated by reference in its entirety. Here, this unique approach to create a first-in-kind therapeutic uAb delivery strategy is taken advantage of; this method involved a recently demonstrated strategy of electrostatics to stabilize pre-formed protein-RNA complexes for delivery. Li et al., “Polyamine-Mediated Stoichiometric Assembly of Ribonucleoproteins for Enhanced mRNA Delivery,” Angew Chem. Int. Ed. Engl. 56(44):13709-12 (2017), which is hereby incorporated by reference in its entirety. Here, synthetic mRNA encoding the GFP-directed GS2-IpaH9.8 chimera was co-assembled with PABPs and the assembled ribonucleoproteins were packaged into nanosized complexes using structurally defined polypeptides bearing cationic aminated side groups. The resulting nanoplexes achieved highly efficient silencing of GFP in vitro and in vivo, thereby demonstrating a new proteome editing paradigm and opening the door to clinical translation of uAb-based therapeutics.

Although preferred embodiments have been depicted and described in detail herein, it will be apparent to those skilled in the relevant art that various modifications, additions, substitutions, and the like can be made without departing from the spirit of the application and these are therefore considered to be within the scope of the present application as defined in the claims which follow.

What is claimed:

1. An isolated chimeric molecule comprising:
a degradation domain comprising an E3 ubiquitin ligase (E3) motif;
a targeting domain capable of specifically directing said degradation domain to a substrate, wherein said targeting domain is heterologous to said degradation domain; and
a linker coupling said degradation domain to said targeting domain.

2. The chimeric molecule of claim 1, wherein said E3 motif comprises a modified binding region which inhibits or decreases binding to said substrate compared to said E3 motif without the modified binding region.

3. The chimeric molecule of claim 2, wherein the modification is a mutation or deletion in said binding region.

4. The chimeric molecule of claim 1, wherein said E3 motif permits proteolysis of said substrate.

5. The chimeric molecule of claim 1, wherein said E3 motif possesses a cell-type specific or tissue specific ligase function.

6. The chimeric molecule of claim 5, wherein said ligase function is cell-type specific and the cell-type is selected from the group consisting of skin cells, muscle cells, epithelial cells, endothelial cells, stem cells, umbilical vessel cells, corneal cells, cardiomyocytes, aortic cells, corneal epithelial cells, somatic cells, fibroblasts, keratinocytes, melanocytes, adipose cells, bone cells, osteoblasts, airway cells, microvascular cells, mammary cells, vascular cells, chondrocytes, placental cells, hepatocytes, glial cells, epidermal cells, limbal stem cells, periodontal stem cells, bone marrow stromal cells, hybridoma cells, kidney cells, pancreatic islets, articular chondrocytes, neuroblasts, lymphocytes, and erythrocytes.

7. The chimeric molecule of claim 1, wherein said degradation domain is from a bacterial pathogen.

8. The chimeric molecule of claim 7, wherein said bacterial pathogen is selected from the group consisting of Shigella, Salmonella, Bacillus, Bartonella, Bordetella, Borrelia, Brucella, Campylobacter, Chlamydia and Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Staphylococcus, Streptococcus, Treponema, Ureaplasma, Vibrio, and Yersinia.

9. The chimeric molecule of claim 7, wherein said degradation domain is from a bacterial pathogen and comprises Shigella flexneri E3 ligase, SspH1, SspH2, SlrP, AvrPtoB, LubX, NleG5-1, NleG2-3, LegU1, LegAU13, NIeL, SopA, SidC, XopL, GobX, VirF, GALA, AnkB, or SidE.

10. The chimeric molecule of claim 1, wherein said degradation domain is a Shigella IpaH protein.

11. The chimeric molecule of claim 10, wherein said Shigella IpaH protein is selected from the group consisting of IpaH9.8, IpaH1.4, IpaH2.5, IpaH4.5, IpaH7.8, IpaH0887, IpaH1389, IpaH2022, IpaH2202, IpaH2610, and IpaH0722.

12. The chimeric molecule of claim 7, wherein when said bacterial pathogen is Shigella flexneri.

13. The chimeric molecule of claim 1, wherein said targeting domain is a monobody, fibronectin type III domain (FN3), antibody, polyclonal antibody, monoclonal antibody, recombinant antibody, antibody fragment, Fab′, F(ab′)2, Fv, scFv, tascFvs, bis-scFvs, sdAb, VH, VL, Vnar, scFvD10, scFv13R4, scFvD10, humanized antibody, chimeric antibody, complementary determining region (CDR), IgA antibody, IgD antibody, IgE antibody, IgG antibody, IgM antibody, nanobody, intrabody, unibody, minibody, non-antibody protein scaffold, Adnectin, Affibody and their two-helix variants, Anticalin, camelid antibody, V_{H}H, knottin, DARPin, or Sso7d.

14. The chimeric molecule of claim 13, wherein said targeting domain is a monobody, said monobody being a fibronectin type III domain (FN3) monobody selected from the group consisting of (with target antigen in parenthesis): GS2 (GFP), Nsa5 (SHP2), RasInI (HRas/KRas), and RasInII (HRas/KRas), 1D10 (CDC34), 1D7 (COPS5), 1C4 (MAP2K5), 2C12 (MAP2K5), 1E2 (SF3A1), 1C2 (USP11), 1A9 (USP11), Ubi4 (ubiquitin), EI1.4.1 (EGFR), EI2.4.6 (EGFR), EI3.4.3 (EGFR), EI4.2.1 (EGFR), EI4.4.2 (EGFR), EI6.2.6 (EGFR), EI6.2.10 (EGFR), E246(EGFR), C743(CEA), IIIa8.2.6 (FcγIIa), IIIa6.2.6 (FcγIIIa), hA2.2.1 (hA33), hA2.2.2 (hA33), hA3.2.1 (hA33), hA3.2.3 (hA33), mA3.2.1 (mA33), mA3.2.2 (mA33), mA3.2.3 (mA33), mA3.2.4 (mA33), mA3.2.5 (mA33), Alb3.2.1 (hAlb), mI2.2.1 (mIgG), HA4 (AblSH2), HA10 (AblSH2), HA16 (AblSH2), HA18 (AblSH2), 159 (vEGFR), MUC16 (MSLN), E2#3 (ERα/EF), E2#4 (ERα/EF), E2#5 (ERα/EF), E2#6 (ERα/EF), E2#7 (ERα/EF), E2#8 (ERα/EF), E2#9 (ERα/EF), E2#10 (ERα/EF), E2#11 (ERα/EF), E2#23 (ERα/EF), E3#2 (ERα/EF), E3#6 (ERα/EF), OHT#31 (ERα/EF), OHT#32 (ERα/EF), OHT#33 (ERα/EF), AB7-A1 (ERα/EF), AB7-B1 (ERα/EF), MBP-74 (MBP), MBP-76 (MBP), MBP-79 (MBP), hSUMO4-33 (hSUMO4), hSUMO-39 (hSUMO4), ySUMO-53 (ySUMO), ySUMO-56 (ySUMO), ySUMO-57 (ySUMO), T14.25 (TNFα), T14.20 (TNFα), FNfn10-3JCL14 (avβ3 integrin), 1C9 (Src SH3), 1F11 (Src SH3), 1F10 (Src SH3), 2G10 (Src SH3), 2B2 (Src SH3), 1E3 (Src SH3), E18 (VEGFR2), E19 (VEGFR2), E26 (VEGFR2), E29 (VEGFR2), FG4.2 (Lysozyme), FG4.1 (Lysozyme), 2L4.1 (Lysozyme), BF4.1 (Lysozyme), BF4.9 (Lysozyme), BF4.4 (Lysozyme), BFs1c4.01 (Lysozyme), BFs1c4.07 (Lysozyme), BFs3_4.02 (Lysozyme), BFs3_4.06 (Lysozyme), BFs3_8.01 (Lysozyme), 10C17C25 (phospho-IκBα), Fn-N22 (SARS N), Fn-N17 (SARS N), FN-N10 (SARS N), gI2.5.3T88I (goat IgG), gI2.5.2 (goat IgG), gI2.5.4 (goat IgG), rI4.5.4 (rabbit IgG), rI4.3.1 (rabbit IgG), rI3.6.6 (rabbit IgG), rI4.3.4 (rabbit IgG), rI3.6.4 (rabbit IgG), and rI4.3.3 (rabbit IgG).

15. The chimeric molecule of claim 1, wherein said substrate is selected from the group consisting of fluorescent protein, histone protein, nuclear localization signal (NLS), H-Ras protein, Src-homology 2 domain-containing phosphatase 2 (SHP2), β-galactosidase, gpD, Hsp70, MBP, CDC34, COPS5, MAP2K5, SF3A1, USP11, ubiquitin, EGFR, CEA, FcγIIa, FcγIIIa hA33, mA33, hAlb, mIgG, AblSH2, vEGFR, MSLN, ERα/EF, hSUMO4, ySUMO, TNFα, avβ3 integrin, Src SH3, Lysozyme, phospho-IκBα, SARS N, goat IgG, rabbit IgG, post-translationally modified proteins, fibrillin, huntingtin, tumorigenic proteins, p53, Rb, adhesion proteins, receptors, cell-cycle proteins, checkpoint proteins, HFE, ATP7B, prion proteins, viral proteins, bacterial proteins, parasitic proteins, fungal proteins, DNA binding proteins, metabolic proteins, regulatory proteins, structural proteins, enzymes, immunogenic proteins, autoimmunogenic proteins, immunogens, antigens, and pathogenic proteins.

16. The chimeric molecule of claim 1, wherein the substrate is a fluorescent protein selected from the group consisting of green fluorescent protein, emerald fluorescent protein, venus fluorescent protein, cerulean fluorescent protein, and enhanced cyan fluorescent protein.

17. The chimeric molecule of claim 1, wherein said targeting domain is binds to a non-native substrate.

18. A method of forming a ribonucleoprotein comprising:
providing a mRNA encoding the isolated chimeric molecule of claim 1;
providing one or more polyadenosine binding proteins (“PABP”); and
assembling a ribonucleoprotein complex from the mRNA and the one or more PABPs.

19. The method of claim 18, wherein said mRNA comprises a 3′-terminal polyadenosine (poly A) tail.

20. A composition comprising:
the chimeric molecule of claim 1; and
a pharmaceutically-acceptable carrier.

21. The composition of claim 20 further comprising:
a second agent selected from the group consisting of an anti-inflammatory agent, an antidiabetic agent, a hypolipidemic agent, a chemotherapeutic agent, an antiviral agent, an antibiotic, a metabolic agent, a small molecule inhibitor, a protein kinase inhibitor, adjuvants, apoptotic agents, a proliferative agent, and organotropic targeting agents, and any combination thereof.

22. A method of treating a disease comprising:
selecting a subject having a disease and
administering the composition of claim 20 to the subject to give the subject an increased expression level of said substrate compared to a subject not afflicted with said disease.

23. The method of claim 22, wherein said disease is selected from the group consisting of cancer, metastatic cancer, stroke, ischemia, peripheral vascular disease, alcoholic liver disease, hepatitis, cirrhosis, Parkinson's disease, Alzheimer's disease, cystic fibrosis diabetes, ALS, pathogenic diseases, idiopathic diseases, viral diseases, bacterial, diseases, prionic diseases, fungal diseases, parasitic diseases, arthritis, wound healing, immunodeficiency, inflammatory disease, aplastic anemia, anemia, genetic disorders, congenital disorders, type 1 diabetes, type 2 diabetes, gestational diabetes, high blood glucose, metabolic syndrome, lipodystrophy syndrome, dyslipidemia, insulin resistance, leptin resistance, atherosclerosis, vascular disease, hypercholesterolemia, hypertriglyceridemia, non-alcoholic fatty liver disease, overweight, and obesity.

24. The method of claim 22, wherein the administering is carried out orally, parenterally, subcutaneously, intravenously, intramuscularly, intraperitoneally, by intranasal instillation, by implantation, by intracavitary or intravesical instillation, intraocularly, intraarterially, intralesionally, transdermally, or by application to mucous membranes.

25. A method for substrate silencing, the method comprising:
selecting a substrate to be silenced;
providing said chimeric molecule of claim 1; and
contacting said substrate with said chimeric molecule under conditions effective to permit the formation of a substrate-molecule complex, wherein said complex mediates the degradation of said substrate to be silenced.

26. The method of claim 25, wherein said substrate is selected from the group consisting of fluorescent protein, histone protein, nuclear localization signal (NLS), H-Ras protein, SHP2 protein, Src-homology 2 domain-containing phosphatase 2 (SHP2), (3-galactosidase, gpD, Hsp70, MBP, CDC34, COPS5, MAP2K5, SF3A1, USP11, ubiquitin, EGFR, CEA, FcγIIa, FcγIIIa, hA33, mA33, hAlb, mIgG, AblSH2, vEGFR, MSLN, ERα/EF, hSUMO4, ySUMO, TNFα, avβ3 integrin, Src SH3, Lysozyme, phospho-IκBα, SARS N, goat IgG, rabbit IgG, post-translationally modified proteins, fibrillin, huntingtin, tumorigenic proteins, p53, Rb, adhesion proteins, receptors, cell-cycle proteins, checkpoint proteins, HFE, ATP7B, prion proteins, viral proteins, bacterial proteins, parasitic proteins, fungal proteins, DNA binding proteins, metabolic proteins, regulatory proteins, structural proteins, enzymes, immunogenic proteins, autoimmunogenic proteins, immunogens, antigens, and pathogenic proteins.

27. The method of claim 25, wherein the substrate is a fluorescent protein selected from the group consisting of green fluorescent protein, emerald fluorescent protein, venus fluorescent protein, cerulean fluorescent protein, and enhanced cyan fluorescent protein.

28. A method of screening agents for therapeutic efficacy against a disease, said method comprising:
providing a biomolecule whose presence mediates a disease state;
providing a test agent comprising (i) a degradation domain comprising an E3 ubiquitin ligase (E3) motif, (ii) a targeting domain capable of specifically directing said degradation domain to said biomolecule, wherein said targeting domain is heterologous to said degradation domain, and (iii) a linker coupling said degradation domain to said targeting domain;
contacting said biomolecule with said test agent under conditions effective for the test agent to facilitate degradation of the biomolecule;
determining the level of said biomolecule as a result of said contacting; and
identifying said test agent which, based on said determining, decreases the level of said biomolecule as being a candidate for therapeutic efficacy against said disease.

29. The method of claim 28, wherein said identifying is carried out with respect to a standard biomolecule level in a subject not afflicted with said disease.

30. The method of claim 28, wherein said identifying is carried out with respect to the biomolecule level absent said contacting.

31. The method of claim 28, wherein the method is carried out with a plurality of test agents.

32. The method of claim 28, wherein said degradation domain is a bacterial pathogen.

33. The method of claim 32, wherein said bacterial pathogen is selected from the group consisting of Shigella, Salmonella, Bacillus, Bartonella, Bordetella, Borrelia, Brucella, Campylobacter, Chlamydia and Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Staphylococcus, Streptococcus, Treponema, Ureaplasma, Vibrio, and Yersinia.

34. The method of claim 32, wherein said degradation domain is from a bacterial pathogen and comprises Shigella flexneri E3 ligase, SspH1, SspH2, SlrP, AvrPtoB, LubX, NleG5-1, NleG2-3, LegU1, LegAU13, NIeL, SopA, SidC, XopL, GobX, VirF, GALA, AnkB, or SidE.

35. The method of claim 28, wherein said degradation domain is a Shigella IpaH protein.

36. The method of claim 35, wherein said Shigella IpaH protein is selected from the group consisting of IpaH9.8, IpaH1.4, IpaH2.5, IpaH4.5, IpaH7.8, IpaH0887, IpaH1389, IpaH2022, IpaH2202, IpaH2610, and IpaH0722.

37. The method of claim 32, wherein when said bacterial pathogen is Shigella flexneri.

38. The method of claim 28, wherein said targeting domain is a monobody, fibronectin type III domain (FN3), antibody, polyclonal antibody, monoclonal antibody, recombinant antibody, antibody fragment, Fab′, F(ab′)2, Fv, scFv, tascFvs, bis-scFvs, sdAb, VH, VL, Vnar, scFvD10, scFv13R4, scFvD10, humanized antibody, chimeric antibody, complementary determining region (CDR), IgA antibody, IgD antibody, IgE antibody, IgG antibody, IgM antibody, nanobody, intrabody, unibody, minibody, non-antibody protein scaffold, Adnectin, Affibody and their two-helix variants, Anticalin, camelid antibody, V_{H}H, knottin, DARPin, or Sso7d.

39. The method of claim 38, wherein said targeting domain is a monobody, said monobody being a fibronectin type III domain (FN3) monobody selected from the group consisting of (with target antigen in parenthesis): GS2 (GFP), Nsa5 (SHP2), RasInI (HRas/KRas), and RasInII (HRas/KRas), 1D10 (CDC34), 1D7 (COPS5), 1C4 (MAP2K5), 2C12 (MAP2K5), 1E2 (SF3A1), 1C2 (USP11), 1A9 (USP11), Ubi4 (ubiquitin), EI1.4.1 (EGFR), EI2.4.6 (EGFR), EI3.4.3 (EGFR), EI4.2.1 (EGFR), EI4.4.2 (EGFR), EI6.2.6 (EGFR), EI6.2.10 (EGFR), E246(EGFR), C743(CEA), IIIa8.2.6 (FcγIIa), IIIa6.2.6 (FcγIIIa), hA2.2.1 (hA33), hA2.2.2 (hA33), hA3.2.1 (hA33), hA3.2.3 (hA33), mA3.2.1 (mA33), mA3.2.2 (mA33), mA3.2.3 (mA33), mA3.2.4 (mA33), mA3.2.5 (mA33), Alb3.2.1 (hAlb), mI2.2.1 (mIgG), HA4 (AblSH2), HA10 (AblSH2), HA16 (AblSH2), HA18 (AblSH2), 159 (vEGFR), MUC16 (MSLN), E2#3 (ERα/EF), E2#4 (ERα/EF), E2#5 (ERα/EF), E2#6 (ERα/EF), E2#7 (ERα/EF), E2#8 (ERα/EF), E2#9 (ERα/EF), E2#10 (ERα/EF), E2#11 (ERα/EF), E2#23 (ERα/EF), E3#2 (ERα/EF), E3#6 (ERα/EF), OHT#31 (ERα/EF), OHT#32 (ERα/EF), OHT#33 (ERα/EF), AB7-A1 (ERα/EF), AB7-B1 (ERα/EF), MBP-74 (MBP), MBP-76 (MBP), MBP-79 (MBP), hSUMO4-33 (hSUMO4), hSUMO-39 (hSUMO4), ySUMO-53 (ySUMO), ySUMO-56 (ySUMO), ySUMO-57 (ySUMO), T14.25 (TNFα), T14.20 (TNFα), FNfn10-3JCL14 (avβ3 integrin), 1C9 (Src SH3), 1F11 (Src SH3), 1F10 (Src SH3), 2G10 (Src SH3), 2B2 (Src SH3), 1E3 (Src SH3), E18 (VEGFR2), E19 (VEGFR2), E26 (VEGFR2), E29 (VEGFR2), FG4.2 (Lysozyme), FG4.1 (Lysozyme), 2L4.1 (Lysozyme), BF4.1 (Lysozyme), BF4.9 (Lysozyme), BF4.4 (Lysozyme), BFs1c4.01 (Lysozyme), BFs1c4.07 (Lysozyme), BFs3_4.02 (Lysozyme), BFs3_4.06 (Lysozyme), BFs3_8.01 (Lysozyme), 10C17C25 (phospho-IκBα), Fn-N22 (SARS N), Fn-N17 (SARS N), FN-N10 (SARS N), gI2.5.3T88I (goat IgG), gI2.5.2 (goat IgG), gI2.5.4 (goat IgG), rI4.5.4 (rabbit IgG), rI4.3.1 (rabbit IgG), rI3.6.6 (rabbit IgG), rI4.3.4 (rabbit IgG), rI3.6.4 (rabbit IgG), and rI4.3.3 (rabbit IgG).

40. The method of claim 28, wherein said substrate is selected from the group consisting of fluorescent protein, histone protein, nuclear localization signal (NLS), H-Ras protein, SHP2 protein, Src-homology 2 domain-containing phosphatase 2 (SHP2), β-galactosidase, gpD, Hsp70, MBP, CDC34, COPS5, MAP2K5, SF3A1, USP11, ubiquitin, EGFR, CEA, FcγIIa, FcγIIIa, hA33, mA33, hAlb, mIgG, AblSH2, vEGFR, MSLN, ERα/EF, hSUMO4, ySUMO, TNFα, avβ3 integrin, Src SH3, Lysozyme, phospho-IκBα, SARS N, goat IgG, rabbit IgG, post-translationally modified proteins, fibrillin, huntingtin, tumorigenic proteins, p53, Rb, adhesion proteins, receptors, cell-cycle proteins, checkpoint proteins, HFE, ATP7B, prion proteins, viral proteins, bacterial proteins, parasitic proteins, fungal proteins, DNA binding proteins, metabolic proteins, regulatory proteins, structural proteins, enzymes, immunogenic proteins, autoimmunogenic proteins, immunogens, antigens, and pathogenic proteins.

41. The method of claim 28, wherein the substrate is a fluorescent protein selected from the group consisting of green fluorescent protein, emerald fluorescent protein, venus fluorescent protein, cerulean fluorescent protein, and enhanced cyan fluorescent protein.

42. The method of claim 28, wherein said linker is a polypeptide linker of sufficient length to prevent the steric disruption of binding between said targeting domain and said biomolecule.

43. The method of claim 28, wherein said biomolecule is associated with cancer, metastatic cancer, stroke, ischemia, peripheral vascular disease, alcoholic liver disease, hepatitis, cirrhosis, Parkinson's disease, Alzheimer's disease, cystic fibrosis diabetes, ALS, pathogenic diseases, idiopathic diseases, viral diseases, bacterial, diseases, prionic diseases, fungal diseases, parasitic diseases, arthritis, wound healing, immunodeficiency, inflammatory disease, aplastic anemia, anemia, genetic disorders, congenital disorders, type 1 diabetes, type 2 diabetes, gestational diabetes, high blood glucose, metabolic syndrome, lipodystrophy syndrome, dyslipidemia, insulin resistance, leptin resistance, atherosclerosis, vascular disease, hypercholesterolemia, hypertriglyceridemia, non-alcoholic fatty liver disease, overweight, or obesity, and any combination thereof.

44. A method of screening for disease biomarkers, said method comprising:
providing a sample of diseased cells expressing one or more ligands;
providing a plurality of chimeric molecules comprising (i) a degradation domain comprising an E3 ubiquitin ligase (E3) motif, (ii) a targeting domain capable of specifically directing said degradation domain to said one or more ligands, wherein said targeting domain is heterologous to said degradation domain, and (iii) a linker coupling said degradation domain to said targeting domain;
contacting said sample with said plurality of chimeric molecules under conditions effective for the diseased cells to fail to proliferate in the absence of the chimeric molecule;
determining which of said chimeric molecules permit the diseased cells to proliferate; and
identifying, as biomarkers for the disease, based on said determining the ligands which bind to the chimeric molecules and permit diseased cells to proliferate.

45. The method of claim 44, wherein said disease is selected from the group consisting of cancer, metastatic cancer, stroke, ischemia, peripheral vascular disease, alcoholic liver disease, hepatitis, cirrhosis, Parkinson's disease, Alzheimer's disease, cystic fibrosis diabetes, ALS, pathogenic diseases, idiopathic diseases, viral diseases, bacterial, diseases, prionic diseases, fungal diseases, parasitic diseases, arthritis, wound healing, immunodeficiency, inflammatory disease, aplastic anemia, anemia, genetic disorders, congenital disorders, type 1 diabetes, type 2 diabetes, gestational diabetes, high blood glucose, metabolic syndrome, lipodystrophy syndrome, dyslipidemia, insulin resistance, leptin resistance, atherosclerosis, vascular disease, hypercholesterolemia, hypertriglyceridemia, non-alcoholic fatty liver disease, overweight, and obesity.

46. The method of claim 44, wherein said degradation domain is a bacterial pathogen.

47. The method of claim 46, wherein said bacterial pathogen is selected from the group consisting of Shigella, Salmonella, Bacillus, Bartonella, Bordetella, Borrelia, Brucella, Campylobacter, Chlamydia and Chlamydophila, Clostridium, Corynebacterium, Enterococcus, Escherichia, Francisella, Haemophilus, Helicobacter, Legionella, Leptospira, Listeria, Mycobacterium, Mycoplasma, Neisseria, Pseudomonas, Rickettsia, Staphylococcus, Streptococcus, Treponema, Ureaplasma, Vibrio, and Yersinia.

48. The method of claim 46, wherein said degradation domain is from a bacterial pathogen and comprises Shigella flexneri E3 ligase, SspH1, SspH2, SlrP, AvrPtoB, LubX, NleG5-1, NleG2-3, LegU1, LegAU13, NIeL, SopA, SidC, XopL, GobX, VirF, GALA, AnkB, or SidE.

49. The method of claim 44, wherein said degradation domain is a Shigella IpaH protein.

50. The method of claim 49, wherein said Shigella IpaH protein is selected from the group consisting of IpaH9.8, IpaH1.4, IpaH2.5, IpaH4.5, IpaH7.8, IpaH0887, IpaH1389, IpaH2022, IpaH2202, IpaH2610, and IpaH0722.

51. The method of claim 46, wherein when said bacterial pathogen is Shigella flexneri.

52. The method of claim 44, wherein said targeting domain is a monobody, fibronectin type III domain (FN3), antibody, polyclonal antibody, monoclonal antibody, recombinant antibody, antibody fragment, Fab′, F(ab′)2, Fv, scFv, tascFvs, bis-scFvs, sdAb, VH, VL, Vnar, scFvD10, scFv13R4, scFvD10, humanized antibody, chimeric antibody, complementary determining region (CDR), IgA antibody, IgD antibody, IgE antibody, IgG antibody, IgM antibody, nanobody, intrabody, unibody, minibody, non-antibody protein scaffold, Adnectin, Affibody and their two-helix variants, Anticalin, camelid antibody, V_{H}H, knottin, DARPin, or Sso7d.

53. The method of claim 50, wherein said targeting domain is a monobody, said monobody being a fibronectin type III domain (FN3) monobody selected from the group consisting of (with target antigen in parenthesis): GS2 (GFP), Nsa5 (SHP2), RasInI (HRas/KRas), and RasInII (HRas/KRas), 1D10 (CDC34), 1D7 (COPS5), 1C4 (MAP2K5), 2C12 (MAP2K5), 1E2 (SF3A1), 1C2 (USP11), 1A9 (USP11), Ubi4 (ubiquitin), EI1.4.1 (EGFR), EI2.4.6 (EGFR), EI3.4.3 (EGFR), EI4.2.1 (EGFR), EI4.4.2 (EGFR), EI6.2.6 (EGFR), EI6.2.10 (EGFR), E246(EGFR), C743(CEA), IIIa8.2.6 (FcγIIa), IIIa6.2.6 (FcγIIIa), hA2.2.1 (hA33), hA2.2.2 (hA33), hA3.2.1 (hA33), hA3.2.3 (hA33), mA3.2.1 (mA33), mA3.2.2 (mA33), mA3.2.3 (mA33), mA3.2.4 (mA33), mA3.2.5 (mA33), Alb3.2.1 (hAlb), mI2.2.1 (mIgG), HA4 (AblSH2), HA10 (AblSH2), HA16 (AblSH2), HA18 (AblSH2), 159 (vEGFR), MUC16 (MSLN), E2#3 (ERα/EF), E2#4 (ERα/EF), E2#5 (ERα/EF), E2#6 (ERα/EF), E2#7 (ERα/EF), E2#8 (ERα/EF), E2#9 (ERα/EF), E2#10 (ERα/EF), E2#11 (ERα/EF), E2#23 (ERα/EF), E3#2 (ERα/EF), E3#6 (ERα/EF), OHT#31 (ERα/EF), OHT#32 (ERα/EF), OHT#33 (ERα/EF), AB7-A1 (ERα/EF), AB7-B1 (ERα/EF), MBP-74 (MBP), MBP-76 (MBP), MBP-79 (MBP), hSUMO4-33 (hSUMO4), hSUMO-39 (hSUMO4), ySUMO-53 (ySUMO), ySUMO-56 (ySUMO), ySUMO-57 (ySUMO), T14.25 (INFO, T14.20 (TNFα), FNfn10-3JCL14 (avβ3 integrin), 1C9 (Src SH3), 1F11 (Src SH3), 1F10 (Src SH3), 2G10 (Src SH3), 2B2 (Src SH3), 1E3 (Src SH3), E18 (VEGFR2), E19 (VEGFR2), E26 (VEGFR2), E29 (VEGFR2), FG4.2 (Lysozyme), FG4.1 (Lysozyme), 2L4.1 (Lysozyme), BF4.1 (Lysozyme), BF4.9 (Lysozyme), BF4.4 (Lysozyme), BFs1c4.01 (Lysozyme), BFs1c4.07 (Lysozyme), BFs3_4.02 (Lysozyme), BFs3_4.06 (Lysozyme), BFs3_8.01 (Lysozyme), 10C17C25 (phospho-IκBα), Fn-N22 (SARS N), Fn-N17 (SARS N), FN-N10 (SARS N), gI2.5.3T88I (goat IgG), gI2.5.2 (goat IgG), gI2.5.4 (goat IgG), rI4.5.4 (rabbit IgG), rI4.3.1 (rabbit IgG), rI3.6.6 (rabbit IgG), rI4.3.4 (rabbit IgG), rI3.6.4 (rabbit IgG), and rI4.3.3 (rabbit IgG).
